(19) United States
(12) Patent Application Publication
(10) Pub. No.: US 2022/0202780 A1
(43) Pub. Date: Jun. 30, 2022

(54) PHARMACEUTICAL COMPOSITIONS AND COMBINATIONS COMPRISING INHIBITORS OF THE ANDROGEN RECEPTOR AND USES THEREOF

(71) Applicant: ESSA Pharma, Inc., Vancouver (CA)

(72) Inventors: Peter Virsik, Portola Valley, CA (US); Han-Jie Zhou, Foster City, CA (US); Ronan Le Moigne, South San Francisco, CA (US)

(21) Appl. No.: 17/599,338

(22) PCT Filed: Mar. 27, 2020
(86) PCT No.: PCT/US20/25539

§ 371 Date: Sep. 28, 2021

Related U.S. Application Data
(60) Provisional application No. 62/825,450, filed on Mar. 28, 2019.
(60) Provisional application No. 62/842,980, filed on May 3, 2019.
(60) Provisional application No. 62/857,519, filed on Jun. 5, 2019.

(51) Int. Cl. A61K 31/4164; A61K 31/4166; A61K 31/4439; A61K 31/4155; A61K 31/277; A61K 31/167; A61K 31/635; A61P 35/00; A61K 31/421; A61K 31/4245; A61K 31/42; A61K 31/415; A61K 31/505

(52) U.S. Cl. CPC A61K 31/4164; A61K 31/4166; A61K 31/4439; A61K 31/4155; A61K 31/277; A61K 31/167; A61K 31/505; A61P 35/00; A61K 31/421; A61K 31/4245; A61K 31/42; A61K 31/415; A61K 31/635

(57) ABSTRACT

The present disclosure generally relates to pharmaceutical compositions and combinations comprising an androgen receptor modulator or an inhibitor and a second therapeutically active agent, such as an antiandrogen. In particular, the present disclosure relates to pharmaceutical compositions and combinations useful for treatment of various cancers, for example breast cancer and prostate cancer.

### CROSS-REFERENCE TO RELATED APPLICATION

U.S. Provisional Application No. 62/825,450, filed Mar. 28, 2019, U.S. Provisional Application No. 62/842,980, filed May 3, 2019, and U.S. Provisional Application No. 62/857,519, filed Jun. 5, 2019, the disclosures of which are hereby incorporated by reference in their entireties for all purposes.

### FIELD OF THE INVENTION

The present disclosure generally relates to pharmaceutical compositions and combinations comprising an androgen receptor (AR) modulator (including an AR inhibitor such as N-terminal domain inhibitor) and an additional therapeutic agent, such as an antiandrogen. In particular, the present disclosure relates to pharmaceutical compositions and combinations useful for treatment of various cancers, for example breast cancer and prostate cancer.

### BACKGROUND OF THE INVENTION

Androgens mediate their effects through the androgen receptor (AR). Androgens play a role in a wide range of developmental and physiological responses and are involved in male sexual differentiation, maintenance of spermatogenesis, and male gonadotropin regulation (R. K. Ross, G. A. Coetzee, C. L. Pearce, J. K. Reichardt, P. Bretsky, L. N. Kolonel, B. E. Henderson, E. Lander, D. Altshuler & G. Daley, Eur Urol 35, 355-361 (1999); A. A. Thomson, Reproduction 121, 187-195 (2001); N. Tanji, K. Aoki & M. Yokoyama, Arch Androl 47, 1-7 (2001)). Several lines of evidence show that androgens are associated with the development of prostate carcinogenesis. Firstly, androgens induce prostatic carcinogenesis in rodent models (R. L. Noble, Cancer Res 37, 1929-1933 (1977); R. L. Noble, Oncology 34, 138-141 (1977)) and men receiving androgens in the form of anabolic steroids have a higher incidence of prostate cancer (J. T. Roberts & D. M. Essenhigh, Lancet 2, 742 (1986); J. A. Jackson, J. Waxman & A. M. Spiekerman, Arch Intern Med 149, 2365-2366 (1989); P. D. Guinan, W. Sadoughi, H. Alsheik, R. J. Ablin, D. Alrenga & I. M. Bush, Am J Surg 131, 599-600 (1976)). Secondly, prostate cancer does not develop if humans or dogs are castrated before puberty (J. D. Wilson & C. Roehrborn, J Clin Endocrinol Metab 84, 4324-4331 (1999); G. Wilding, Cancer Surv 14, 113-130 (1992)). Castration of adult males causes involution of the prostate and apoptosis of prostatic epithelium while eliciting no effect on other male external genitalia (E. M. Bruckheimer & N. Kyprianou, Cell Tissue Res 301, 153-162 (2000); J. T. Isaacs, Prostate 5, 545-557 (1984)). This dependency on androgens provides the underlying rationale for treating prostate cancer with chemical or surgical castration also known as androgen ablation therapy (ABT) or androgen deprivation therapy (ADT).

Androgen receptor (AR) is a transcription factor that plays dual roles in breast cancer cells: promoting or inhibiting proliferation depending on expression and activity of estrogen receptor-alpha. Expression of AR is detected in up to 90% of all breast cancers.

Androgens also play a role in female diseases such as polycystic ovary syndrome as well as cancers. One example is ovarian cancer where elevated levels of androgens are associated with an increased risk of developing ovarian cancer (K. J. Helzlsouer, A. J. Alberg, G. B. Gordon, C. Longcope, T. L. Bush, S. C. Hoffman & G. W. Comstock, JAMA 274, 1926-1930 (1995); R. J. Edmondson, J. M. Monaghan & B. R. Davies, Br J Cancer 86, 879-885 (2002)). The AR has been detected in a majority of ovarian cancers (H. A. Risch, J Natl Cancer Inst 90, 1774-1786 (1998); B. R. Rao & B. J. Slotman, Endocr Rev 12, 14-26 (1991); G. M. Clinton & W. Hua, Crit Rev Oncol Hematol 25, 1-9 (1997)), whereas estrogen receptor-alpha (ERa) and the progesterone receptor are detected in less than 50% of ovarian tumors.

The only effective treatment available for advanced prostate cancer is the withdrawal of androgens which are essential for the survival of prostate luminal cells. Androgen ablation therapy causes a temporary reduction in tumor burden concomitant with a decrease in serum prostate-specific antigen (PSA). Unfortunately prostate cancer can eventually grow again in the absence of testicular androgens (castration-resistant disease) (Huber et al 1987 Scand J. Urol Nephrol. 104, 33-39). Castration-resistant prostate cancer that is still driven by AR is biochemically characterized before the onset of symptoms by a rising titre of serum PSA (Miller et al 1992 J. Urol. 147, 956-961). Once the disease becomes castration-resistant most patients succumb to their disease within two years.

The AR has distinct functional domains that include the carboxy-terminal ligand-binding domain (LBD), a DNA-binding domain (DBD) comprising two zinc finger motifs, and an N-terminus domain (NTD) that contains two transcriptional activation units (tau1 and tau5) within activation function-1 (AF-1). Binding of androgen (ligand) to the LBD of the AR results in its activation such that the receptor can effectively bind to its specific DNA consensus site, termed the androgen response element (ARE), on the promoter and enhancer regions of “normally” androgen regulated genes, such as PSA, to initiate transcription. The AR can be activated in the absence of androgen by stimulation of the cAMP-dependent protein kinase (PKA) pathway, with interleukin-6 (IL-6) and by various growth factors (Culig et al 1994 Cancer Res. 54, 5474-5478; Nazareth et al 1996 J. Biol. Chem. 271, 19900-19907; Sadar 1999 J Biol. Chem. 274, 7777-7783; Ueda et al 2002 A J Biol. Chem. 277, 7076-7085; and Ueda et al 2002 B J Biol. Chem. 277, 38087-38094). The mechanism of ligand-independent transformation of the AR has been shown to involve: 1) increased nuclear AR protein suggesting nuclear translocation; 2) increased AR/ARE complex formation; and 3) the AR-NTD (Sadar 1999 J Biol. Chem. 274, 7777-7783; Ueda et al 2002 A J Biol. Chem. 277, 7076-7085; and Ueda et al 2002 B J Biol. Chem. 277, 38087-38094). The AR can be activated in the absence of testicular androgens by alternative signal transduction pathways in castration-resistant disease, which is consistent with the finding that nuclear AR protein is present in secondary prostate cancer tumors (Kim et al 2002 Am. J. Pathol. 160, 219-226; and van der Kwast et al 1991 Inter. J. Cancer 48, 189-193).

Clinically available inhibitors of the AR include nonsteroidal antiandrogens such as bicalutamide (Casodex™), nilutamide, flutamide, and enzalutamide. There is also a class of steroidal antiandrogens, such as cyproterone acetate and spironolactone. Both steroidal and non-steroidal antiandrogens target the LBD of the AR and predominantly fail presumably due to poor affinity and mutations that lead to activation of the AR by these same antiandrogens (Taplin, M. E., Bubley, G. J., Kom Y. J., Small E. J., Uptonm M., Rajeshkumarm B., Balkm S. P., Cancer Res., 59, 2511-2515 (1999)), and constitutively active AR splice variants. Antiandrogens have no effect on the constitutively active AR splice variants that lack the ligand-binding domain (LBD) and are associated with castration-recurrent prostate cancer (Dehm S M, Schmidt L J, Heemers H V, Vessella R L, Tindall D J., Cancer Res 68, 5469-77, 2008; Guo Z, Yang X, Sun F, Jiang R, Linn D E, Chen H, Chen H, Kong X, Melamed J, Tepper C G, Kung H J, Brodie A M, Edwards J, Qiu Y., Cancer Res. 69, 2305-13, 2009; Hu et al 2009 Cancer Res. 69, 16-22; Sun et al 2010 J Clin Invest. 2010 120, 2715-30) and resistant to abiraterone and enzalutamide (Antonarakis et al., N Engl J Med. 2014, 371, 1028-38; Scher et al JAMA Oncol. 2016 doi: 10.1001). Conventional therapy has concentrated on androgen-dependent activation of the AR through its C-terminal domain.

Other relevant AR antagonists previously reported (see, WO 2010/000066, WO 2011/082487; WO 2011/082488; WO 2012/145330; WO 2015/031984; WO 2016/058080; and WO 2016/058082) that bind to full-length AR and/or truncated AR splice variants that are currently being developed include: AR degraders such as niclosamide (Liu C et al 2014), galeterone (Njar et al 2015; Yu Z at al 2014), and ARV-330/Androgen receptor PROTAC (Neklesa et al 2016 J Clin Oncol 34 suppl 2S; abstr 267); AR DBD inhibitor VPC-14449 (Dalal K et al 2014 J Biol Chem. 289(38):26417-29; Li H et al 2014 J Med Chem. 57(15):6458-67); antiandrogens apalutamide (Clegg N J et al 2012), ODM-201 (Moilanen A M et al 2015), ODM-204 (Kallio et al J Clin Oncol 2016 vol. 34 no. 2_suppl 230), TAS3681 (Minamiguchi et al 2015 J Clin Oncol 33, suppl 7; abstr 266); and AR NTD inhibitors 3E10-AR441bsAb (Goicochea N L et al 2015), and sintokamide (Sadar et al 2008; Banuelos et al 2016).

The AR-NTD is also a target for drug development (e.g. WO 2000/001813; Myung et al. J. Clin. Invest 2013, 123, 2948), since the NTD contains Activation-Function-1 (AF-1) which is the essential region required for AR transcriptional activity (Jenster et al 1991. Mol Endocrinol. 5, 1396-404). The AR-NTD importantly plays a role in activation of the AR in the absence of androgens (Sadar, M. D. 1999 J Biol. Chem. 274, 7777-7783; Sadar M D et al 1999 Endocr Relat Cancer. 6, 487-502; Ueda et al 2002 J. Biol. Chem. 277, 7076-7085; Ueda 2002 J. Biol. Chem. 277, 38087-38094; Blaszczyk et al 2004 Clin Cancer Res. 10, 1860-9; Dehm et al 2006 J Biol Chem. 28, 27882-93; Gregory et al 2004 J Biol Chem. 279, 7119-30). The AR-NTD is important in hormonal progression of prostate cancer as shown by application of decoy molecules (Quayle et al 2007, Proc Natl Acad Sci USA. 104, 1331-1336).

While the crystal structure has been resolved for the AR C-terminus LBD, this has not been the case for the NTD due to its high flexibility and intrinsic disorder in solution (Reid et al 2002 J. Biol. Chem. 277, 20079-20086) thereby hampering virtual docking drug discovery approaches. Compounds that modulate AR, potentially through interaction with NTD domain, include the bisphenol compounds disclosed in published PCT Nos: WO 2010/000066, WO 2011/082487; WO 2011/082488; WO 2012/145330; WO 2012/139039; WO 2012/145328; WO 2013/028572; WO 2013/028791; WO 2014/179867; WO 2015/031984; WO 2016/058080; WO 2016/058082; WO 2016/112455; WO 2016/141458; WO 2017/177307; WO 2017/210771; and WO 2018/045450, and which are hereby incorporated by reference in their entireties.

Transcriptionally active androgen receptor plays a major role in CRPC in spite of reduced blood levels of androgen (Karantanos, T. et al Oncogene 2013, 32, 5501-5511; Harris, W. P. et al Nature Clinical Practice Urology, 2009, 6, 76-85). AR mechanisms of resistance to ADT include: overexpression of AR (Visakorpi, T. et al Nature Genetics 1995, 9, 401-406; Koivisto, P. et al Scandinavian Journal of Clinical and Laboratory Investigation Supplementum 1996, 226, 57-63); gain-of-function mutations in the AR LBD (Culig Z. et al Molecular Endocrinology 1993, 7, 1541-1550); intratumoral androgen synthesis (Cai, C. et al Cancer Research 2011, 71, 6503-6513); altered expression and function of AR coactivators (Ueda, T. et al The Journal of Biological Chemistry 2002, 277, 38087-38094; Xu J. et al Nature Reviews Cancer 2009, 9, 615-630); aberrant post-translational modifications of AR (Gioeli D. et al Molecular and Cellular Endocrinology 2012, 352, 70-78; van der Steen T. et al International Journal of Molecular Sciences 2013, 14, 14833-14859); and expression of AR splice variants (AR-Vs) which lack the ligand-binding domain (LBD) (Karantanos, T. et al Oncogene 2013, 32, 5501-5511; Andersen R. J. et al Cancer Cell 2010, 17, 535-546; Myung J. K. et al The Journal of Clinical Investigation 2013, 123, 2948-2960; Sun S. et al The Journal of Clinical Investigation 2010, 120, 2715-2730). Anti-androgens such as bicalutamide and enzalutamide target AR LBD, but have no effect on truncated constitutively active AR-Vs such as AR-V7 (Li Y. et al Cancer Research 2013, 73, 483-489). Expression of AR-V7 is associated with resistance to current hormone therapies (Li Y. et al Cancer Research 2013, 73, 483-489; Antonarakis E. S. et al The New England Journal of Medicine 2014, 371, 1028-1038).

While significant advances have been made in this field, there remains a need for improved treatment for AR-mediated disorders including breast cancer and prostate cancer. Currently there is no FDA-approved targeted therapy for triple-negative breast cancer (TNBC). AR plays a role in the proliferation of breast cancer cells by either promoting proliferation or inhibiting proliferation depending on the expression of estrogen receptor (ER) and human epidermal growth factor receptor 2 (HER2). AR expression is detected in up to 90% of all breast cancers and in up to approximately 35% of TNBC. AR-Vs have been detected in primary breast cancer specimens and in breast cancer cell lines. AR-V7 expression was detected in circulating-tumor cells of patients with metastatic breast cancer and was associated with bone metastases. Targeting AR is a potential therapeutic strategy for AR-positive TNBC.

### SUMMARY OF THE INVENTION

The present disclosure relates to pharmaceutical compositions and combinations comprising an androgen receptor modulator and a second therapeutically active agent. In one embodiment, the pharmaceutical composition comprises an androgen receptor N-terminal domain inhibitor and an androgen receptor ligand-binding domain inhibitor.

In one embodiment, the present disclosure provides a pharmaceutical composition wherein the androgen receptor modulator is a compound of formula (IIIA):

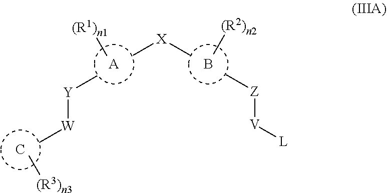

or a pharmaceutically acceptable salt, tautomer, stereoisomer or prodrug thereof, wherein:
A and B are each independently selected from phenyl, pyridyl, pyrimidyl, or thiophene;
C is a 3- to 10-membered ring;
X is a bond, —(CR⁵R⁶)ₜ—, or —NR⁷;
Y is a bond, —(CR⁸R⁹)ₘ—, —O—, —S—, —S(═O)—, —SO₂—, —NR⁷—, or —N(COCH₃)—;
W is a bond, —(CR^{8a}R^{9a})ₘ—, —C(═O)—, —N(R⁷)CO—, —CONR⁷—, or —NSO₂R⁷—;
Z is a bond, —CH₂—, —C(CH₃)H—, —O—, —S—, —NH—, —NCH₃—, or —N(COCH₃)—;
V is —CH₂— and L is halogen, —NH₂, —CHCl₂, —CCl₃, or —CF₃; or
V is —CH₂CH₂— and L is halogen or —NH₂;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;
R³ is selected from halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);
R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;
R⁷ is hydrogen, C₁-C₆ alkyl, C₂-C₆ alkenyl, or C₂-C₆ alkynyl;
R⁸ and R⁹ are each independently hydrogen, halogen, or C₁-C₃ alkyl;
R^{8a} and R^{9a} are each independently hydrogen, —OH, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵; or R^{8a} and R^{8b} taken together form an optionally substituted 3- to 6-membered carbocyclyl or heterocyclyl;
R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form a 3- to 6-membered heterocyclyl;
R¹⁶ is hydrogen, optionally substituted C₁-C₃ alkyl, optionally substituted C₂-C₃ alkenyl, optionally substituted C₂-C₃ alkynyl, C₃-C₆ cycloalky, or phenyl;
each m is independently 0, 1, or 2;
n1 and n2 are each independently 0, 1, or 2;
R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form a 3- to 6-membered heterocyclyl;
R¹⁶ is hydrogen, optionally substituted C₁-C₃ alkyl, optionally substituted C₂-C₃ alkenyl, optionally substituted C₂-C₃ alkynyl, C₃-C₆ cycloalky, or phenyl;
each m is independently 0, 1, or 2;
n1 and n2 are each independently 0, 1, or 2;
n3 is 1, 2, 3, 4 or 5; and
t is 0, 1 or 2.

In one embodiment, the present disclosure provides a pharmaceutical composition wherein the androgen receptor modulator is a compound of formula (IVA):

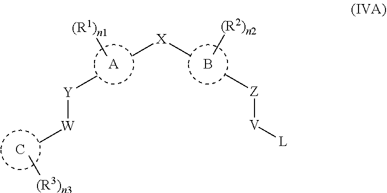

or a pharmaceutically acceptable salt, tautomer, stereoisomer or prodrug thereof, wherein:
A and B are each independently selected from phenyl, pyridyl, pyrimidyl, or thiophene;
C is a 3- to 10-membered ring;
X is a bond, —(CR⁵R⁶)ₜ—, or —NR⁷—;
Y and Z are each independently a bond, —CH₂—, —C(CH₃)H—, —O—, —S—, —NH—, —NCH₃—, or —N(COCH₃)—;
W is a bond, —CH₂—, —C(CH₃)H—, —C(═O)—, —N(R⁷)CO—, or —CONR⁷—;
V is —CH₂— and L is halogen, —NH₂, or —CF₃; or
V is —CH₂CH₂— and L is halogen or —NH₂;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;
R³ is selected from halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);
R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;
R⁷ is H or C₁-C₆ alkyl;
R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form a 3- to 6-membered heterocyclyl;
R¹⁶ is hydrogen, C₁-C₃ alkyl, C₂-C₃ alkenyl, or C₂-C₃ alkynyl;
n1 and n2 are each independently 0, 1, or 2;
n3 is 1, 2, 3, 4 or 5; and
t is 0, 1 or 2.

In one embodiment of the pharmaceutical composition comprising a compound of formula (IVA), C is 5- to 10-membered heteroaryl or aryl. In some embodiments, C is 5- to 7-membered heteroaryl comprising 1, 2, or 3 heteroatoms selected from O, S, or N as a ring member. In some embodiments, C, which is substituted with (R³)n3, is pyrazole, imidazole, oxazole, oxadiazole, oxazolone, isoxazole, thiazole, pyridyl, pyrazine, furan or pyrimidyl. In some embodiments, C, which is substituted with (R³)n3, is selected from

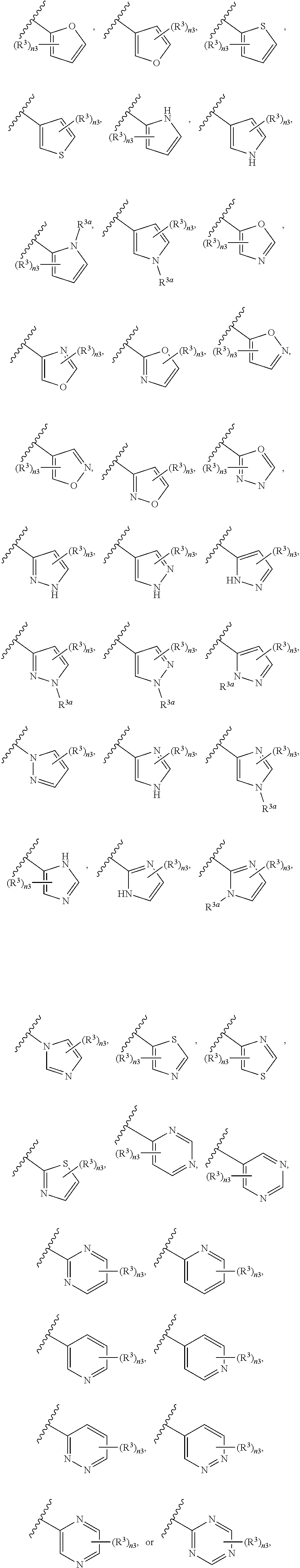

wherein R^{3a} id C₁-C₃ alkyl.

In one embodiment of the compound of formula (IVA), R¹ and R² are each independently Cl, —CN, —CF₃, —OH, methyl, methoxy, or —CONH₂.
In one embodiment of the compound of formula (IVA),
A and B are phenyl;
X is —(CR⁵R⁶)ₜ—;
Y and Z are each —O—;
V is —CH₂— or —CH₂CH₂—;
L is halogen;
R¹ and R² are each independently hydrogen, halogen, —CN, —CF₃, —OH, or optionally substituted C₁-C₆ alkyl;
R⁵ and R⁶ are each independently hydrogen, halogen, —OH, or C₁-C₃ alkyl; and
R¹⁶ is hydrogen, C₁-C₃ alkyl, C₂-C₃ alkenyl, or C₂-C₃ alkynyl.

In one embodiment of the compound of formula (IVA),
A and B are phenyl;
X is —(CR⁵R⁶)ₜ—;
W is —CH₂— or —C(CH₃)H—;
Y and Z are each —O—;
V is —CH₂CH₂—;
L is halogen;
R¹ and R² are each independently hydrogen, halogen, or —CN;
R⁵ and R⁶ are each independently hydrogen, or C₁-C₃ alkyl; and
R¹⁶ is hydrogen, C₁-C₃ alkyl, C₂-C₃ alkenyl, or C₂-C₃ alkynyl.

In one embodiment, the present disclosure provides a pharmaceutical composition wherein the androgen receptor modulator is a compound of formula (A-I):

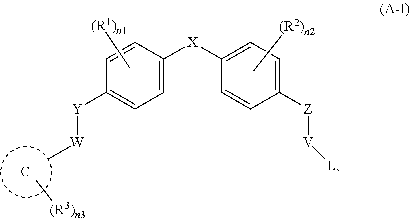

or a pharmaceutically acceptable salt, tautomer, stereoisomer or prodrug thereof, wherein:
C is a phenyl or a 5- to 7-membered monocyclic heteroaryl comprising 1, 2, or 3 heteroatoms selected from O, S, or N as a ring member;
X is a bond, —(CR⁵R⁶)ₜ—, or —NR⁷—;
Y is a bond, —CH₂—, —C(CH₃)H—, —O—, —S—, —NH—, —NCH₃—, or —N(COCH₃)—;
Z is a bond, —CH₂—, —O—, or —NH—;
W is a bond, —CH₂—, —C(CH₃)H—, —C(═O)—, —N(R⁷)CO—, or —CONR⁷—;
V is —CH₂— and L is halogen, —NH₂, or —CF₃; or
V is —CH₂CH₂— and L is halogen or —NH₂;
R¹ and R² are each independently hydrogen, halogen, —CN, —CF₃, methyl, or —CONH₂;
R³ is selected from from —CN, C₁-C₃ alkoxy, —CF₃, C₁-C₃ alkyl, C₂-C₃ alkenyl, C₂-C₃ alkynyl, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);
R⁵ and R⁶ are each independently hydrogen, halogen, —OH, or C₁-C₃ alkyl;
R⁷ is H or C₁-C₆ alkyl;
n1 and n2 are each independently 0, 1, or 2;
n3 is 1, 2, 3, 4 or 5; and
t is 0, 1 or 2.

In one embodiment of the compound of formula (IVA),

In one embodiment of the compound of formula (A-I), at least one R³ is selected from —CN, C₁-C₃ alkoxy, —CONH₂, —NHSO₂CH₃, —N(CH₃)SO₂CH₃, —NHSO₂CH₂CH₃, —N(CH₃)SO₂CH₂CH₃, or —SO₂CH₃ and the other R³, if present, is selected from —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl).

In one embodiment of the compound of formula (A-I),
X is a bond or —(CR⁵R⁶)ₜ;
W is a bond, —CH₂—, or —C(CH₃)H—;
Y is —O—;
Z is —O—;
V is —CH₂— or —CH₂CH₂—; and
L is halogen

In one embodiment, the present disclosure provides a pharmaceutical composition wherein the androgen receptor modulator is a compound of formula (G-II):

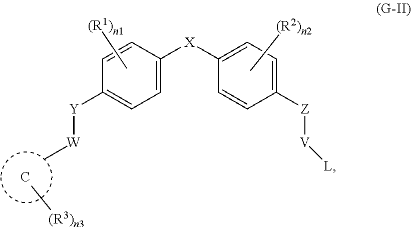

or a pharmaceutically acceptable salt, tautomer, stereoisomer or prodrug thereof, wherein:
C is

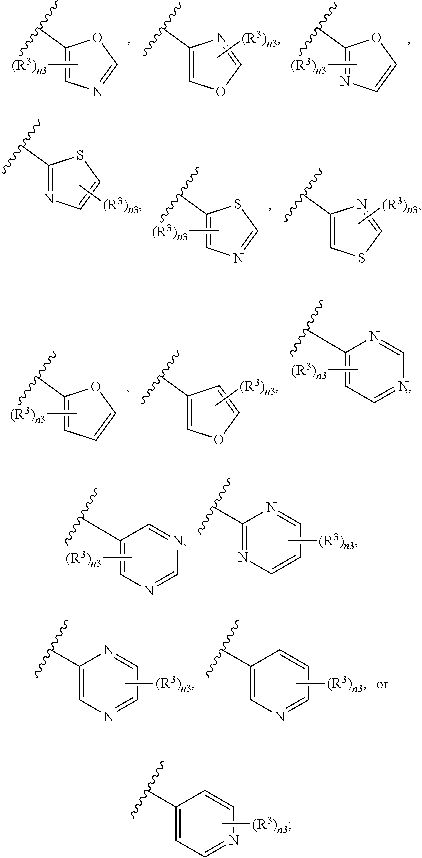

X is —(CR⁵R⁶)ₜ—;
Y is —O—;
Z is —O—;
W is —CH₂— or —C(CH₃)H—;
V is —CH₂CH₂—;
L is halogen;
R¹ and R² are each independently Cl or —CN;
at least one R³ is selected from —CN, C₁-C₃ alkoxy, —CONH₂, —NHSO₂CH₃, —N(CH₃)SO₂CH₃, —NHSO₂CH₂CH₃, —N(CH₃)SO₂CH₂CH₃, or —SO₂CH₃ and the other R³, if present, is selected from —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);
R⁵ and R⁶ are each independently hydrogen or methyl;
n1 and n2 are each independently 0, 1, or 2;
n3 is 1 or 2; and
t is 1.

In one embodiment of the compound of formula (G-II), at least one R³ is selected from —NHSO₂CH₃, —NHSO₂CH₂CH₃, or —SO₂CH₃ and the other R³, if present, is selected from —CN, C₁-C₃ alkyl, C₁-C₃ alkoxy, —SO₂(C₁-C₃ alkyl), —NH₂, —(C₁-C₃ alkyl)NH₂, —NHSO₂CH₃, —N(CH₃)SO₂CH₃, —NHSO₂CH₂CH₃, —N(CH₃)SO₂CH₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl).

In one embodiment of the pharmaceutical composition of the present disclosure, the androgen receptor modulator is selected from Table A. In one embodiment of the pharmaceutical composition of the present disclosure, the androgen receptor modulator is selected from Table B. In one embodiment of the pharmaceutical composition of the present disclosure, the androgen receptor modulator is selected from Compounds A1-A96, A98-A116, A118-A159, A161-A175, and A177-A234, or a pharmaceutically acceptable salt, tautomer, stereoisomer or prodrug thereof. In one embodiment of the pharmaceutical composition of the present disclosure, the androgen receptor modulator is selected from Compounds A13, A57, A74, A93, A109, A112, A122, A126, A131, A134, A136, A137, A164, A168, A169, A170, A171, A172, A184, A185, A195, and/or A204, or a pharmaceutically acceptable salt, tautomer, stereoisomer or prodrug thereof.

In one embodiment, the compounds in Table A and Table B are androgen receptor N-terminal domain inhibitors.

In one embodiment of the pharmaceutical composition of the present disclosure, the second therapeutically active agent is selected from a poly (ADP-ribose) polymerase (PARP) inhibitor, an androgen receptor ligand-binding domain inhibitor, an inhibitor of CYP17, a microtubule inhibitor, a modulator of PD-1 or PD-L1, a gonadotropin releasing hormone agonist, a 5-alpha reductase inhibitor, a vascular endothelial growth factor inhibitor, a histone deacetylase inhibitor, an integrin alpha-v-beta-3 inhibitor, a receptor tyrosine kinase, a phosphoinositide 3-kinase inhibitor, an anaplastic lymphoma kinase (ALK) inhibitor, an endothelin receptor A antagonist, an anti-CTLA4 inhibitor, an heat shock protein 27 (HSP27) inhibitor, an androgen receptor degrader, a androgen receptor DNA-binding domain inhibitor, a bromodomain and extra-terminal motif (BET) inhibitor, an androgen receptor N-terminal domain inhibitor, an alpha-particle emitting radioactive therapeutic agent, niclosamide, a selective estrogen receptor modulator (SERM), a selective estrogen receptor degrader (SERD), an aromatase inhibitor, selective progesterone receptor modulator (SPRM), a glucocorticoid receptor inhibitor, a HER2 receptor antagonist, a mammalian target of rapamycin (mTOR) inhibitor, an AKT inhibitor, a B-cell lymphoma-2 (Bcl-2) inhibitor, an aurora kinase inhibitor, a Wnt-targeting antagonist, a CYP11a inhibitor, a selective androgen receptor modulator, or enhancer of zeste homolog 2 (EZH2) inhibitor.

In another embodiment, the second therapeutically active agent is a nonsteroidal antiandrogen (NSAA).

In one embodiment of the pharmaceutical composition of the present disclosure, the androgen receptor ligand-binding domain inhibitor is enzalutamide, apalutamide, darolutamide, bicalutamide, nilutamide, flutamide, ODM-204, or TAS3681. In one embodiment, the androgen receptor ligand-binding domain inhibitor is enzalutamide.

In one embodiment of the pharmaceutical composition of the present disclosure, the Bcl-2 inhibitor is venetoclax.

In one embodiment, the present disclosure relates to a pharmaceutical composition comprising an androgen receptor ligand-binding domain inhibitor and a compound is selected from:

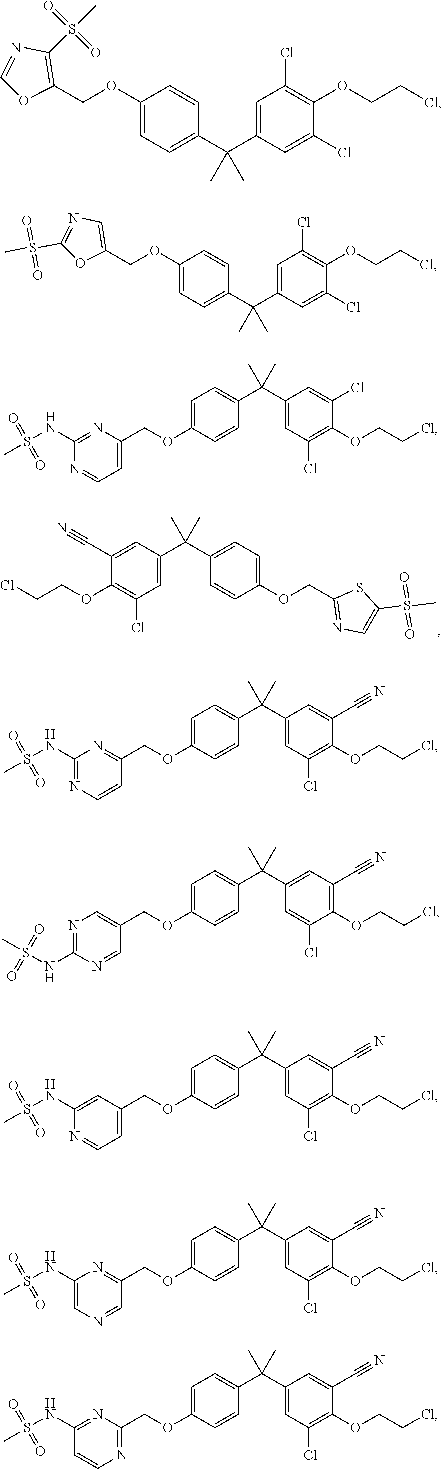

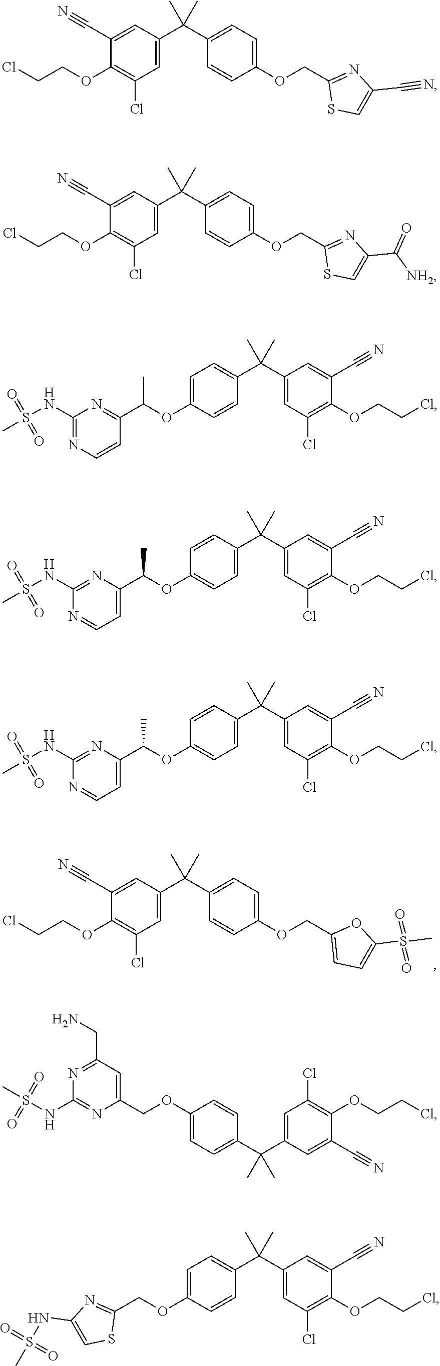

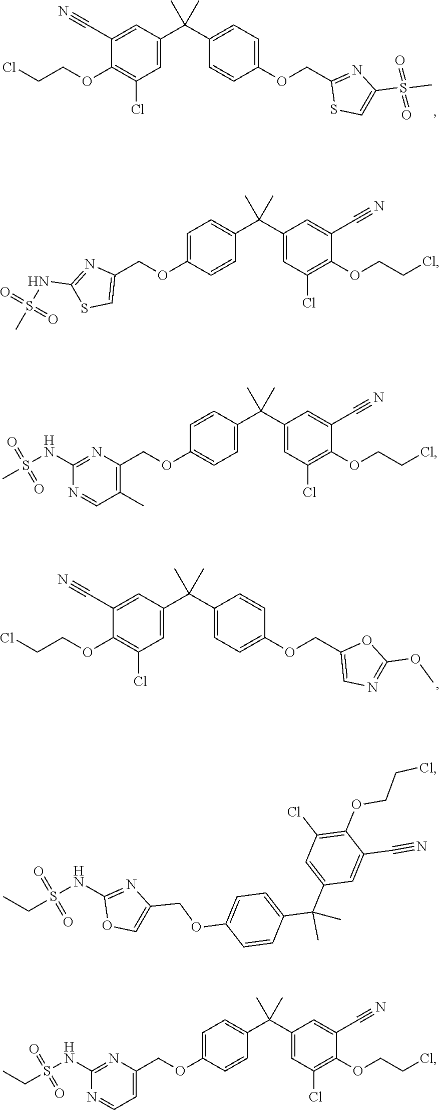

or a pharmaceutically acceptable salt, tautomer, stereoisomer or prodrug thereof. In one embodiment, the androgen receptor ligand-binding domain inhibitor is enzalutamide.

In one embodiment of any one of the pharmaceutical compositions disclosed herein, the composition further comprising a pharmaceutically acceptable carrier.

In one embodiment of the present disclosure, a method for modulating androgen receptor activity comprising administering any one of the pharmaceutical composition as disclosed herein is provided.

In one embodiment of the method of the present disclosure, the modulating androgen receptor activity is for treating a condition or disease selected from prostate cancer, breast cancer, ovarian cancer, bladder cancer, pancreatic cancer, hepatocellular cancer, endometrial cancer, salivary gland carcinoma, hair loss, acne, hirsutism, ovarian cysts, polycystic ovary disease, precocious puberty, spinal and bulbar muscular atrophy, or age-related macular degeneration.

In one embodiment of the present disclosure, a method of treating cancer, comprising administering any one of the pharmaceutical composition as disclosed herein is provided. In one embodiment, the cancer is breast cancer. In one embodiment, the breast cancer is triple negative breast cancer. In one embodiment, the cancer is selected from prostate cancer, breast cancer, ovarian cancer, bladder cancer, pancreatic cancer, hepatocellular cancer, endometrial cancer, or salivary gland carcinoma.

In one embodiment of the method of the present disclosure, the cancer is prostate cancer. In one embodiment, the prostate cancer is primary or localized prostate cancer, locally advanced prostate cancer, recurrent prostate cancer, advanced prostate cancer, metastatic prostate cancer, metastatic castration-resistant prostate cancer, and hormone-sensitive prostate cancer. In one embodiment, the prostate cancer is metastatic castration-resistant prostate cancer. In one embodiment, the prostate cancer expresses full-length androgen receptor or truncated androgen receptor splice variant. In one embodiment, the prostate cancer is resistant to enzalutamide monotherapy. In one embodiment, the prostate cancer is resistant to enzalutamide used with LHRH (luteinizing hormone-releasing hormone) analogues.

### BRIEF DESCRIPTION OF THE DRAWINGS

FIG. 1A, FIG. 1B, and FIG. 1C show change in tumor volume in male SCID Beige mice bearing VCaP tumors after oral administration of enzalutamide, Compound A109, or combination of enzalutamide and Compound A109.

FIG. 2A and FIG. 2B shows individual tumor volume change from baseline measured at the end of experiment for oral administration of enzalutamide, Compound A109, or combination of enzlutamide and Compound A109 to male SCID Beige mice bearing VCaP tumors.

FIG. 3 shows serum prostate-specific antigen (PSA) at the end of treatment with enzalutamide, Compound A109, or combination of enzalutamide and Compound A109 in male SCID Beige mice bearing VCaP tumors.

FIG. 4 shows change in % body weight in male SCID Beige mice bearing VCaP tumors after oral administration of representative compounds.

FIG. 5A shows log-ratio of a gene's or a transcript's expression values in LNCaP cells treated with enzalutamide compared to synthetic androgen (R1881). FIG. 5B shows log-ratio of a gene's or a transcript's expression values in LNCaP cells treated with Compound A109 compared to synthetic androgen (R1881).

FIG. 6A shows relative mRNA expression of androgen responsive genes in LNCaP cells treated with enzalutamide (Enza), Compound A109, or combinations of the two.

FIG. 6B shows the number of genes significantly down-regulated with Fold change of 4 or greater in LNCaP cells treated with enzalutamide (Enza), Compound A109, or combinations of the two.

FIG. 6C shows the top 10 down-regulated genes in enzalutamide (Enza)/Compound A109 combination.

FIG. 7A shows the number of genes significantly down-regulated with Fold change of 4 or greater in LNCaP cells treated with apalutamide (Apa), darolutamide (Daro), Compound A109, or combinations thereof.

FIG. 7B shows the top 10 down-regulated genes in darolutamide (Daro)/Compound A109 combination.

FIG. 8A shows log-ratio of a gene's or a transcript's expression values in LNCaP95 cells treated with enzalutamide compared to synthetic androgen (R1881), FIG. 8B shows log-ratio of a gene's or a transcript's expression values in LNCaP cells treated with Compound A109 compared to synthetic androgen (R1881).

FIG. 9A shows relative mRNA expression of androgen responsive genes in LNCaP95 (+R1881) cells treated with enzalutamide (Enza), Compound A109, or combinations of the two.

FIG. 9B shows relative mRNA expression of androgen responsive genes in LNCaP95 (+R1881) cells treated with enzalutamide (Enza), Compound A109, or combinations of the two.

### DETAILED DESCRIPTION

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

### Definitions

While the following terms are believed to be well understood by one of ordinary skill in the art, the following definitions are set forth to facilitate explanation of the presently disclosed subject matter.

Throughout the present specification, the terms “about” and/or “approximately” may be used in conjunction with numerical values and/or ranges. The term “about” is understood to mean those values near to a recited value. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity; for example, “a androgen receptor modulator” refers to one or more androgen receptor modulators or at least one androgen receptor modulator. As such, the terms “a” (or “an”), “one or more” and “at least one” are used interchangeably herein. In addition, reference to “an inhibitor” by the indefinite article “a” or “an” does not exclude the possibility that more than one of the inhibitors is present, unless the context clearly requires that there is one and only one of the inhibitors.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

The compounds of the invention, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present disclosure is meant to include all such possible isomers, as well as their racemic and optically pure forms whether or not they are specifically depicted herein. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposable mirror images of one another.

A “tautomer” refers to a proton shift from one atom of a molecule to another atom of the same molecule. The present disclosure includes tautomers of any said compounds.

A “prodrug” refers to a derivative of a compound of the present disclosure that will be converted to the compound in vivo. In one embodiment of the present disclosure, a prodrug includes a compound of formula (I)-(III), (IIIA), (VI), (IVA), (V)-(X), (A-I), or (G-II) having a free hydroxyl group (—OH) that is acetylated (—OCOMe) or acylated at one or more positions.

An “effective amount” means the amount of a formulation according to the invention that, when administered to a patient for treating a state, disorder or condition is sufficient to effect such treatment. The “effective amount” will vary depending on the active ingredient, the state, disorder, or condition to be treated and its severity, and the age, weight, physical condition and responsiveness of the mammal to be treated.

The term “therapeutically effective” applied to dose or amount refers to that quantity of a compound or pharmaceutical formulation that is sufficient to result in a desired clinical benefit after administration to a patient in need thereof.

As used herein, a “subject” can be a human, non-human primate, mammal, rat, mouse, cow, horse, pig, sheep, goat, dog, cat and the like. The subject can be suspected of having or at risk for having a cancer, such as prostate cancer, breast cancer, ovarian cancer, salivary gland carcinoma, or endometrial cancer, or suspected of having or at risk for having acne, hirsutism, alopecia, benign prostatic hyperplasia, ovarian cysts, polycystic ovary disease, precocious puberty, spinal and bulbar muscular atrophy, or age-related macular degeneration. Diagnostic methods for various cancers, such as prostate cancer, breast cancer, ovarian cancer, bladder cancer, pancreatic cancer, hepatocellular cancer, salivary gland carcinoma, or endometrial cancer, and diagnostic methods for acne, hirsutism, alopecia, benign prostatic hyperplasia, ovarian cysts, polycystic ovary disease, precocious puberty, spinal and bulbar muscular atrophy, or age-related macular degeneration and the clinical delineation of cancer, such as prostate cancer, breast cancer, ovarian cancer, bladder cancer, pancreatic cancer, hepatocellular cancer, salivary gland carcinoma, or endometrial cancer, diagnoses and the clinical delineation of acne, hirsutism, alopecia, benign prostatic hyperplasia, ovarian cysts, polycystic ovary disease, precocious puberty, spinal and bulbar muscular atrophy, or age-related macular degeneration are known to those of ordinary skill in the art.

“Mammal” includes humans and both domestic animals such as laboratory animals (e.g., mice, rats, monkeys, dogs, etc.) and household pets (e.g., cats, dogs, swine, cattle, sheep, goats, horses, rabbits), and non-domestic animals such as wildlife and the like.

All weight percentages (i.e., “% by weight” and “wt. %” and w/w) referenced herein, unless otherwise indicated, are measured relative to the total weight of the pharmaceutical composition.

As used herein, “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” other active agents would either completely lack other active agents, or so nearly completely lack other active agents that the effect would be the same as if it completely lacked other active agents. In other words, a composition that is “substantially free of” an ingredient or element or another active agent may still contain such an item as long as there is no measurable effect thereof

“Electrophile” as used herein relates to species that is attracted to an electron rich center. In chemistry, an electrophile is a reagent attracted to electrons. It participates in a chemical reaction by accepting an electron pair in order to bond to a nucleophile. Because electrophiles accept electrons, they are Lewis acids. Most electrophiles are positively charged, have an atom that carries a partial positive charge, or have an atom that does not have an octet of electrons.

The terms below, as used herein, have the following meanings, unless indicated otherwise:

“Amino” refers to the —NH₂ radical.

“Cyano” refers to the —CN radical.

“Halo” or “halogen” refers to bromo, chloro, fluoro or iodo radical, including their radioisotopes. “¹²³I” refers to the radioactive isotope of iodine having atomic mass 123. The compounds of Formula I can comprise at least one ¹²³I moiety. Throughout the present application, where structures depict a ¹²³I moiety at a certain position it is meant that the I moiety at this position is enriched for ¹²³I. In other words, the compounds contain more than the natural abundance of ¹²³I at the indicated position(s). It is not required that the compounds comprise 100% ¹²³I at the indicated positions, provided ¹²³I is present in more than the natural abundance. Typically the ¹²³I isotope is enriched to greater than 50%, greater than 60%, greater than 70%, greater than, 80% or greater than 90%, relative to ¹²⁷I. “¹⁸F” refers to the radioactive isotope of fluorine having atomic mass 18. “F” or “¹⁹F” refers to the abundant, non-radioactive fluorine isotope having atomic mass 19. The compounds of Formula I can comprise at least one ¹⁸F moiety. Throughout the present application, where structures depict a ¹⁸F moiety at a certain position it is meant that the F moiety at this position is enriched for ¹⁸F. In other words, the compounds contain more than the natural abundance of ¹⁸F at the indicated position(s). It is not required that the compounds comprise 100% ¹⁸F at the indicated positions, provided ¹⁸F is present in more than the natural abundance. Typically the ¹⁸F isotope is enriched to greater than 50%, greater than 60%, greater than 70%, greater than 80% or greater than 90%, relative to ¹⁹F.

“Hydroxy” or “hydroxyl” refers to the —OH radical.

“Imino” refers to the ═NH substituent.

“Nitro” refers to the —NO₂ radical.

“Oxo” refers to the ═O substituent.

“Thioxo” refers to the ═S substituent.

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to a straight or branched divalent hydrocarbon chain radical, having from two to twelve carbon atoms, and having one or more carbon-carbon double bonds. Non-limiting examples of C₂-C₁₂ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain radical, having from two to twelve carbon atoms, and having one or more carbon-carbon triple bonds. Non-limiting examples of C₂-C₁₂ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Alkoxy” refers to a radical of the formula —ORₐ where Rₐ is an alkyl, alkenyl or alknyl radical as defined above containing one to twelve carbon atoms. Unless stated otherwise specifically in the specification, an alkoxy group can be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Alkylcarbonyl” refers to the —C(═O)Rₐ moiety, wherein Rₐ is an alkyl, alkenyl or alkynyl radical as defined above. A non-limiting example of an alkyl carbonyl is the methyl carbonyl (“acetal”) moiety. Alkylcarbonyl groups can also be referred to as “Cw-Cz acyl” where w and z depicts the range of the number of carbon in Rₐ, as defined above. For example, “C₁-C₁₀ acyl” refers to alkylcarbonyl group as defined above, where Rₐ is C₁-C₁₀ alkyl, C₁-C₁₀ alkenyl, or C₁-C₁₀ alkynyl radical as defined above. Unless stated otherwise specifically in the specification, an alkyl carbonyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aralkyl” or “arylalkyl” refers to a radical of the formula —R_{b}-R_{c} where R_{b} is an alkylene group as defined above and R_{c} is one or more aryl radicals as defined above, for example, benzyl, diphenylmethyl and the like. Unless stated otherwise specifically in the specification, an aralkyl group can be optionally substituted.

“Aralkenyl” or “arylalkenyl” refers to a radical of the formula —R_{b}-R_{c} where R_{b} is an alkenylene o group as defined above and R_{c} is one or more aryl radicals as defined above. Unless stated otherwise specifically in the specification, an aralkenyl group can be optionally substituted.

“Aralkynyl” or “arylalkynyl” refers to a radical of the formula —R_{b}-R_{c} where R_{b} is an alkynylene group as defined above and R_{c} is one or more aryl radicals as defined above. Unless stated otherwise specifically in the specification, an aralkynyl group can be optionally substituted.

“Carbocyclyl,” “carbocyclic ring” or “carbocycle” refers to a rings structure, wherein the atoms which form the ring are each carbon. Carbocyclic rings can comprise from 3 to 20 carbon atoms in the ring. Carbocyclic rings include aryls and cycloalkyl. cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused or bridged ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Cycloalkylalkyl” refers to a radical of the formula —R_{b}-R_{d} where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above, that is substituted by one or more halo radicals, as defined above, e.g., 1-fluoropropynyl, 1-fluorobutynyl, and the like. Unless stated otherwise specifically in the specification, a haloalkenyl group can be optionally substituted.

“Heterocyclyl,” “heterocyclic ring” or “heterocycle” refers to a stable 3- to 20-membered non-aromatic, partially aromatic, or aromatic ring radical which consists of two to twelve carbon atoms and from one to six heteroatoms selected from the group consisting of nitrogen, oxygen and sulfur. Heterocyclycl or heterocyclic rings include heteroaryls as defined below. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Heterocyclylalkyl” refers to a radical of the formula —R_{b}-Rₑ where R_{b} is an alkylene group as defined above and Rₑ is a heterocyclyl radical as defined above. Unless stated otherwise specifically in the specification, a heterocycloalkylalkyl group can be optionally substituted.

“Heterocyclylalkenyl” refers to a radical of the formula —R_{b}-Rₑ where R_{b} is an alkenylene group as defined above and & is a heterocyclyl radical as defined above. Unless stated otherwise specifically in the specification, a heterocycloalkylalkenyl group can be optionally substituted.

“Heterocyclylalkynyl” refers to a radical of the formula —R_{b}-Rₑ where R_{b} is an alkynylene group as defined above and Rₑ is a heterocyclyl radical as defined above. Unless stated otherwise specifically in the specification, a heterocycloalkylalkynyl group can be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Heteroaryl” refers to a 5- to 20-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophene), benzotriazolyl, benzo[4,6]imidazo[1,2-a]pyridinyl, carbazolyl, cinnolinyl, dibenzofuranyl, dibenzothiophene, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophene (i.e. thienyl). Unless stated otherwise specifically in the specification, a heteroaryl group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Heteroarylalkyl” refers to a radical of the formula —R_{b}-R_{f} where R_{b} is an alkylene chain as defined above and R_{f} is a heteroaryl radical as defined above. Unless stated otherwise specifically in the specification, a heteroarylalkyl group can be optionally substituted.

“Heteroarylalkenyl” refers to a radical of the formula —R_{b}-R_{f} where R_{b} is an alkenylene, chain as defined above and R_{f} is a heteroaryl radical as defined above. Unless stated otherwise specifically in the specification, a heteroarylalkenyl group can be optionally substituted.

“Heteroarylalkynyl” refers to a radical of the formula —R_{b}-R_{f} where R_{b} is an alkynylene chain as defined above and R_{f} is a heteroaryl radical as defined above. Unless stated otherwise specifically in the specification, a heteroarylalkynyl group can be optionally substituted.

“Ring” refers to a cyclic group which can be fully saturated, partially saturated, or fully unsaturated. A ring can be monocyclic, bicyclic, tricyclic, or tetracyclic. Unless stated otherwise specifically in the specification, a ring can be optionally substituted.

“Thioalkyl” refers to a radical of the formula —SRₐ where Rₐ is an alkyl, alkenyl, or alkynyl radical as defined above containing one to twelve carbon atoms. Unless stated otherwise specifically in the specification, a thioalkyl group can be optionally substituted.

The term “substituted” used herein means any of the above groups (i.e., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups.

“Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g} Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol

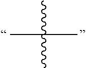

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

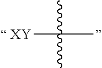

indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or

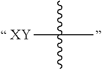

infers that when R³ is “XY”, the point of attachment bond is the same bond as the bond by which R³ is depicted as being bonded to CH₃.

“Fused” refers to any ring structure described herein which is fused to an existing ring structure in the compounds of the invention. When the fused ring is a heterocyclyl ring or a heteroaryl ring, any carbon atom on the existing ring structure which becomes part of the fused heterocyclyl ring or the fused heteroaryl ring can be replaced with a nitrogen atom.

The following description includes information that may be useful in understanding the present invention. It is not an admission that any of the information provided herein is prior art or relevant to the presently claimed inventions, or that any publication specifically or implicitly referenced is prior art.

### Androgen Receptor Modulators of the Present Disclosure

The pharmaceutical compositions or combinations of the present disclosure can be useful for modulating androgen receptors. In one embodiment, an androgen receptor modulator is an androgen receptor inhibitor. In one embodiment, an androgen receptor modulator is an androgen receptor N-terminal domain inhibitor. Further, the pharmaceutical compositions or combinations of the present disclosure can be useful for treating various diseases and conditions including, but not limited to, cancer.

The androgen receptor modulators of the present disclosure can, alone, be useful for treating various diseases and conditions including, but not limited to, cancer. In some embodiments, the cancer is prostate cancer.

In one embodiment the present disclosure provides androgen receptor modulators selected from compounds of formula (I):

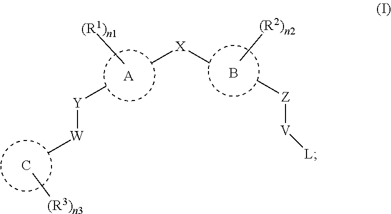

or a pharmaceutically acceptable salt, tautomer, stereoisomer or prodrug thereof, wherein:
A and B are each independently aryl or heteroaryl;
C is a 3- to 10-membered ring;
X is a bond, —(CR⁵R⁶)ₜ—, —O—, —C(═O)—, —S—, —S(═O)—, —SO₂—, —NR⁷—, —N(R⁷)CO—, —CON(R⁷)—, or —NSO₂R⁷—;
Y and Z are each independently a bond, —(CR⁸R⁹)ₘ—, —O—, —C(═O)—, —S—, —S(═O)—, —SO₂—, or —NR⁷—;
W and V are each independently a bond, —(CR^{8a}R^{9a})ₘ—, —C(═O)—, —N(R⁷)CO—, —CONR⁷—, or —NSO₂R⁷—;
L is hydrogen, halogen, —CF₂R¹⁰, —CF₃, —CN, —OR¹⁰; —NR¹¹R¹², or —CONR¹¹R¹²;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, optionally substituted —(C₁-C₆ alkyl)-SO₂R¹⁶, optionally substituted carbocyclyl, optionally substituted heterocyclyl, optionally substituted aryl, or optionally substituted heteroaryl;
R³ is hydrogen, halogen, oxo, ═S, ═NR¹⁶, —CN, —CF₃, —OH, —SR¹⁶, optionally substituted C₁-C₆ alkyl, optionally substituted C₂-C₆ alkenyl, optionally substituted C₂-C₆ alkynyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COOR¹⁶, —NR¹⁴COR¹⁶, —NR¹⁴CONR¹⁴R¹⁵, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, optionally substituted —(C₁-C₆ alkyl)-SO₂R¹⁶, optionally substituted carbocyclyl, optionally substituted heterocyclyl, optionally substituted aryl, or optionally substituted heteroaryl;
R⁵ and R⁶ are each independently hydrogen, halogen, —OH, optionally substituted C₁-C₆ alkyl, optionally substituted C₂-C₆ alkenyl, optionally substituted C₂-C₆ alkynyl, optionally substituted C₁-C₆ alkoxy, —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl; or R⁵ and R⁶ taken together form an optionally substituted carbocyclyl or optionally substituted heterocyclyl;
R⁸ and R⁹ are each independently hydrogen, halogen, or C₁-C₃ alkyl;
R^{8a} and R^{9a} are each independently hydrogen, —OH, halogen, optionally substituted C₁-C₆ alkyl, optionally substituted C₂-C₆ alkenyl, optionally substituted C₂-C₆ alkynyl, optionally substituted C₁-C₆ alkoxy, optionally substituted —OCO(C₁-C₆ alkyl), —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl; or R^{8a} and R^{8b} taken together form an optionally substituted carbocyclyl or optionally substituted heterocyclyl;
R⁷, R¹⁰ and R¹⁶ are each independently hydrogen, optionally substituted C₁-C₆ alkyl, optionally substituted C₂-C₆ alkenyl, optionally substituted C₂-C₆ alkynyl, C₁-C₆ haloalkyl, C₂-C₆ haloalkenyl, C₂-C₆ haloalkynyl, optionally substituted carbocyclyl, optionally substituted —CO(C₁-C₆ alkyl), —CO(optionally substituted heterocyclyl), optionally substituted heterocyclyl, optionally substituted aryl, or optionally substituted heteroaryl; or R⁷ and R^{8a} taken together form an optionally substituted heterocyclyl;
R¹¹, R¹², R¹³, R¹⁴ and R¹⁵ are each independently hydrogen, optionally substituted C₁-C₆ alkyl, optionally substituted C₂-C₆ alkenyl, optionally substituted C₂-C₆ alkynyl, optionally substituted —COO(C₁-C₆ alkyl), optionally substituted carbocyclyl, optionally substituted heterocyclyl, optionally substituted aryl, or optionally substituted heteroaryl; or (R¹¹ and R¹²) or (R¹⁴ and R¹⁵) taken together form an optionally substituted heterocyclyl;
each m is independently 0, 1 or 2;
n1 and n2 are each independently 0, 1, 2, 3, or 4;
n3 is 0, 1, 2, 3, 4 or 5; and
each t is independently 0, 1 or 2.

In one embodiment the present disclosure provides compounds comprising the structure of formula (IB):

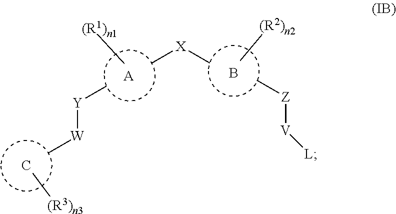

or a pharmaceutically acceptable salt, tautomer, stereoisomer or prodrug thereof, wherein:
A and B are each independently aryl or heteroaryl;
C is a 3- to 10-membered ring;
X is a bond, —(CR⁵R⁶)ₜ—, —O—, —C(═O)—, —S—, —S(═O)—, —SO₂—, —NR⁷—, —N(R⁷)CO—, —CON(R⁷)—, or —NSO₂R⁷—;
Y is a bond, —(CR⁸R⁹)ₘ—, —O—, —S—, —S(═O)—, —SO₂—, or —NR⁷—;
W is a bond, —(CR^{8a}R^{9a})ₘ—, —N(R⁷)CO—, —CONR⁷—, or —NSO₂R⁷—;
Z is a bond, —CH₂—, —C(CH₃)H—, —O—, —S—, —NH—, —NCH₃—, or —N(COCH₃)—;
V is —CH₂—, —CH₂CH₂—, —CH(CH₃)CH₂—, —CH₂CH(CH₃)—, or —CH₂CH₂CH₂—;
L is hydrogen, halogen, —CF₂R¹⁰, —CF₃, —CN, —OR¹⁰; —NR¹¹R¹², or —CONR¹¹R¹²;
R¹ and R² are each independently hydrogen, deuterium, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, optionally substituted —(C₁-C₆ alkyl)-SO₂R¹⁶, optionally substituted carbocyclyl, optionally substituted heterocyclyl, optionally substituted aryl, or optionally substituted heteroaryl;
R³ is hydrogen, halogen, oxo, ═S, ═NR¹⁶, —CN, —CF₃, —OH, —SR¹⁶, optionally substituted C₁-C₆ alkyl, optionally substituted C₂-C₆ alkenyl, optionally substituted C₂-C₆ alkynyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COOR¹⁶, —NR¹⁴COR¹⁶, —NR¹⁴CONR¹⁴R¹⁵, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, optionally substituted —(C₁-C₆ alkyl)-SO₂R¹⁶, optionally substituted carbocyclyl, optionally substituted heterocyclyl, optionally substituted aryl, or optionally substituted heteroaryl;
R⁵ and R⁶ are each independently hydrogen, halogen, —OH, optionally substituted C₁-C₆ alkyl, optionally substituted C₂-C₆ alkenyl, optionally substituted C₂-C₆ alkynyl, optionally substituted C₁-C₆ alkoxy, —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl; or R⁵ and R⁶ taken together form an optionally substituted carbocyclyl or optionally substituted heterocyclyl;
R⁸ and R⁹ are each independently hydrogen, halogen, or C₁-C₃ alkyl;
R^{8a} and R^{9a} are each independently hydrogen, halogen, optionally substituted C₁-C₆ alkyl, optionally substituted C₂-C₆ alkenyl, optionally substituted C₂-C₆ alkynyl, optionally substituted C₁-C₆ alkoxy, —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl; or R^{8a} and R^{8b} taken together form an optionally substituted carbocyclyl or optionally substituted heterocyclyl;
R⁷, R¹⁰ and R¹⁶ are each independently hydrogen, optionally substituted C₁-C₆ alkyl, optionally substituted C₂-C₆ alkenyl, optionally substituted C₂-C₆ alkynyl, optionally substituted C₁-C₆ alkyl-NH₂, C₁-C₆ haloalkyl, C₂-C₆ haloalkenyl, C₂-C₆ haloalkynyl, optionally substituted carbocyclyl, optionally substituted —CO(C₁-C₆ alkyl), —CO(optionally substituted heterocyclyl), optionally substituted heterocyclyl, optionally substituted aryl, or optionally substituted heteroaryl; or R⁷ and R^{8a} taken together form an optionally substituted heterocyclyl;
R¹¹, R¹², R¹³, R¹⁴ and R¹⁵ are each independently hydrogen, optionally substituted C₁-C₆ alkyl, optionally substituted C₂-C₆ alkenyl, optionally substituted C₂-C₆ alkynyl, optionally substituted —COO(C₁-C₆ alkyl), optionally substituted carbocyclyl, optionally substituted heterocyclyl, optionally substituted aryl, or optionally substituted heteroaryl; or (R¹¹ and R¹²) or (R¹⁴ and R¹⁵) or (R¹⁴ and R¹⁶) taken together form an optionally substituted heterocyclyl;
each m is independently 0, 1 or 2;
n1 and n2 are each independently 0, 1, 2, 3, or 4;
n3 is 0, 1, 2, 3, 4 or 5; and

each t is independently 0, 1 or 2. In one embodiment the present disclosure provides androgen receptor modulators selected from compounds of formula (II):

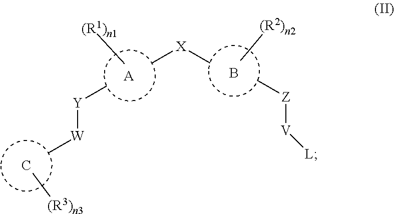

or a pharmaceutically acceptable salt, tautomer, stereoisomer or prodrug thereof, wherein:
A and B are each independently selected from phenyl, pyridyl, pyrimidyl, or thiophene;
C is a 5- to 10-membered heteroaryl or aryl;
X is a bond, —(CR⁵R⁶)ₜ—, or —NR⁷;
Y is a bond, —CH₂—, —C(CH₃)H—, —O—, —S—, —NH—, —NCH₃—, or —N(COCH₃)—;
Z is a bond, —CH₂—, —C(CH₃)H—, —O—, —S—, —NH—, —NCH₃—, or —N(COCH₃)—;
W is a bond, —CH₂—, —C(CH₃)H—, —C(═O)—, —N(R⁷)CO—, or —CONR⁷—;
V is —CH₂—, —CH₂CH₂—, —CH(CH₃)CH₂—, —CH₂CH(CH₃)—, or —CH₂CH₂CH₂—;
L is hydrogen, halogen, —OH, —NH₂, or —CF₃;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;
R³ is selected from hydrogen, halogen, oxo, ═S, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —NR¹⁴COOR¹⁶, —NR¹⁴CONR¹⁴R¹⁵, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);
R⁵ and R⁶ are each independently hydrogen, halogen, —OH, —NH₂, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;
R⁷ is H, C₁-C₆ alkyl, —CO(C₁-C₆ alkyl);
R¹³, R¹⁴ and R¹⁵ are each independently hydrogen, C₁-C₃ alkyl, C₂-C₃ alkenyl, C₂-C₃ alkynyl, or —COO(C₁-C₆ alkyl); or R¹⁴ and R¹⁵ taken together form a 3- to 6-membered heterocyclyl;
R¹⁶ is hydrogen, C₁-C₃ alkyl, C₁-C₃ haloalkyl, C₂-C₃ alkenyl, or C₂-C₃ alkynyl;
n1 and n2 are each independently 0, 1, or 2;
n3 is 0, 1, 2, 3, 4 or 5; and
t is 0, 1 or 2.

In one embodiment, the present disclosure provides androgen receptor modulators selected from compounds of formula (III):

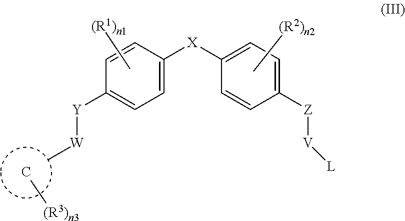

or a pharmaceutically acceptable salt, tautomer, stereoisomer or prodrug thereof, wherein:
C is a phenyl or a 5- to 7-membered monocyclic heteroaryl comprising 1, 2, or 3 heteroatoms selected from O, S, or N as a ring member;
X is a bond, —(CR⁵R⁶)ₜ—, or —NR⁷—;
Y is a bond, —CH₂—, —C(CH₃)H—, —O—, —S—, —NH—, —NCH₃—, or —N(COCH₃)—;
Z is a bond, —CH₂—, —O—, or —NH—;
W is a bond, —CH₂—, —C(CH₃)H—, —C(═O)—, —N(R⁷)CO—, or —CONR⁷—;
V is —CH₂—, —CH₂CH₂—, or —CH₂CH₂CH₂—;
L is halogen, —NH₂, or —CF₃;
R¹ and R² are each independently hydrogen, halogen, —CN, —CF₃, methyl, or —CONH₂;
R³ is selected from hydrogen, F, Cl, Br, I, oxo, —CN, —CF₃, —OH, C₁-C₃ alkyl, C₂-C₃ alkenyl, C₂-C₃ alkynyl, —S(C₁-C₃ alkyl), —SO₂(C₁-C₃ alkyl), —NHSO₂(C₁-C₃ alkyl), —N(CH₃)SO₂(C₁-C₃ alkyl), —CH₂NHSO₂(C₁-C₃ alkyl), —CH₂N(CH₃)SO₂(C₁-C₃ alkyl), —SO₂NH₂, —CONH₂, —CON(C₁-C₃ alkyl)₂, —CONH(C₁-C₃ alkyl), —NHCO(C₁-C₃ alkyl), or —N(CH₃)CO(C₁-C₃ alkyl);
R⁵ and R⁶ are each independently hydrogen, halogen, —OH, or C₁-C₃ alkyl;
R⁷ is H or C₁-C₆ alkyl;
n1 and n2 are each independently 0, 1, or 2;
n3 is 0, 1, 2, 3, 4 or 5; and
t is 0, 1 or 2.

In one embodiment the present disclosure provides androgen receptor modulators selected from compounds of formula (IV):

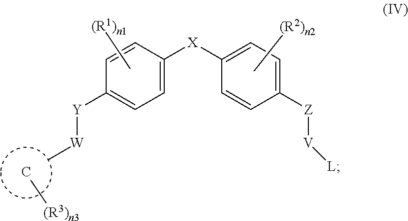

or a pharmaceutically acceptable salt, tautomer, stereoisomer or prodrug thereof, wherein:

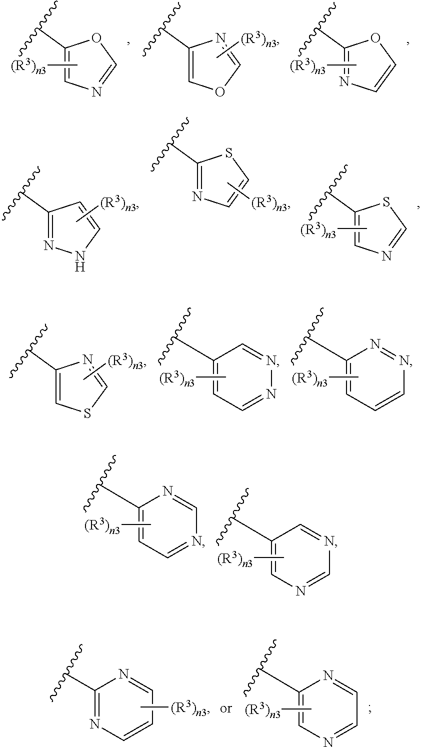

C is
X is —(CR⁵R⁶)ₜ— or —NR⁷—;
Y is a bond, —CH₂—, —O—, or —NH—;
Z is a bond, —CH₂—, —O—, or —NH—;
W is a bond, —CH₂—, or —C(CH₃)H—;
V is —CH₂—, —CH₂CH₂—, —CH₂CH₂CH₂—, or —CH₂CHClCH₂—;
L is hydrogen, —OH, or halogen;
R¹ and R² are each independently hydrogen, halogen, —CN, —CF₃, methyl, or —CONH₂;
R³ is selected from hydrogen, F, Cl, Br, I, oxo, —CN, —CF₃, —OH, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —NHSO₂(C₁-C₃ alkyl), —NHSO₂CF₃, —N(CH₃)SO₂(C₁-C₃ alkyl), —CH₂NHSO₂(C₁-C₃ alkyl), —CH₂N(CH₃)SO₂(C₁-C₃ alkyl), —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);
R⁵ and R⁶ are each independently hydrogen or C₁-C₃ alkyl;
R⁷ is H or C₁-C₆ alkyl;
n1 and n2 are each independently 0, 1, or 2;
n3 is 0, 1, or 2; and
t is 1 or 2.

In one embodiment of the compounds of formula (III)-(V), R³ is selected from hydrogen, F, Cl, Br, I, —CN, —CF₃, —OH, methyl, methoxy, —S(C₁-C₃ alkyl), —SO₂(C₁-C₃ alkyl), —NH₂, —NHSO₂CH₃, —NHSO₂CF₃, —N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), or —NHCO(C₁-C₃ alkyl).

In one embodiment the present disclosure provides androgen receptor modulators selected from compounds of formula (V)

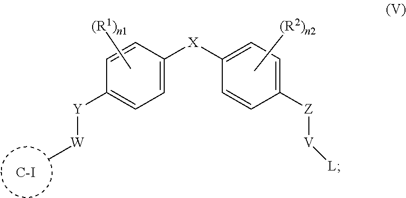

or a pharmaceutically acceptable salt, tautomer, stereoisomer or prodrug thereof, wherein:
C-I is

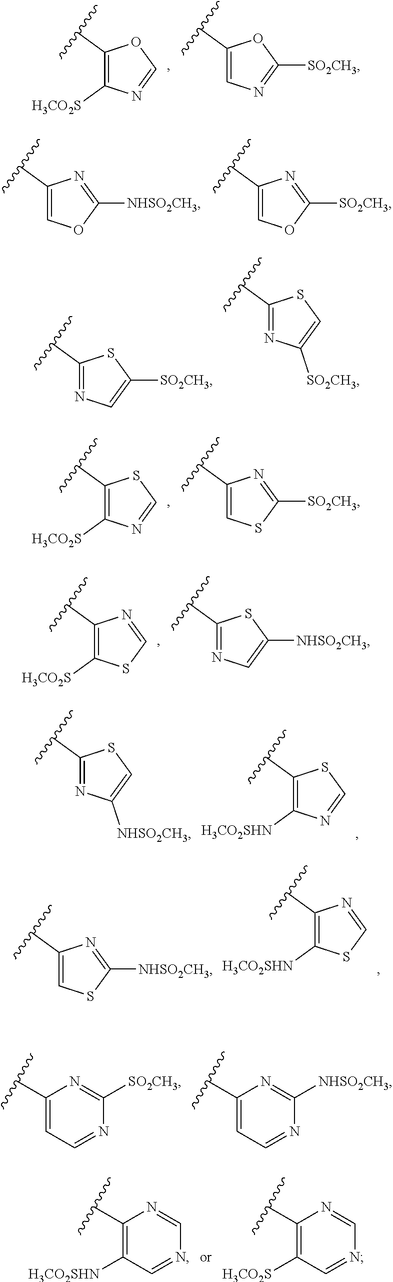

X is —(CR⁵R⁶)ₜ—;
Y is —O—;
Z is —O—;
W is —CH₂— or —C(CH₃)H—;
V is —CH₂—, —CH₂CH₂— or —CH₂CH₂CH₂—;
L is halogen;
R¹ and R² are each independently methyl, Cl or —CN;
R⁵ and R⁶ are each independently hydrogen or methyl;
R⁷ is H or C₁-C₆ alkyl;
n1 and n2 are each independently 0, 1, or 2; and
t is 1.

In one embodiment of the compounds of formula (I) or (II), rings A and B are each independently 5- or 6-membered aryl or heteroaryl. In one embodiment, rings A and B are each independently selected from phenyl, pyridyl, pyrimidyl, or thiophene. In one embodiment, rings A and B are each phenyl.

In another embodiment, ring A has a meta or para connectivity with X and Y. In some embodiments, ring B has a meta or para connectivity with X and Z.

In one embodiment of the compounds of formula (I) or (II), rings A and B are phenyl and has one of the connectivity as shown:

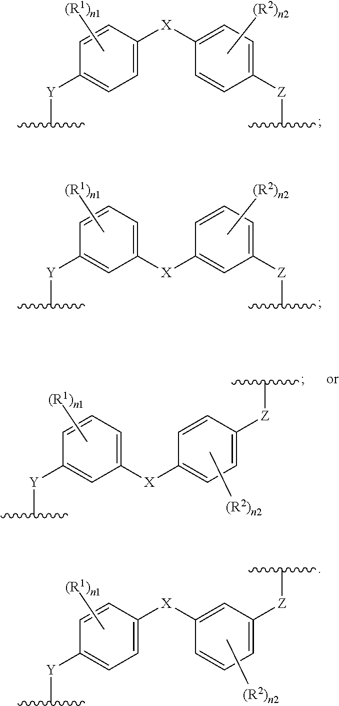

In one embodiment of the compounds of formula (I)-(III), ring C is aryl or heteroaryl. In some embodiments, ring C is 5- to 10-membered aryl or heteroaryl. In other embodiments, ring C is aryl. In some embodiments, ring C is phenyl or naphthyl. In other embodiments, ring C is aryl. In some embodiments, ring C is phenyl or naphthyl. In some embodiments, ring C is phenyl.

In one embodiment of the compounds of formula (I)-(III), ring C is heteroaryl. In one embodiment, ring C is monocyclic or bicyclic heteroaryl. In another embodiment, ring C is monocyclic heteroaryl. In some embodiments, ring C is 5- or 10-membered heteroaryl. In some embodiments, ring C is 5- or 6-membered heteroaryl, which is optionally substituted with 1, 2, 3, 4, or 5 R³. In some embodiments, ring C is 5- or 6-membered heteroaryl containing 1, 2, or 3 heteroatoms selected from O, S, or N, wherein the heteroaryl is optionally substituted with 1, 2, 3, 4, or 5 R³. In some embodiments, ring C is 5- or 6-membered heteroaryl containing 1 or 2 heteroatoms selected from O, S, or N, wherein the heteroaryl is optionally substituted with 1, 2, 3, 4, or 5 R³.

In one embodiment of the compounds of formula (I)-(III), ring ring C is pyrazole, imidazole, oxazole, oxadiazole, oxazolone, isoxazole, thiazole, pyridyl, or pyrimidyl, which are each optionally substituted with 1, 2, 3, 4, or 5 R³.

In one embodiment of the compounds of formula (I)-(III), ring C is selected from

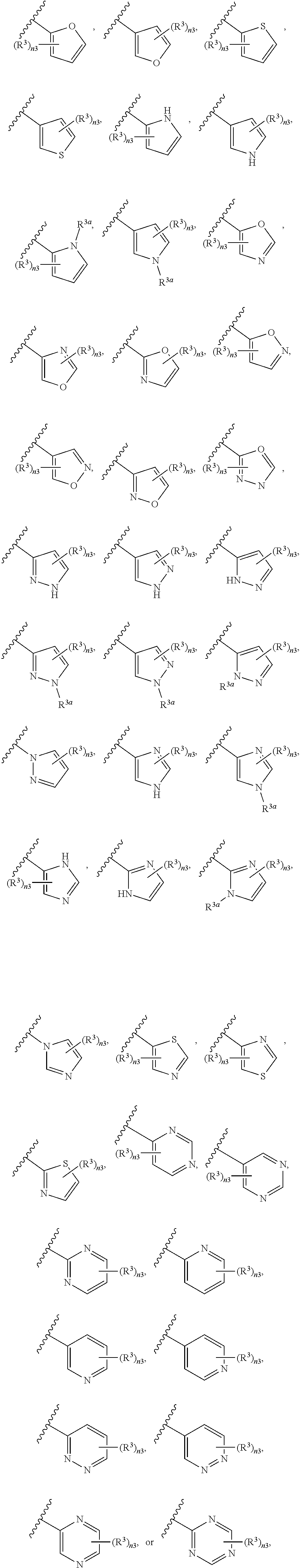

wherein R^{3a} is C₁-C₃ alkyl.

In one embodiment of the compounds of formula (I)-(V), ring C is N

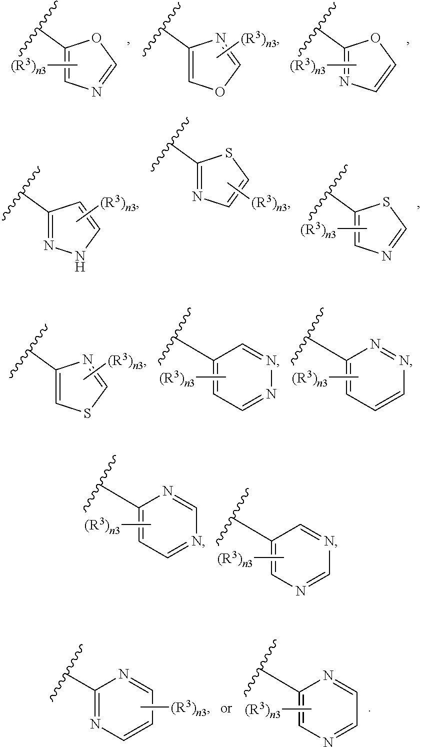

In one embodiment, ring C is

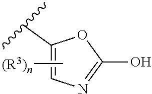

or in its tautomeric form

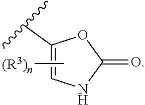

In one embodiment, ring C is

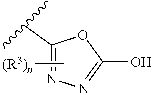

or in its tautomeric form

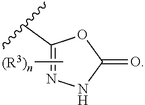

In one embodiment of the compounds of formula (I) or (II), ring C is heterocyclyl. In one embodiment, ring C is saturated or partially saturated heterocycle. In some embodiments, ring C is monocyclic or bicyclic. In some embodiments, ring C is 5- to 7-membered heterocyclyl comprising 1, 2, or 3 heteroatoms selected from O, S, or N as a ring member.

In one embodiment of the compounds of formula (I) or (II), ring C is imidazolidine, imidazolidine-dione, or dihydrooxazole. In one embodiment, ring C is selected from

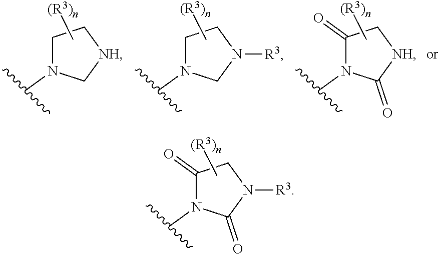

In one embodiment of the compounds of formula (I) or (II), ring C is

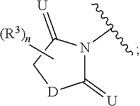

D is —O—, —NH— or —NR³—; and U is each independently O, S, or NR¹⁶. In one embodiment, D is —NH— or —NR³—. In some embodiments, at least one U is O. In other embodiments, each U is O. In some embodiments, at least one R³ is —SO₂CH₃, —NHSO₂CH₃, —CH₂NHSO₂CH₃, —SO₂NH₂, —CONH₂, or —NHCOCH₃.

In one embodiment of the compounds of formula (I), Z is a bond, —(CR⁸R⁹)ₘ—, —O—, —C(═O)—, —S—, —S(═O)—, —SO₂—, or —NR⁷—. In one embodiment, Z is —(CR⁸R⁹)ₘ—, —O—, —C(═O)—, —S—, —S(═O)—, —SO₂—, or —NR⁷—. In some embodiments, Z is —C(═O)—.

In one embodiment of the compounds of formula (I)-(III), Z is a bond, —CH₂—, —C(CH₃)H—, —O—, —S—, —NH—, —NCH₃—, or —N(COCH₃)—. In one embodiment, Z is —CH₂—, —C(CH₃)H—, —O—, —S—, —NH—, —NCH₃—, or —N(COCH₃)—. In some embodiments, Z is a bond, —CH₂—, —O—, or —NCH₃—. In some embodiments, Z is a bond, —CH₂—, —O—, or —NH—. In some embodiments, Z is —O—.

In one embodiment of the compounds of formula (I), Y is a bond, —(CR⁸R⁹)ₘ—, —O—, —C(═O)—, —S—, —S(═O)—, —SO₂—, or —NR⁷—. In one embodiment, Y is —(CR⁸R⁹)ₘ—, —O—, —C(═O)—, —S—, —S(═O)—, —SO₂—, or —NR⁷—. In some embodiments, Y is —C(═O)—.

In one embodiment of the compounds of formula (I)-(III), Y is a bond, —CH₂—, —C(CH₃)H—, —O—, —S—, —NH—, —NCH₃—, or —N(COCH₃)—. In one embodiment, Y is —CH₂—, —C(CH₃)H—, —O—, —S—, —NH—, —NCH₃—, or —N(COCH₃)—. In some embodiments, Y is a bond, —CH₂—, —O—, or —NCH₃—. In some embodiments, Y is a bond, —CH₂—, —O—, or —NH—. In some embodiments, Y is —O—.

In one embodiment of the compounds of formula (I)-(III), V is a bond, —(CR^{8a}R^{9a})ₘ—, or —C(═O)—. In some embodiments, V is bond, or —(CR^{8a}R^{9a})ₘ—.

In one embodiment of the compounds of formula (I)-(III), V is —(CR^{8a}R^{9a})ₘ—, wherein m is 1, 2, or 3. In one embodiment of the compounds of formula (I)-(III), V is —(CR^{8a}R^{9a})ₘ—, wherein R^{8a} and R^{8b} are each independently hydrogen, —OH, halogen, optionally substituted C₁-C₃ alkyl, optionally substituted C₂-C₃ alkenyl, optionally substituted C₂-C₃ alkynyl, C₁-C₃ alkoxy, —NR¹³R¹⁴, optionally substituted —(C₁-C₃ alkyl)-NR¹³R¹⁴, —NR¹⁴COR¹⁶, optionally substituted —(C₁-C₃ alkyl)-NR¹⁴COR¹⁶, —CONR¹⁴R¹⁵, or optionally substituted —(C₁-C₃ alkyl)-CONR¹⁴R¹⁵; or R^{8a} and R^{8b} taken together form an optionally substituted 3- to 6-membered carbocyclyl or heterocyclyl. In one embodiment, V is —(CR^{8a}R^{9a})ₘ—, wherein R^{8a} and R^{8b} are each independently hydrogen, —OH, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵; or R^{8a} and R^{8b}, on the same carbon atom or on a different carbon atom, taken together form an optionally substituted 3- to 6-membered carbocyclyl or heterocyclyl.

In one embodiment of the compounds of formula (I)-(III), V is —CH₂—, —CH₂CH₂—, or —CH₂CH₂CH₂—, each optionally substituted with one or more of —OH, halogen, or C₁-C₃ alkyl. In other embodiments, V is —CH₂—, —CH₂CH₂—, —CH₂CH(OH)CH₂— or —CH₂CH₂CH₂—. In some embodiments, V is —CH₂—, —CH₂CH₂—, or —CH₂CH₂CH₂—.

In one embodiment of the compounds of formula (I)-(III), L is hydrogen, halogen, —CF₂H, —CF₃, —CN, —O(C₁-C₃ alkyl), —NR¹¹R¹², or —CONR¹¹R¹². In one embodiment, L is hydrogen, halogen, —CF₂H, —CF₃, —CN, —O(C₁-C₃ alkyl), —NH₂, —NH(C₁-C₃ alkyl), —N(C₁-C₃ alkyl)₂, —CONH₂, —CONH(C₁-C₃ alkyl), or —CON(C₁-C₃ alkyl)₂. In some embodiments, L is hydrogen, halogen, —CF₃, or —NH₂. In some embodiments, L is halogen, —CF₃, or —NH₂. In one embodiment, L is hydrogen or halogen. In one embodiment, L is halogen. In other embodiments, L is Cl, or Br. In one embodiment, L is Cl.

In one embodiment of the compounds of formula (I)-(III), W is a bond. In one embodiment, W is —(CR^{8a}R^{9a})ₘ—, —C(═O)—, —N(R⁷)CO—, —CONR⁷—, or —NSO₂R⁷—. In one embodiment, W is —(CR^{8a}R^{9a})ₘ—, wherein m is 1, 2, or 3. In some embodiments, W is a bond, —CH₂—, —C(CH₃)H—, —C(═O)—, —N(R⁷)CO—, or —CONR⁷—, wherein R⁷ is H or C₁-C₆ alkyl. In some embodiments, W is a bond, —CH₂—, —C(CH₃)H—, —C(═O)—, —N(R⁷)CO—, or —CONR⁷—, wherein R⁷ is H or C₁-C₃ alkyl. In one embodiment, W is a bond, —CH₂—, —C(CH₃)H—, —C(═O)—, —NHCO—, —N(C₁-C₃ alkyl)CO—, —CONH—, or —CON(C₁-C₃ alkyl)-. In one embodiment, W is a bond, —CH₂—, or —C(CH₃)H—. In one embodiment, W is a —CH₂— or —C(CH₃)H—.

In one embodiment of the compounds of formula (I)-(III), —Y—W— is a bond, —OCH₂—, —OCH₂CH₂—, —OCH(CH₃)—, —NH—, —NHCH₂—, —NHC(═O)—, or —C(═O)NH—.

In one embodiment of the compounds of formula (I)-(III),
Z is a bond, —CH₂—, —C(CH₃)H—, —O—, —S—, —NH—, —NCH₃—, or —N(COCH₃)—; and
V is —CH₂—, —CH₂CH₂—, or —CH₂CH₂CH₂—.

In one embodiment of the compounds of formula (I)-(III),
Z is a bond, —CH₂—, —C(CH₃)H—, —O—, —S—, —NH—, —NCH₃—, or —N(COCH₃)—;
V is —CH₂—, —CH₂CH₂—, or —CH₂CH₂CH₂—; and
L is halogen, —NH₂, or —CF₃.

In one embodiment of the compounds of formula (I)-(III), —Z—V-L is —Z—CH₂CH₂Cl, —Z—CH₂CH₂CH₂Cl, —Z—CH₂CH₂NH₂, or —Z—CH₂CH₂CH₂NH₂, wherein Z is a bond, —O—, —NH—, or —N(COCH₃)—. In one embodiment, —Z—V-L is —OCH₃.

In one embodiment of the compounds of formula (I)-(III), —V-L is —CH₂CH₂Cl, —CH₂CH₂CH₂Cl, —CH₂CH₂NH₂, or —CH₂CH₂CH₂NH₂. In one embodiment, —V-L is —CH₃.

In one embodiment of the compounds of formula (I)-(III), X is a bond, —(CR⁵R⁶)ₜ—, —O—, —C(═O)—, —S—, —S(═O)—, —SO₂—, or —NR⁷—. In one embodiment, X is a bond, —(CR⁵R⁶)ₜ—, —O—, —C(═O)—, —S—, —S(═O)—, —SO₂—, or —NR⁷—, wherein R⁷ is H or C₁-C₆ alkyl. In some embodiments, X is a bond, —(CR⁵R⁶)ₜ—, or —NR⁷—. In some embodiments, X is a bond or —(CR⁵R⁶)ₜ—. In some embodiments, X is a bond, —CH₂—, —C(CH₃)H—, —C(CH₃)₂—, —CH₂CH₂—, —NH—, or —N(C₁-C₆ alkyl)-. In some embodiments, X is a bond, —CH₂—, —C(CH₃)H—, —C(CH₃)₂—, —CH₂CH₂—, —NH—, —N(CH₃)—, —N(CH₂CH₃)—, —N(iPr)-, or —N(tBu)-. In some embodiments, X is a bond, —CH₂—, —C(CH₃)H—, —C(CH₃)₂—, or —CH₂CH₂—.

In one embodiment of the compounds of formula (I)-(III), R¹ and R² are each independently halogen, —CN, —CF₃, —OH, C₁-C₃ alkyl, C₁-C₃ alkoxy, —(C₁-C₃ alkyl)-(C₁-C₃ alkoxy), —(C₁-C₃ alkyl)-OH, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₃ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂R¹⁶, —(C₁-C₃ alkyl)-SO₂R¹⁶, 3- to 7-membered carbocyclyl, 3- to 7-membered heterocyclyl, phenyl, or 5- to 6-membered heteroaryl.

In one embodiment of the compounds of formula (I)-(III), R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, or optionally substituted —(C₁-C₆ alkyl)-SO₂R¹⁶. In one embodiment, R¹ and R² are each independently hydrogen, halogen, —CN, —CF₃, —OH, optionally substituted C₁-C₃ alkyl, C₁-C₃ alkoxy, optionally substituted —(C₁-C₃ alkyl)-(C₁-C₃ alkoxy), optionally substituted —(C₁-C₃ alkyl)-OH, —NR¹³R¹⁴, —(C₁-C₃ alkyl)-NR¹³R¹⁴, —NR¹⁴SO₂R¹⁶, optionally substituted —(C₁-C₃ alkyl)NR¹⁴SO₂R¹⁶, —NR¹⁴COR¹⁶, optionally substituted —(C₁-C₃ alkyl)-NR¹⁴COR¹⁶, —CONR¹⁴R¹⁵, optionally substituted —(C₁-C₃ alkyl)-CONR¹⁴R¹⁵, —SO₂NR¹⁴R¹⁵, optionally substituted —(C₁-C₃ alkyl)-SO₂NR¹⁴R¹⁵, —SO₂R¹⁶, or optionally substituted —(C₁-C₃ alkyl)-SO₂R¹⁶. In one embodiment, R¹ and R² are each independently hydrogen, halogen, —CN, —CF₃, —OH, C₁-C₃ alkyl, C₁-C₃ alkoxy, —(C₁-C₃ alkyl)-(C₁-C₃ alkoxy), —(C₁-C₃ alkyl)-OH, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₃ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂R¹⁶, or —(C₁-C₃ alkyl)-SO₂R¹⁶.

In one embodiment of the compounds of formula (I)-(III), R¹ and R² are each independently hydrogen, halogen, —CN, —CF₃, —OH, C₁-C₃ alkyl, or —CONR¹⁴R¹⁵. In some embodiments, R¹ and R² are each independently hydrogen, halogen, —CN, —CF₃, —OH, methyl, methoxy, or —CONH₂. In one embodiment, R¹ and R² are each independently hydrogen, Cl, —CN, —CF₃, —OH, methyl, methoxy, or —CONH₂. In one embodiment, R¹ and R² are each independently hydrogen, halogen, —CN, —CF₃, —OH, or methyl. In one embodiment, R¹ and R² are each independently Cl, —CN, —CF₃, —OH, methyl, methoxy, or —CONH₂. In one embodiment, R¹ and R² are each independently halogen, —CN, —CF₃, —OH, or methyl.

In one embodiment of the compounds of formula (I)-(III), R¹ and R² are each independently optionally substituted carbocyclyl, optionally substituted heterocyclyl, optionally substituted aryl, or optionally substituted heteroaryl. In one embodiment, R¹ and R² are each independently 3- to 7-membered carbocyclyl, 3- to 7-membered heterocyclyl, phenyl, or 5- to 6-membered heteroaryl.

In one embodiment of the compounds of formula (I)-(III), R¹ have one of the connectivity as shown below with respect to X and Y:

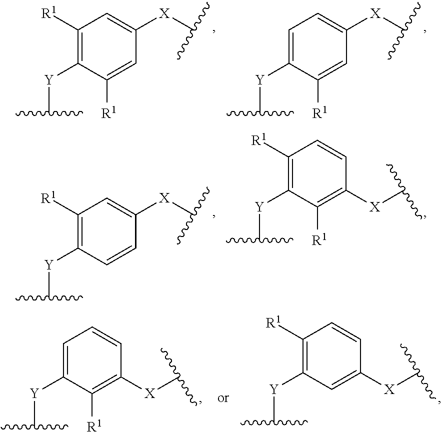

In one embodiment of the compounds of formula (I)-(III), R² have one of the connectivity as shown below with respect to X and Z:

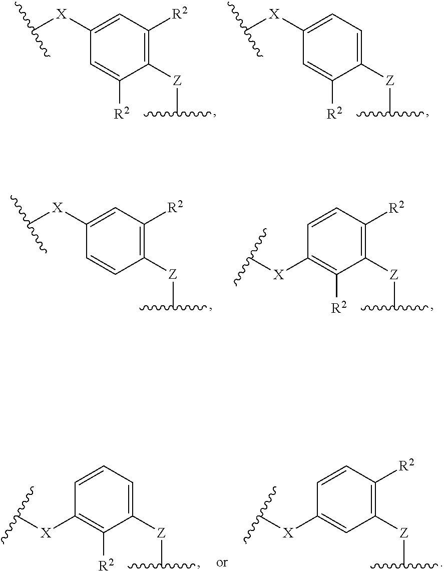

In one embodiment of the compounds of formula (I)-(III), n1 is 0, 1, or 2. In some embodiments, n1 is 0 or 1. In other embodiments, n1 is 0. In some embodiments, n1 is 1. In one embodiment, the sum of n1 and n2 is 0, 1, 2, 3, or 4. In some embodiments, the sum of n1 and n2 is 1, 2, 3, or 4. In one embodiment, the sum of n1 and n2 is 2.

In one embodiment of the compounds of formula (I)-(III), n2 is 0, 1, or 2. In some embodiments, n2 is 1 or 2. In other embodiments, n2 is 0. In some embodiments, n2 is 1. In some embodiments, n2 is 2.

In one embodiment of the compounds of formula (I)-(III), R³ is selected from hydrogen, halogen, oxo, ═S, ═NR¹⁶, —CN, —CF₃, —OH, —SR¹⁶, optionally substituted C₁-C₆ alkyl, optionally substituted C₂-C₆ alkenyl, optionally substituted C₂-C₆ alkynyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, optionally substituted —(C₁-C₆ alkyl)-SO₂R¹⁶, optionally substituted carbocyclyl, optionally substituted heterocyclyl, optionally substituted aryl, or optionally substituted heteroaryl. In some embodiments, R³ is selected from hydrogen, halogen, oxo, ═S, ═NR¹⁶, —CN, —CF₃, —OH, —SR¹⁶, optionally substituted C₁-C₆ alkyl, optionally substituted C₂-C₆ alkenyl, optionally substituted C₂-C₆ alkynyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, or optionally substituted —(C₁-C₆ alkyl)-SO₂R¹⁶. In another embodiment, R³ is hydrogen, halogen, oxo, ═S, ═NR¹⁶, —CN, —CF₃, —OH, —SR¹⁶ optionally substituted C₁-C₃ alkyl, optionally substituted C₂-C₃ alkenyl, optionally substituted C₂-C₃ alkynyl, optionally substituted C₁-C₃ alkoxy, optionally substituted —(C₁-C₃ alkyl)-(C₁-C₃ alkoxy), optionally substituted —(C₁-C₃ alkyl)-OH, —NR¹³R¹⁴, optionally substituted —(C₁-C₃ alkyl)-NR¹³R¹⁴, —NR¹⁴SO₂R¹⁶, optionally substituted —(C₁-C₃ alkyl)NR¹⁴SO₂R¹⁶, —NR¹⁴COR¹⁶, optionally substituted —(C₁-C₃ alkyl)-NR¹⁴COR¹⁶, —CONR¹⁴R¹⁵, optionally substituted —(C₁-C₃ alkyl)-CONR¹⁴R¹⁵, —SO₂NR¹⁴R¹⁵, optionally substituted —(C₁-C₃ alkyl)-SO₂NR¹⁴R¹⁵, optionally substituted —SO₂R¹⁶, or optionally substituted —(C₁-C₃ alkyl)-SO₂R¹⁶.

In one embodiment of the compounds of formula (I)-(III), R³ is selected from hydrogen, halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —(C₁-C₃ alkyl)-(C₁-C₃ alkoxy), —(C₁-C₃ alkyl)-OH, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO(C₁-C₃ alkyl), —SO₂(C₁-C₃ alkyl), or —(C₁-C₆ alkyl)-SO₂(C₁-C₃ alkyl). In some embodiments, R³ is selected from hydrogen, F, Cl, Br, I, oxo, ═S, ═NR¹⁶, —CN, —CF₃, —OH, C₁-C₃ alkyl, C₂-C₃ alkenyl, C₂-C₃ alkynyl, —S(C₁-C₃ alkyl), —SO(C₁-C₃ alkyl), —SO₂(C₁-C₃ alkyl), —NHSO₂CH₃, —N(CH₃)SO₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), or —N(CH₃)CO(C₁-C₃ alkyl). In one embodiment, R³ is selected from hydrogen, F, Cl, Br, I, oxo, ═S, ═NR¹⁶, —CN, —CF₃, —OH, methyl, —SCH₃, —SO₂CH₃, —NHSO₂CH₃, —CH₂NHSO₂CH₃, —SO₂NH₂, —CONH₂, or —NHCOCH₃. In some embodiments, R³ is selected from F, Cl, Br, I, oxo, ═S, ═NR¹⁶, —CN, —CF₃, —OH, C₁-C₃ alkyl, C₂-C₃ alkenyl, C₂-C₃ alkynyl, —S(C₁-C₃ alkyl), —SO(C₁-C₃ alkyl), —SO₂(C₁-C₃ alkyl), —NHSO₂CH₃, —N(CH₃)SO₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), or —N(CH₃)CO(C₁-C₃ alkyl). In one embodiment, R³ is selected from F, Cl, Br, I, oxo, ═S, ═NR¹⁶, —CN, —CF₃, —OH, methyl, —SCH₃, —SO₂CH₃, —NHSO₂CH₃, —CH₂NHSO₂CH₃, —SO₂NH₂, —CONH₂, or —NHCOCH₃. In another embodiment, R³ is —SO₂CH₃, —NHSO₂CH₃, —CH₂NHSO₂CH₃, —SO₂NH₂, —CONH₂, or —NHCOCH₃. In one embodiment, R³ is oxo, ═S, ═NR¹⁶, C₁-C₃ alkyl, —SO₂(C₁-C₃ alkyl), or —NHSO₂(C₁-C₃ alkyl). In one embodiment, at least one of R³ is oxo, ═S, or ═NR¹⁶. In one embodiment, at least one of R³ is oxo, ═S, or ═NR¹⁶, wherein R¹⁶ is H or C₁-C₃ alkyl.

In one embodiment of the compounds of formula (I)-(III), R³ on a sp³ carbon is each selected from hydrogen, halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —(C₁-C₃ alkyl)-(C₁-C₃ alkoxy), —(C₁-C₃ alkyl)-OH, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO(C₁-C₃ alkyl), —SO₂(C₁-C₃ alkyl), or —(C₁-C₆ alkyl)-SO₂(C₁-C₃ alkyl). When R³ on a sp³ carbon is oxo, ═S, or ═NR¹⁶, the carbon becomes sp².

In one embodiment of the compounds of formula (I)-(III), R³ on a sp² carbon is each selected from hydrogen, halogen, —CN, —CF₃, —OH, —S(C₁-C₃ alkyl), C₁-C₃ alkyl, C₂-C₃ alkenyl, C₂-C₃ alkynyl, C₁-C₃ alkoxy, —(C₁-C₃ alkyl)-(C₁-C₃ alkoxy), —(C₁-C₃ alkyl)-OH, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO(C₁-C₃ alkyl), —SO₂(C₁-C₃ alkyl), or —(C₁-C₆ alkyl)-SO₂(C₁-C₃ alkyl).

In one embodiment of the compounds of formula (I)-(III), R³ on a nitrogen atom is each selected from C₁-C₃ alkyl, C₂-C₃ alkenyl, C₂-C₃ alkynyl, C₁-C₃ alkoxy, —(C₁-C₃ alkyl)-(C₁-C₃ alkoxy), —(C₁-C₃ alkyl)-OH, —(C₁-C₃ alkyl)-NR¹³R¹⁴, —(C₁-C₃ alkyl)NR¹⁴SO₂R¹⁶, —(C₁-C₆ alkyl)-NR¹⁴COR¹⁶, —CONR¹⁴R¹⁵, —(C₁-C₃ alkyl)-CONR¹⁴R¹⁵, —(C₁-C₃ alkyl)-SO₂NR¹⁴R¹⁵, or —(C₁-C₆ alkyl)-SO₂(C₁-C₃ alkyl).

In one embodiment of the compounds of formula (I)-(III), at least one R³ is —SO₂CH₃, —NHSO₂CH₃, or —NCH₃SO₂CH₃.

In one embodiment of the compounds of formula (I)-(III), R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl. In some embodiments, R⁵ and R⁶ are each independently hydrogen, halogen, —OH, optionally substituted C₁-C₆ alkyl, optionally substituted C₂-C₆ alkenyl, optionally substituted C₂-C₆ alkynyl. In one embodiment, R⁵ and R⁶ are hydrogen, halogen, —OH, or C₁-C₃ alkyl. In one embodiment, R⁵ and R⁶ are each independently hydrogen, F, —OH, or C₁-C₃ alkyl. In one embodiment, R⁵ and R⁶ are each independently, hydrogen, F, —OH, or methyl. In one embodiment, R⁵ and R⁶ are each H. In one embodiment, R⁵ and R⁶ are each methyl. In one embodiment, R⁵ and R⁶ are each H or methyl.

In one embodiment of the compounds of formula (I)-(III), R⁷ is hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl. In some embodiments, R⁷ is hydrogen, optionally substituted C₁-C₃ alkyl, optionally substituted C₂-C₃ alkenyl, optionally substituted C₂-C₃ alkynyl, optionally substituted carbocyclyl, optionally substituted heterocyclyl, optionally substituted aryl, or optionally substituted heteroaryl. In some embodiments, R⁷ is hydrogen, C₁-C₆ alkyl, C₂-C₆ alkenyl, C₂-C₆ alkynyl, carbocyclyl, heterocyclyl, aryl, or heteroaryl. In some embodiments, R⁷ is hydrogen, C₁-C₃ alkyl, C₂-C₃ alkenyl, C₂-C₃ alkynyl, carbocyclyl, heterocyclyl, aryl, or heteroaryl. In some embodiments, R⁷ is hydrogen, C₁-C₆ alkyl, C₂-C₆ alkenyl, or C₂-C₆ alkynyl. In some embodiments, R⁷ is hydrogen, C₁-C₃ alkyl, C₂-C₃ alkenyl, or C₂-C₃ alkynyl. In some embodiments, R⁷ is hydrogen or C₁-C₆ alkyl. In some embodiments, R⁷ is hydrogen or C₁-C₄ alkyl. In some embodiments, R⁷ is hydrogen or C₁-C₃ alkyl.

In one embodiment of the compounds of formula (I)-(III), R^{8a} and R^{9a} are each independently hydrogen, halogen, optionally substituted C₁-C₆ alkyl, optionally substituted C₂-C₆ alkenyl, optionally substituted C₂-C₆ alkynyl, optionally substituted C₁-C₆ alkoxy, —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl, or R^{8a} and R^{9a} taken together form an optionally substituted carbocyclyl or optionally substituted heterocyclyl. In some embodiments, R^{8a} and R^{8b} are each independently hydrogen, —OH, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵; or R^{8a} and R^{8b} taken together form an optionally substituted 3- to 6-membered carbocyclyl or heterocyclyl. In one embodiment, R^{8a} and R^{9a} are each independently hydrogen, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵. In one embodiment, R^{8a} and R^{9a} are not —OH.

In one embodiment of the compounds of formula (I)-(III), R⁷ and R^{8a} taken together form an optionally substituted heterocyclyl. In one embodiment, R⁷ and R^{8a} taken together form an optionally substituted 3- to 7-membered heterocycle.

In one embodiment of the compounds of formula (I)-(III), R⁸ and R⁹ are each independently hydrogen, halogen, or C₁-C₃ alkyl.

In one embodiment of the compounds of formula (I)-(III), R¹⁰ is hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl. In some embodiments, R¹⁰ is hydrogen, optionally substituted C₁-C₃ alkyl, optionally substituted C₂-C₃ alkenyl, optionally substituted C₂-C₃ alkynyl, optionally substituted carbocyclyl, optionally substituted heterocyclyl, optionally substituted aryl, or optionally substituted heteroaryl. In some embodiments, R¹⁰ is hydrogen, C₁-C₃ alkyl, C₂-C₃ alkenyl, C₂-C₃ alkynyl, carbocyclyl, heterocyclyl, aryl, or heteroaryl. In some embodiments, R¹⁰ is hydrogen, C₁-C₃ alkyl, C₂-C₃ alkenyl, C₂-C₃ alkynyl. In some embodiments, R¹⁰ is hydrogen or C₁-C₃ alkyl.

In one embodiment of the compounds of formula (I)-(III), R¹¹ and R¹² are each independently hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl. In one embodiment, R¹¹ and R¹² are each independently hydrogen, optionally substituted C₁-C₆ alkyl, optionally substituted C₂-C₆ alkenyl, or optionally substituted C₂-C₆ alkynyl. In some embodiments. R¹¹ and R¹² are each independently hydrogen, C₁-C₆ alkyl, C₂-C₆ alkenyl, or C₂-C₆ alkynyl. In some embodiments. R¹¹ and R¹² are each independently hydrogen or C₁-C₃ alkyl.

In one embodiment of the compounds of formula (I)-(III), R¹¹ and R¹² taken together form an optionally substituted heterocyclyl. In one embodiment, R¹¹ and R¹² taken together form an optionally substituted 3- to 7-membered heterocyclyl. In other embodiments, ¹¹ and R¹² taken together form 3- to 7-membered heterocyclyl.

In one embodiment of the compounds of formula (I)-(III), R¹³ and R¹⁴ are each independently hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl. In one embodiment, R¹³ and R¹⁴ are each independently hydrogen, optionally substituted C₁-C₆ alkyl, optionally substituted C₂-C₆ alkenyl, or optionally substituted C₂-C₆ alkynyl. In some embodiments R¹³ and R¹⁴ are each independently hydrogen, C₁-C₆ alkyl, C₂-C₆ alkenyl, or C₂-C₆ alkynyl. In some embodiments R¹³ and R¹⁴ are each independently hydrogen or C₁-C₃ alkyl.

In one embodiment of the compounds of formula (I)-(III), R¹⁵ is hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl. In one embodiment, R¹⁵ is hydrogen, optionally substituted C₁-C₆ alkyl, optionally substituted C₂-C₆ alkenyl, or optionally substituted C₂-C₆ alkynyl. In some embodiments, R¹⁵ is hydrogen, C₁-C₆ alkyl, C₂-C₆ alkenyl, or C₂-C₆ alkynyl. In some embodiments R¹⁵ is hydrogen or C₁-C₃ alkyl.

In one embodiment of the compounds of formula (I)-(III), R¹⁴ and R¹⁵ taken together form an optionally substituted heterocyclyl. In one embodiment, R¹⁴ and R¹⁵ taken together form an optionally substituted 3- to 7-membered heterocyclyl. In other embodiments, R¹⁴ and R¹⁵ taken together form 3- to 7-membered heterocyclyl.

In one embodiment of the compounds of formula (I)-(III), R¹⁶ is hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl. In some embodiments, R¹⁶ is hydrogen, optionally substituted C₁-C₃ alkyl, optionally substituted C₂-C₃ alkenyl, optionally substituted C₂-C₃ alkynyl, optionally substituted carbocyclyl, optionally substituted heterocyclyl, optionally substituted aryl, or optionally substituted heteroaryl. In some embodiments, R¹⁶ is hydrogen, C₁-C₃ alkyl, C₂-C₃ alkenyl, C₂-C₃ alkynyl, carbocyclyl, heterocyclyl, aryl, or heteroaryl. In some embodiments, R¹⁶ is hydrogen, C₁-C₃ alkyl, C₂-C₃ alkenyl, or C₂-C₃ alkynyl. In some embodiments, R¹⁶ is hydrogen or C₁-C₃ alkyl.

In one embodiment of the compounds of formula (I)-(III), m is 1 or 2.

In one embodiment of the compounds of formula (I)-(III), t is 1 or 2.

In one embodiment of the compounds of formula (I)-(III), optional substituent is selected from halogen, —CN, —CF₃, —OH, —S(C₁-C₃ alkyl), C₁-C₃ alkyl, C₂-C₃ alkenyl, C₂-C₃ alkynyl, C₁-C₃ alkoxy, —(C₁-C₃ alkyl)-(C₁-C₃ alkoxy), —(C₁-C₃ alkyl)-OH, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO(C₁-C₃ alkyl), —SO₂(C₁-C₃ alkyl), or —(C₁-C₆ alkyl)-SO₂(C₁-C₃ alkyl). In another embodiment, the optional substituent is selected from halogen, —CN, —CF₃, —OH, C₁-C₃ alkyl, C₁-C₃ alkoxy, —NH₂, —SCH₃, —SO₂CH₃, —NHSO₂CH₃, —CH₂NHSO₂CH₃, —SO₂NH₂, —CONH₂, or —NHCOCH₃.

In one embodiment of the compounds of formula (I), (IB), or (II), A and B are each monocyclic ring.

In one embodiment of the compounds of formula (I), (IB), or (II), B is phenyl, pyridyl, or pyrimidyl.

In one embodiment of the compounds of formula (I), (IB), or (II), Z and V are not both a bond.

In one embodiment of the compounds of formula (I)-(III), Y and W are not both a bond.

In one embodiment of the compounds of formula (I), (IB), or (II), C is a 4- to 10-membered ring.

In one embodiment of the compounds of formula (IV) or (V), X is a bond, —CH₂—, —C(CH₃)H—, —C(CH₃)₂—, or —CH₂CH₂—. In one embodiment, X is —CH₂—, —C(CH₃)H—, or —C(CH₃)₂—. In some embodiments, X is —C(CH₃)₂—.

In one embodiment of the compounds of formula (IV) or (V), X is —NR⁷—. In one embodiment, X is —NH—, —N(CH₃)—, —N(CH₂CH₃)—, —N(iPr)-, or —N(tBu)-.

In one embodiment of the compounds of formula (IV) or (V), Y is —O—. In one embodiment of the compounds of formula (IV) or (V), Z is —O—. In one embodiment of the compounds of formula (IV) or (V), Y and Z are both —O—.

In one embodiment of the compounds of formula (IV) or (V), —V-L is CH₂CH₂Cl, —CH₂CH₂CH₂Cl, or —CH₃. In some embodiments, —V-L is CH₂CH₂Cl or —CH₂CH₂CH₂Cl.

In one embodiment of the compounds of formula (IV) or (V), n1 is 0.

In one embodiment of the compounds of formula (IV) or (V), n2 is 0, 1, or 2. In some embodiments, n2 is 2. In some embodiments, n2 is 2 and R² are each ortho to Z. In other embodiments, n2 is 2 and R² are each ortho to Z, wherein R² is halogen or —CN.

In one embodiment of the compounds of formula (I)-(V), R³ is selected from hydrogen, F, Cl, Br, I, —CN, —CF₃, —OH, methyl, methoxy, —S(C₁-C₃ alkyl), —SO₂(C₁-C₃ alkyl), —NH₂, —NHSO₂CH₃, —NHSO₂CF₃, —N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), or —NHCO(C₁-C₃ alkyl).

In one embodiment of the compounds of formula (I)-(V), the compounds can be in a specific stereoisomer form. For example, if X is —(CR⁵R⁶)— and R⁵ and R⁶ are different, the carbon attached to R⁵ and R⁶ can be in an S configuration or an R configuration.

In one embodiment of the pharmaceutical compositions or combinations or the methods of the present disclosure, the androgen receptor modulator has the structure of formula (VI):

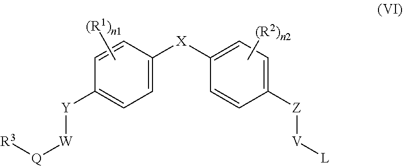

or a pharmaceutically acceptable salt, tautomer, stereoisomer or prodrug thereof, wherein:
Q is a bond, C₁-C₃ alkylene, arylene, heteroarylene, carbocyclylene, or heterocyclylene, each optionally substituted with —OH or R¹;
X is —(CR⁵R⁶)ₜ— or —NR⁷—;
Y is a bond, —CH₂—, —O—, or —NH—;
Z is a bond, —CH₂—, —O—, or —NH—;
W is a bond, or —(CR^{8a}R^{9a})ₘ—;
V is a bond, —(CR^{8a}R^{9a})ₘ—, —C(═O)—, —N(R⁷)CO—, —CONR⁷—, or —NSO₂R⁷—;
L is hydrogen, —OH, or halogen;
R¹ and R² are each independently hydrogen, halogen, —CN, —CF₃, methyl, or —CONH₂;
R³ is selected from —S(C₁-C₃ alkyl), —SO₂(C₁-C₃ alkyl), —NH₂, —NHSO₂(C₁-C₃ alkyl), —NHSO₂CF₃, —N(CH₃)SO₂(C₁-C₃ alkyl), —CH₂NHSO₂(C₁-C₃ alkyl), —CH₂N(CH₃)SO₂(C₁-C₃ alkyl), —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);
R⁵ and R⁶ are each independently hydrogen, halogen, —OH, —NH₂, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;
R⁷ is H or C₁-C₆ alkyl;
R^{8a} and R^{9a} are each independently hydrogen, —OH, halogen, optionally substituted C₁-C₆ alkyl, optionally substituted C₂-C₆ alkenyl, optionally substituted C₂-C₆ alkynyl, optionally substituted C₁-C₆ alkoxy, optionally substituted —OCO(C₁-C₆ alkyl), —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl;
R¹³, R¹⁴ and R¹⁵ are each independently hydrogen, C₁-C₃ alkyl, C₂-C₃ alkenyl, C₂-C₃ alkynyl, or —COO(C₁-C₆ alkyl); or R¹⁴ and R¹⁵ taken together form a 3- to 6-membered heterocyclyl;
R¹⁶ is hydrogen, C₁-C₃ alkyl, C₁-C₃ haloalkyl, C₂-C₃ alkenyl, or C₂-C₃ alkynyl;
n1 and n2 are each independently 0, 1, or 2;
m is 1, 2, 3, or 4; and
t is 1 or 2.

In one embodiment of the pharmaceutical compositions or combinations or the methods of the present disclosure, the androgen receptor modulator has the structure of formula (VII):

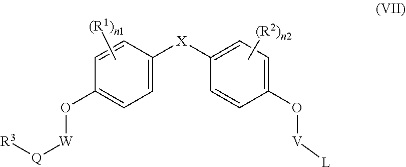

or a pharmaceutically acceptable salt, tautomer, stereoisomer or prodrug thereof, wherein:
Q is a bond, C₁-C₃ alkylene, arylene, or heteroarylene, each optionally substituted with —OH or R¹;
X is —(CR⁵R⁶)ₜ—;
W is a bond, —CH₂—, —C(CH₃)H—, —CH₂CH₂—, —CH₂CH₂CH₂—, or —CH₂CH(OH)CH₂—;
V is a bond or —(CR^{8a}R^{9a})ₘ—;
L is hydrogen, —OH, or halogen;
R¹ and R² are each independently hydrogen, halogen, —CN, —CF₃, methyl, or —CONH₂;
R³ is selected from —S(C₁-C₃ alkyl), —SO₂(C₁-C₃ alkyl), —NH₂, —NHSO₂(C₁-C₃ alkyl), —NHSO₂CF₃, —N(CH₃)SO₂(C₁-C₃ alkyl), —CH₂NHSO₂(C₁-C₃ alkyl), —CH₂N(CH₃)SO₂(C₁-C₃ alkyl), —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);
R⁵ and R⁶ are each independently H, halogen, —OH, —NH₂, or C₁-C₃ alkyl;
R⁷ is H or C₁-C₆ alkyl;
R^{8a} and R^{9a} are each independently hydrogen, —OH, halogen, or C₁-C₃ alkyl;
n1 is 0, 1, or 2;
n2 is 1 or 2; m is 1, 2, 3, or 4; and
t is 1 or 2.

In one embodiment of the pharmaceutical compositions or combinations or the methods of the present disclosure, the androgen receptor modulator has the structure of formula (VIII):

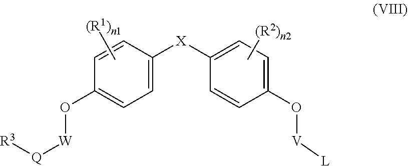

or a pharmaceutically acceptable salt, tautomer, stereoisomer or prodrug thereof, wherein:
Q is a bond, C₁-C₃ alkylene, phenylene, or 5- or 6-membered heteroarylene, each optionally substituted with —OH or R¹;
X is —(CR⁵R⁶)ₜ—;
Y is a bond, —CH₂—, —O—, or —NH—;
Z is a bond, —CH₂—, —O—, or —NH—;
W is a bond, —CH₂—, —C(CH₃)H—, —CH₂CH₂—, —CH₂CH₂CH₂—, or —CH₂CH(OH)CH₂—;
V is a bond, —CH₂—, —CH₂CH₂—, —CH(CH₃)CH₂—, —CH(OH)CH₂—, —CH₂CH(CH₃)—, —CH₂CH₂CH₂—, or —CH₂CH(OH)CH₂—;
L is halogen;
R¹ and R² are each independently hydrogen, halogen, —CN, —CF₃, methyl, or —CONH₂;
R³ is selected from —S(C₁-C₃ alkyl), —SO₂(C₁-C₃ alkyl), —NH₂, —NHSO₂(C₁-C₃ alkyl), —NHSO₂CF₃, —N(CH₃)SO₂(C₁-C₃ alkyl), —CH₂NHSO₂(C₁-C₃ alkyl), —CH₂N(CH₃)SO₂(C₁-C₃ alkyl), —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);
R⁵ and R⁶ are each independently H, halogen, —OH, —NH₂, or C₁-C₃ alkyl;
R⁷ is H or C₁-C₆ alkyl;
R^{8a} and R^{9a} are each independently hydrogen, —OH, halogen, or C₁-C₃ alkyl;
n1 is 0, 1, or 2;
n2 is 1 or 2;
m is 1, 2, 3, or 4; and
t is 1 or 2.

In one embodiment of the pharmaceutical compositions or combinations or the methods of the present disclosure, the androgen receptor modulator has the structure of formula (IX):

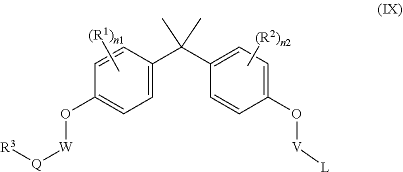

or a pharmaceutically acceptable salt, tautomer, stereoisomer or prodrug thereof, wherein:

Q is a —CH₂—, —CH₂CH₂—, —CH₂CH₂CH₂—,

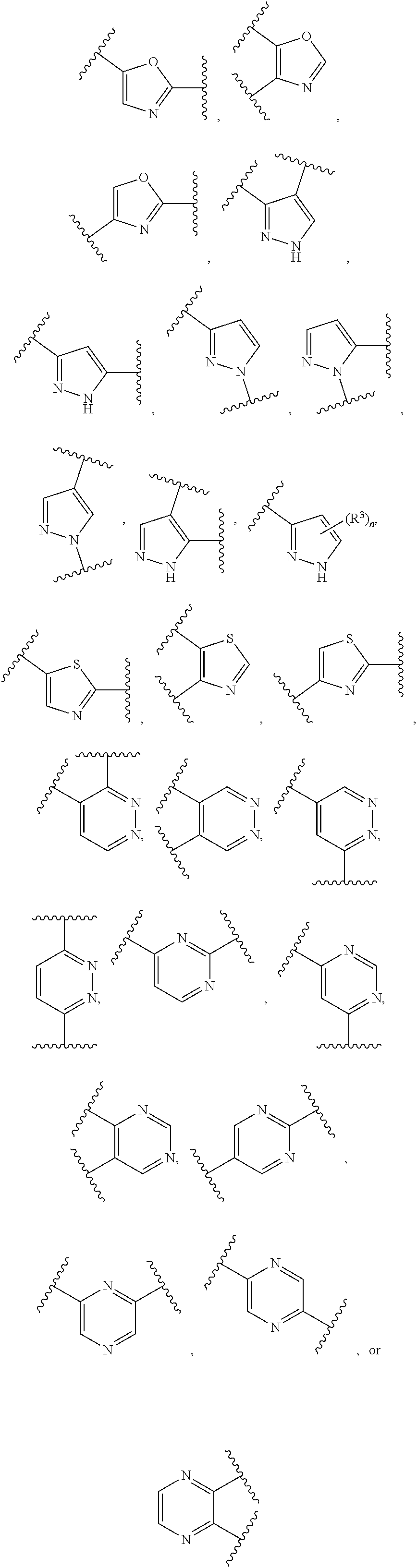

each optionally substituted with —OH or R¹;
W is a bond, —CH₂—, —C(CH₃)H—, —CH₂CH₂—, —CH₂CH₂CH₂—, or —CH₂CH(OH)CH₂—;
V is a bond, —CH₂CH₂—, —CH(CH₃)CH₂—, —CH(OH)CH₂—, —CH₂CH(CH₃)—, —CH₂CH₂CH₂—, or —CH₂CH(OH)CH₂—;
L is Cl;
R¹ and R² are each independently hydrogen, halogen, —CN, —CF₃, methyl, or —CONH₂;
R³ is selected from —S(C₁-C₃ alkyl), —SO₂(C₁-C₃ alkyl), —NH₂, —NHSO₂(C₁-C₃ alkyl), —NHSO₂CF₃, —N(CH₃)SO₂(C₁-C₃ alkyl), —CH₂NHSO₂(C₁-C₃ alkyl), —CH₂N(CH₃)SO₂(C₁-C₃ alkyl), —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);
n1 is 0, 1, or 2; and
n2 is 1 or 2.

In one embodiment of the pharmaceutical compositions or combinations or the methods of the present disclosure, the androgen receptor modulator has the structure of formula (X):

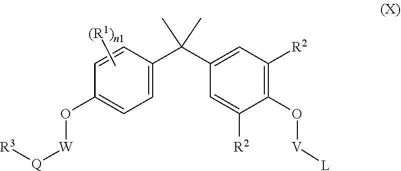

or a pharmaceutically acceptable salt, tautomer, stereoisomer or prodrug thereof, wherein:

Q is a —CH₂—, —CH₂CH₂—, —CH₂CH(OH)CH₂—,

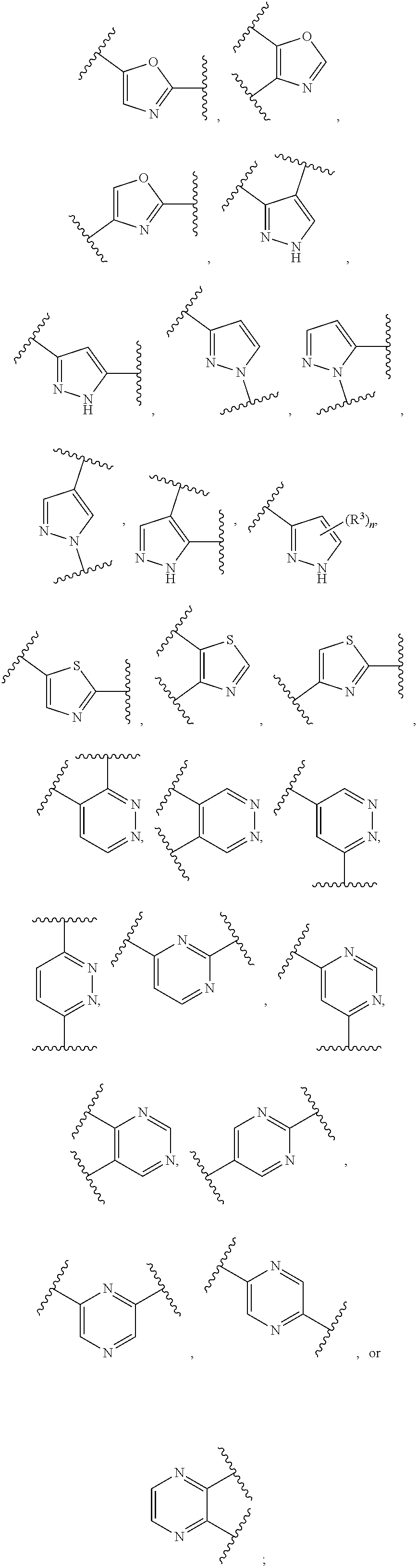

W is a bond, —CH₂—, —C(CH₃)H—, —CH₂CH₂—, —CH₂CH₂CH₂—, or —CH₂CH(OH)CH₂—;
V is a bond, —CH₂CH₂—, —CH(CH₃)CH₂—, —CH(OH)CH₂—, —CH₂CH(CH₃)—, —CH₂CH₂CH₂—, or —CH₂CH(OH)CH₂—;
L is Cl;
R¹ and R² are each independently hydrogen, halogen, —CN, or methyl;
R³ is selected from —SO₂(C₁-C₃ alkyl), —NHSO₂(C₁-C₃ alkyl), —NHSO₂CF₃, —N(CH₃)SO₂(C₁-C₃ alkyl), —CH₂NHSO₂(C₁-C₃ alkyl), —CH₂N(CH₃)SO₂(C₁-C₃ alkyl), or —SO₂NH₂; and
n1 is 0, 1, or 2.

In one embodiment of the compounds of formula (VI)-(X), R³ is —SO₂CH₃, —NHSO₂CH₃, —N(CH₃)SO₂CH₃, —CH₂NHSO₂CH₃, —CH₂N(CH₃)SO₂CH₃, or —SO₂NH₂. In one embodiment, R³ is —SO₂CH₃ or —NHSO₂CH₃.

In one embodiment of the compounds of formula (VI)-(X), Q is —CH₂—, —CH₂CH₂—, or —CH₂CH₂CH₂—, each optionally substituted with —OH, halogen, or C₁-C₃ alkyl. In one embodiment, Q is —CH₂CH₂CH₂—, optionally substituted with —OH, halogen, or C₁-C₃ alkyl. In one embodiment, Q is —CH₂CHOHCH₂—.

In one embodiment of the compounds of formula (VI)-(X), Q is

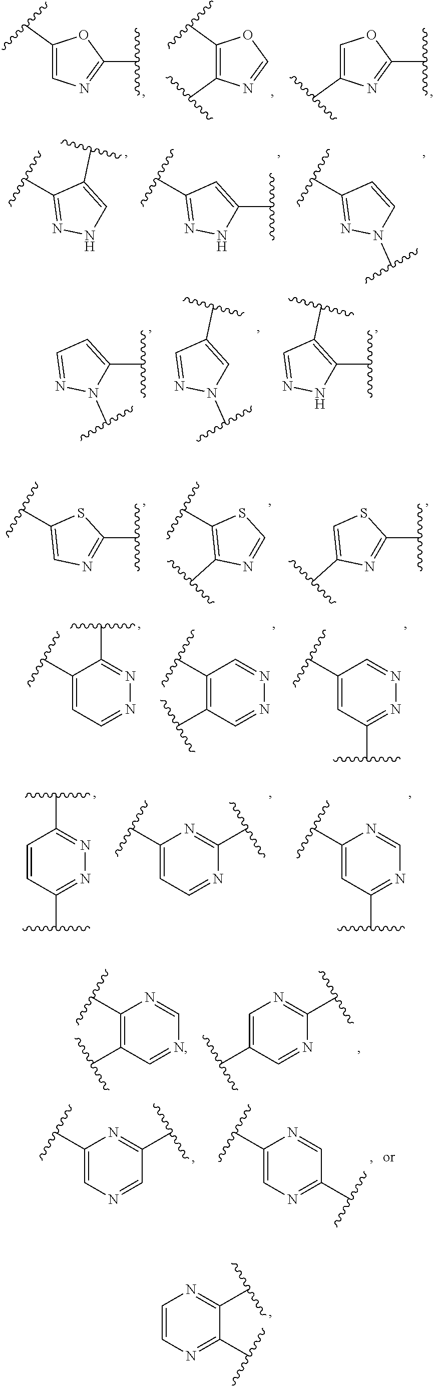

each optionally substituted with —OH, halogen, or C₁-C₃ alkyl. In one embodiment Q is

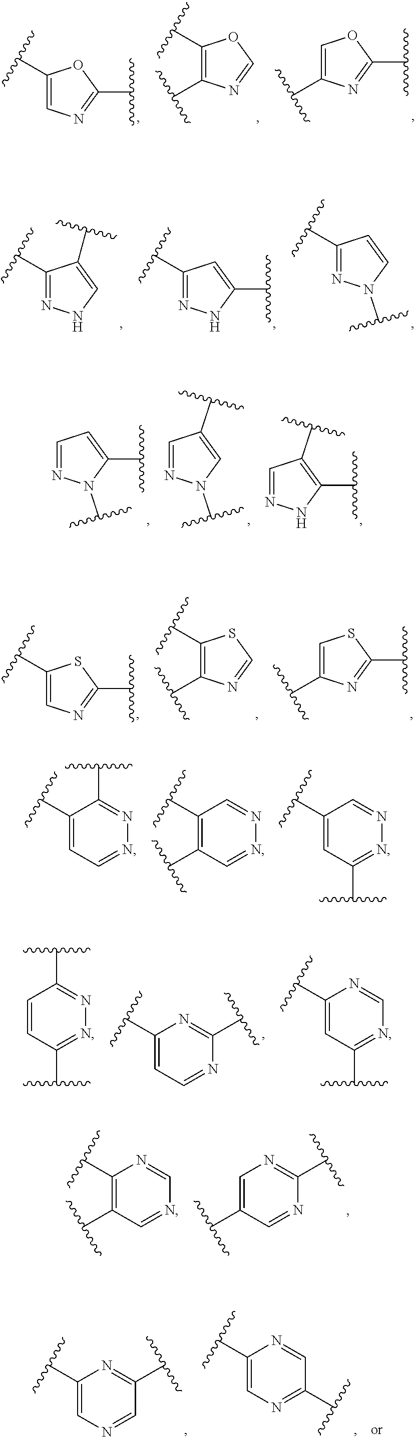

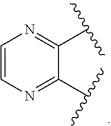 [SMILES: *c1nccnc1*]

In one embodiment of the compounds of formula (VI)-(X), Q is —CH₂CHOHCH₂—,

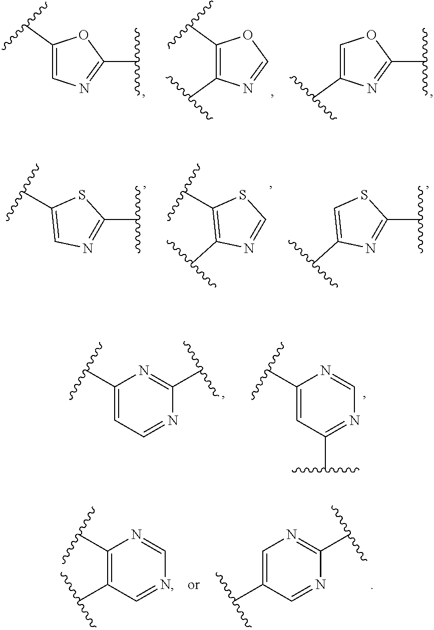

In one embodiment, Q is —CH₂CHOHCH₂—,

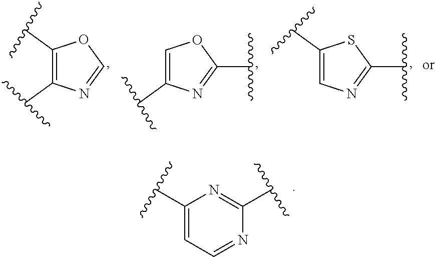 [SMILES: *c1ncoc1* ; *c1coc(*)n1 ; *c1cnc(*)s1 ; *c1ccnc(*)n1]

In one embodiment of the compounds of formula (VI)-(IX), n2 is 2 and R² is Cl, methyl, or —CN. In one embodiment, at least one R² is ortho to —O—V-L. In one embodiment, two R² is each ortho to —O—V-L. In one embodiment, R² is Cl or —CN.

In one embodiment of the compounds of formula (VI)-(IX), R² is Cl or —CN.

In one embodiment of the compounds of formula (VI)-(IX), n1 is 0.

In one embodiment of the compounds of formula (VI)-(IX), L is Cl.

In one embodiment of the compounds of formula (VI)-(IX), V is —CH₂CH₂—, —CH₂CH(OH)CH₂—, or —CH₂CH(OH)CH₂—.

In one embodiment of the compounds of formula (VI)-(IX), W is —CH₂— or —CH₂CH(OH)CH₂—.

In one embodiment of the compounds of formula (VI)-(VIII), X is —C(CH₃)₂—.

In one embodiment of the pharmaceutical compositions or combinations or the methods of the present disclosure, the androgen receptor modulator has the structure of formula (IIIA):

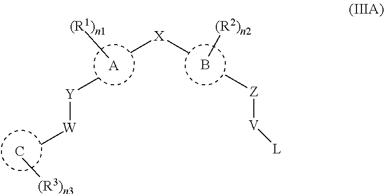

or a pharmaceutically acceptable salt, tautomer, stereoisomer or prodrug thereof, wherein:
A and B are each independently selected from phenyl, pyridyl, pyrimidyl, or thiophene;
C is a 3- to 10-membered ring;
X is a bond, —(CR⁵R⁶)ₜ—, or —NR⁷;
Y is a bond, —(CR⁸R⁹)ₘ—, —O—, —S—, —S(═O)—, —SO₂—, —NR⁷—, or —N(COCH₃)—;
W is a bond, —(CR^{8a}R^{9a})ₘ—, —C(═O)—, —N(R⁷)CO—, —CONR⁷—, or —NSO₂R⁷—;
Z is a bond, —CH₂—, —C(CH₃)H—, —O—, —S—, —NH—, —NCH₃—, or —N(COCH₃)—;
V is —CH₂— and L is halogen, —NH₂, —CHCl₂, —CCl₃, or —CF₃; or
V is —CH₂CH₂— and L is halogen or —NH₂;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;
R³ is selected from halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);
R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;
R⁷ is hydrogen, C₁-C₆ alkyl, C₂-C₆ alkenyl, or C₂-C₆ alkynyl;
R⁸ and R⁹ are each independently hydrogen, halogen, or C₁-C₃ alkyl;
R^{8a} and R^{9a} are each independently hydrogen, —OH, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵; or R^{8a} and R^{8b} taken together form an optionally substituted 3- to 6-membered carbocyclyl or heterocyclyl;
R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form a 3- to 6-membered heterocyclyl;
R¹⁶ is hydrogen, optionally substituted C₁-C₃ alkyl, optionally substituted C₂-C₃ alkenyl, optionally substituted C₂-C₃ alkynyl, C₃-C₆ cycloalky, or phenyl;
each m is independently 0, 1, or 2;
n1 and n2 are each independently 0, 1, or 2;
R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form a 3- to 6-membered heterocyclyl;
R¹⁶ is hydrogen, optionally substituted C₁-C₃ alkyl, optionally substituted C₂-C₃ alkenyl, optionally substituted C₂-C₃ alkynyl, C₃-C₆ cycloalky, or phenyl;
each m is independently 0, 1, or 2;
n1 and n2 are each independently 0, 1, or 2;
n3 is 1, 2, 3, 4 or 5; and
t is 0, 1 or 2.

In one embodiment of the pharmaceutical compositions or combinations or the methods of the present disclosure, the androgen receptor modulator has the structure of formula (IVA):

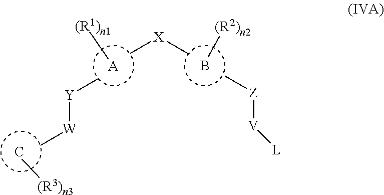

or a pharmaceutically acceptable salt, tautomer, stereoisomer or prodrug thereof, wherein:
A and B are each independently selected from phenyl, pyridyl, pyrimidyl, or thiophene;
C is a 3- to 10-membered ring;
X is a bond, —(CR⁵R⁶)ₜ—, or —NR⁷—;
Y and Z are each independently a bond, —CH₂—, —C(CH₃)H—, —O—, —S—, —NH—, —NCH₃—, or —N(COCH₃)—;
W is a bond, —CH₂—, —C(CH₃)H—, —C(═O)—, —N(R⁷)CO—, or —CONR⁷—;
V is —CH₂— and L is halogen, —NH₂, or —CF₃; or
V is —CH₂CH₂— and L is halogen or —NH₂;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;
R³ is selected from halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);
R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;
R⁷ is H or C₁-C₆ alkyl;
R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form a 3- to 6-membered heterocyclyl;
R¹⁶ is hydrogen, C₁-C₃ alkyl, C₂-C₃ alkenyl, or C₂-C₃ alkynyl;
n1 and n2 are each independently 0, 1, or 2;
n3 is 1, 2, 3, 4 or 5; and
t is 0, 1 or 2.

In one embodiment of the compound of formula (IVA), C is 5- to 10-membered heteroaryl or aryl. In some embodiments, C is 5- to 7-membered heteroaryl comprising 1, 2, or 3 heteroatoms selected from O, S, or N as a ring member. In some embodiments, C, which is substituted with (R³)n3, is pyrazole, imidazole, oxazole, oxadiazole, oxazolone, isoxazole, thiazole, pyridyl, pyrazine, furan or pyrimidyl. In some embodiments, C, which is substituted with (R³)n3, is selected from

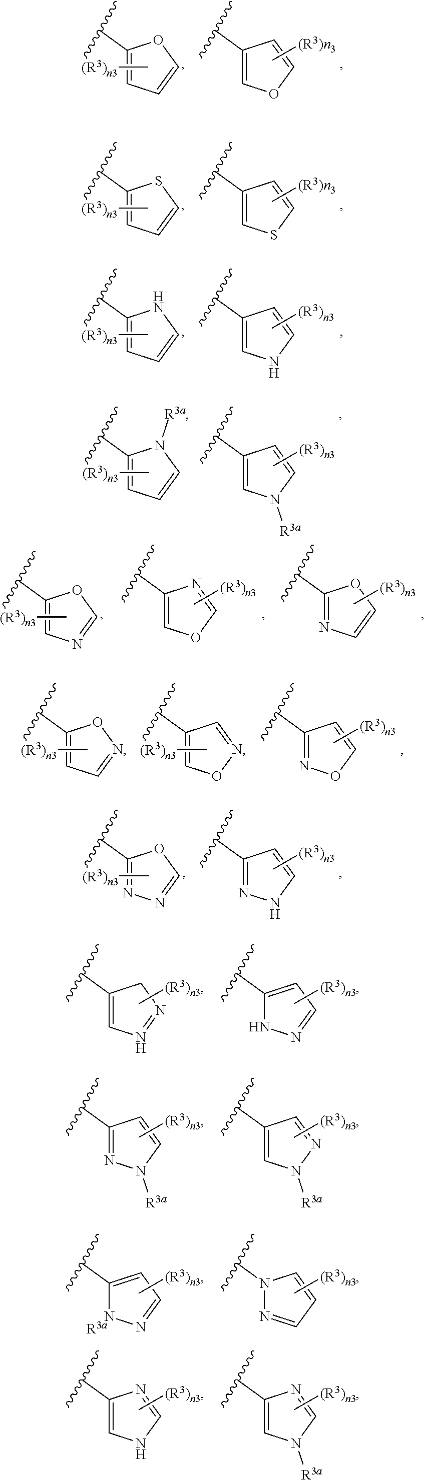

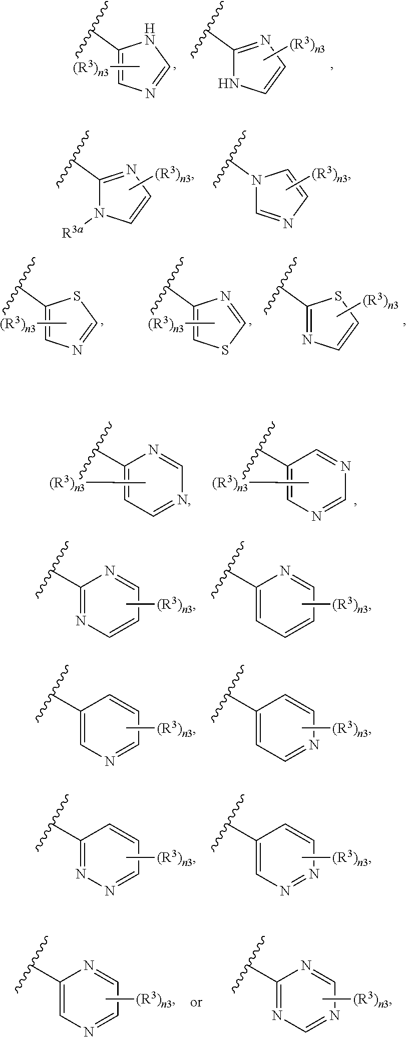

wherein R^{3a} is C₁-C₃ alkyl.

In one embodiment of the compound of formula (IVA), R¹ and R² are each independently Cl, —CN, —CF₃, —OH, methyl, methoxy, or —CONH₂.

In one embodiment of the compound of formula (IVA),
A and B are phenyl;
X is —(CR⁵R⁶)ₜ—;
Y and Z are each —O—;
V is —CH₂— or —CH₂CH₂—;
L is halogen;
R¹ and R² are each independently hydrogen, halogen, —CN, —CF₃, —OH, or optionally substituted C₁-C₆ alkyl;
R⁵ and R⁶ are each independently hydrogen, halogen, —OH, or C₁-C₃ alkyl; and
R¹⁶ is hydrogen, C₁-C₃ alkyl, C₂-C₃ alkenyl, or C₂-C₃ alkynyl.

In one embodiment of the compound of formula (IVA),
A and B are phenyl;
X is —(CR⁵R⁶)ₜ—;
W is —CH₂— or —C(CH₃)H—;
Y and Z are each —O—;
V is —CH₂CH₂—;
L is halogen;
R¹ and R² are each independently hydrogen, halogen, or —CN;
R⁵ and R⁶ are each independently hydrogen, or C₁-C₃ alkyl; and
R¹⁶ is hydrogen, C₁-C₃ alkyl, C₂-C₃ alkenyl, or C₂-C₃ alkynyl.

In one embodiment of the pharmaceutical compositions or combinations or the methods of the present disclosure, the androgen receptor modulator has the structure of formula (A-I):

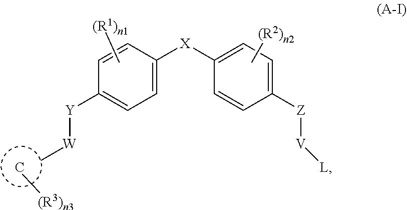

or a pharmaceutically acceptable salt, tautomer, stereoisomer or prodrug thereof, wherein:
C is a phenyl or a 5- to 7-membered monocyclic heteroaryl comprising 1, 2, or 3 heteroatoms selected from O, S, or N as a ring member;
X is a bond, —(CR⁵R⁶)ₜ—, or —NR⁷—;
Y is a bond, —CH₂—, —C(CH₃)H—, —O—, —S—, —NH—, —NCH₃—, or —N(COCH₃)—;
Z is a bond, —CH₂—, —O—, or —NH—;
W is a bond, —CH₂—, —C(CH₃)H—, —C(═O)—, —N(R⁷)CO—, or —CONR⁷—;
V is —CH₂— and L is halogen, —NH₂, or —CF₃; or
V is —CH₂CH₂— and L is halogen or —NH₂;
R¹ and R² are each independently hydrogen, halogen, —CN, —CF₃, methyl, or —CONH₂;
R³ is selected from from —CN, C₁-C₃ alkoxy, —CF₃, C₁-C₃ alkyl, C₂-C₃ alkenyl, C₂-C₃ alkynyl, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);
R⁵ and R⁶ are each independently hydrogen, halogen, —OH, or C₁-C₃ alkyl;
R⁷ is H or C₁-C₆ alkyl;
n1 and n2 are each independently 0, 1, or 2;
n3 is 1, 2, 3, 4 or 5; and
t is 0, 1 or 2.

In one embodiment of the compound of formula (A-I), at least one R³ is selected from —CN, C₁-C₃ alkoxy, —CONH₂, —NHSO₂CH₃, —N(CH₃)SO₂CH₃, —NHSO₂CH₂CH₃, —N(CH₃)SO₂CH₂CH₃, or —SO₂CH₃ and the other R³, if present, is selected from —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl).

In one embodiment of the compound of formula (A-I),
X is a bond or —(CR⁵R⁶)ₜ;
W is a bond, —CH₂—, or —C(CH₃)H—;
Y is —O—;
Z is —O—;
V is —CH₂— or —CH₂CH₂—; and
L is halogen

In one embodiment of the pharmaceutical compositions or combinations or the methods of the present disclosure, the androgen receptor modulator has the structure of formula (G-II):

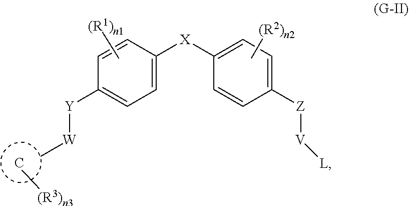

or a pharmaceutically acceptable salt, tautomer, stereoisomer or prodrug thereof, wherein:
C is

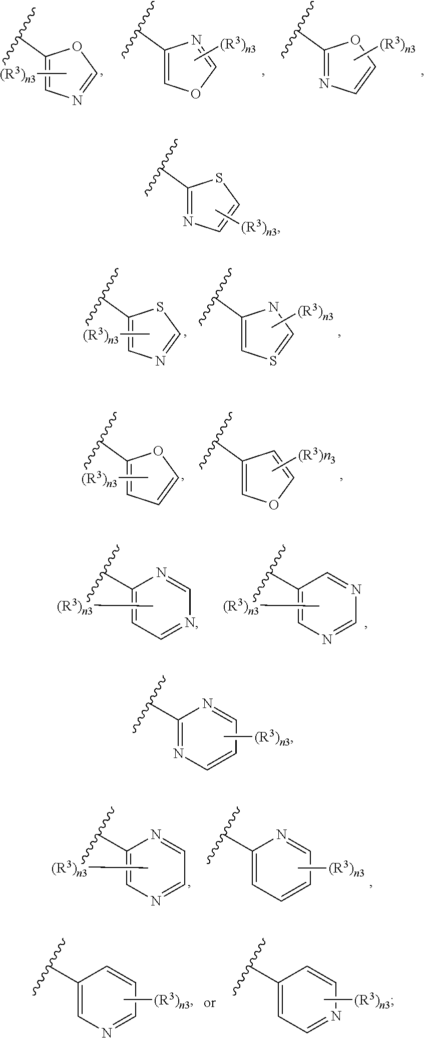

X is —(CR⁵R⁶)ₜ—;
Y is —O—;
Z is —O—;
W is —CH₂— or —C(CH₃)H—;
V is —CH₂CH₂—;
L is halogen;
R¹ and R² are each independently Cl or —CN;
at least one R³ is selected from —CN, C₁-C₃ alkoxy, —CONH₂, —NHSO₂CH₃, —N(CH₃)SO₂CH₃, —NHSO₂CH₂CH₃, —N(CH₃)SO₂CH₂CH₃, or —SO₂CH₃ and the other R³, if present, is selected from —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);
R⁵ and R⁶ are each independently hydrogen or methyl;
n1 and n2 are each independently 0, 1, or 2;
n3 is 1 or 2; and
t is 1.

In one embodiment of the compound of formula (G-II), at least one R³ is selected from —NHSO₂CH₃, —NHSO₂CH₂CH₃, or —SO₂CH₃ and the other R³, if present, is selected from —CN, C₁-C₃ alkyl, C₁-C₃ alkoxy, —SO₂(C₁-C₃ alkyl), —NH₂, —(C₁-C₃ alkyl)NH₂, —NHSO₂CH₃, —N(CH₃)SO₂CH₃, —NHSO₂CH₂CH₃, —N(CH₃)SO₂CH₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl).

In one embodiment of the compounds of formula (I)-(III), (IIIA), (VI), (IVA), (V)-(X), (A-I), or (G-II), the compound is selected from Table A below, or a pharmaceutically acceptable salt, tautomer, stereoisomer or prodrug thereof. In one embodiment of the compounds of formula (I)-(III), (IIIA), (VI), (IVA), (V)-(X), (A-I), or (G-II), the compound is selected from Compounds A1-A186, or a pharmaceutically acceptable salt, tautomer, stereoisomer or prodrug thereof. In one embodiment of the compounds of formula (I)-(III), (IIIA), (VI), (IVA), (V)-(X), (A-I), or (G-II), the compound is selected from Compounds A187-A211, or a pharmaceutically acceptable salt, tautomer, stereoisomer or prodrug thereof. In one embodiment of the compounds of formula (I)-(III), (IIIA), (VI), (IVA), (V)-(X), (A-I), or (G-II), the compound is selected from Compounds A1-A211 or a pharmaceutically acceptable salt, tautomer, stereoisomer or prodrug thereof.

The compounds disclosed in PCT/US2019/057034 can be useful compounds for the present invention. The disclosure of PCT/US2019/057034 is incorporated by reference in its entirety for all purposes.

TABLE A
Androgen Receptor Modulators

| Compound | |
|---|---|
| ID | Structure |
| A1 | 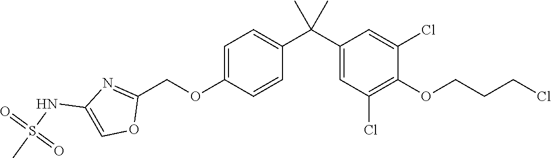 [SMILES: CC(C)(c1ccc(OCc2nc(NS(C)(=O)=O)co2)cc1)c1cc(Cl)c(OCCCCl)c(Cl)c1] |
| A2 | 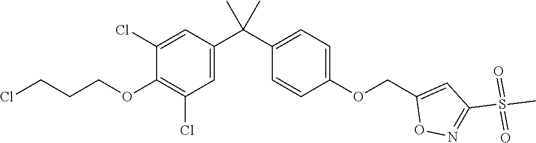 [SMILES: CC(C)(c1ccc(OCc2cc(S(C)(=O)=O)no2)cc1)c1cc(Cl)c(OCCCCl)c(Cl)c1] |
| A3 | 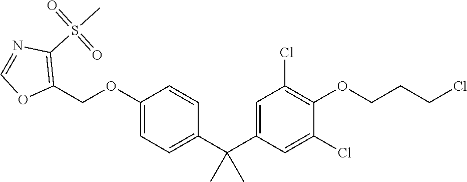 [SMILES: CC(C)(c1ccc(OCc2ocnc2S(C)(=O)=O)cc1)c1cc(Cl)c(OCCCCl)c(Cl)c1] |
| A4 | 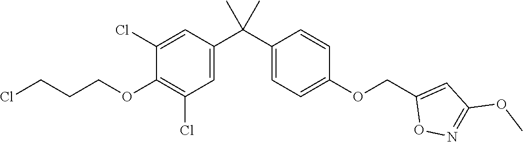 [SMILES: COc1cc(COc2ccc(C(C)(C)c3cc(Cl)c(OCCCCl)c(Cl)c3)cc2)on1] |
| A5 | 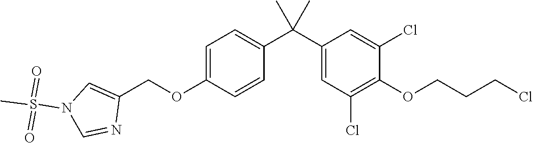 [SMILES: CC(C)(c1ccc(OCc2cn(S(C)(=O)=O)cn2)cc1)c1cc(Cl)c(OCCCCl)c(Cl)c1] |
| A6 | 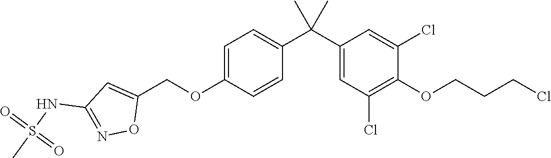 [SMILES: CC(C)(c1ccc(OCc2cc(NS(C)(=O)=O)no2)cc1)c1cc(Cl)c(OCCCCl)c(Cl)c1] |
| A7 | 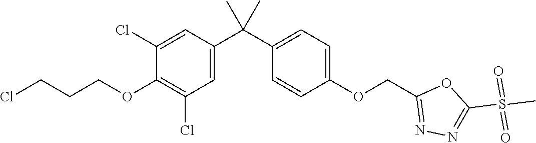 [SMILES: CC(C)(c1ccc(OCc2nnc(S(C)(=O)=O)o2)cc1)c1cc(Cl)c(OCCCCl)c(Cl)c1] |
| A8 | 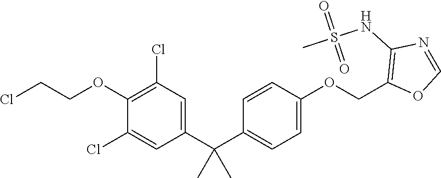 [SMILES: CC(C)(c1ccc(OCc2ocnc2NS(C)(=O)=O)cc1)c1cc(Cl)c(OCCCl)c(Cl)c1] |
| A9 | 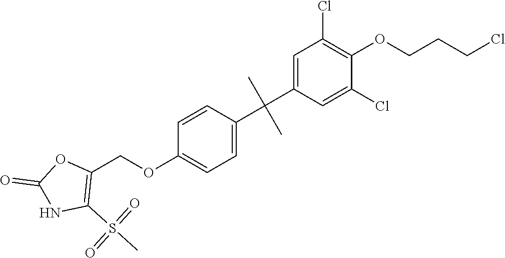 [SMILES: CC(C)(c1ccc(OCc2oc(=O)[nH]c2S(C)(=O)=O)cc1)c1cc(Cl)c(OCCCCl)c(Cl)c1] |
| A10 | 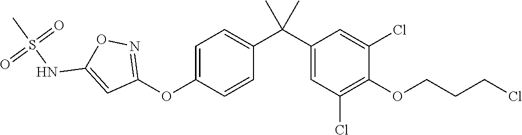 [SMILES: CC(C)(c1ccc(Oc2cc(NS(C)(=O)=O)on2)cc1)c1cc(Cl)c(OCCCCl)c(Cl)c1] |
| A11 | 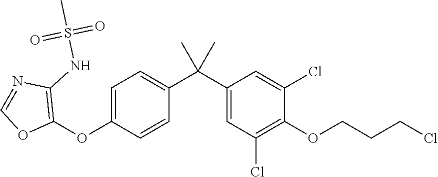 [SMILES: CC(C)(c1ccc(Oc2ocnc2NS(C)(=O)=O)cc1)c1cc(Cl)c(OCCCCl)c(Cl)c1] |
| A12 | 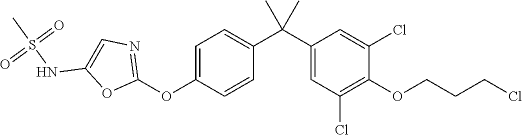 [SMILES: CC(C)(c1ccc(Oc2ncc(NS(C)(=O)=O)o2)cc1)c1cc(Cl)c(OCCCCl)c(Cl)c1] |
| A13 | 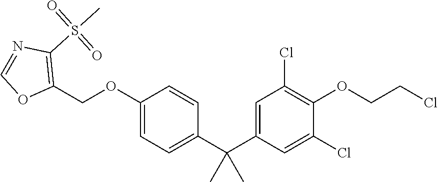 [SMILES: CC(C)(c1ccc(OCc2ocnc2S(C)(=O)=O)cc1)c1cc(Cl)c(OCCCl)c(Cl)c1] |
| A14 | 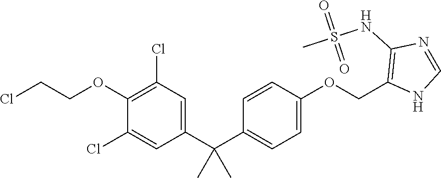 [SMILES: CC(C)(c1ccc(OCc2[nH]cnc2NS(C)(=O)=O)cc1)c1cc(Cl)c(OCCCl)c(Cl)c1] |
| A15 | 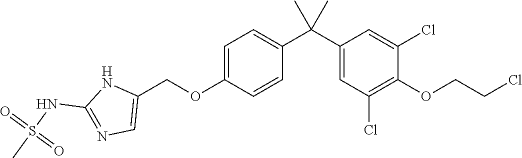 [SMILES: CC(C)(c1ccc(OCc2cnc(NS(C)(=O)=O)[nH]2)cc1)c1cc(Cl)c(OCCCl)c(Cl)c1] |
| A16 | 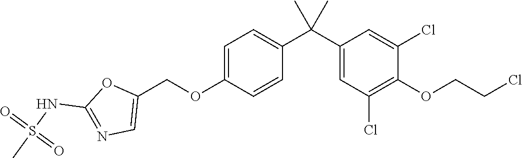 [SMILES: CC(C)(c1ccc(OCc2cnc(NS(C)(=O)=O)o2)cc1)c1cc(Cl)c(OCCCl)c(Cl)c1] |
| A17 | 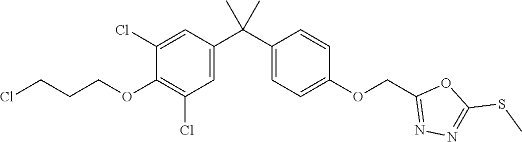 [SMILES: CSc1nnc(COc2ccc(C(C)(C)c3cc(Cl)c(OCCCCl)c(Cl)c3)cc2)o1] |
| A18 | 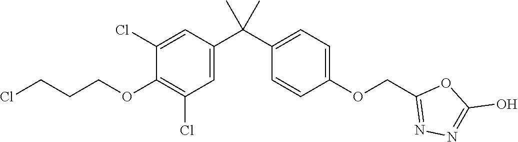 [SMILES: CC(C)(c1ccc(OCc2nnc(O)o2)cc1)c1cc(Cl)c(OCCCCl)c(Cl)c1] |
| A19 | 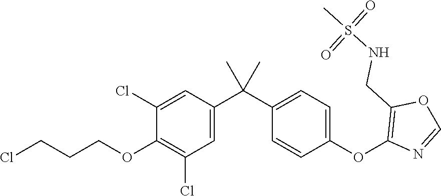 [SMILES: CC(C)(c1ccc(Oc2ncoc2CNS(C)(=O)=O)cc1)c1cc(Cl)c(OCCCCl)c(Cl)c1] |
| A20 | 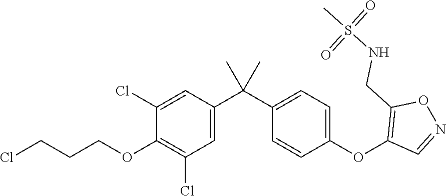 [SMILES: CC(C)(c1ccc(Oc2cnoc2CNS(C)(=O)=O)cc1)c1cc(Cl)c(OCCCCl)c(Cl)c1] |
| A21 | 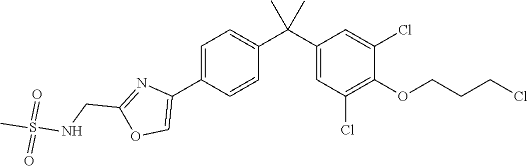 [SMILES: CC(C)(c1ccc(-c2coc(CNS(C)(=O)=O)n2)cc1)c1cc(Cl)c(OCCCCl)c(Cl)c1] |
| A22 | 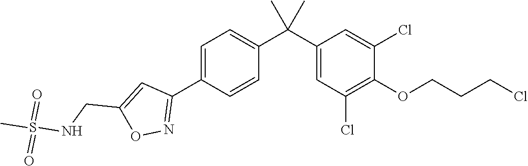 [SMILES: CC(C)(c1ccc(-c2cc(CNS(C)(=O)=O)on2)cc1)c1cc(Cl)c(OCCCCl)c(Cl)c1] |
| A23 | 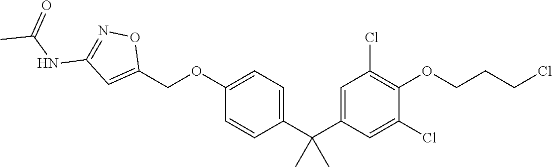 [SMILES: CC(=O)Nc1cc(COc2ccc(C(C)(C)c3cc(Cl)c(OCCCCl)c(Cl)c3)cc2)on1] |
| A24 | 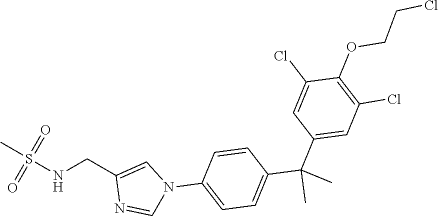 [SMILES: CC(C)(c1ccc(-n2cnc(CNS(C)(=O)=O)c2)cc1)c1cc(Cl)c(OCCCl)c(Cl)c1] |
| A25 | 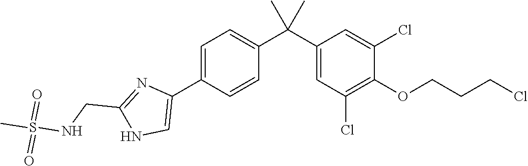 [SMILES: CC(C)(c1ccc(-c2c[nH]c(CNS(C)(=O)=O)n2)cc1)c1cc(Cl)c(OCCCCl)c(Cl)c1] |
| A26 | 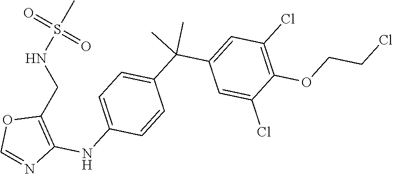 [SMILES: CC(C)(c1ccc(Nc2ncoc2CNS(C)(=O)=O)cc1)c1cc(Cl)c(OCCCl)c(Cl)c1] |
| A27 | 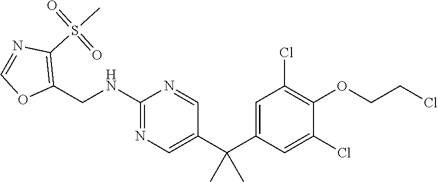 [SMILES: CC(C)(c1cnc(NCc2ocnc2S(C)(=O)=O)nc1)c1cc(Cl)c(OCCCl)c(Cl)c1] |
| A28 | 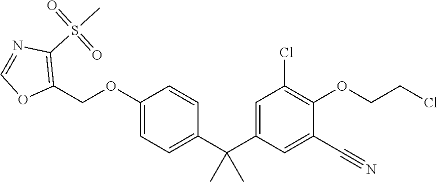 [SMILES: CC(C)(c1ccc(OCc2ocnc2S(C)(=O)=O)cc1)c1cc(Cl)c(OCCCl)c(C#N)c1] |
| A29 | 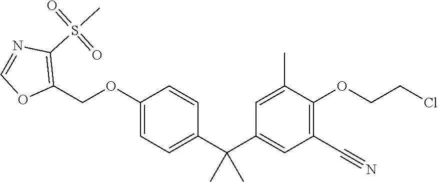 [SMILES: Cc1cc(C(C)(C)c2ccc(OCc3ocnc3S(C)(=O)=O)cc2)cc(C#N)c1OCCCl] |
| A30 | 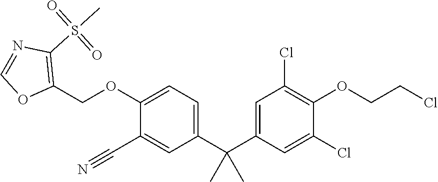 [SMILES: CC(C)(c1cc(Cl)c(OCCCl)c(Cl)c1)c1ccc(OCc2ocnc2S(C)(=O)=O)c(C#N)c1] |
| A31 | 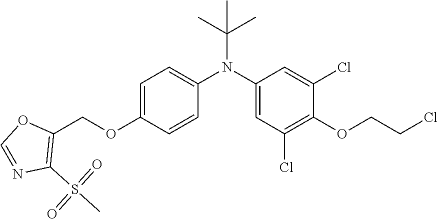 [SMILES: CC(C)(C)N(c1ccc(OCc2ocnc2S(C)(=O)=O)cc1)c1cc(Cl)c(OCCCl)c(Cl)c1] |
| A32 | 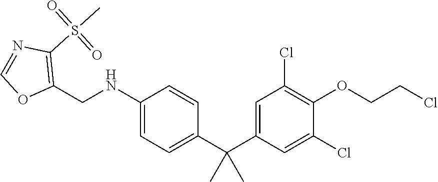 [SMILES: CC(C)(c1ccc(NCc2ocnc2S(C)(=O)=O)cc1)c1cc(Cl)c(OCCCl)c(Cl)c1] |
| A33 | 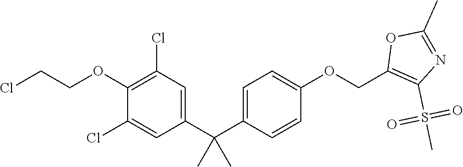 [SMILES: Cc1nc(S(C)(=O)=O)c(COc2ccc(C(C)(C)c3cc(Cl)c(OCCCl)c(Cl)c3)cc2)o1] |
| A34 | 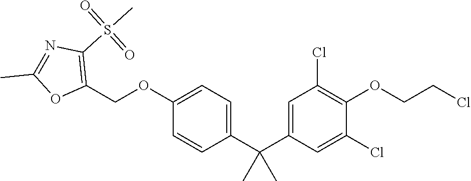 [SMILES: Cc1nc(S(C)(=O)=O)c(COc2ccc(C(C)(C)c3cc(Cl)c(OCCCl)c(Cl)c3)cc2)o1] |
| A35 | 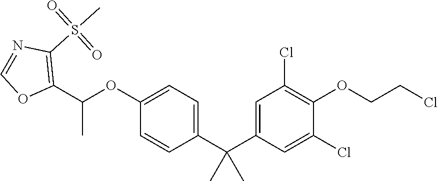 [SMILES: CC(Oc1ccc(C(C)(C)c2cc(Cl)c(OCCCl)c(Cl)c2)cc1)c1ocnc1S(C)(=O)=O] |
| A36 | 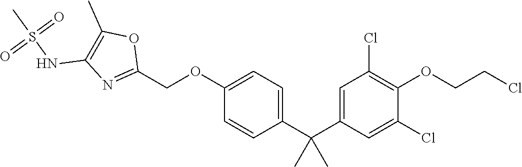 [SMILES: Cc1oc(COc2ccc(C(C)(C)c3cc(Cl)c(OCCCl)c(Cl)c3)cc2)nc1NS(C)(=O)=O] |
| A37 | 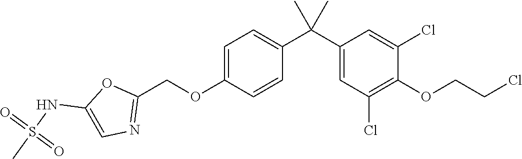 [SMILES: CC(C)(c1ccc(OCc2ncc(NS(C)(=O)=O)o2)cc1)c1cc(Cl)c(OCCCl)c(Cl)c1] |
| A38 | 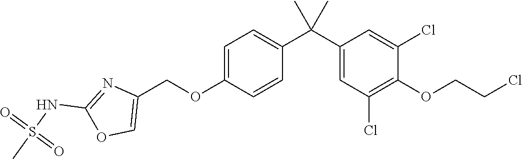 [SMILES: CC(C)(c1ccc(OCc2coc(NS(C)(=O)=O)n2)cc1)c1cc(Cl)c(OCCCl)c(Cl)c1] |
| A39 | 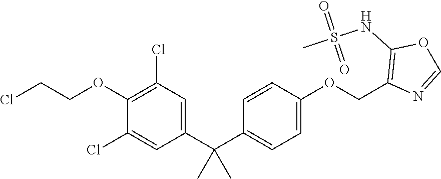 [SMILES: CC(C)(c1ccc(OCc2ncoc2NS(C)(=O)=O)cc1)c1cc(Cl)c(OCCCl)c(Cl)c1] |
| A40 | 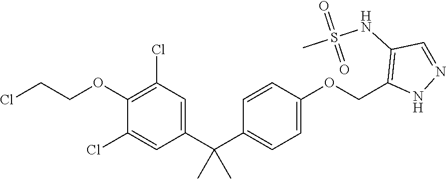 [SMILES: CC(C)(c1ccc(OCc2[nH]ncc2NS(C)(=O)=O)cc1)c1cc(Cl)c(OCCCl)c(Cl)c1] |
| A41 | 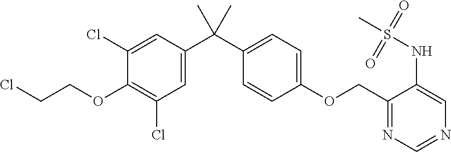 [SMILES: CC(C)(c1ccc(OCc2ncncc2NS(C)(=O)=O)cc1)c1cc(Cl)c(OCCCl)c(Cl)c1] |
| A42 | 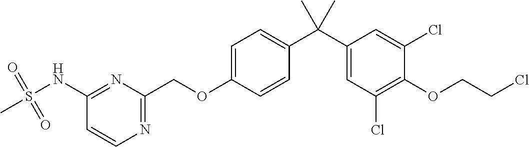 [SMILES: CC(C)(c1ccc(OCc2nccc(NS(C)(=O)=O)n2)cc1)c1cc(Cl)c(OCCCl)c(Cl)c1] |
| A43 | 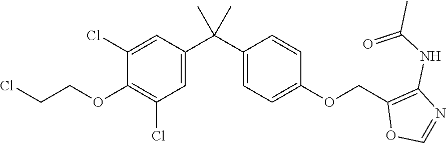 [SMILES: CC(=O)Nc1ncoc1COc1ccc(C(C)(C)c2cc(Cl)c(OCCCl)c(Cl)c2)cc1] |
| A44 | 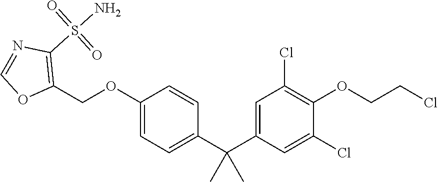 [SMILES: CC(C)(c1ccc(OCc2ocnc2S(N)(=O)=O)cc1)c1cc(Cl)c(OCCCl)c(Cl)c1] |
| A45 | 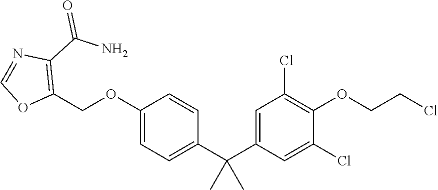 [SMILES: CC(C)(c1ccc(OCc2ocnc2C(N)=O)cc1)c1cc(Cl)c(OCCCl)c(Cl)c1] |
| A46 | 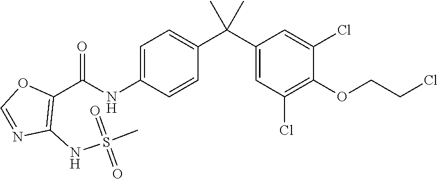 [SMILES: CC(C)(c1ccc(NC(=O)c2ocnc2NS(C)(=O)=O)cc1)c1cc(Cl)c(OCCCl)c(Cl)c1] |
| A47 | 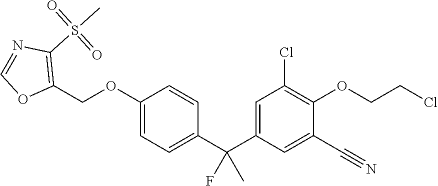 [SMILES: CC(F)(c1ccc(OCc2ocnc2S(C)(=O)=O)cc1)c1cc(Cl)c(OCCCl)c(C#N)c1] |
| A48 | 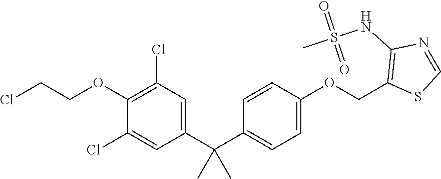 [SMILES: CC(C)(c1ccc(OCc2scnc2NS(C)(=O)=O)cc1)c1cc(Cl)c(OCCCl)c(Cl)c1] |
| A49 | 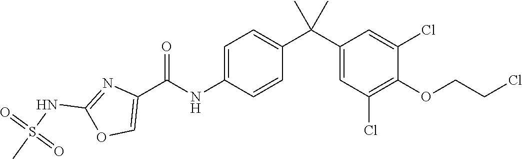 [SMILES: CC(C)(c1ccc(NC(=O)c2coc(NS(C)(=O)=O)n2)cc1)c1cc(Cl)c(OCCCl)c(Cl)c1] |
| A50 | 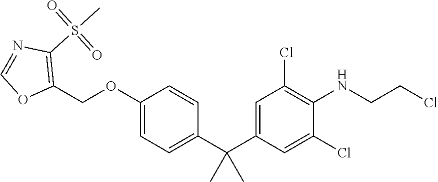 [SMILES: CC(C)(c1ccc(OCc2ocnc2S(C)(=O)=O)cc1)c1cc(Cl)c(NCCCl)c(Cl)c1] |
| A51 | 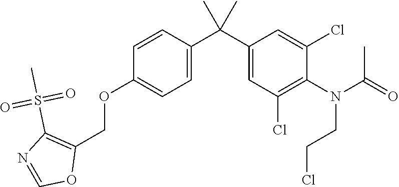 [SMILES: CC(=O)N(CCCl)c1c(Cl)cc(C(C)(C)c2ccc(OCc3ocnc3S(C)(=O)=O)cc2)cc1Cl] |
| A52 | 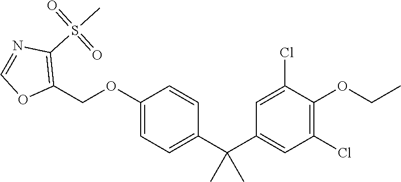 [SMILES: CCOc1c(Cl)cc(C(C)(C)c2ccc(OCc3ocnc3S(C)(=O)=O)cc2)cc1Cl] |
| A53 | 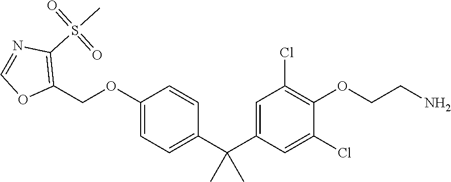 [SMILES: CC(C)(c1ccc(OCc2ocnc2S(C)(=O)=O)cc1)c1cc(Cl)c(OCCN)c(Cl)c1] |
| A54 | 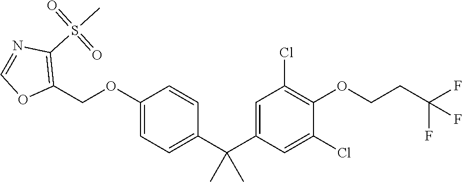 [SMILES: CC(C)(c1ccc(OCc2ocnc2S(C)(=O)=O)cc1)c1cc(Cl)c(OCCC(F)(F)F)c(Cl)c1] |
| A55 | 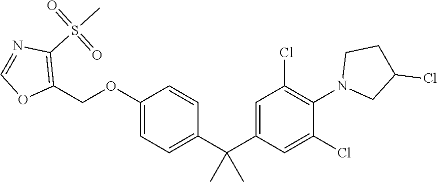 [SMILES: CC(C)(c1ccc(OCc2ocnc2S(C)(=O)=O)cc1)c1cc(Cl)c(N2CCC(Cl)C2)c(Cl)c1] |
| A56 | 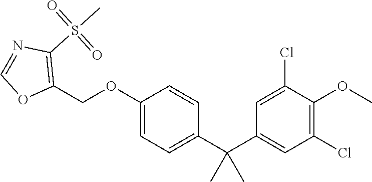 [SMILES: COc1c(Cl)cc(C(C)(C)c2ccc(OCc3ocnc3S(C)(=O)=O)cc2)cc1Cl] |
| A57 | 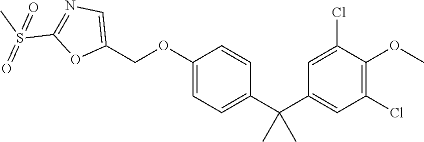 |
| A58 | 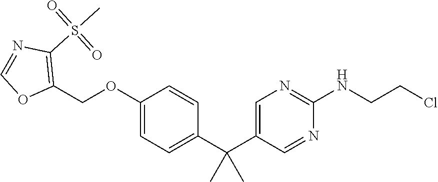 [SMILES: CC(C)(c1ccc(OCc2ocnc2S(C)(=O)=O)cc1)c1cnc(NCCCl)nc1] |
| A59 | 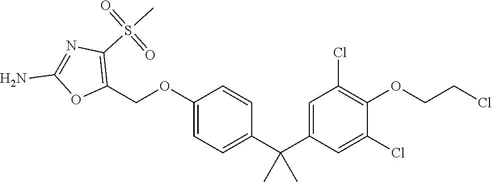 [SMILES: CC(C)(c1ccc(OCc2oc(N)nc2S(C)(=O)=O)cc1)c1cc(Cl)c(OCCCl)c(Cl)c1] |
| A60 | 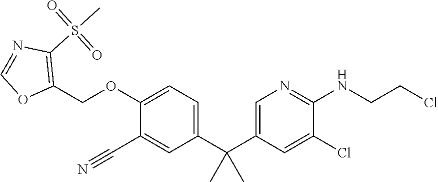 [SMILES: CC(C)(c1cnc(NCCCl)c(Cl)c1)c1ccc(OCc2ocnc2S(C)(=O)=O)c(C#N)c1] |
| A61 | 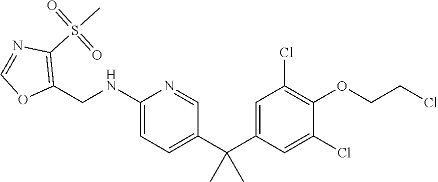 [SMILES: CC(C)(c1ccc(NCc2ocnc2S(C)(=O)=O)nc1)c1cc(Cl)c(OCCCl)c(Cl)c1] |
| A62 | 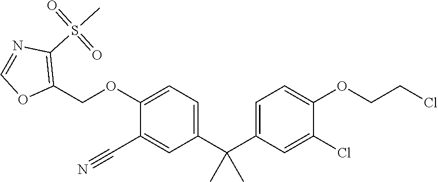 [SMILES: CC(C)(c1ccc(OCCCl)c(Cl)c1)c1ccc(OCc2ocnc2S(C)(=O)=O)c(C#N)c1] |
| A63 | 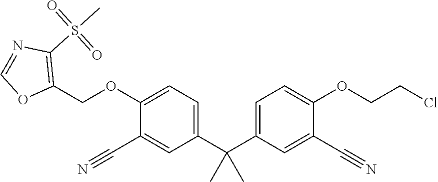 [SMILES: CC(C)(c1ccc(OCCCl)c(C#N)c1)c1ccc(OCc2ocnc2S(C)(=O)=O)c(C#N)c1] |
| A64 | 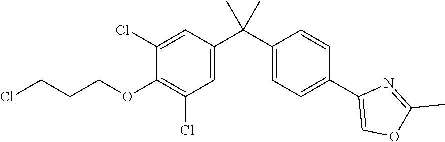 [SMILES: Cc1nc(-c2ccc(C(C)(C)c3cc(Cl)c(OCCCCl)c(Cl)c3)cc2)co1] |
| A65 | 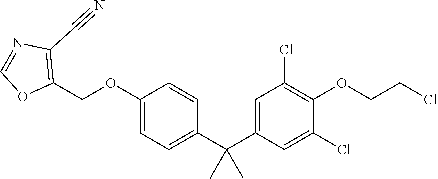 [SMILES: CC(C)(c1ccc(OCc2ocnc2C#N)cc1)c1cc(Cl)c(OCCCl)c(Cl)c1] |
| A66 | 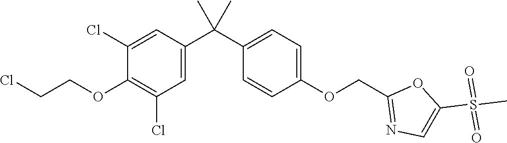 [SMILES: CC(C)(c1ccc(OCc2ncc(S(C)(=O)=O)o2)cc1)c1cc(Cl)c(OCCCl)c(Cl)c1] |
| A67 | 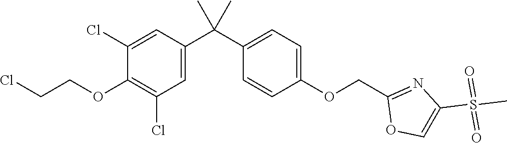 [SMILES: CC(C)(c1ccc(OCc2nc(S(C)(=O)=O)co2)cc1)c1cc(Cl)c(OCCCl)c(Cl)c1] |
| A68 | 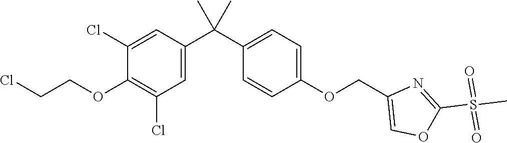 [SMILES: CC(C)(c1ccc(OCc2coc(S(C)(=O)=O)n2)cc1)c1cc(Cl)c(OCCCl)c(Cl)c1] |
| A69 | 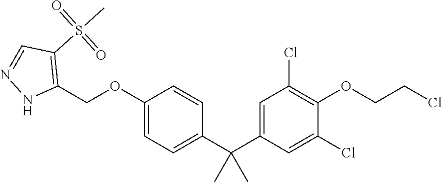 [SMILES: CC(C)(c1ccc(OCc2[nH]ncc2S(C)(=O)=O)cc1)c1cc(Cl)c(OCCCl)c(Cl)c1] |
| A70 | 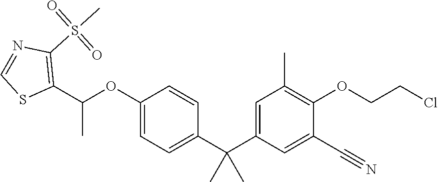 [SMILES: Cc1cc(C(C)(C)c2ccc(OC(C)c3scnc3S(C)(=O)=O)cc2)cc(C#N)c1OCCCl] |
| A71 | 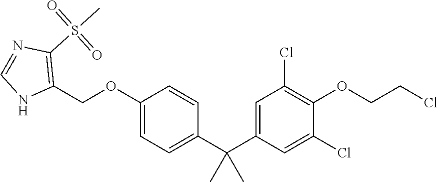 [SMILES: CC(C)(c1ccc(OCc2[nH]cnc2S(C)(=O)=O)cc1)c1cc(Cl)c(OCCCl)c(Cl)c1] |
| A72 | 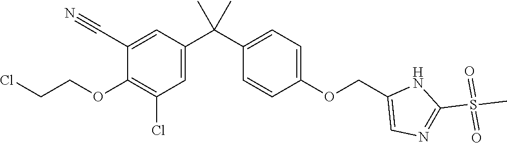 [SMILES: CC(C)(c1ccc(OCc2cnc(S(C)(=O)=O)[nH]2)cc1)c1cc(Cl)c(OCCCl)c(C#N)c1] |
| A73 | 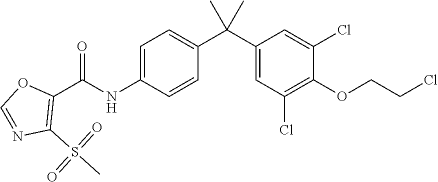 [SMILES: CC(C)(c1ccc(NC(=O)c2ocnc2S(C)(=O)=O)cc1)c1cc(Cl)c(OCCCl)c(Cl)c1] |
| A74 | 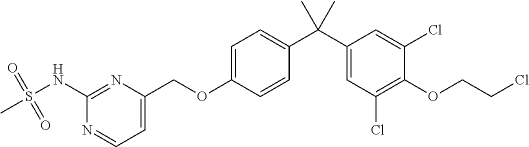 [SMILES: CC(C)(c1ccc(OCc2ccnc(NS(C)(=O)=O)n2)cc1)c1cc(Cl)c(OCCCl)c(Cl)c1] |
| A75 | 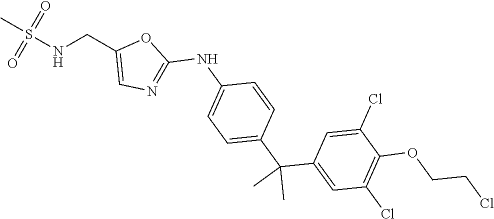 [SMILES: CC(C)(c1ccc(Nc2ncc(CNS(C)(=O)=O)o2)cc1)c1cc(Cl)c(OCCCl)c(Cl)c1] |
| A76 | 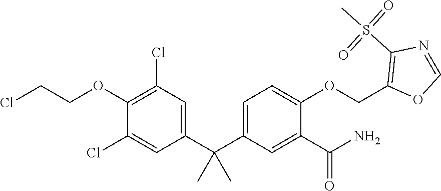 [SMILES: CC(C)(c1cc(Cl)c(OCCCl)c(Cl)c1)c1ccc(OCc2ocnc2S(C)(=O)=O)c(C(N)=O)c1] |
| A77 | 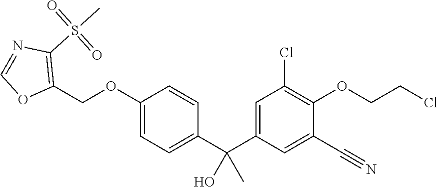 [SMILES: CC(O)(c1ccc(OCc2ocnc2S(C)(=O)=O)cc1)c1cc(Cl)c(OCCCl)c(C#N)c1] |
| A78 | 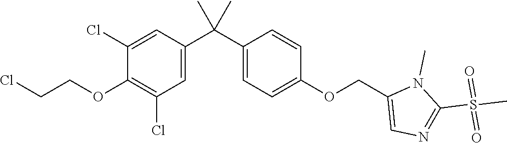 [SMILES: Cn1c(COc2ccc(C(C)(C)c3cc(Cl)c(OCCCl)c(Cl)c3)cc2)cnc1S(C)(=O)=O] |
| A79 | 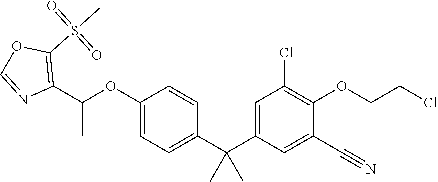 [SMILES: CC(Oc1ccc(C(C)(C)c2cc(Cl)c(OCCCl)c(C#N)c2)cc1)c1ncoc1S(C)(=O)=O] |
| A80 | 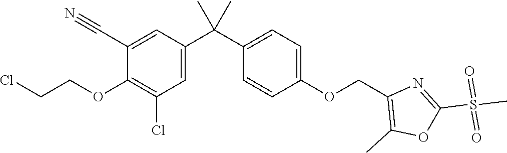 [SMILES: Cc1oc(S(C)(=O)=O)nc1COc1ccc(C(C)(C)c2cc(Cl)c(OCCCl)c(C#N)c2)cc1] |
| A81 | 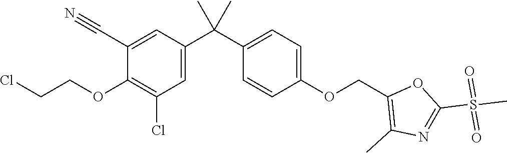 [SMILES: Cc1nc(S(C)(=O)=O)oc1COc1ccc(C(C)(C)c2cc(Cl)c(OCCCl)c(C#N)c2)cc1] |
| A82 | 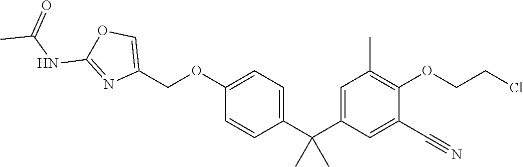 [SMILES: CC(=O)Nc1nc(COc2ccc(C(C)(C)c3cc(C)c(OCCCl)c(C#N)c3)cc2)co1] |
| A83 | 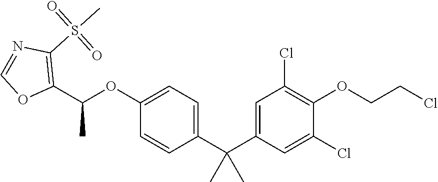 [SMILES: C[C@H](Oc1ccc(C(C)(C)c2cc(Cl)c(OCCCl)c(Cl)c2)cc1)c1ocnc1S(C)(=O)=O] |
| A84 | 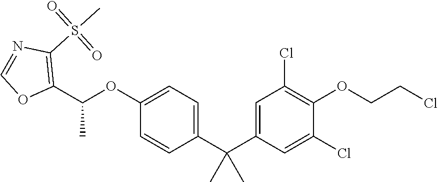 [SMILES: C[C@@H](Oc1ccc(C(C)(C)c2cc(Cl)c(OCCCl)c(Cl)c2)cc1)c1ocnc1S(C)(=O)=O] |
| A85 | 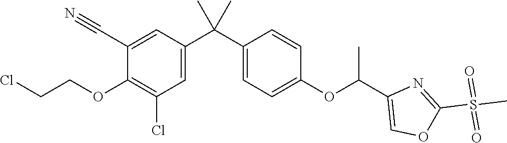 [SMILES: CC(Oc1ccc(C(C)(C)c2cc(Cl)c(OCCCl)c(C#N)c2)cc1)c1coc(S(C)(=O)=O)n1] |
| A86 | 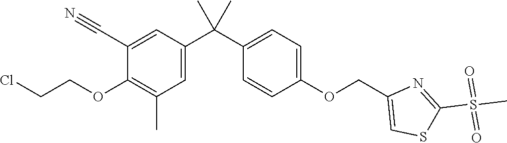 [SMILES: Cc1cc(C(C)(C)c2ccc(OCc3csc(S(C)(=O)=O)n3)cc2)cc(C#N)c1OCCCl] |
| A87 | 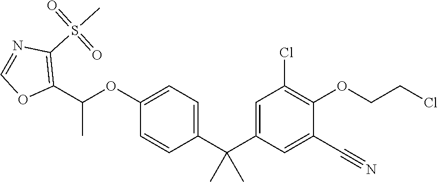 [SMILES: CC(Oc1ccc(C(C)(C)c2cc(Cl)c(OCCCl)c(C#N)c2)cc1)c1ocnc1S(C)(=O)=O] |
| A88 | 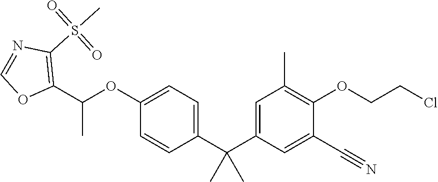 [SMILES: Cc1cc(C(C)(C)c2ccc(OC(C)c3ocnc3S(C)(=O)=O)cc2)cc(C#N)c1OCCCl] |
| A89 | 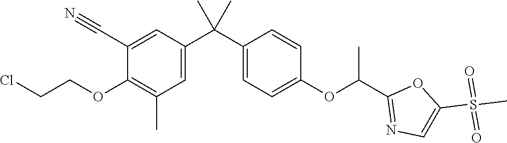 [SMILES: Cc1cc(C(C)(C)c2ccc(OC(C)c3ncc(S(C)(=O)=O)o3)cc2)cc(C#N)c1OCCCl] |
| A90 | 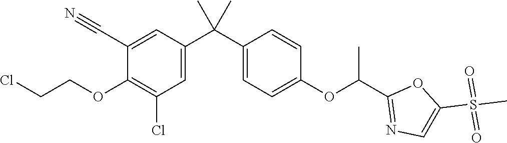 [SMILES: CC(Oc1ccc(C(C)(C)c2cc(Cl)c(OCCCl)c(C#N)c2)cc1)c1ncc(S(C)(=O)=O)o1] |
| A91 | 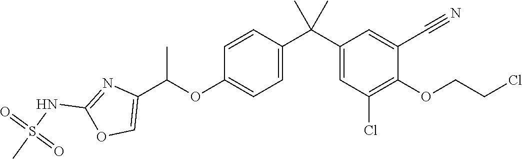 [SMILES: CC(Oc1ccc(C(C)(C)c2cc(Cl)c(OCCCl)c(C#N)c2)cc1)c1coc(NS(C)(=O)=O)n1] |
| A92 | 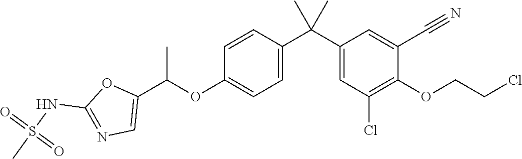 [SMILES: CC(Oc1ccc(C(C)(C)c2cc(Cl)c(OCCCl)c(C#N)c2)cc1)c1cnc(NS(C)(=O)=O)o1] |
| A93 | 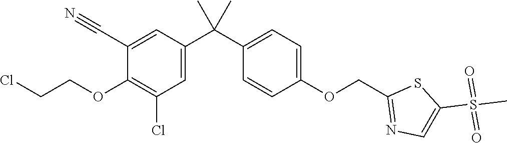 [SMILES: CC(C)(c1ccc(OCc2ncc(S(C)(=O)=O)s2)cc1)c1cc(Cl)c(OCCCl)c(C#N)c1] |
| A94 | 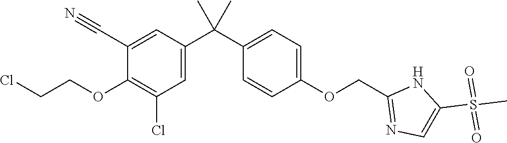 [SMILES: CC(C)(c1ccc(OCc2ncc(S(C)(=O)=O)[nH]2)cc1)c1cc(Cl)c(OCCCl)c(C#N)c1] |
| A95 | 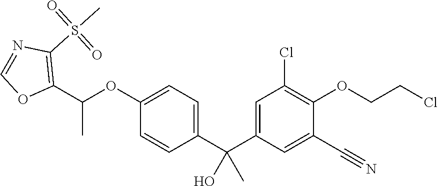 [SMILES: CC(Oc1ccc(C(C)(O)c2cc(Cl)c(OCCCl)c(C#N)c2)cc1)c1ocnc1S(C)(=O)=O] |
| A96 | 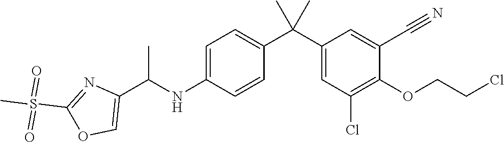 [SMILES: CC(Nc1ccc(C(C)(C)c2cc(Cl)c(OCCCl)c(C#N)c2)cc1)c1coc(S(C)(=O)=O)n1] |
| A97 | 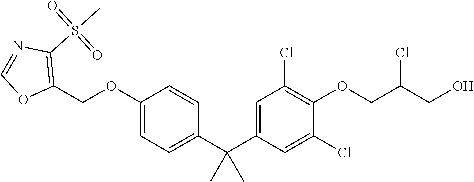 [SMILES: CC(C)(c1ccc(OCc2ocnc2S(C)(=O)=O)cc1)c1cc(Cl)c(OCC(Cl)CO)c(Cl)c1] |
| A98 | 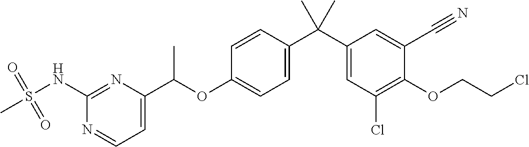 [SMILES: CC(Oc1ccc(C(C)(C)c2cc(Cl)c(OCCCl)c(C#N)c2)cc1)c1ccnc(NS(C)(=O)=O)n1] |
| A99 | 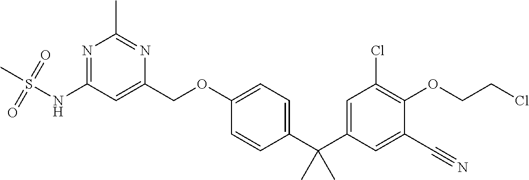 [SMILES: Cc1nc(COc2ccc(C(C)(C)c3cc(Cl)c(OCCCl)c(C#N)c3)cc2)cc(NS(C)(=O)=O)n1] |
| A100 | 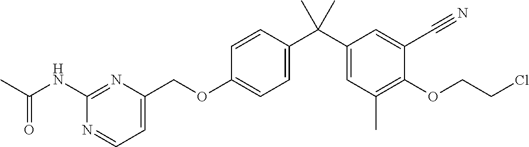 [SMILES: CC(=O)Nc1nccc(COc2ccc(C(C)(C)c3cc(C)c(OCCCl)c(C#N)c3)cc2)n1] |
| A101 | 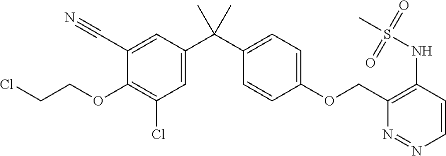 [SMILES: CC(C)(c1ccc(OCc2nnccc2NS(C)(=O)=O)cc1)c1cc(Cl)c(OCCCl)c(C#N)c1] |
| A102 | 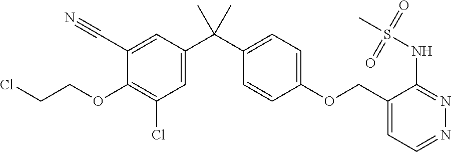 [SMILES: CC(C)(c1ccc(OCc2ccnnc2NS(C)(=O)=O)cc1)c1cc(Cl)c(OCCCl)c(C#N)c1] |
| A103 | 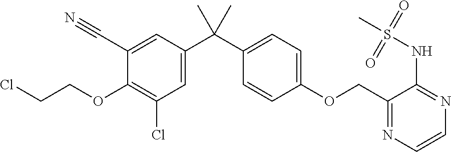 [SMILES: CC(C)(c1ccc(OCc2nccnc2NS(C)(=O)=O)cc1)c1cc(Cl)c(OCCCl)c(C#N)c1] |
| A104 | 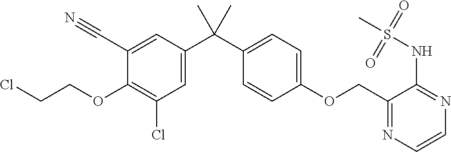 [SMILES: CC(C)(c1ccc(OCc2nccnc2NS(C)(=O)=O)cc1)c1cc(Cl)c(OCCCl)c(C#N)c1] |
| A105 | 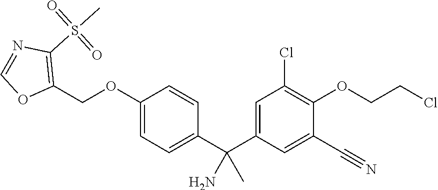 [SMILES: CC(N)(c1ccc(OCc2ocnc2S(C)(=O)=O)cc1)c1cc(Cl)c(OCCCl)c(C#N)c1] |
| A106 | 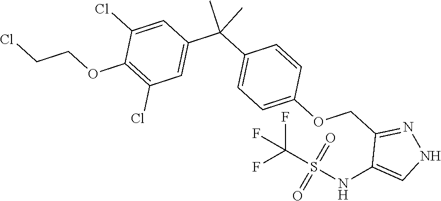 [SMILES: CC(C)(c1ccc(OCc2n[nH]cc2NS(=O)(=O)C(F)(F)F)cc1)c1cc(Cl)c(OCCCl)c(Cl)c1] |
| A107 | 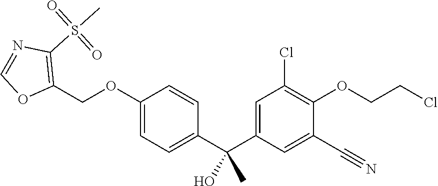 [SMILES: C[C@@](O)(c1ccc(OCc2ocnc2S(C)(=O)=O)cc1)c1cc(Cl)c(OCCCl)c(C#N)c1] |
| A108 | 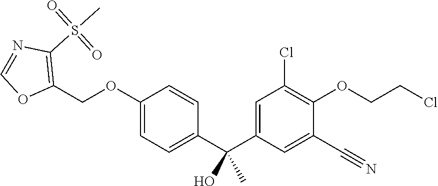 [SMILES: C[C@](O)(c1ccc(OCc2ocnc2S(C)(=O)=O)cc1)c1cc(Cl)c(OCCCl)c(C#N)c1] |
| A109 | 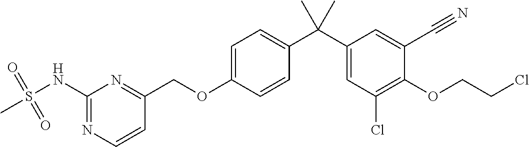 [SMILES: CC(C)(c1ccc(OCc2ccnc(NS(C)(=O)=O)n2)cc1)c1cc(Cl)c(OCCCl)c(C#N)c1] |
| A110 | 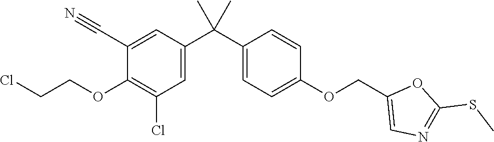 [SMILES: CSc1ncc(COc2ccc(C(C)(C)c3cc(Cl)c(OCCCl)c(C#N)c3)cc2)o1] |
| A111 | 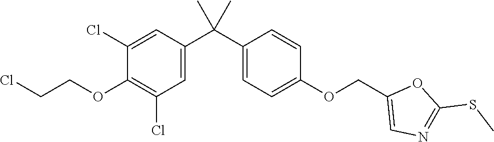 [SMILES: CSc1ncc(COc2ccc(C(C)(C)c3cc(Cl)c(OCCCl)c(Cl)c3)cc2)o1] |
| A112 | 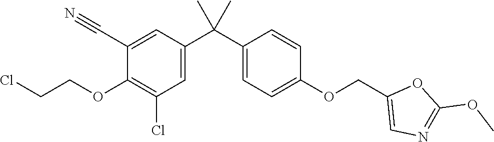 [SMILES: COc1ncc(COc2ccc(C(C)(C)c3cc(Cl)c(OCCCl)c(C#N)c3)cc2)o1] |
| A113 | 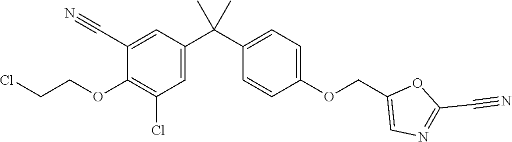 [SMILES: CC(C)(c1ccc(OCc2cnc(C#N)o2)cc1)c1cc(Cl)c(OCCCl)c(C#N)c1] |
| A114 | 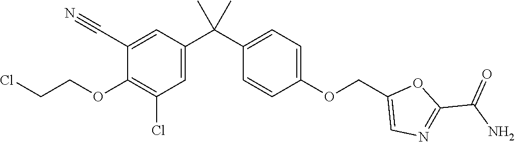 [SMILES: CC(C)(c1ccc(OCc2cnc(C(N)=O)o2)cc1)c1cc(Cl)c(OCCCl)c(C#N)c1] |
| A115 | 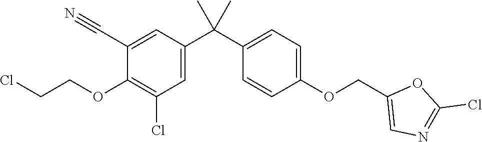 [SMILES: CC(C)(c1ccc(OCc2cnc(Cl)o2)cc1)c1cc(Cl)c(OCCCl)c(C#N)c1] |
| A116 | 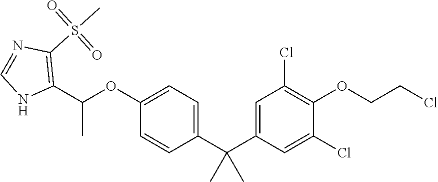 [SMILES: CC(Oc1ccc(C(C)(C)c2cc(Cl)c(OCCCl)c(Cl)c2)cc1)c1[nH]cnc1S(C)(=O)=O] |
| A117 | 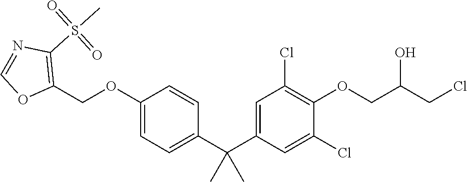 [SMILES: CC(C)(c1ccc(OCc2ocnc2S(C)(=O)=O)cc1)c1cc(Cl)c(OCC(O)CCl)c(Cl)c1] |
| A118 | 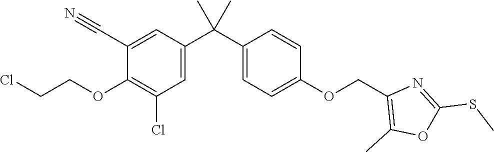 [SMILES: CSc1nc(COc2ccc(C(C)(C)c3cc(Cl)c(OCCCl)c(C#N)c3)cc2)c(C)o1] |
| A119 | 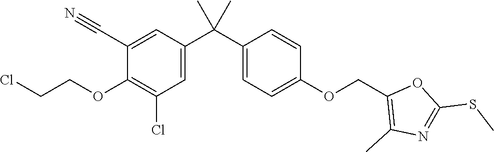 [SMILES: CSc1nc(C)c(COc2ccc(C(C)(C)c3cc(Cl)c(OCCCl)c(C#N)c3)cc2)o1] |
| A120 | 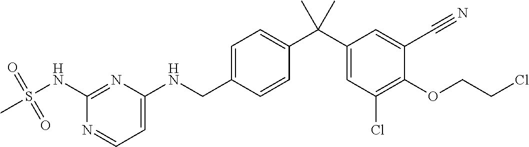 [SMILES: CC(C)(c1ccc(CNc2ccnc(NS(C)(=O)=O)n2)cc1)c1cc(Cl)c(OCCCl)c(C#N)c1] |
| A121 | 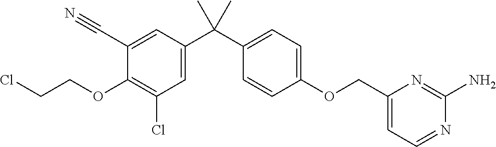 [SMILES: CC(C)(c1ccc(OCc2ccnc(N)n2)cc1)c1cc(Cl)c(OCCCl)c(C#N)c1] |
| A122 | 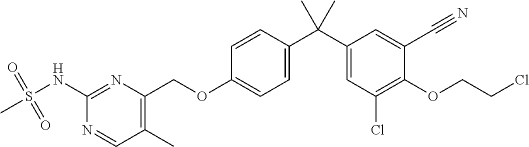 [SMILES: Cc1cnc(NS(C)(=O)=O)nc1COc1ccc(C(C)(C)c2cc(Cl)c(OCCCl)c(C#N)c2)cc1] |
| A123 | 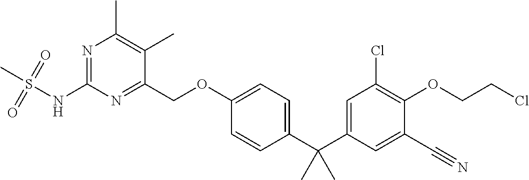 [SMILES: Cc1nc(NS(C)(=O)=O)nc(COc2ccc(C(C)(C)c3cc(Cl)c(OCCCl)c(C#N)c3)cc2)c1C] |
| A124 | 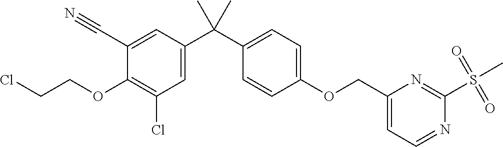 [SMILES: CC(C)(c1ccc(OCc2ccnc(S(C)(=O)=O)n2)cc1)c1cc(Cl)c(OCCCl)c(C#N)c1] |
| A125 | 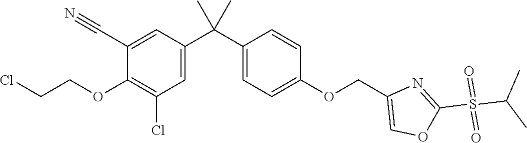 [SMILES: CC(C)S(=O)(=O)c1nc(COc2ccc(C(C)(C)c3cc(Cl)c(OCCCl)c(C#N)c3)cc2)co1] |
| A126 | 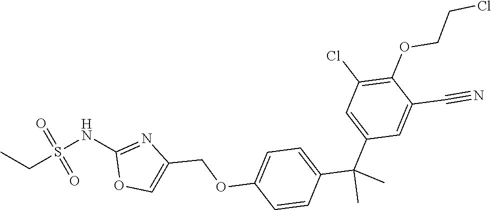 [SMILES: CCS(=O)(=O)Nc1nc(COc2ccc(C(C)(C)c3cc(Cl)c(OCCCl)c(C#N)c3)cc2)co1] |
| A127 | 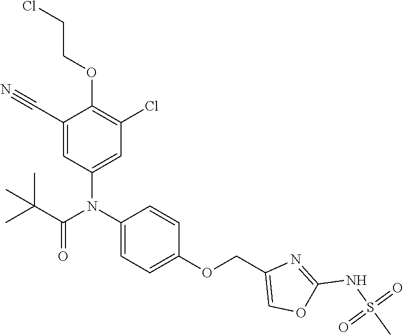 [SMILES: CC(C)(C)C(=O)N(c1ccc(OCc2coc(NS(C)(=O)=O)n2)cc1)c1cc(Cl)c(OCCCl)c(C#N)c1] |
| A128 | 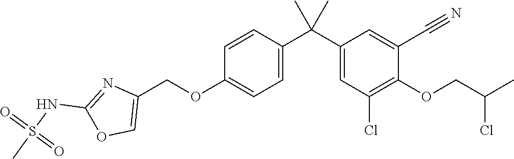 [SMILES: CC(Cl)COc1c(Cl)cc(C(C)(C)c2ccc(OCc3coc(NS(C)(=O)=O)n3)cc2)cc1C#N] |
| A129 | 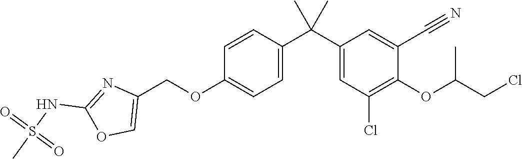 [SMILES: CC(CCl)Oc1c(Cl)cc(C(C)(C)c2ccc(OCc3coc(NS(C)(=O)=O)n3)cc2)cc1C#N] |
| A130 | 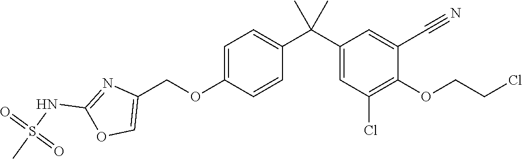 [SMILES: CC(C)(c1ccc(OCc2coc(NS(C)(=O)=O)n2)cc1)c1cc(Cl)c(OCCCl)c(C#N)c1] |
| A131 | 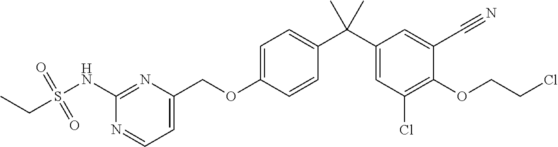 [SMILES: CCS(=O)(=O)Nc1nccc(COc2ccc(C(C)(C)c3cc(Cl)c(OCCCl)c(C#N)c3)cc2)n1] |
| A132 | 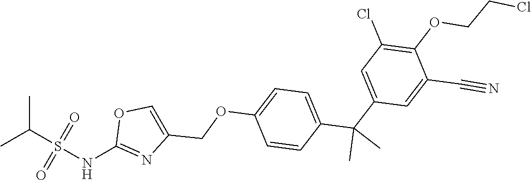 [SMILES: CC(C)S(=O)(=O)Nc1nc(COc2ccc(C(C)(C)c3cc(Cl)c(OCCCl)c(C#N)c3)cc2)co1] |
| A133 | 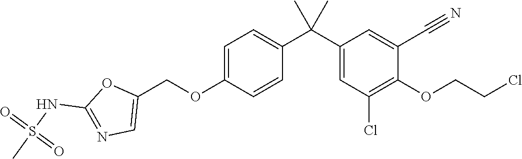 [SMILES: CC(C)(c1ccc(OCc2cnc(NS(C)(=O)=O)o2)cc1)c1cc(Cl)c(OCCCl)c(C#N)c1] |
| A134 | 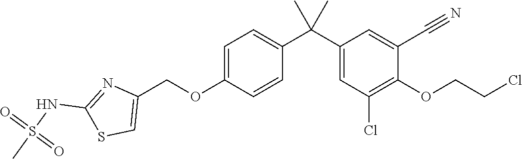 [SMILES: CC(C)(c1ccc(OCc2csc(NS(C)(=O)=O)n2)cc1)c1cc(Cl)c(OCCCl)c(C#N)c1] |
| A135 | 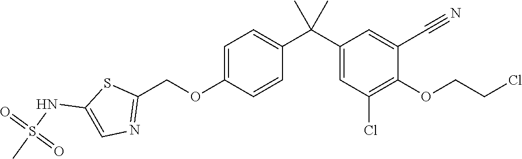 [SMILES: CC(C)(c1ccc(OCc2ncc(NS(C)(=O)=O)s2)cc1)c1cc(Cl)c(OCCCl)c(C#N)c1] |
| A136 | 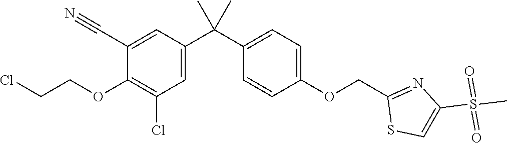 [SMILES: CC(C)(c1ccc(OCc2nc(S(C)(=O)=O)cs2)cc1)c1cc(Cl)c(OCCCl)c(C#N)c1] |
| A137 | 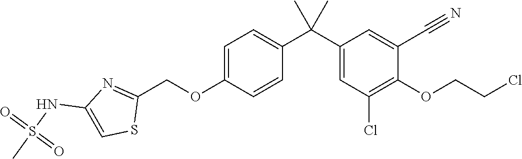 [SMILES: CC(C)(c1ccc(OCc2nc(NS(C)(=O)=O)cs2)cc1)c1cc(Cl)c(OCCCl)c(C#N)c1] |
| A138 | 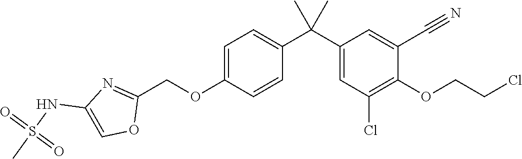 [SMILES: CC(C)(c1ccc(OCc2nc(NS(C)(=O)=O)co2)cc1)c1cc(Cl)c(OCCCl)c(C#N)c1] |
| A139 | 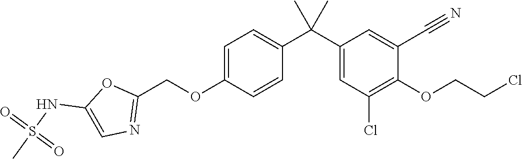 [SMILES: CC(C)(c1ccc(OCc2ncc(NS(C)(=O)=O)o2)cc1)c1cc(Cl)c(OCCCl)c(C#N)c1] |
| A140 | 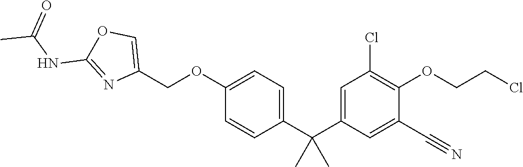 [SMILES: CC(=O)Nc1nc(COc2ccc(C(C)(C)c3cc(Cl)c(OCCCl)c(C#N)c3)cc2)co1] |
| A141 | 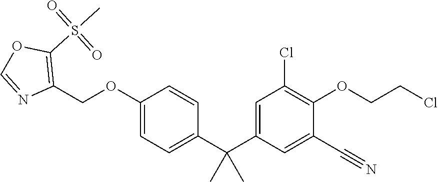 [SMILES: CC(C)(c1ccc(OCc2ncoc2S(C)(=O)=O)cc1)c1cc(Cl)c(OCCCl)c(C#N)c1] |
| A142 | 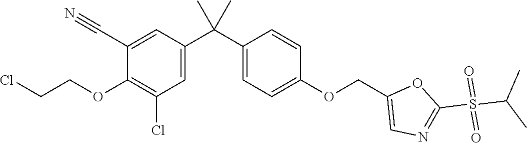 [SMILES: CC(C)S(=O)(=O)c1ncc(COc2ccc(C(C)(C)c3cc(Cl)c(OCCCl)c(C#N)c3)cc2)o1] |
| A143 | 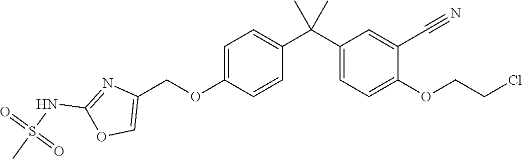 [SMILES: CC(C)(c1ccc(OCc2coc(NS(C)(=O)=O)n2)cc1)c1ccc(OCCCl)c(C#N)c1] |
| A144 | 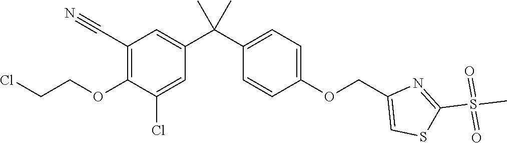 [SMILES: CC(C)(c1ccc(OCc2csc(S(C)(=O)=O)n2)cc1)c1cc(Cl)c(OCCCl)c(C#N)c1] |
| A145 | 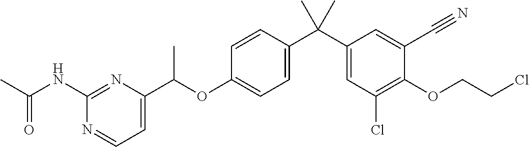 [SMILES: CC(=O)Nc1nccc(C(C)Oc2ccc(C(C)(C)c3cc(Cl)c(OCCCl)c(C#N)c3)cc2)n1] |
| A146 | 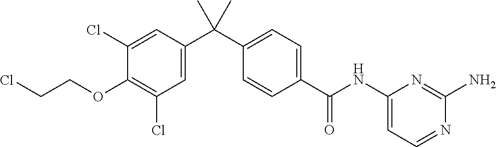 [SMILES: CC(C)(c1ccc(C(=O)Nc2ccnc(N)n2)cc1)c1cc(Cl)c(OCCCl)c(Cl)c1] |
| A147 | 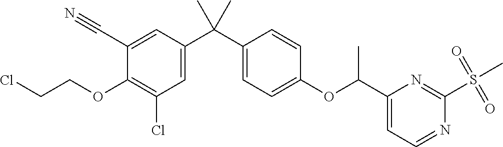 [SMILES: CC(Oc1ccc(C(C)(C)c2cc(Cl)c(OCCCl)c(C#N)c2)cc1)c1ccnc(S(C)(=O)=O)n1] |
| A148 | 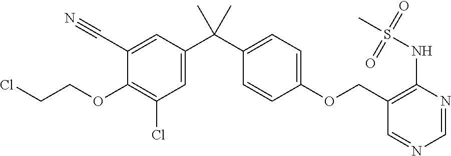 [SMILES: CC(C)(c1ccc(OCc2cncnc2NS(C)(=O)=O)cc1)c1cc(Cl)c(OCCCl)c(C#N)c1] |
| A149 | 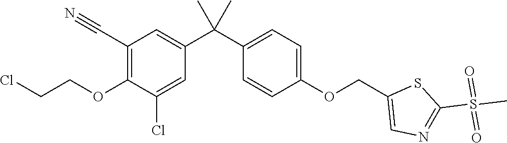 [SMILES: CC(C)(c1ccc(OCc2cnc(S(C)(=O)=O)s2)cc1)c1cc(Cl)c(OCCCl)c(C#N)c1] |
| A150 | 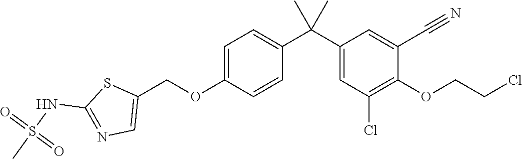 [SMILES: CC(C)(c1ccc(OCc2cnc(NS(C)(=O)=O)s2)cc1)c1cc(Cl)c(OCCCl)c(C#N)c1] |
| A151 | 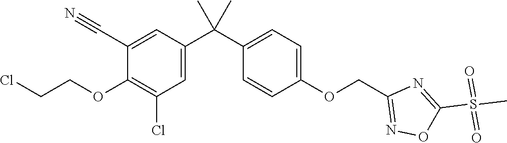 [SMILES: CC(C)(c1ccc(OCc2noc(S(C)(=O)=O)n2)cc1)c1cc(Cl)c(OCCCl)c(C#N)c1] |
| A152 | 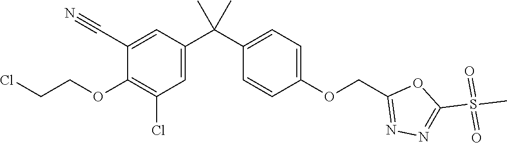 [SMILES: CC(C)(c1ccc(OCc2nnc(S(C)(=O)=O)o2)cc1)c1cc(Cl)c(OCCCl)c(C#N)c1] |
| A153 | 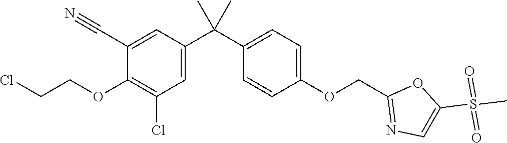 [SMILES: CC(C)(c1ccc(OCc2ncc(S(C)(=O)=O)o2)cc1)c1cc(Cl)c(OCCCl)c(C#N)c1] |
| A154 | 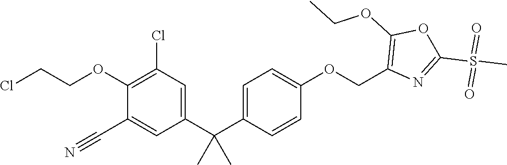 [SMILES: CCOc1oc(S(C)(=O)=O)nc1COc1ccc(C(C)(C)c2cc(Cl)c(OCCCl)c(C#N)c2)cc1] |
| A155 | 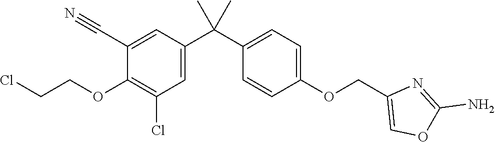 [SMILES: CC(C)(c1ccc(OCc2coc(N)n2)cc1)c1cc(Cl)c(OCCCl)c(C#N)c1] |
| A156 | 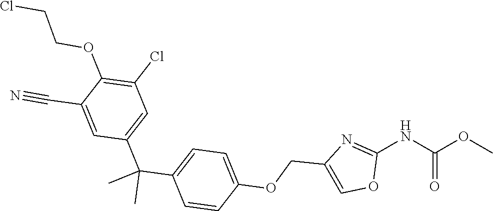 [SMILES: COC(=O)Nc1nc(COc2ccc(C(C)(C)c3cc(Cl)c(OCCCl)c(C#N)c3)cc2)co1] |
| A157 | 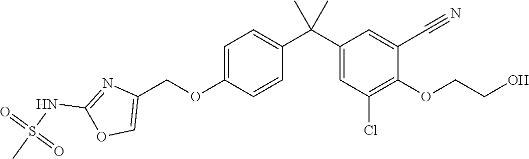 [SMILES: CC(C)(c1ccc(OCc2coc(NS(C)(=O)=O)n2)cc1)c1cc(Cl)c(OCCO)c(C#N)c1] |
| A158 | 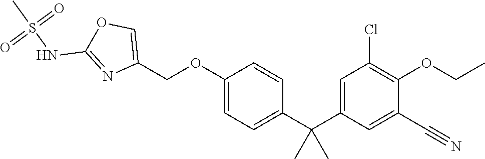 [SMILES: CCOc1c(Cl)cc(C(C)(C)c2ccc(OCc3coc(NS(C)(=O)=O)n3)cc2)cc1C#N] |
| A159 | 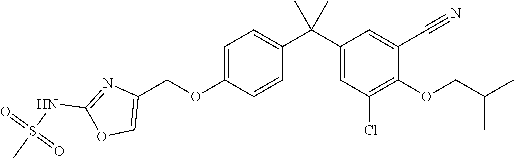 [SMILES: CC(C)COc1c(Cl)cc(C(C)(C)c2ccc(OCc3coc(NS(C)(=O)=O)n3)cc2)cc1C#N] |
| A160 | 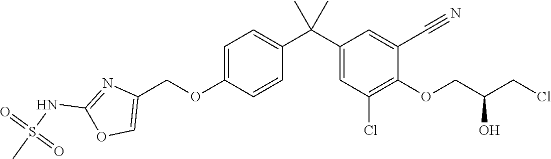 [SMILES: CC(C)(c1ccc(OCc2coc(NS(C)(=O)=O)n2)cc1)c1cc(Cl)c(OC[C@H](O)CCl)c(C#N)c1] |
| A161 | 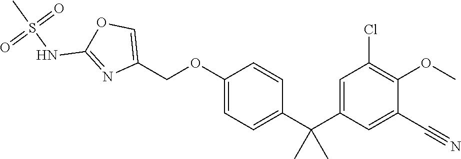 [SMILES: COc1c(Cl)cc(C(C)(C)c2ccc(OCc3coc(NS(C)(=O)=O)n3)cc2)cc1C#N] |
| A162 | 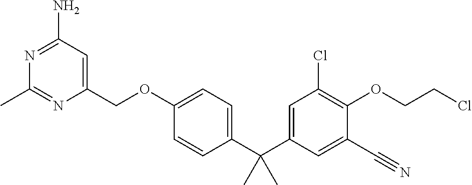 [SMILES: Cc1nc(N)cc(COc2ccc(C(C)(C)c3cc(Cl)c(OCCCl)c(C#N)c3)cc2)n1] |
| A163 | 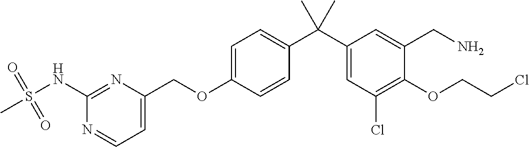 [SMILES: CC(C)(c1ccc(OCc2ccnc(NS(C)(=O)=O)n2)cc1)c1cc(Cl)c(OCCCl)c(CN)c1] |
| A164 | 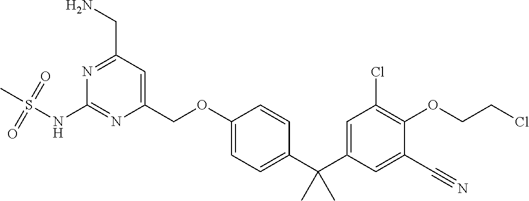 [SMILES: CC(C)(c1ccc(OCc2cc(CN)nc(NS(C)(=O)=O)n2)cc1)c1cc(Cl)c(OCCCl)c(C#N)c1] |
| A165 | 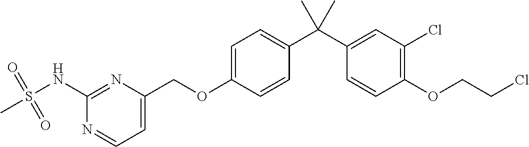 [SMILES: CC(C)(c1ccc(OCc2ccnc(NS(C)(=O)=O)n2)cc1)c1ccc(OCCCl)c(Cl)c1] |
| A166 | 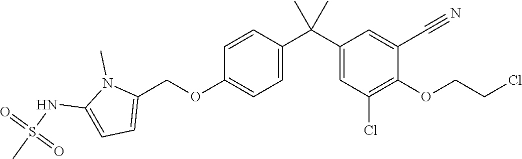 [SMILES: Cn1c(COc2ccc(C(C)(C)c3cc(Cl)c(OCCCl)c(C#N)c3)cc2)ccc1NS(C)(=O)=O] |
| A167 | 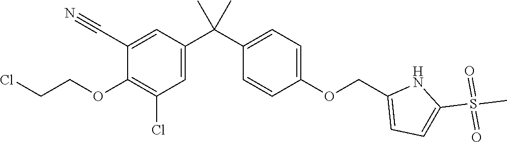 [SMILES: CC(C)(c1ccc(OCc2ccc(S(C)(=O)=O)[nH]2)cc1)c1cc(Cl)c(OCCCl)c(C#N)c1] |
| A168 | 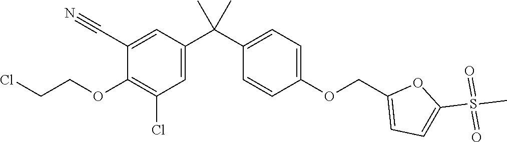 [SMILES: CC(C)(c1ccc(OCc2ccc(S(C)(=O)=O)o2)cc1)c1cc(Cl)c(OCCCl)c(C#N)c1] |
| A169 | 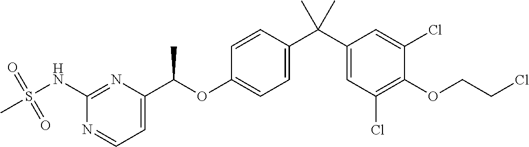 [SMILES: C[C@@H](Oc1ccc(C(C)(C)c2cc(Cl)c(OCCCl)c(Cl)c2)cc1)c1ccnc(NS(C)(=O)=O)n1] |
| A170 | 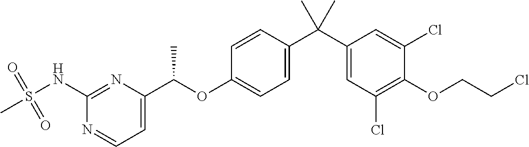 [SMILES: C[C@H](Oc1ccc(C(C)(C)c2cc(Cl)c(OCCCl)c(Cl)c2)cc1)c1ccnc(NS(C)(=O)=O)n1] |
| A171 | 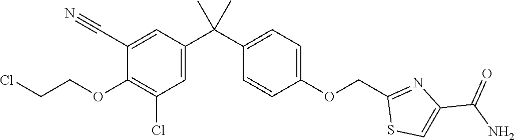 [SMILES: CC(C)(c1ccc(OCc2nc(C(N)=O)cs2)cc1)c1cc(Cl)c(OCCCl)c(C#N)c1] |
| A172 | 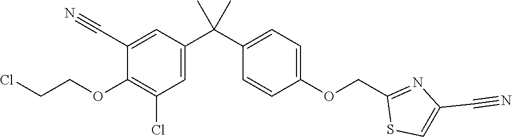 [SMILES: CC(C)(c1ccc(OCc2nc(C#N)cs2)cc1)c1cc(Cl)c(OCCCl)c(C#N)c1] |
| A173 | 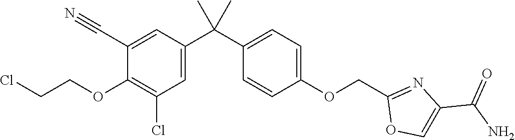 [SMILES: CC(C)(c1ccc(OCc2nc(C(N)=O)co2)cc1)c1cc(Cl)c(OCCCl)c(C#N)c1] |
| A174 | 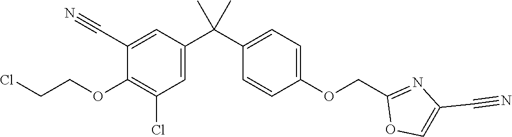 [SMILES: CC(C)(c1ccc(OCc2nc(C#N)co2)cc1)c1cc(Cl)c(OCCCl)c(C#N)c1] |
| A175 | 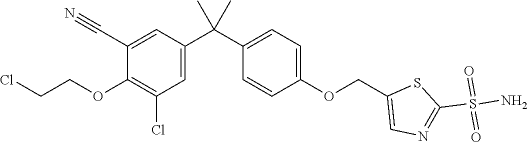 [SMILES: CC(C)(c1ccc(OCc2cnc(S(N)(=O)=O)s2)cc1)c1cc(Cl)c(OCCCl)c(C#N)c1] |
| A176 | 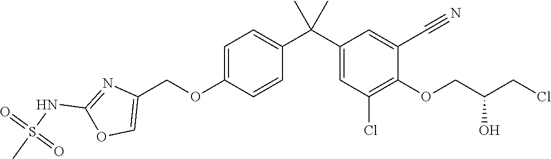 [SMILES: CC(C)(c1ccc(OCc2coc(NS(C)(=O)=O)n2)cc1)c1cc(Cl)c(OC[C@@H](O)CCl)c(C#N)c1] |
| A177 | 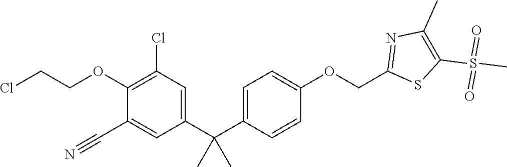 [SMILES: Cc1nc(COc2ccc(C(C)(C)c3cc(Cl)c(OCCCl)c(C#N)c3)cc2)sc1S(C)(=O)=O] |
| A178 | 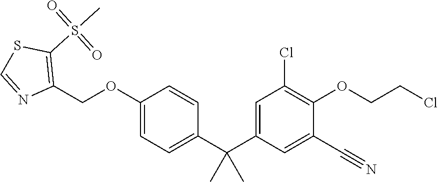 [SMILES: CC(C)(c1ccc(OCc2ncsc2S(C)(=O)=O)cc1)c1cc(Cl)c(OCCCl)c(C#N)c1] |
| A179 | 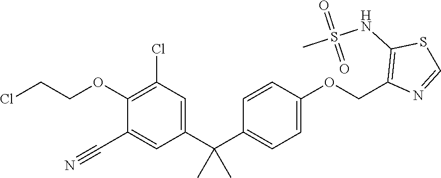 [SMILES: CC(C)(c1ccc(OCc2ncsc2NS(C)(=O)=O)cc1)c1cc(Cl)c(OCCCl)c(C#N)c1] |
| A180 | 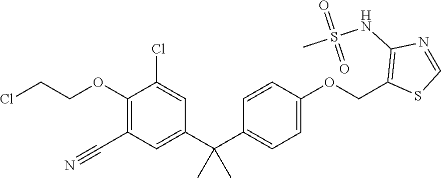 [SMILES: CC(C)(c1ccc(OCc2scnc2NS(C)(=O)=O)cc1)c1cc(Cl)c(OCCCl)c(C#N)c1] |
| A181 | 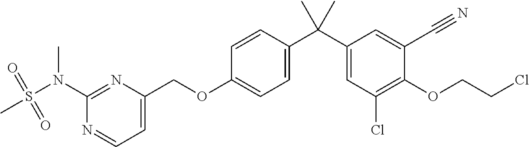 [SMILES: CN(c1nccc(COc2ccc(C(C)(C)c3cc(Cl)c(OCCCl)c(C#N)c3)cc2)n1)S(C)(=O)=O] |
| A182 | 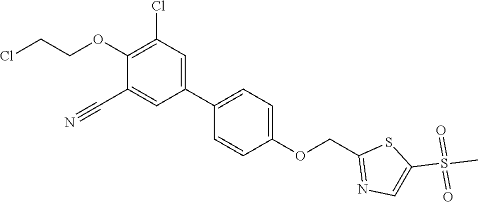 [SMILES: CS(=O)(=O)c1cnc(COc2ccc(-c3cc(Cl)c(OCCCl)c(C#N)c3)cc2)s1] |
| A183 | 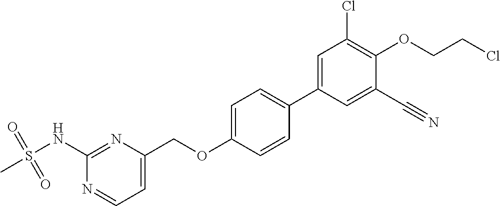 [SMILES: CS(=O)(=O)Nc1nccc(COc2ccc(-c3cc(Cl)c(OCCCl)c(C#N)c3)cc2)n1] |
| A184 | 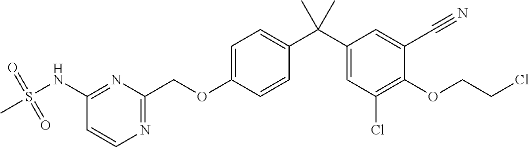 [SMILES: CC(C)(c1ccc(OCc2nccc(NS(C)(=O)=O)n2)cc1)c1cc(Cl)c(OCCCl)c(C#N)c1] |
| A185 | 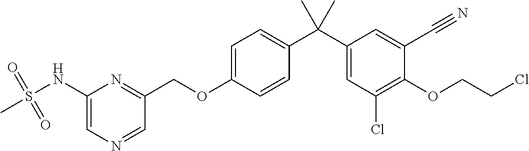 [SMILES: CC(C)(c1ccc(OCc2cncc(NS(C)(=O)=O)n2)cc1)c1cc(Cl)c(OCCCl)c(C#N)c1] |
| A186 | 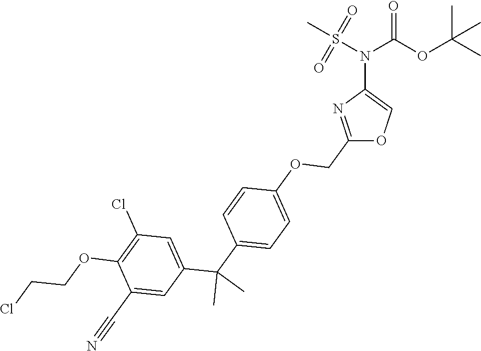 [SMILES: CC(C)(C)OC(=O)N(c1coc(COc2ccc(C(C)(C)c3cc(Cl)c(OCCCl)c(C#N)c3)cc2)n1)S(C)(=O)=O] |
| A187 | 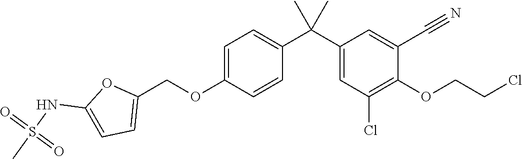 [SMILES: CC(C)(c1ccc(OCc2ccc(NS(C)(=O)=O)o2)cc1)c1cc(Cl)c(OCCCl)c(C#N)c1] |
| A188 | 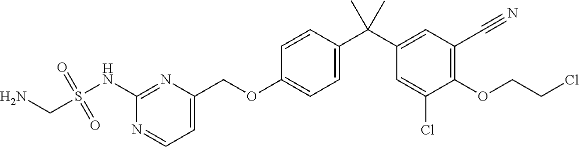 [SMILES: CC(C)(c1ccc(OCc2ccnc(NS(=O)(=O)CN)n2)cc1)c1cc(Cl)c(OCCCl)c(C#N)c1] |
| A189 | 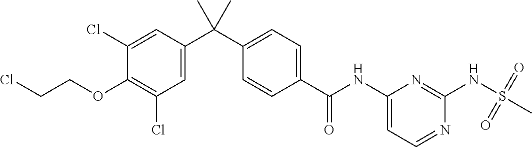 [SMILES: CC(C)(c1ccc(C(=O)Nc2ccnc(NS(C)(=O)=O)n2)cc1)c1cc(Cl)c(OCCCl)c(Cl)c1] |
| A190 | 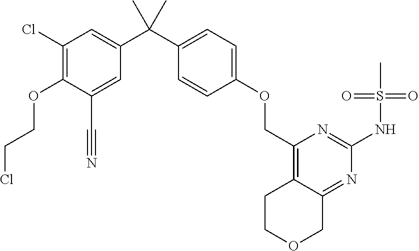 [SMILES: CC(C)(c1ccc(OCc2nc(NS(C)(=O)=O)nc3c2CCOC3)cc1)c1cc(Cl)c(OCCCl)c(C#N)c1] |
| A191 | 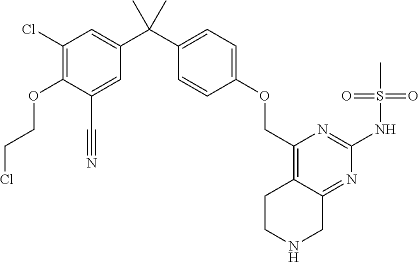 [SMILES: CC(C)(c1ccc(OCc2nc(NS(C)(=O)=O)nc3c2CCNC3)cc1)c1cc(Cl)c(OCCCl)c(C#N)c1] |
| A192 | 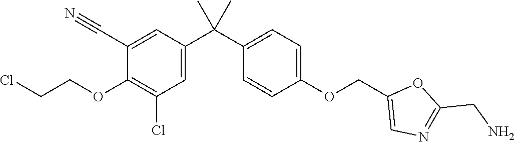 [SMILES: CC(C)(c1ccc(OCc2cnc(CN)o2)cc1)c1cc(Cl)c(OCCCl)c(C#N)c1] |
| A193 | 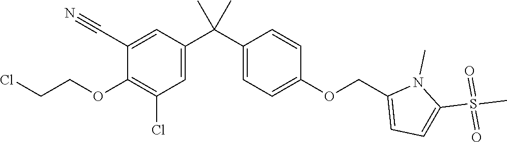 [SMILES: Cn1c(COc2ccc(C(C)(C)c3cc(Cl)c(OCCCl)c(C#N)c3)cc2)ccc1S(C)(=O)=O] |
| A194 | 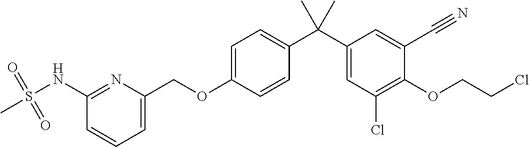 [SMILES: CC(C)(c1ccc(OCc2cccc(NS(C)(=O)=O)n2)cc1)c1cc(Cl)c(OCCCl)c(C#N)c1] |
| A195 | 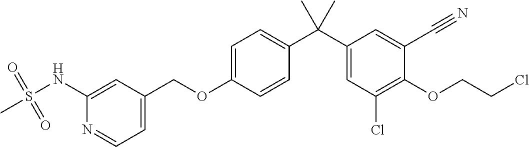 [SMILES: CC(C)(c1ccc(OCc2ccnc(NS(C)(=O)=O)c2)cc1)c1cc(Cl)c(OCCCl)c(C#N)c1] |
| A196 | 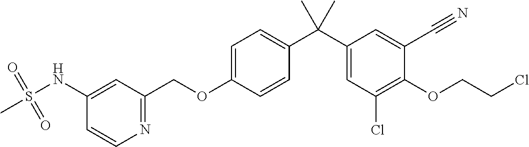 [SMILES: CC(C)(c1ccc(OCc2cc(NS(C)(=O)=O)ccn2)cc1)c1cc(Cl)c(OCCCl)c(C#N)c1] |
| A197 | 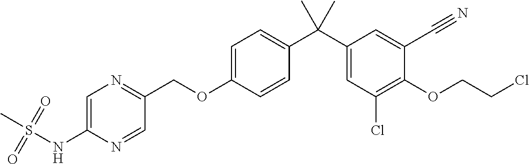 [SMILES: CC(C)(c1ccc(OCc2cnc(NS(C)(=O)=O)cn2)cc1)c1cc(Cl)c(OCCCl)c(C#N)c1] |
| A198 | 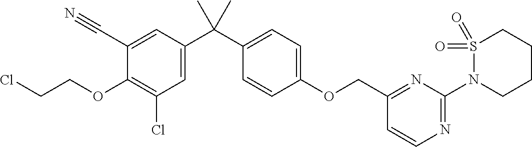 [SMILES: CC(C)(c1ccc(OCc2ccnc(N3CCCCS3(=O)=O)n2)cc1)c1cc(Cl)c(OCCCl)c(C#N)c1] |
| A199 | 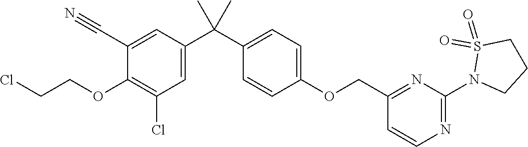 [SMILES: CC(C)(c1ccc(OCc2ccnc(N3CCCS3(=O)=O)n2)cc1)c1cc(Cl)c(OCCCl)c(C#N)c1] |
| A200 | 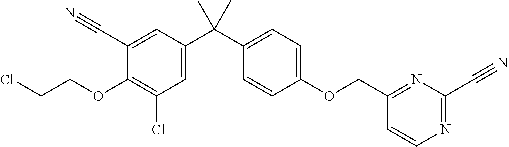 [SMILES: CC(C)(c1ccc(OCc2ccnc(C#N)n2)cc1)c1cc(Cl)c(OCCCl)c(C#N)c1] |
| A201 | 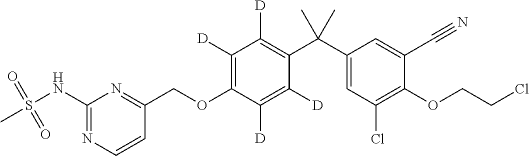 [SMILES: [2H]c1c([2H])c(C(C)(C)c2cc(Cl)c(OCCCl)c(C#N)c2)c([2H])c([2H])c1OCc1ccnc(NS(C)(=O)=O)n1] |
| A202 | 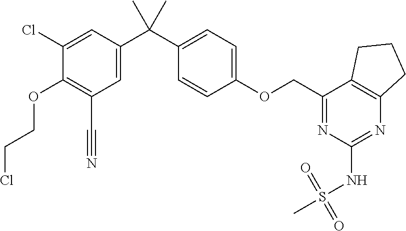 [SMILES: CC(C)(c1ccc(OCc2nc(NS(C)(=O)=O)nc3c2CCC3)cc1)c1cc(Cl)c(OCCCl)c(C#N)c1] |
| A203 | 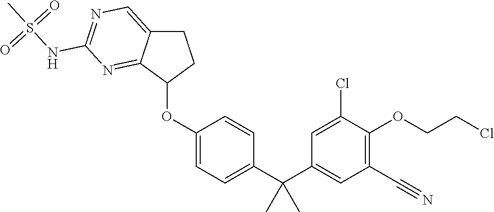 [SMILES: CC(C)(c1ccc(OC2CCc3cnc(NS(C)(=O)=O)nc32)cc1)c1cc(Cl)c(OCCCl)c(C#N)c1] |
| A204 | 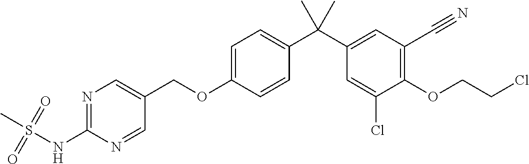 [SMILES: CC(C)(c1ccc(OCc2cnc(NS(C)(=O)=O)nc2)cc1)c1cc(Cl)c(OCCCl)c(C#N)c1] |
| A205 | 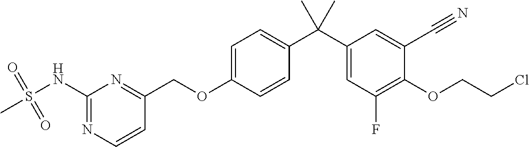 [SMILES: CC(C)(c1ccc(OCc2ccnc(NS(C)(=O)=O)n2)cc1)c1cc(F)c(OCCCl)c(C#N)c1] |
| A206 | 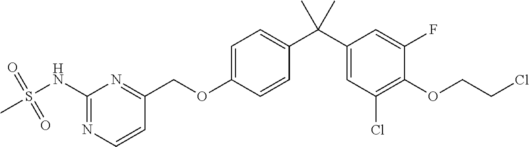 [SMILES: CC(C)(c1ccc(OCc2ccnc(NS(C)(=O)=O)n2)cc1)c1cc(F)c(OCCCl)c(Cl)c1] |
| A207 | 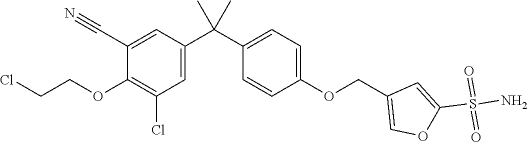 [SMILES: CC(C)(c1ccc(OCc2coc(S(N)(=O)=O)c2)cc1)c1cc(Cl)c(OCCCl)c(C#N)c1] |
| A208 | 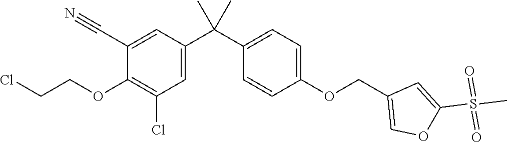 [SMILES: CC(C)(c1ccc(OCc2coc(S(C)(=O)=O)c2)cc1)c1cc(Cl)c(OCCCl)c(C#N)c1] |
| A209 | 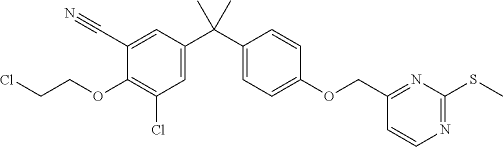 [SMILES: CSc1nccc(COc2ccc(C(C)(C)c3cc(Cl)c(OCCCl)c(C#N)c3)cc2)n1] |
| A210 | 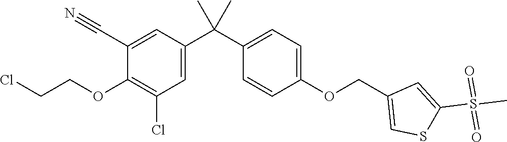 [SMILES: CC(C)(c1ccc(OCc2csc(S(C)(=O)=O)c2)cc1)c1cc(Cl)c(OCCCl)c(C#N)c1] |
| A211 | 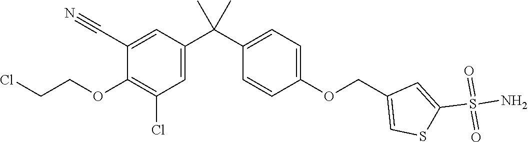 [SMILES: CC(C)(c1ccc(OCc2csc(S(N)(=O)=O)c2)cc1)c1cc(Cl)c(OCCCl)c(C#N)c1] |
| A212 | 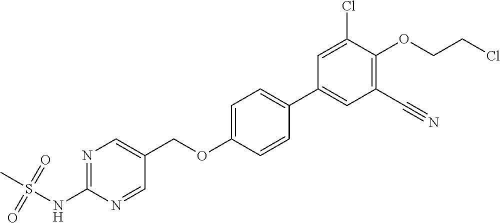 [SMILES: CS(=O)(=O)Nc1ncc(COc2ccc(-c3cc(Cl)c(OCCCl)c(C#N)c3)cc2)cn1] |
| A213 | 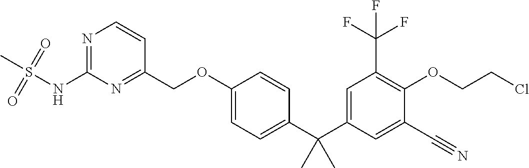 [SMILES: CC(C)(c1ccc(OCc2ccnc(NS(C)(=O)=O)n2)cc1)c1cc(C#N)c(OCCCl)c(C(F)(F)F)c1] |
| A214 | 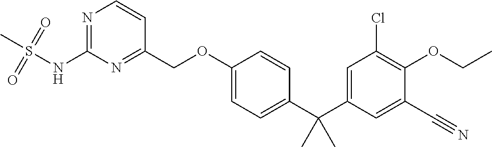 [SMILES: CCOc1c(Cl)cc(C(C)(C)c2ccc(OCc3ccnc(NS(C)(=O)=O)n3)cc2)cc1C#N] |
| A215 | 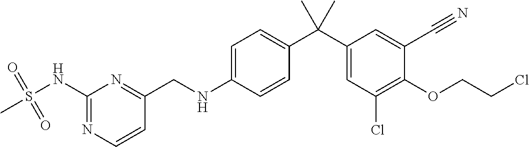 [SMILES: CC(C)(c1ccc(NCc2ccnc(NS(C)(=O)=O)n2)cc1)c1cc(Cl)c(OCCCl)c(C#N)c1] |
| A216 | 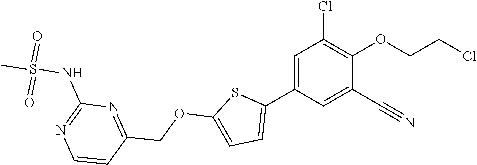 [SMILES: CS(=O)(=O)Nc1nccc(COc2ccc(-c3cc(Cl)c(OCCCl)c(C#N)c3)s2)n1] |
| A217 | 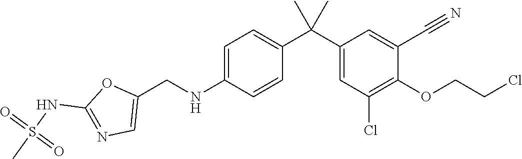 [SMILES: CC(C)(c1ccc(NCc2cnc(NS(C)(=O)=O)o2)cc1)c1cc(Cl)c(OCCCl)c(C#N)c1] |
| A218 | 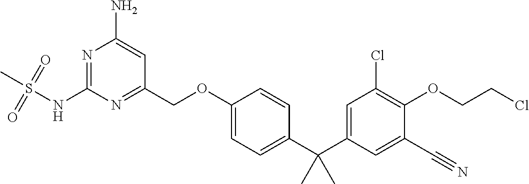 [SMILES: CC(C)(c1ccc(OCc2cc(N)nc(NS(C)(=O)=O)n2)cc1)c1cc(Cl)c(OCCCl)c(C#N)c1] |
| A219 | 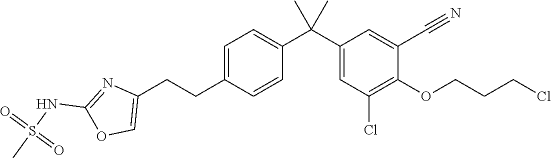 [SMILES: CC(C)(c1ccc(CCc2coc(NS(C)(=O)=O)n2)cc1)c1cc(Cl)c(OCCCCl)c(C#N)c1] |
| A220 | 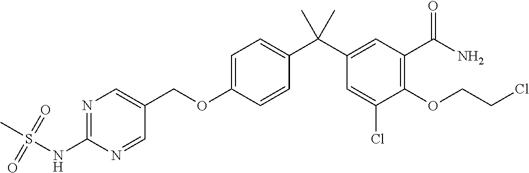 [SMILES: CC(C)(c1ccc(OCc2cnc(NS(C)(=O)=O)nc2)cc1)c1cc(Cl)c(OCCCl)c(C(N)=O)c1] |
| A221 | 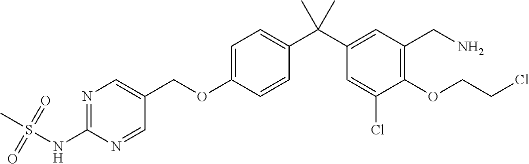 [SMILES: CC(C)(c1ccc(OCc2cnc(NS(C)(=O)=O)nc2)cc1)c1cc(Cl)c(OCCCl)c(CN)c1] |
| A222 | 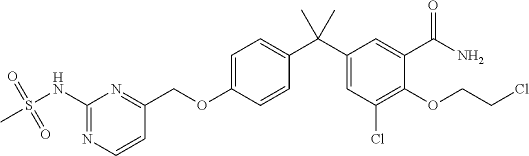 [SMILES: CC(C)(c1ccc(OCc2ccnc(NS(C)(=O)=O)n2)cc1)c1cc(Cl)c(OCCCl)c(C(N)=O)c1] |
| A223 | 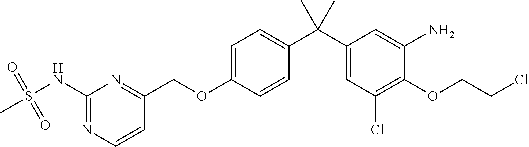 [SMILES: CC(C)(c1ccc(OCc2ccnc(NS(C)(=O)=O)n2)cc1)c1cc(N)c(OCCCl)c(Cl)c1] |
| A224 | 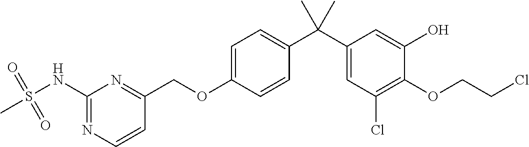 [SMILES: CC(C)(c1ccc(OCc2ccnc(NS(C)(=O)=O)n2)cc1)c1cc(O)c(OCCCl)c(Cl)c1] |
| A225 | 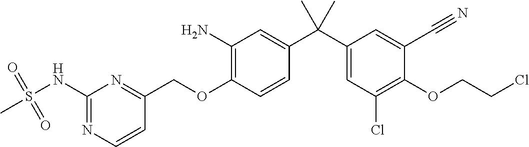 [SMILES: CC(C)(c1ccc(OCc2ccnc(NS(C)(=O)=O)n2)c(N)c1)c1cc(Cl)c(OCCCl)c(C#N)c1] |
| A226 | 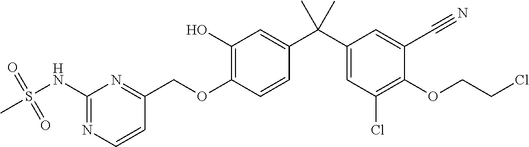 [SMILES: CC(C)(c1ccc(OCc2ccnc(NS(C)(=O)=O)n2)c(O)c1)c1cc(Cl)c(OCCCl)c(C#N)c1] |
| A227 | 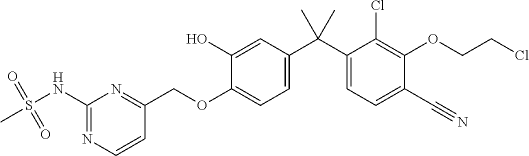 [SMILES: CC(C)(c1ccc(OCc2ccnc(NS(C)(=O)=O)n2)c(O)c1)c1ccc(C#N)c(OCCCl)c1Cl] |
| A228 | 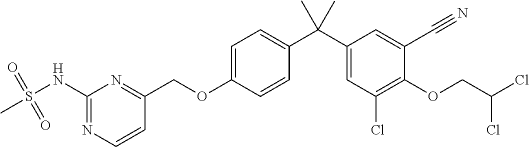 [SMILES: CC(C)(c1ccc(OCc2ccnc(NS(C)(=O)=O)n2)cc1)c1cc(Cl)c(OCC(Cl)Cl)c(C#N)c1] |
| A229 | 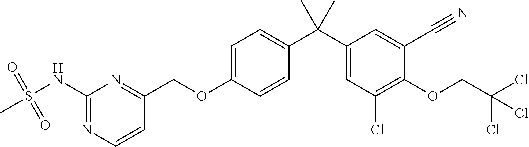 [SMILES: CC(C)(c1ccc(OCc2ccnc(NS(C)(=O)=O)n2)cc1)c1cc(Cl)c(OCC(Cl)(Cl)Cl)c(C#N)c1] |
| A230 | 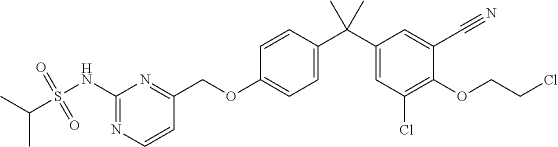 [SMILES: CC(C)S(=O)(=O)Nc1nccc(COc2ccc(C(C)(C)c3cc(Cl)c(OCCCl)c(C#N)c3)cc2)n1] |
| A231 | 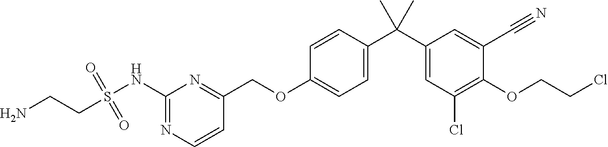 [SMILES: CC(C)(c1ccc(OCc2ccnc(NS(=O)(=O)CCN)n2)cc1)c1cc(Cl)c(OCCCl)c(C#N)c1] |
| A232 | 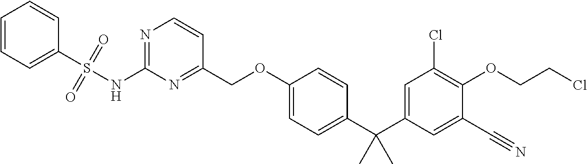 [SMILES: CC(C)(c1ccc(OCc2ccnc(NS(=O)(=O)c3ccccc3)n2)cc1)c1cc(Cl)c(OCCCl)c(C#N)c1] |
| A233 | 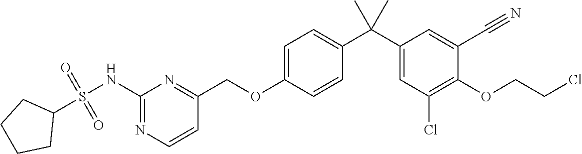 [SMILES: CC(C)(c1ccc(OCc2ccnc(NS(=O)(=O)C3CCCC3)n2)cc1)c1cc(Cl)c(OCCCl)c(C#N)c1] |
| A234 | 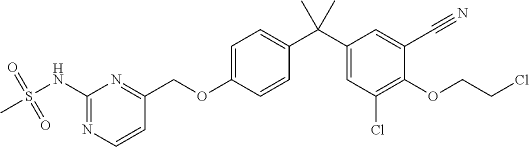 [SMILES: CC(C)(c1ccc(OCc2ccnc(NS(C)(=O)=O)n2)cc1)c1cc(Cl)c(OCCCl)c(C#N)c1] |

In one embodiment of the compounds of formula (I)-(III), (IIIA), (VI), (IVA), (V)-(X), (A-I), or (G-II), the compound is selected from Table B below, or a pharmaceutically acceptable salt, tautomer, stereoisomer or prodrug thereof. In one embodiment of the compounds of formula (I)-(III), (IIIA), (VI), (IVA), (V)-(X), (A-I), or (G-II), the compound is selected from Compounds B1-B11 or a pharmaceutically acceptable salt, tautomer, stereoisomer or prodrug thereof.

TABLE B
Androgen Receptor Modulators

| Compound | |
|---|---|
| ID | Structure |
| B1 | 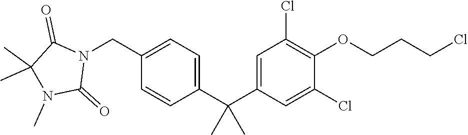 [SMILES: CN1C(=O)N(Cc2ccc(C(C)(C)c3cc(Cl)c(OCCCCl)c(Cl)c3)cc2)C(=O)C1(C)C] |
| B2 | 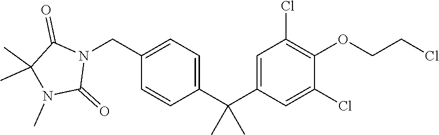 [SMILES: CN1C(=O)N(Cc2ccc(C(C)(C)c3cc(Cl)c(OCCCl)c(Cl)c3)cc2)C(=O)C1(C)C] |
| B3 | 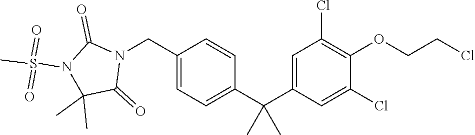 [SMILES: CC(C)(c1ccc(CN2C(=O)N(S(C)(=O)=O)C(C)(C)C2=O)cc1)c1cc(Cl)c(OCCCl)c(Cl)c1] |
| B4 | 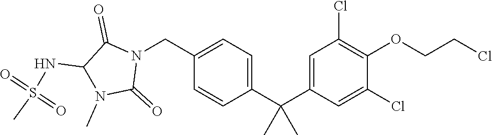 [SMILES: CN1C(=O)N(Cc2ccc(C(C)(C)c3cc(Cl)c(OCCCl)c(Cl)c3)cc2)C(=O)C1NS(C)(=O)=O] |
| B5 | 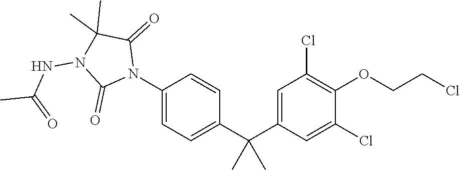 [SMILES: CC(=O)NN1C(=O)N(c2ccc(C(C)(C)c3cc(Cl)c(OCCCl)c(Cl)c3)cc2)C(=O)C1(C)C] |
| B6 | 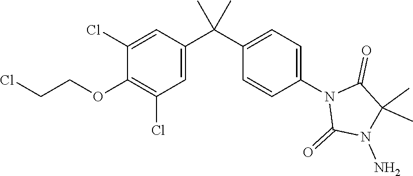 [SMILES: CC(C)(c1ccc(N2C(=O)N(N)C(C)(C)C2=O)cc1)c1cc(Cl)c(OCCCl)c(Cl)c1] |
| B7 | 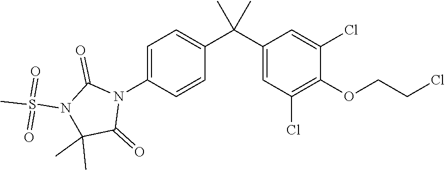 [SMILES: CC(C)(c1ccc(N2C(=O)N(S(C)(=O)=O)C(C)(C)C2=O)cc1)c1cc(Cl)c(OCCCl)c(Cl)c1] |
| B8 | 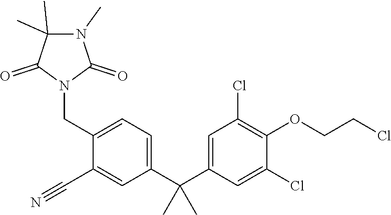 [SMILES: CN1C(=O)N(Cc2ccc(C(C)(C)c3cc(Cl)c(OCCCl)c(Cl)c3)cc2C#N)C(=O)C1(C)C] |
| B9 | 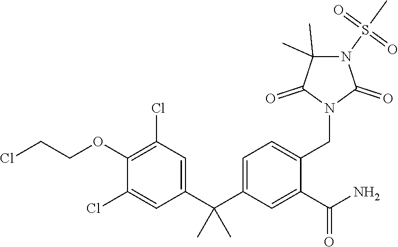 [SMILES: CC(C)(c1cc(Cl)c(OCCCl)c(Cl)c1)c1ccc(CN2C(=O)N(S(C)(=O)=O)C(C)(C)C2=O)c(C(N)=O)c1] |
| B10 | 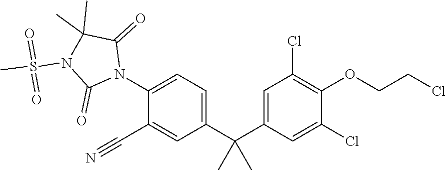 [SMILES: CC(C)(c1cc(Cl)c(OCCCl)c(Cl)c1)c1ccc(N2C(=O)N(S(C)(=O)=O)C(C)(C)C2=O)c(C#N)c1] |
| B11 | 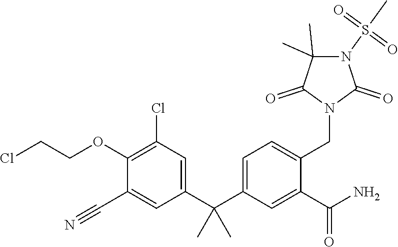 [SMILES: CC(C)(c1cc(Cl)c(OCCCl)c(C#N)c1)c1ccc(CN2C(=O)N(S(C)(=O)=O)C(C)(C)C2=O)c(C(N)=O)c1] |

In one embodiment, the compounds as disclosed herein is an androgen receptor modulator. In one embodiment, the compounds as disclosed herein binds to androgen receptor. In another embodiment, the compounds as disclosed herein binds to androgen receptor N-terminal domain.

In one embodiment, the pharmaceutical compositions and the pharmaceutical combinations of present disclosure comprises a compound of formula (I)-(III), (IIIA), (VI), (IVA), (V)-(X), (A-I), or (G-II) or a pharmaceutically acceptable salt thereof and a second therapeutically active agent. In one embodiment, the pharmaceutical compositions and the pharmaceutical combinations of present disclosure comprises a compound of formula (I)-(III), (IIIA), (VI), (IVA), (V)-(X), (A-I), or (G-II) or compounds of Tables A and B, or a pharmaceutically acceptable salt, tautomer, stereoisomer or prodrug thereof, and an androgen receptor ligand-binding domain inhibitor. In one embodiment, the pharmaceutical compositions and the pharmaceutical combinations of present disclosure comprises a compound selected from Compounds A1-A186 or B1-B11, or a pharmaceutically acceptable salt, tautomer, stereoisomer or prodrug thereof, and an androgen receptor ligand-binding domain inhibitor. In one embodiment, the pharmaceutical compositions and the pharmaceutical combinations of present disclosure comprises a compound selected from Compounds A1-A211 or B1-B11, or a pharmaceutically acceptable salt, tautomer, stereoisomer or prodrug thereof, and a an androgen receptor ligand-binding domain inhibitor. In one embodiment, the pharmaceutical compositions and the pharmaceutical combinations of present disclosure comprises a compound selected from Compounds A1-A234 or B1-B11, or a pharmaceutically acceptable salt, tautomer, stereoisomer or prodrug thereof, and a an androgen receptor ligand-binding domain inhibitor. In one embodiment, the pharmaceutical compositions and the pharmaceutical combinations of present disclosure further comprises a pharmaceutically acceptable cater or a pharmaceutically acceptable excipient.

Androgen Receptor Ligand-Binding Domain Inhibitor

In one embodiment, the androgen receptor modulator compound of formula (I)-(III), (IIIA), (VI), (IVA), (V)-(X), (A-I), or (G-II) or a pharmaceutically acceptable salt thereof are androgen receptor N-terminal domain inhibitors. In one embodiment, androgen receptor N-terminal domain inhibitors can be useful when used in combination with an androgen receptor ligand-binding domain inhibitor. In some embodiment, an androgen receptor N-terminal domain inhibitor and an androgen receptor ligand-binding domain inhibitor can act synergistically.

In one embodiment, the androgen receptor ligand-binding domain inhibitor is enzalutamide, apalutamide, darolutamide, bicalutamide, nilutamide, flutamide, ODM-204, or TAS3681. In one embodiment, the androgen receptor ligand-binding domain inhibitor is enzalutamide.

Other Therapeutically Active Agents

The pharmaceutical composition of the present disclosure comprises a second therapeutically active agent. The second therapeutically active agent can be selected from from a poly (ADP-ribose) polymerase (PARP) inhibitor, an androgen receptor ligand-binding domain inhibitor, an inhibitor of CYP17, a microtubule inhibitor, a modulator of PD-1 or PD-L1, a gonadotropin releasing hormone agonist, a 5-alpha reductase inhibitor, a vascular endothelial growth factor inhibitor, a histone deacetylase inhibitor, an integrin alpha-v-beta-3 inhibitor, a receptor tyrosine kinase, a phosphoinositide 3-kinase inhibitor, an anaplastic lymphoma kinase (ALK) inhibitor, an endothelin receptor A antagonist, an anti-CTLA4 inhibitor, an heat shock protein 27 (HSP27) inhibitor, an androgen receptor degrader, a androgen receptor DNA-binding domain inhibitor, a bromodomain and extra-terminal motif (BET) inhibitor, an androgen receptor N-terminal domain inhibitor, an alpha-particle emitting radioactive therapeutic agent, niclosamide, a selective estrogen receptor modulator (SERM), a selective estrogen receptor degrader (SERD), an aromatase inhibitor, selective progesterone receptor modulator (SPRM), a glucocorticoid receptor inhibitor, a HER2 receptor antagonist, a mammalian target of rapamycin (mTOR) inhibitor, an AKT inhibitor, a B-cell lymphoma-2 (Bcl-2) inhibitor, an aurora kinase inhibitor, a Wnt-targeting antagonist, a CYP11a inhibitor, a selective androgen receptor modulator, or enhancer of zeste homolog 2 (EZH2) inhibitor.

The pharmaceutical composition of the present disclosure comprises a second therapeutically active agent. The second therapeutically active agent can be selected from from a poly (ADP-ribose) polymerase (PARP) inhibitor, an androgen receptor ligand-binding domain inhibitor, an inhibitor of CYP17, a microtubule inhibitor, a modulator of PD-1 or PD-L1, a gonadotropin releasing hormone agonist, a 5-alpha reductase inhibitor, a vascular endothelial growth factor inhibitor, a histone deacetylase inhibitor, an integrin alpha-v-beta-3 inhibitor, a receptor tyrosine kinase, a phosphoinositide 3-kinase inhibitor, an anaplastic lymphoma kinase (ALK) inhibitor, an endothelin receptor A antagonist, an anti-CTLA4 inhibitor, an heat shock protein 27 (HSP27) inhibitor, an androgen receptor degrader, a androgen receptor DNA-binding domain inhibitor, a bromodomain and extra-terminal motif (BET) inhibitor, an androgen receptor N-terminal domain inhibitor, an alpha-particle emitting radioactive therapeutic agent, niclosamide, a selective estrogen receptor modulator (SERM), a selective estrogen receptor degrader (SERD), an aromatase inhibitor, selective progesterone receptor modulator (SPRM), a glucocorticoid receptor inhibitor, a HER2 receptor antagonist, or a mammalian target of rapamycin (mTOR) inhibitor.

In one embodiment, the second therapeutically active agent is selected from a poly (ADP-ribose) polymerase (PARP) inhibitor including but not limited to olaparib, niraparib, rucaparib, talazoparib; an androgen receptor ligand-binding domain inhibitor including but not limited to enzalutamide, apalutamide, darolutamide, bicalutamide, nilutamide, flutamide, ODM-204, TAS3681; an inhibitor of CYP17 including but not limited to galeterone, abiraterone, abiraterone acetate; a microtubule inhibitor including but not limited to docetaxel, paclitaxel, cabazitaxel (XRP-6258); a modulator of PD-1 or PD-L1 including but not limited to pembrolizumab, durvalumab, nivolumab, atezolizumab; a gonadotropin releasing hormone agonist including but not limited to cyproterone acetate, leuprolide; a 5-alpha reductase inhibitor including but not limited to finasteride, dutasteride, turosteride, bexlosteride, izonsteride, FCE 28260, SKF105,111; a vascular endothelial growth factor inhibitor including but not limited to bevacizumab (Avastin); a histone deacetylase inhibitor including but not limited to OSU-HDAC42; an integrin alpha-v-beta-3 inhibitor including but not limited to VITAXIN; a receptor tyrosine kinase including but not limited to sunitumib; a phosphoinositide 3-kinase inhibitor including but not limited to alpelisib, buparlisib, idealisib; an anaplastic lymphoma kinase (ALK) inhibitor including but not limited to crizotinib, alectinib; an endothelin receptor A antagonist including but not limited to ZD-4054; an anti-CTLA4 inhibitor including but not limited to MDX-010 (ipilimumab); an heat shock protein 27 (HSP27) inhibitor including but not limited to OGX 427; an androgen receptor degrader including but not limited to ARV-330, ARV-110; a androgen receptor DNA-binding domain inhibitor including but not limited to VPC-14449; a bromodomain and extra-terminal motif (BET) inhibitor including but not limited to BI-894999, GSK25762, GS-5829; an N-terminal domain inhibitor including but not limited to a sintokamide; an alpha-particle emitting radioactive therapeutic agent including but not limited to radium 233 or a salt thereof; niclosamide; or related compounds thereof; a selective estrogen receptor modulator (SERM) including but not limited to tamoxifen, raloxifene, toremifene, arzoxifene, bazedoxifene, pipindoxifene, lasofoxifene, enclomiphene; a selective estrogen receptor degrader (SERD) including but not limited to fulvestrant, ZB716, OP-1074, elacestrant, AZD9496, GDC0810, GDC0927, GW5638, GW7604; an aromatase inhibitor including but not limited to anastrazole, exemestane, letrozole; selective progesterone receptor modulators (SPRM) including but not limited to mifepristone, lonaprison, onapristone, asoprisnil, lonaprisnil, ulipristal, telapristone; a glucocorticoid receptor inhibitor including but not limited to mifepristone, COR108297, COR125281, ORIC-101, PT150; HER2 receptor antagonist including but not limited to trastuzumab, neratinib; or a mammalian target of rapamycin (mTOR) inhibitor including but not limited to everolimus, temsirolimus, an AKT inhibitor including but not limited to MK-2206; a Bcl-2 inhibitor including but not limited to venetoclax; an aurora kinase inhibitor including but not limited to alisertib; a Wnt-targeting antagonist including but not limited to DKK-1-4 proteins (Dikhopf), secreted Frazzle related proteins (sFRP); a CYP11a inhibitor including but not limited to ODM-208; a selective androgen receptor N-terminal domain inhibitor including but not limited to LY2452473; or EZH2 inhibitor including but not limited to CPI-1205. In another embodiment, the second therapeutically active agent is a nonsteroidal antiandrogen (NSAA).

In one embodiment, the second therapeutically active agent is selected from a poly (ADP-ribose) polymerase (PARP) inhibitor including but not limited to olaparib, niraparib, rucaparib, talazoparib; an androgen receptor ligand-binding domain inhibitor including but not limited to enzalutamide, apalutamide, darolutamide, bicalutamide, nilutamide, flutamide, ODM-204, TAS3681; an inhibitor of CYP17 including but not limited to galeterone, abiraterone, abiraterone acetate; a microtubule inhibitor including but not limited to docetaxel, paclitaxel, cabazitaxel (XRP-6258); a modulator of PD-1 or PD-L1 including but not limited to pembrolizumab, durvalumab, nivolumab, atezolizumab; a gonadotropin releasing hormone agonist including but not limited to cyproterone acetate, leuprolide; a 5-alpha reductase inhibitor including but not limited to finasteride, dutasteride, turosteride, bexlosteride, izonsteride, FCE 28260, SKF105,111; a vascular endothelial growth factor inhibitor including but not limited to bevacizumab (Avastin); a histone deacetylase inhibitor including but not limited to OSU-HDAC42; an integrin alpha-v-beta-3 inhibitor including but not limited to VITAXIN; a receptor tyrosine kinase including but not limited to sunitumib; a phosphoinositide 3-kinase inhibitor including but not limited to alpelisib, buparlisib, idealisib; an anaplastic lymphoma kinase (ALK) inhibitor including but not limited to crizotinib, alectinib; an endothelin receptor A antagonist including but not limited to ZD-4054; an anti-CTLA4 inhibitor including but not limited to MDX-010 (ipilimumab); an heat shock protein 27 (HSP27) inhibitor including but not limited to OGX 427; an androgen receptor degrader including but not limited to ARV-330, ARV-110; a androgen receptor DNA-binding domain inhibitor including but not limited to VPC-14449; a bromodomain and extra-terminal motif (BET) inhibitor including but not limited to BI-894999, GSK25762, GS-5829; an N-terminal domain inhibitor including but not limited to a sintokamide; an alpha-particle emitting radioactive therapeutic agent including but not limited to radium 233 or a salt thereof, niclosamide; or related compounds thereof, a selective estrogen receptor modulator (SERM) including but not limited to tamoxifen, raloxifene, toremifene, arzoxifene, bazedoxifene, pipindoxifene, lasofoxifene, enclomiphene; a selective estrogen receptor degrader (SERD) including but not limited to fulvestrant, ZB716, OP-1074, elacestrant, AZD9496, GDC0810, GDC0927, GW5638, GW7604; an aromatase inhibitor including but not limited to anastrazole, exemestane, letrozole; selective progesterone receptor modulators (SPRM) including but not limited to mifepristone, lonaprison, onapristone, asoprisnil, lonaprisnil, ulipristal, telapristone; a glucocorticoid receptor inhibitor including but not limited to mifepristone, COR108297, COR125281, ORIC-101, PT150; HER2 receptor antagonist including but not limited to trastuzumab, neratinib; or a mammalian target of rapamycin (mTOR) inhibitor including but not limited to everolimus, temsirolimus.

In one embodiment, the second therapeutically active agent is a microtubule inhibitor. In one embodiment, the microtubule inhibitor is selected from docetaxel, paclitaxel, or cabazitaxel (XRP-6258). In one embodiment, the microtubule inhibitor is docetaxel.

In one embodiment, the second therapeutically active agent is a Bcl-2 inhibitor. In one embodiment, the Bcl-2 inhibitor is venetoclax.

### Therapeutic Use

The pharmaceutical compositions and combinations of the present disclosure find use in any number of methods. For example, in some embodiments the compounds are useful in methods for modulating androgen receptor (AR). In some embodiments, modulating androgen receptor (AR) activity is in a mammalian cell. In some embodiments, modulating androgen receptor (AR) can be in a subject in need thereof (e.g., a mammalian subject) and for treatment of any of the described conditions or diseases.

In one embodiment, the modulating AR is binding to AR. In other embodiments, the modulating AR is inhibiting AR.

In one embodiment, the modulating AR is modulating AR N-terminal domain (NTD). In one embodiment, the modulating AR is binding to AR NTD. In other embodiments, the modulating AR is inhibiting AR NTD. In one embodiment, the modulating AR is modulating AR N-terminal domain (NTD). In some embodiments, modulating the AR is inhibiting transactivation of androgen receptor N-terminal domain (NTD).

In other embodiments, modulating androgen receptor (AR) activity is for treatment of at least one indication selected from the group consisting of: prostate cancer, breast cancer, ovarian cancer, bladder cancer, pancreatic cancer, hepatocellular cancer, endometrial cancer, salivary gland carcinoma, hair loss, acne, hirsutism, ovarian cysts, polycystic ovary disease, precocious puberty, spinal and bulbar muscular atrophy, age related macular degeneration, and combinations thereof. For example in some embodiments, the indication is prostate cancer. In other embodiments, the prostate cancer is primary/localized prostate cancer, locally advanced prostate cancer, recurrent prostate cancer, metastatic prostate cancer, advanced prostate cancer, or metastatic castration-resistant prostate cancer (CRPC), or hormone-sensitive prostate cancer. While in other embodiments, the prostate cancer is androgen dependent prostate cancer. In other embodiments, the spinal and bulbar muscular atrophy is Kennedy's disease.

In one embodiment of the present disclosure, a method of treating a condition associated with cell proliferation in a patient in need thereof is provided. In one embodiment, the present invention provides a method of treating cancer or tumors. In another embodiment, the present invention provides a method of treating prostate cancer or breast cancer. In another embodiment, the present invention provides a method of treating prostate cancer.

In one embodiment of the present disclosure, a method of reducing, inhibiting, or ameliorating cell proliferation in a patient in need thereof is provided. In one embodiment, the reducing, inhibiting, or ameliorating in the method disclosed herein, is in vivo. In another embodiment, the reducing, inhibiting, or ameliorating is in vitro.

In one embodiment, the cells in the method disclosed herein, are a cancer cells. In one embodiment, the cancer cells are a prostate cancer cells. In one embodiment, the prostate cancer cells are cells of primary/localized prostate cancer (newly diagnosed or early stage), locally advanced prostate cancer, recurrent prostate cancer (e.g., prostate cancer which was not cured with primary therapy), metastatic prostate cancer, advanced prostate cancer (e.g., after castration for recurrent prostate cancer), metastatic castration-resistant prostate cancer (CRPC), or hormone-sensitive prostate cancer. In another embodiment, the prostate cancer cells are cells of a metastatic castration-resistant prostate cancer. In other embodiments, the prostate cancer cells are an androgen-dependent prostate cancer cells or an androgen-independent prostate cancer cells. In one embodiment, the cancer cells are breast cancer cells.

In one embodiment, the condition or disease associated with cell proliferation is cancer. In one embodiment of any one of the methods disclosed herein, the cancer is selected from the group consisting of: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy, and age-related macular degeneration. In one embodiment, the condition or disease is prostate cancer. In one embodiment, prostate cancer is selected from primary/localized prostate cancer, locally advanced prostate cancer, recurrent prostate cancer, metastatic prostate cancer, advanced prostate cancer, metastatic castration-resistant prostate cancer (CRPC), or hormone-sensitive prostate cancer. In another embodiment, the prostate cancer is a metastatic castration-resistant prostate cancer. In some embodiments, the prostate cancer is an androgen-dependent prostate cancer cells or an androgen-independent prostate cancer. In one embodiment, the condition or disease is breast cancer. In one embodiment, the breast cancer is AR-positive triple negative breast cancer.

In another embodiment of the present disclosure, a method for reducing or preventing tumor growth, comprising contacting tumor cells with a pharmaceutical composition or a combination as disclosed herein.

In one embodiment, reducing or preventing tumor growth includes reduction in tumor volume. In one embodiment, reducing or preventing tumor growth includes complete elimination of tumors. In one embodiment, reducing or preventing tumor growth includes stopping or halting the existing tumor to grow. In one embodiment, reducing or preventing tumor growth includes reduction in the rate of tumor growth. In one embodiment, reducing or preventing tumor growth includes reduction in the rate of tumor growth such that the rate of tumor growth before treating a patient with the methods disclosed herein (r1) is faster than the rate of tumor growth after said treatment (r2) such that r1>r2.

In one embodiment, the reducing or preventing in the method disclosed herein is in vivo. In another embodiment, the treating is in vitro.

In one embodiment, the tumor cell in the method disclosed herein is selected from prostate cancer, breast cancer, ovarian cancer, endometrial cancer, or salivary gland carcinoma. In one embodiment, the tumor cells are prostate cancer tumor cells. In one embodiment, the prostate cancer tumor cells are tumor cells of primary/localized prostate cancer, locally advanced prostate cancer, recurrent prostate cancer, metastatic prostate cancer, advanced prostate cancer, metastatic castration-resistant prostate cancer (CRPC), or hormone-sensitive prostate cancer. In other embodiments, the prostate cancer is a metastatic castration-resistant prostate cancer. In some embodiments, the prostate cancer is androgen-dependent prostate cancer or androgen-independent prostate cancer. In another embodiment, the tumor cells are is breast cancer tumor cells.

Therapeutic Use Related to Androgen Receptor Driven Gene Expression

In one embodiment, the present disclosure provides a method for treating a subject having a cancer, comprising, obtaining a sample of the cancer before and/or after treatment of the subject with an an androgen receptor modulator.

In one embodiment of the present disclosure, a method of treating a patient with abnormal androgen receptor driven gene activity with androgen receptor modulator alone or in combination with a second therapeutic agent is provided.

In one embodiment, the present disclosure provides a method for treating a subject having a cancer, comprising, obtaining a sample of the cancer before treatment with an androgen receptor modulator, and determining in the sample, the expression level of an androgen receptor driven genes. In another specific embodiment, after testing the expression level of androgen receptor driven genes, the subject is administered an androgen receptor modulator alone and or in combination with a second therapeutically active agent as disclosed herein. In a specific embodiment, the genes are one or more selected from the group consisting of KLK2, FKBP5, TMPRSS2, KLK3, NCAPD3, NKX3-1, NDRG1, STEAP4, FAM105A, AKAP12, PMEPA1, PLPP1, SNAl2, ACSL3, ERRFl1, CDC6, ELL2, CENPN, RHOU, EAF2, SGK1, SLC16A6, TIPARP, IGF1R, CCND1, ADAMTS1, and PRR15L.

In one embodiment, the present disclosure provides a method of treating cancer in a subject having abnormal gene expression of one or more androgen receptor driven genes, comprising administering to the subject an androgen receptor modulator is any compound as discussed herein, including a compound of formula (I)-(III), (IIIA), (VI), (IVA), (V)-(X), (A-I), or (G-II) or a compound in Tables A-B, or a pharmaceutically acceptable salt, tautomer, stereoisomer or prodrug thereof. In one embodiment, the androgen receptor modulator is selected from Compounds A13, A57, A74, A93, A109, A112, A122, A126, A131, A134, A136, A137, A164, A168, A169, A170, A171, A172, A184, A185, A195, and/or A204, or a pharmaceutically acceptable salt, tautomer, stereoisomer or prodrug thereof. In one embodiment of any one of the methods disclosed herein, the androgen receptor driven gene is an androgen receptor full-length driven gene. In one embodiment, the androgen receptor driven gene is an androgen receptor V7 driven gene. In one embodiment of any one of the methods disclosed herein, the gene with an abnormal activity is selected from KLK2, FKBP5, TMPRSS2, KLK3, NCAPD3, NKX3-1, NDRG1, STEAP4, FAM105A, AKAP12, PMEPA1, PLPP1, SNAl2, ACSL3, ERRFl1, CDC6, ELL2, CENPN, RHOU, EAF2, SGK1, SLC16A6, TIPARP, IGF1R, CCND1, ADAMTS1, or PRR15L. In one embodiment of the methods disclosed herein, cancer is selected from prostate cancer, breast cancer, ovarian cancer, endometrial cancer, or salivary gland carcinoma. In one embodiment, the cancer is prostate cancer. In one embodiment, the prostate cancer is selected from primary/localized prostate cancer, locally advanced prostate cancer, recurrent prostate cancer, metastatic prostate cancer, advanced prostate cancer, metastatic castration-resistant prostate cancer (CRPC), or hormone-sensitive prostate cancer. In other embodiments, the prostate cancer is a metastatic castration-resistant prostate cancer. In some embodiments, the prostate cancer is androgen-dependent prostate cancer or androgen-independent prostate cancer. In another embodiment, the cancer is breast cancer. In a specific embodiment, the androgen receptor modulator is Compound A109.

In one embodiment, the present disclosure provides a method of treating cancer in a subject having abnormal gene expression of one or more androgen receptor driven genes, comprising administering to the subject an androgen receptor modulator is any compound as discussed herein, including a compound of formula (I)-(III), (IIIA), (VI), (IVA), (V)-(X), (A-I), or (G-II) or a compound in Tables A-B, or a pharmaceutically acceptable salt, tautomer, stereoisomer or prodrug thereof. In one embodiment, the androgen receptor modulator is selected from Compounds A13, A57, A74, A93, A109, A112, A122, A126, A131, A134, A136, A137, A164, A168, A169, A170, A171, A172, A184, A185, A195, and/or A204, or a pharmaceutically acceptable salt, tautomer, stereoisomer or prodrug thereof in combination with a second therapeutically active agent as disclosed herein. In a specific embodiment, the second therapeutically active agent is a nonsteroidal antiandrogen (NSAA). In one embodiment of the pharmaceutical composition of the present disclosure, the androgen receptor ligand-binding domain inhibitor is enzalutamide, apalutamide, darolutamide, bicalutamide, nilutamide, flutamide, ODM-204, or TAS3681. In one embodiment, the androgen receptor ligand-binding domain inhibitor is enzalutamide.

In one embodiment of any one of the methods disclosed herein, the androgen receptor driven gene is an androgen receptor full-length driven gene. In one embodiment, the androgen receptor driven gene is an androgen receptor V7 driven gene. In one embodiment of any one of the methods disclosed herein, the gene with an abnormal activity is selected from KLK2, FKBP5, TMPRSS2, KLK3, NCAPD3, NKX3-1, NDRG1, STEAP4, FAM105A, AKAP12, PMEPA1, PLPP1, SNAl2, ACSL3, ERRFl1, CDC6, ELL2, CENPN, RHOU, EAF2, SGK1, SLC16A6, TIPARP, IGF1R, CCND1, ADAMTS1, or PRR15L. In one embodiment of the methods disclosed herein, cancer is selected from prostate cancer, breast cancer, ovarian cancer, endometrial cancer, or salivary gland carcinoma. In one embodiment, the cancer is prostate cancer. In one embodiment, the prostate cancer is selected from primary/localized prostate cancer, locally advanced prostate cancer, recurrent prostate cancer, metastatic prostate cancer, advanced prostate cancer, metastatic castration-resistant prostate cancer (CRPC), or hormone-sensitive prostate cancer. In other embodiments, the prostate cancer is a metastatic castration-resistant prostate cancer. In some embodiments, the prostate cancer is androgen-dependent prostate cancer or androgen-independent prostate cancer. In another embodiment, the cancer is breast cancer. In a specific embodiment, the androgen receptor modulator is Compound A109 and the second therapeutically active agent is enzalutamide.

In one embodiment, the present disclosure provides a method for treating a subject having a cancer, comprising, obtaining a sample of the cancer after treatment with an androgen receptor modulator, and determining, in the sample, the expression level of an androgen receptor driven gene, where if the gene expression level, when compared to a reference standard level, is decreased before or after treatment with the androgen receptor modulator, then proceeding with or resuming treatment of the subject with a therapeutically effective amount of the androgen receptor modulator and/or a second therapeutically active agent. In a specific embodiment, the gene is selected from one or more of the group consisting of KLK2, FKBP5, TMPRSS2, KLK3, NCAPD3, NKX3-1, NDRG1, STEAP4, FAM105A, AKAP12, PMEPA1, PLPP1, SNAl2, ACSL3, ERRFl1, CDC6, ELL2, CENPN, RHOU, EAF2, SGK1, SLC16A6, TIPARP, IGF1R, CCND1, ADAMTS1, and PRR15L. In one embodiment, an androgen receptor modulator administered before the sample of cancer is obtained can be the same or different from an androgen receptor modulator administered after the androgen receptor driven gene expression levels are assessed.

In one embodiment, the present disclosure provides a method for treating a subject having a cancer, comprising, obtaining a sample of the cancer after treatment with an androgen receptor modulator, and determining, in the sample, the expression level of an androgen receptor driven gene, where if the gene expression level, when compared to a reference standard level, is decreased before or after treatment with the androgen receptor modulator, then proceeding with or resuming treatment of the subject with a therapeutically effective amount of the androgen receptor modulator or a different androgen receptor modulator and a second therapeutic agent, wherein the gene is selected from one or more of the group consisting of KLK2, FKBP5, TMPRSS2, KLK3, NCAPD3, NKX3-1, NDRG1, STEAP4, FAM105A, AKAP12, PMEPA1, PLPP1, SNAl2, ACSL3, ERRFl1, CDC6, ELL2, CENPN, RHOU, EAF2, SGK1, SLC16A6, TIPARP, IGF1R, CCND1, ADAMTS1, and PRR15L. In one embodiment, the second therapeutic agent is an androgen receptor ligand-binding domain inhibitor is enzalutamide, apalutamide, darolutamide, bicalutamide, nilutamide, flutamide, ODM-204, or TAS3681. In one embodiment, the androgen receptor ligand-binding domain inhibitor is enzalutamide. In one embodiment, the second therapeutic agent is a Bcl-2 inhibitor. In one embodiment, the Bcl-2 inhibitor is venetoclax. In one embodiment, the second therapeutic agent is an androgen receptor N-terminal domain inhibitor. In one embodiment, the second therapeutic agent is an androgen receptor N-terminal domain inhibitor selected from Tables A-B.

In one embodiment, the present disclosure provides a method for treating a subject having a cancer, comprising, obtaining a sample of the cancer after treatment with an androgen receptor modulator, and determining, in the sample, the expression level of an androgen receptor driven genes, where if the gene expression level, when compared to a reference standard level, is decreased before or after treatment with the androgen receptor modulator, then proceeding with or resuming treatment of the subject with a therapeutically effective amount of Compound A109 and enzalutamide, wherein the gene is selected from KLK2, FKBP5, TMPRSS2, KLK3, NCAPD3, NKX3-1, NDRG1, STEAP4, FAM105A, AKAP12, PMEPA1, PLPP1, SNAl2, ACSL3, ERRFl1, CDC6, ELL2, CENPN, RHOU, EAF2, SGK1, SLC16A6, TIPARP, IGF1R, CCND1, ADAMTS1, or PRR15L.

In one embodiment, the present disclosure provides a method for treating a subject having a cancer, comprising, obtaining a sample of the cancer after treatment with Compound A109, and determining, in the sample, the expression level of an androgen receptor driven genes, where if the gene expression level, when compared to a reference standard level, is decreased before or after treatment with Compound A109, then proceeding with or resuming treatment of the subject with a therapeutically effective amount of Compound A109. In a specific embodiment, enzalutamide may be co-adminstered as second therapeutic agent. In another specific embodiment, the gene is selected from KLK2, FKBP5, TMPRSS2, KLK3, NCAPD3, NKX3-1, NDRG1, STEAP4, FAM105A, AKAP12, PMEPA1, PLPP1, SNAl2, ACSL3, ERRFl1, CDC6, ELL2, CENPN, RHOU, EAF2, SGK1, SLC16A6, TIPARP, IGF1R, CCND1, ADAMTS1, or PRR15L.

In one embodiment, the present disclosure provides a method for treating a subject having a cancer, comprising, obtaining a sample of the cancer after treatment with an androgen receptor modulator, and determining, in the sample, the expression level of an androgen receptor driven genes, where if the gene expression level, when compared to a reference standard level, is decreased before or after treatment with the androgen receptor modulator, then proceeding with or resuming treatment of the subject with a therapeutically effective amount of Compound A109, wherein the gene is selected from one or more selected from the group consisting of KLK2, FKBP5, TMPRSS2, KLK3, NCAPD3, NKX3-1, NDRG1, STEAP4, FAM105A, AKAP12, PMEPA1, PLPP1, SNAl2, ACSL3, ERRFl1, CDC6, ELL2, CENPN, RHOU, EAF2, SGK1, SLC16A6, TIPARP, IGF1R, CCND1, ADAMTS1, and PRR15L.

In one embodiment, the present disclosure provides a method for treating a subject having a cancer, comprising, obtaining a sample of the cancer after treatment with Compound A109, and determining, in the sample, the expression level of an androgen receptor driven genes, where if the gene expression level, when compared to a reference standard level, is decreased before or after treatment with Compound A109, then proceeding with or resuming treatment of the subject with a therapeutically effective amount of Compound A109, wherein the gene is selected from one or more selected from the group consisting of KLK2, FKBP5, TMPRSS2, KLK3, NCAPD3, NKX3-1, NDRG1, STEAP4, FAM105A, AKAP12, PMEPA1, PLPP1, SNAl2, ACSL3, ERRFl1, CDC6, ELL2, CENPN, RHOU, EAF2, SGK1, SLC16A6, TIPARP, IGF1R, CCND1, ADAMTS1, and PRR15L.

In one embodiment of the methods disclosed herein, cancer is selected from prostate cancer, breast cancer, ovarian cancer, endometrial cancer, or salivary gland carcinoma. In one embodiment, the cancer is prostate cancer. In one embodiment, the prostate cancer is selected from primary/localized prostate cancer, locally advanced prostate cancer, recurrent prostate cancer, metastatic prostate cancer, advanced prostate cancer, metastatic castration-resistant prostate cancer (CRPC), or hormone-sensitive prostate cancer. In other embodiments, the prostate cancer is a metastatic castration-resistant prostate cancer. In some embodiments, the prostate cancer is androgen-dependent prostate cancer or androgen-independent prostate cancer. In another embodiment, the cancer is breast cancer.

In one embodiment of any one of the methods disclosed herein, the androgen receptor modulator is any compound as discussed herein, including a compound of formula (I)-(III), (IIIA), (VI), (IVA), (V)-(X), (A-I), or (G-II) or a compound in Tables A-B, or a pharmaceutically acceptable salt, tautomer, stereoisomer or prodrug thereof. In one embodiment, the patient has cancer. In one embodiment, the androgen receptor modulator is selected from Compounds A13, A57, A74, A93, A109, A112, A122, A126, A131, A134, A136, A137, A164, A168, A169, A170, A171, A172, A184, A185, A195, and/or A204, or a pharmaceutically acceptable salt, tautomer, stereoisomer or prodrug thereof.

In one embodiment of any one of the methods disclosed herein, the androgen receptor driven gene is an androgen receptor full-length driven gene. In one embodiment, the androgen receptor driven gene is an androgen receptor V7 driven gene.

In one embodiment of any one of the methods disclosed herein, the gene with an abnormal activity is selected from KLK2, FKBP5, TMPRSS2, KLK3, NCAPD3, NKX3-1, NDRG1, STEAP4, FAM105A, AKAP12, PMEPA1, PLPP1, SNAl2, ACSL3, ERRFl1, CDC6, ELL2, CENPN, RHOU, EAF2, SGK1, SLC16A6, TIPARP, IGF1R, CCND1, ADAMTS1, or PRR15L.

### Pharmaceutical Compositions and Formulations

The pharmaceutical composition or a combination as disclosed herein, can further comprise a pharmaceutically acceptable carrier or excipient.

In one embodiment, pharmaceutical composition or a combination comprises an androgen receptor modulator selected from Table A, or a pharmaceutically acceptable salt or solvate thereof, a second therapeutically effective agent, and a pharmaceutically acceptable carrier or excipient. In one embodiment, the pharmaceutical composition or the combination comprises a Bcl-2 inhibitor, an androgen receptor modulator selected from Table A, or a pharmaceutically acceptable salt or solvate thereof, and a pharmaceutically acceptable carrier or excipient. In one embodiment, the pharmaceutical composition or the combination comprises venetoclax, an androgen receptor modulator selected from Table A, or a pharmaceutically acceptable salt or solvate thereof, and a pharmaceutically acceptable carrier or excipient. In one embodiment, the pharmaceutical composition or the combination comprises an androgen receptor ligand-binding domain inhibitor, an androgen receptor modulator selected from Table A, or a pharmaceutically acceptable salt or solvate thereof, and a pharmaceutically acceptable carrier or excipient. In one embodiment, the pharmaceutical composition or the combination comprises a) enzalutamide, apalutamide, or darolutamide, b) an androgen receptor modulator selected from Table A, or a pharmaceutically acceptable salt or solvate thereof, and c) a pharmaceutically acceptable carrier or excipient. In one embodiment, the pharmaceutical composition or the combination comprises a microtubule inhibitor, an androgen receptor modulator selected from Table A, or a pharmaceutically acceptable salt or solvate thereof, and a pharmaceutically acceptable carrier or excipient. In one embodiment, the pharmaceutical composition or the combination comprises docetaxel, an androgen receptor modulator selected from Table A, or a pharmaceutically acceptable salt or solvate thereof, and a pharmaceutically acceptable carrier or excipient.

In one embodiment, pharmaceutical composition or a combination comprises an androgen receptor modulator selected from Table B, or a pharmaceutically acceptable salt or solvate thereof, a second therapeutically effective agent, and a pharmaceutically acceptable carrier or excipient. In one embodiment, the pharmaceutical composition or the combination comprises a Bcl-2 inhibitor, an androgen receptor modulator selected from Table B, or a pharmaceutically acceptable salt or solvate thereof, and a pharmaceutically acceptable carrier or excipient. In one embodiment, the pharmaceutical composition or the combination comprises venetoclax, an androgen receptor modulator selected from Table B, or a pharmaceutically acceptable salt or solvate thereof, and a pharmaceutically acceptable carrier or excipient. In one embodiment, the pharmaceutical composition or the combination comprises an androgen receptor ligand-binding domain inhibitor, an androgen receptor modulator selected from Table B, or a pharmaceutically acceptable salt or solvate thereof, and a pharmaceutically acceptable carrier or excipient. In one embodiment, the pharmaceutical composition or the combination comprises a) enzalutamide, apalutamide, or darolutamide, b) an androgen receptor modulator selected from Table B, or a pharmaceutically acceptable salt or solvate thereof, and c) a pharmaceutically acceptable carrier or excipient. In one embodiment, the pharmaceutical composition or the combination comprises a microtubule inhibitor, an androgen receptor modulator selected from Table B, or a pharmaceutically acceptable salt or solvate thereof, and a pharmaceutically acceptable carrier or excipient. In one embodiment, the pharmaceutical composition or the combination comprises docetaxel, an androgen receptor modulator selected from Table B, or a pharmaceutically acceptable salt or solvate thereof, and a pharmaceutically acceptable carrier or excipient.

In one embodiment, pharmaceutical composition or a combination comprises venetoclax, Compound A109, or a pharmaceutically acceptable salt or solvate thereof, and a pharmaceutically acceptable carrier or excipient.

In one embodiment, pharmaceutical composition or a combination comprises enzalutamide, Compound A109, or a pharmaceutically acceptable salt or solvate thereof, and a pharmaceutically acceptable carrier or excipient.

In one embodiment, a pharmaceutical composition, as described herein, further comprises one or more additional therapeutically active agents. In one embodiment, one or more additional therapeutically active agents are selected from therapeutics useful for treating cancer, neurological disease, a disorder characterized by abnormal accumulation of α-synuclein, a disorder of an aging process, cardiovascular disease, bacterial infection, viral infection, mitochondrial related disease, mental retardation, deafness, blindness, diabetes, obesity, autoimmune disease, glaucoma, Leber's Hereditary Optic Neuropathy, and rheumatoid arthritis.

In some embodiments, the one or more additional therapeutic agents is a poly (ADP-ribose) polymerase (PARP) inhibitor including but not limited to olaparib, niraparib, rucaparib, talazoparib; an androgen receptor ligand-binding domain inhibitor including but not limited to enzalutamide, apalutamide, darolutamide, bicalutamide, nilutamide, flutamide, ODM-204, TAS3681; an inhibitor of CYP17 including but not limited to galeterone, abiraterone, abiraterone acetate; a microtubule inhibitor including but not limited to docetaxel, paclitaxel, cabazitaxel (XRP-6258); a modulator of PD-1 or PD-L1 including but not limited to pembrolizumab, durvalumab, nivolumab, atezolizumab; a gonadotropin releasing hormone agonist including but not limited to cyproterone acetate, leuprolide; a 5-alpha reductase inhibitor including but not limited to finasteride, dutasteride, turosteride, bexlosteride, izonsteride, FCE 28260, SKF105,111; a vascular endothelial growth factor inhibitor including but not limited to bevacizumab (Avastin); a histone deacetylase inhibitor including but not limited to OSU-HDAC42; an integrin alpha-v-beta-3 inhibitor including but not limited to VITAXIN; a receptor tyrosine kinase including but not limited to sunitumib; a phosphoinositide 3-kinase inhibitor including but not limited to alpelisib, buparlisib, idealisib; an anaplastic lymphoma kinase (ALK) inhibitor including but not limited to crizotinib, alectinib; an endothelin receptor A antagonist including but not limited to ZD-4054; an anti-CTLA4 inhibitor including but not limited to MDX-010 (ipilimumab); an heat shock protein 27 (HSP27) inhibitor including but not limited to OGX 427; an androgen receptor degrader including but not limited to ARV-330, ARV-110; a androgen receptor DNA-binding domain inhibitor including but not limited to VPC-14449; a bromodomain and extra-terminal motif (BET) inhibitor including but not limited to BI-894999, GSK25762, GS-5829; an N-terminal domain inhibitor including but not limited to a sintokamide; an alpha-particle emitting radioactive therapeutic agent including but not limited to radium 233 or a salt thereof, niclosamide; or related compounds thereof, a selective estrogen receptor modulator (SERM) including but not limited to tamoxifen, raloxifene, toremifene, arzoxifene, bazedoxifene, pipindoxifene, lasofoxifene, enclomiphene; a selective estrogen receptor degrader (SERD) including but not limited to fulvestrant, ZB716, OP-1074, elacestrant, AZD9496, GDC0810, GDC0927, GW5638, GW7604; an aromatase inhibitor including but not limited to anastrazole, exemestane, letrozole; selective progesterone receptor modulators (SPRM) including but not limited to mifepristone, lonaprison, onapristone, asoprisnil, lonaprisnil, ulipristal, telapristone; a glucocorticoid receptor inhibitor including but not limited to mifepristone, COR108297, COR125281, ORIC-101, PT150; HER2 receptor antagonist including but not limited to trastuzumab, neratinib; a mammalian target of rapamycin (mTOR) inhibitor including but not limited to everolimus, temsirolimus.

In a further embodiment of the present disclosure, a pharmaceutical composition or combination as disclosed herein comprises a pharmaceutically acceptable carrier, excipient or adjuvant is provided. The pharmaceutically acceptable carriers, excipients and adjuvants are added to the composition or formulation for a variety of purposes. In one embodiment, a pharmaceutically acceptable carrier includes a pharmaceutically acceptable excipient, binder, and/or diluent. In one embodiment, suitable pharmaceutically acceptable excipients include, but are not limited to, water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, the pharmaceutical compositions of the present disclosure may additionally contain other adjunct components conventionally found in pharmaceutical compositions, at their art-established usage levels. Thus, for example, the pharmaceutical compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the oligonucleotide(s) of the formulation.

For the purposes of this disclosure, the compounds of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

The compounds disclosed herein can be formulated in accordance with the routine procedures adapted for desired administration route. Accordingly, the compounds disclosed herein can take such forms as suspensions, solutions or emulsions in oily or aqueous vehicles, and can contain formulatory agents such as suspending, stabilizing and/or dispersing agents. The compounds disclosed herein can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). Alternatively, the active ingredient can be in powder form for constitution with a suitable vehicle, e.g., sterile pyrogen-free water, before use. Suitable formulations for each of these methods of administration can be found, for example, in Remington: The Science and Practice of Pharmacy, A. Gennaro, ed., 20th edition, Lippincott, Williams & Wilkins, Philadelphia, Pa.

In certain embodiments, a pharmaceutical composition of the present disclosure is prepared using known techniques, including, but not limited to mixing, dissolving, granulating, dragee-making, levigating, emulsifying, encapsulating, entrapping or tableting processes.

In one embodiment, suitable pharmaceutically acceptable carriers include, but are not limited to, inert solid fillers or diluents and sterile aqueous or organic solutions. Pharmaceutically acceptable carriers are well known to those skilled in the art and include, but are not limited to, from about 0.01 to about 0.1 M and preferably 0.05M phosphate buffer or 0.8% saline. Such pharmaceutically acceptable carriers can be aqueous or non-aqueous solutions, suspensions and emulsions. Examples of non-aqueous solvents suitable for use in the present application include, but are not limited to, propylene glycol, polyethylene glycol, vegetable oils such as olive oil, and injectable organic esters such as ethyl oleate.

Aqueous carriers suitable for use in the present application include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the present application can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the present application include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellent.

Solid carriers suitable for use in the present application include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

Parenteral carriers suitable for use in the present application include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the present application can be mixed as needed with disintegrants, diluents, granulating agents, lubricants, binders and the like using conventional techniques known in the art. The carriers can also be sterilized using methods that do not deleteriously react with the compounds, as is generally known in the art.

Diluents may be added to the formulations of the present invention. Diluents increase the bulk of a solid pharmaceutical composition and/or combination, and may make a pharmaceutical dosage form containing the composition and/or combination easier for the patient and care giver to handle. Diluents for solid compositions and/or combinations include, for example, microcrystalline cellulose (e.g., AVICEL), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT®), potassium chloride, powdered cellulose, sodium chloride, sorbitol, and talc.

Additional embodiments relate to the pharmaceutical formulations wherein the formulation is selected from the group consisting of a solid, powder, liquid and a gel. In certain embodiments, a pharmaceutical composition of the present invention is a solid (e.g., a powder, tablet, a capsule, granulates, and/or aggregates). In certain of such embodiments, a solid pharmaceutical composition comprising one or more ingredients known in the art, including, but not limited to, starches, sugars, diluents, granulating agents, lubricants, binders, and disintegrating agents.

Solid pharmaceutical compositions that are compacted into a dosage form, such as a tablet, may include excipients whose functions include helping to bind the active ingredient and other excipients together after compression. Binders for solid pharmaceutical compositions and/or combinations include acacia, alginic acid, carbomer (e.g., carbopol), carboxymethylcellulose sodium, dextrin, ethyl cellulose, gelatin, guar gum, gum tragacanth, hydrogenated vegetable oil, hydroxyethyl cellulose, hydroxypropyl cellulose (e.g., KLUCEL), hydroxypropyl methyl cellulose (e.g., METHOCEL), liquid glucose, magnesium aluminum silicate, maltodextrin, methylcellulose, polymethacrylates, povidone (e.g., KOLLIDON, PLASDONE), pregelatinized starch, sodium alginate, and starch.

The dissolution rate of a compacted solid pharmaceutical composition in the patient's stomach may be increased by the addition of a disintegrant to the composition and/or combination. Disintegrants include alginic acid, carboxymethylcellulose calcium, carboxymethylcellulose sodium (e.g., AC-DI-SOL and PRIMELLOSE), colloidal silicon dioxide, croscarmellose sodium, crospovidone (e.g., KOLLIDON and POLYPLASDONE), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB), potato starch, and starch.

Glidants can be added to improve the flowability of a non-compacted solid composition and/or combination and to improve the accuracy of dosing. Excipients that may function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition and/or combination to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that may be included in the composition and/or combination of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In certain embodiments, a pharmaceutical composition of the present invention is a liquid (e.g., a suspension, elixir and/or solution). In certain of such embodiments, a liquid pharmaceutical composition is prepared using ingredients known in the art, including, but not limited to, water, glycols, oils, alcohols, flavoring agents, preservatives, and coloring agents.

Liquid pharmaceutical compositions can be prepared where the solid or amorphous components are dissolved or suspended in a liquid carrier such as water, vegetable oil, alcohol, polyethylene glycol, propylene glycol, or glycerin.

For example, formulations for parenteral administration can contain as common excipients sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers can be useful excipients to control the release of active compounds. Other potentially useful parenteral delivery systems include ethylene-vinyl acetate copolymer particles, osmotic pumps, implantable infusion systems, and liposomes. Formulations for inhalation administration contain as excipients, for example, lactose, or can be aqueous solutions containing, for example, polyoxyethylene-9-auryl ether, glycocholate and deoxycholate, or oily solutions for administration in the form of nasal drops, or as a gel to be applied intranasally. Formulations for parenteral administration can also include glycocholate for buccal administration, methoxysalicylate for rectal administration, or citric acid for vaginal administration.

Liquid pharmaceutical compositions can contain emulsifying agents to disperse uniformly throughout the composition and/or combination an active ingredient or other excipient that is not soluble in the liquid carrier. Emulsifying agents that may be useful in liquid compositions and/or combinations of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as aspartame, lactose, sorbitol, saccharin, sodium saccharin, sucrose, aspartame, fructose, mannitol, and invert sugar may be added to improve the taste.

Preservatives and chelating agents such as alcohol, sodium benzoate, butylated hydroxyl toluene, butylated hydroxyanisole, and ethylenediamine tetraacetic acid may be added at levels safe for ingestion to improve storage stability.

A liquid composition can also contain a buffer such as guconic acid, lactic acid, citric acid or acetic acid, sodium guconate, sodium lactate, sodium citrate, or sodium acetate. Selection of excipients and the amounts used may be readily determined by the formulation scientist based upon experience and consideration of standard procedures and reference works in the field.

In one embodiment, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions.

The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables. Formulations for intravenous administration can comprise solutions in sterile isotonic aqueous buffer. Where necessary, the formulations can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where the compound is to be administered by infusion, it can be dispensed in a formulation with an infusion bottle containing sterile pharmaceutical grade water, saline or dextrose/water. Where the compound is administered by injection, an ampule of sterile water for injection or saline can be provided so that the ingredients can be mixed prior to administration.

Suitable formulations further include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

In certain embodiments, a pharmaceutical composition of the present invention is formulated as a depot preparation. Certain such depot preparations are typically longer acting than non-depot preparations. In certain embodiments, such preparations are administered by implantation (for example subcutaneously or intramuscularly) or by intramuscular injection. In certain embodiments, depot preparations are prepared using suitable polymeric or hydrophobic materials (for example an emulsion in an acceptable oil) or ion exchange resins, or as sparingly soluble derivatives, for example, as a sparingly soluble salt.

In certain embodiments, a pharmaceutical composition of the present invention comprises a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, a pharmaceutical composition of the present invention comprises a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80 and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, a pharmaceutical composition of the present invention comprises a sustained-release system. A non-limiting example of such a sustained-release system is a semi-permeable matrix of solid hydrophobic polymers. In certain embodiments, sustained-release systems may, depending on their chemical nature, release pharmaceutical agents over a period of hours, days, weeks or months.

Appropriate pharmaceutical compositions of the present disclosure can be determined according to any clinically-acceptable route of administration of the composition to the subject. The manner in which the composition is administered is dependent, in part, upon the cause and/or location. One skilled in the art will recognize the advantages of certain routes of administration. The method includes administering an effective amount of the agent or compound (or composition comprising the agent or compound) to achieve a desired biological response, e.g., an amount effective to alleviate, ameliorate, or prevent, in whole or in part, a symptom of a condition to be treated, e.g., oncology and neurology disorders. In various aspects, the route of administration is systemic, e.g., oral or by injection. The agents or compounds, or pharmaceutically acceptable salts or derivatives thereof, are administered orally, nasally, transdermally, pulmonary, inhalationally, buccally, sublingually, intraperintoneally, subcutaneously, intramuscularly, intravenously, rectally, intrapleurally, intrathecally, intraportally, and parenterally. Alternatively or in addition, the route of administration is local, e.g., topical, intra-tumor and peri-tumor. In some embodiments, the compound is administered orally.

In certain embodiments, a pharmaceutical composition of the present disclosure is prepared for oral administration. In certain of such embodiments, a pharmaceutical composition is formulated by combining one or more agents and pharmaceutically acceptable carriers. Certain of such carriers enable pharmaceutical compositions to be formulated as tablets, pills, dragees, capsules, liquids, gels, syrups, slurries, suspensions and the like, for oral ingestion by a subject. Suitable excipients include, but are not limited to, fillers, such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In certain embodiments, such a mixture is optionally ground and auxiliaries are optionally added. In certain embodiments, pharmaceutical compositions are formed to obtain tablets or dragee cores. In certain embodiments, disintegrating agents (e.g., cross-linked polyvinyl pyrrolidone, agar, or alginic acid or a salt thereof, such as sodium alginate) are added.

In certain embodiments, dragee cores are provided with coatings. In certain such embodiments, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to tablets or dragee coatings.

In certain embodiments, pharmaceutical compositions for oral administration are push-fit capsules made of gelatin. Certain of such push-fit capsules comprise one or more pharmaceutical agents of the present invention in admixture with one or more filler such as lactose, binders such as starches, and/or lubricants such as talc or magnesium stearate and, optionally, stabilizers. In certain embodiments, pharmaceutical compositions for oral administration are soft, sealed capsules made of gelatin and a plasticizer, such as glycerol or sorbitol. In certain soft capsules, one or more pharmaceutical agents of the present invention are be dissolved or suspended in suitable liquids, such as fatty oils, liquid paraffin, or liquid polyethylene glycols. In addition, stabilizers may be added.

In certain embodiments, pharmaceutical compositions are prepared for buccal administration. Certain of such pharmaceutical compositions are tablets or lozenges formulated in conventional manner.

In certain embodiments, a pharmaceutical composition is prepared for transmucosal administration. In certain of such embodiments penetrants appropriate to the barrier to be permeated are used in the formulation. Such penetrants are generally known in the art.

In certain embodiments, a pharmaceutical composition is prepared for administration by inhalation. Certain of such pharmaceutical compositions for inhalation are prepared in the form of an aerosol spray in a pressurized pack or a nebulizer. Certain of such pharmaceutical compositions comprise a propellant, e.g., dichlorodifluoromethane, trichlorofluoromethane, dichlorotetrafluoroethane, carbon dioxide or other suitable gas. In certain embodiments using a pressurized aerosol, the dosage unit may be determined with a valve that delivers a metered amount. In certain embodiments, capsules and cartridges for use in an inhaler or insufflator may be formulated. Certain of such formulations comprise a powder mixture of a pharmaceutical agent of the invention and a suitable powder base such as lactose or starch.

In other embodiments the compound of the present disclosure are administered by the intravenous route. In further embodiments, the parenteral administration may be provided in a bolus or by infusion.

In certain embodiments, a pharmaceutical composition is prepared for rectal administration, such as a suppository or retention enema. Certain of such pharmaceutical compositions comprise known ingredients, such as cocoa butter and/or other glycerides.

In certain embodiments, a pharmaceutical composition is prepared for topical administration. Certain of such pharmaceutical compositions comprise bland moisturizing bases, such as ointments or creams. Exemplary suitable ointment bases include, but are not limited to, petrolatum, petrolatum plus volatile silicones, and lanolin and water in oil emulsions. Exemplary suitable cream bases include, but are not limited to, cold cream and hydrophilic ointment.

In certain embodiments, the therapeutically effective amount is sufficient to prevent, alleviate or ameliorate symptoms of a disease or to prolong the survival of the subject being treated. Determination of a therapeutically effective amount is well within the capability of those skilled in the art.

In certain embodiments, one or more compounds of formula (I)-(III), (IIIA), (VI), (IVA), (V)-(X), (A-I), or (G-II), or a pharmaceutically acceptable salt or solvate thereof are formulated as a prodrug. In certain embodiments, upon in vivo administration, a prodrug is chemically converted to the biologically, pharmaceutically or therapeutically more active form. In certain embodiments, prodrugs are useful because they are easier to administer than the corresponding active form. For example, in certain instances, a prodrug may be more bioavailable (e.g., through oral administration) than is the corresponding active form. In certain instances, a prodrug may have improved solubility compared to the corresponding active form. In certain embodiments, prodrugs are less water soluble than the corresponding active form. In certain instances, such prodrugs possess superior transmittal across cell membranes, where water solubility is detrimental to mobility. In certain embodiments, a prodrug is an ester. In certain such embodiments, the ester is metabolically hydrolyzed to carboxylic acid upon administration. In certain instances the carboxylic acid containing compound is the corresponding active form. In certain embodiments, a prodrug comprises a short peptide (polyaminoacid) bound to an acid group. In certain of such embodiments, the peptide is cleaved upon administration to form the corresponding active form.

In certain embodiments, a prodrug is produced by modifying a pharmaceutically active compound such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

In various aspects, the androgen receptor modulators in the pharmaceutical composition or combination as disclosed herein can be administered at about 0.001 mg/kg to about 100 mg/kg body weight (e.g., about 0.01 mg/kg to about 10 mg/kg or about 0.1 mg/kg to about 5 mg/kg).

The concentration of a disclosed compound in a pharmaceutically acceptable mixture will vary depending on several factors, including the dosage of the compound to be administered, the pharmacokinetic characteristics of the compound(s) employed, and the route of administration. The agent may be administered in a single dose or in repeat doses. The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound or salt thereof employed. Treatments may be administered daily or more frequently depending upon a number of factors, including the overall health of a patient, and the formulation and route of administration of the selected compound(s). An ordinarily skilled physician or veterinarian can readily determine and prescribe the effective amount of the drug required to prevent, counter or arrest the progress of the condition.

The compounds or pharmaceutical compositions of the present disclosure may be manufactured and/or administered in single or multiple unit dose forms.

Having now generally described the invention, the same will be more readily understood through reference to the following examples, which are provided by way of illustration and are not intended to be limiting of the present invention.

### EXAMPLES

The disclosure now being generally described, it will be more readily understood by reference to the following examples which are included merely for purposes of illustration of certain aspects and embodiments of the present invention, and are not intended to limit the invention.

Synthetic Preparation

The novel compounds of the present invention can be prepared in a variety of ways known to one skilled in the art of organic synthesis. The compounds of the present invention can be synthesized using the methods as hereinafter described below, together with synthetic methods known in the art of synthetic organic chemistry or variations thereon as appreciated by those skilled in the art.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene and Wuts, Protective Groups in Organic Synthesis, 44th. Ed., Wiley & Sons, 2006, as well as in Jerry March, Advanced Organic Chemistry, 4^{th} edition, John Wiley & Sons, publisher, New York, 1992 which are incorporated herein by reference in their entirety.

Compounds of the present invention can be prepared by the literature methods cited in the following text. The following schemes depict established, known syntheses of these scaffolds.

The groups and/or the substituents of the compounds of the present invention can be synthesized and attached to these scaffolds by the literature methods cited in the following text. The following schemes depict the known techniques for accomplishing this joinder.

General Synthesis

Compounds of the present invention can be synthesized using the following methods. General reaction conditions are given, and reaction products can be purified by general known methods including crystallization, silica gel chromatography using various organic solvents such as hexane, cyclohexane, ethyl acetate, methanol and the like, preparative high pressure liquid chromatography or preparative reverse phase high pressure liquid chromatography.

Representative Synthesis of Androgen Receptor Modulators

For synthesis of Compounds in Tables A and B, see PCT/US2019/057034 for procedures. The disclosures of PCT/US2019/057034 are hereby incorporated by reference in their entireties.

### Example 1: Synthesis of 5-[[4-[1-[3,5-dichloro-4-(3-chloropropoxy)phenyl]-1-methyl-ethyl]phenoxy]methyl]-4-methylsulfonyl-oxazole (A3)

To a suspension of 4-[1-[3,5-dichloro-4-(3-chloropropoxy)phenyl]-1-methyl-ethyl]phenol (7) (0.135 g, 0.36 mmol) and Cs₂CO₃ (0.197 g, 0.6 mmol) in DMF (3 mL) was added (4-methylsulfonyloxazol-5-yl)methyl 4-methylbenzenesulfonate (2) (0.1 g, 0.3 mmol) at 25° C. The mixture was stirred at 60° C. for 6 hours. LCMS showed the reaction was completed. The resulting mixture was poured into H₂O (8 mL) and extracted with EtOAc (5 mL×3). The combined organic layers were washed with brine (5 mL×2), dried over Na₂SO₄, filtered, and concentrated under reduced pressure. The residue was purified by prep-HPLC (HCl) to give 5-[[4-[1-[3,5-dichloro-4-(3-chloropropoxy)phenyl]-1-methyl-ethyl]phenoxy]methyl]-4-methylsulfonyl-oxazole (A1) (37.9 mg, yield: 23.6%) as yellow oil. HPLC purity (220 nm): 96.25%. ¹H NMR (400 MHz, CHCl₃-d) δ 7.99 (s, 1H), 7.16-7.10 (m, 4H), 6.94 (d, J=8.82 Hz, 2H), 5.42 (s, 2H), 4.15 (t, J=5.73 Hz, 2H), 3.86 (t, J=6.50 Hz, 2H) 3.18 (s, 3H), 2.28 (quin, J=6.17 Hz, 2H), 1.62 (s, 6H). LCMS (M+23) m/z: calcd 533; found 556.

### Example 2: Synthesis of 4-((4-(2-(3,5-dichloro-4-(3-chloropropoxy)phenyl)propan-2-yl)phenoxy) methyl)-1-(methylsulfonyl)-1H-imidazole (A5)

To a mixture of 4-((4-(2-(3,5-dichloro-4-(3-chloropropoxy)phenyl)propan-2-yl)phenoxy)methyl)-1H-imidazole (6) (80 mg, 0.2 mmol) and TEA (0.1 mL, 0.5 mmol) in DCM (2 mL) was added methanesulfonyl chloride (41 mg, 0.4 mmol) dropwise at 0° C., and the mixture was stirred at 25° C. for 2 hours. TLC showed the reaction was completed. The mixture was diluted with water (20 mL), extracted with DCM (5 mL×3), and the combined organic layers were washed with brine (10 mL), dried over Na₂SO₄, filtered and concentrated under reduced pressure. The residue was purified by prep-HPLC (NH₄HCO₃) to give 4-((4-(2-(3,5-dichloro-4-(3-chloropropoxy)phenyl)propan-2-yl)phenoxy)methyl)-1-(methyl sulfonyl)-imidazole (A5) (16 mg, yield: 16.6%) as colorless oil. ¹H NMR (400 MHz, CHCl₃-d) δ=7.99 (d, J=1.3 Hz, 1H), 7.40 (s, 1H), 7.15-7.12 (m, 4H), 6.95-6.90 (m, 2H), 5.05 (s, 2H), 4.15 (t, J=5.7 Hz, 2H), 3.86 (t, J=6.4 Hz, 2H), 3.30 (s, 3H), 2.31-2.26 (m, 2H), 1.63 (s, 6H). LCMS (220 nm): 95.2%. LCMS (M+1) m/z: calcd 530.1; found 531.0.

### Example 3: Synthesis of 2-((4-(2-(3,5-dichloro-4-(3-chloropropoxy)phenyl)propan-2-yl)phenoxy)methyl)-5-(methylsulfonyl)-1, 3, 4-oxadiazole (A7)

To a solution of 3-(1-(4-(2-(3,5-dichloro-4-(3-chloropropoxy)phenyl)propan-2-yl)phenoxy)ethyl)-5-(methylthio)-4H-pyrazole (5) (220 mg, 0.49 mmol) in DCM (5 mL) was added m-CPBA (85% purity, 226 mg, 4.03 mmol) at 0° C. The reaction was stirred at 20° C. for 4 hours. LCMS showed the reaction was completed. The mixture was quenched with saturated aqueous Na₂S₂O₃ (5 mL) and saturated aqueous NaHCO₃ (5 mL), then extracted with DCM (10 mL×3). The combined organic layers were washed with brine (10 mL), dried over Na₂SO₄, filtered and concentrated under reduced pressure. The residue was purified by prep-HPLC (TFA) to give 2-((4-(2-(3,5-Dichloro-4-(3-chloropropoxy)phenyl)propan-2-yl)phenoxy)methyl)-5-(methylsulfonyl)-1,3,4-oxadiazole (A7) (74 mg, yield: 31.6%) as colorless oil. ¹H NMR (400 MHz, DMSO-d6) δ ppm 7.14 (d, J=8.9 Hz, 2H), 7.11 (s, 2H), 6.95 (d, J=8.8 Hz, 2H), 5.35 (s, 2H), 4.15 (t, J=5.73 Hz, 2H), 3.86 (t, J=6.39 Hz, 2H), 3.50 (s, 3H), 2.28 (t, J=6.06 Hz, 2H), 1.63 (s, 6H). LCMS (220 nm): 97%. LCMS M+H⁺) m/z: calcd 532.04, found 533.1.

### Example 4: Synthesis of 5-((4-(2-(3,5-dichloro-4-(2-chloroethoxy)phenyl)propan-2-yl)phenoxy)methyl)-4-(methylsulfonyl)oxazole (A13)

A solution of 5-(chloromethyl)-4-methylsulfonyl-oxazole (6) (500 mg, 2.56 mmol), 4-[1-[3,5-dichloro-4-(2-chloroethoxy)phenyl]-1-methyl-ethyl]phenol (11) (919 mg, 2.56 mmol) and Cs₂CO₃ (1.67 g, 5.11 mmol) in DMF (20 mL) was stirred at 25° C. for 2 hours. Then the resulting solution was stirred at 40° C. for 0.5 hr. The reaction was completed detected by TLC. The reaction was quenched with water (50 mL), and the mixture was extracted with EtOAc (20 mL×3). The combined organic layers were washed with brine (30 mL×2), dried over Na₂SO₄, filtered, and concentrated under reduced pressure. The residue was purified by MPLC to give 5-[[4-[1-[3,5-dichloro-4-(2-chloroethoxy)phenyl]-1-methyl-ethyl]phenoxy]methyl]-4-methylsulfonyl-oxazole (528 mg, yield: 39.8%) as white solid. ¹H NMR (400 MHz, CHLOROFORM-d) δ=7.92 (s, 1H), 7.09-7.02 (m, 4H), 6.89-6.83 (m, 2H), 5.34 (s, 2H), 4.18 (t, J=6.4 Hz, 2H), 3.79 (t, J=6.4 Hz, 2H), 3.11 (s, 3H), 1.54 (s, 6H). MS(M+H⁺) m/z: clcd. 517.0; found 518.1, 540.0.

### Example 5: Synthesis of N-((3-(4-(2-(3,5-dichloro-4-(3-chloropropoxy)phenyl)propan-2-yl)phenyl) isoxazol-5-yl)methyl)methanesulfonamide (A22)

To a solution of [3-[4-[1-[3,5-dichloro-4-(3-chloropropoxy)phenyl]-1-methyl-ethyl]phenyl]isoxazol-5-yl]methanamine (7) (60 mg, 0.13 mmol) in DCM (3 mL) was added TEA (40 mg, 0.40 mmol) and MsCl (18 mg, 0.16 mmol) under N2 atmosphere at 0° C. The reaction was stirred at 20° C. for 5 hrs. TLC showed the reaction was completed. The mixture was poured into H₂O (5 mL) and extracted with DCM (10 mL×3). The combined organic layers were washed with brine (10 mL), dried over Na₂SO₄, filtered and concentrated under reduced pressure. The residue was purified by prep-HPLC (TFA) to give N-((3-(4-(2-(3,5-dichloro-4-(3-chloropropoxy)phenyl)propan-2-yl)phenyl)isoxazol-5-yl)methyl) methanesulfonamide (A22) (5 mg, yield: 7.11%) as brown oil. LCMS purity (220 nm): 89.4%. ¹H NMR (400 MHz, CHCl₃-d) δ=7.73 (br d, J=7.9 Hz, 2H), 7.31 (br d, J=8.2 Hz, 2H), 7.14 (s, 2H), 6.60 (s, 1H), 4.89-4.80 (m, 1H), 4.54 (d, J=6.2 Hz, 2H), 4.16 (t, J=5.6 Hz, 2H), 3.86 (t, J=6.4 Hz, 2H), 2.99 (s, 3H), 2.33-2.25 (m, 2H), 1.68 (s, 6H). LCMS(M+H⁺) m/z: clcd. 530.0; found 531.0.

### Example 6: Synthesis of N-(tert-Butyl)-3,5-dichloro-4-(2-chloroethoxy)-N-(4-((4-(methyl-sulfonyl)oxazol-5-yl)methoxy)phenyl)aniline (A31)

To a mixture of 4-[N-tert-butyl-3,5-dichloro-4-(2-chloroethoxy)anilino]phenol (9) (110 mg, 0.283 mmol) and Cs₂CO₃ (277 mg, 0.85 mmol) in DMF (5 mL) was added 5-(chloromethyl)-4-methylsulfonyl-oxazole (10) (83 mg, 0.42 mmol). Then the resulting mixture was stirred at 40° C. for 2 hours. LCMS showed the reaction was completed. The mixture was cooled down, quenched with water (5 mL) and extracted with EtAOc (5 mL×3). The combined organic layers were washed with brine (5 mL×3), dried over Na₂SO₄, filtered and concentrated under reduced pressure. The residue was purified by p-HPLC (TFA) to give the N-tert-butyl-3,5-dichloro-4-(2-chloroethoxy)-N-[4-[(4-methylsulfonyl-oxazol-5-yl)-methoxy]-phenyl]aniline (A31) (36.5 mg, yield: 23.5%) as yellow solid. HPLC purity (220 nm): 91.7%. ¹H NMR (400 MHz, CHCl₃-d) δ 8.01 (s, 1H), 7.06-7.02 (m, 2H), 6.99-6.94 (m, 2H), 6.73 (s, 2H), 5.41 (s, 2H), 4.17 (t, J=6.4 Hz, 2H), 3.82 (t, J=6.4 Hz, 2H), 3.20 (s, 3H), 1.35 (s, 9H). LCMS (M+Na⁺) m/z: calcd 546.1; found 569.1.

### Example 7: Synthesis of 4-(2-(3,5-dichloro-4-(2-chloroethoxy)phenyl)propan-2-yl)-N-((4-(methylsulfonyl)oxazol-5-yl)methyl)aniline hydrochloride (A32)

To a suspension of 5-(chloromethyl)-4-(methylsulfonyl)oxazole (5) (200 mg, 0.5 mmol) and Ag₂CO₃ (564 mg, 0.2 mmol) in DMF (2 mL) was added 4-(2-(3,5-dichloro-4-(2-chloroethoxy)phenyl)propan-2-yl)aniline (4) (382 mg, 0.1 mmol), and the mixture was stirred at 65° C. for 2 hours. TLC showed the reaction was completed. The resulting mixture was cooled down, poured into H₂O (6 mL), extracted with EtOAc (2 mL×2). The combined organic layers were washed with brine (4 mL×3), dried over Na₂SO₄, filtered and concentrated under reduced pressure. The residue was purified by prep-HPLC (HCl) to give 4-(2-(3,5-dichloro-4-(2-chloroethoxy)phenyl)propan-2-yl)-N-((4-(methylsulfonyl)oxazol-5-yl) methyl)aniline hydrochloride (A32) (20 mg, yield: 3.8%) as white solid. ¹H NMR (400 MHz, CHCl₃-d) δ 7.85 (s, 1H), 7.16-7.09 (m, 4H), 7.04-6.93 (m, 2H), 4.80 (s, 2H), 4.26 (t, J=6.4 Hz, 2H), 3.86 (t, J=6.4 Hz, 2H), 3.16 (s, 3H), 1.61 (s, 6H). LCMS (M+H⁺) m/z: calcd: 516.0; found 517.0.

### Example 8: Synthesis of 5-(1-(4-(2-(3,5-dichloro-4-(2-chloroethoxy)phenyl)propan-2-yl) phenoxy)ethyl)-4-(methylsulfonyl)oxazole (A35)

To a mixture of 5-(1-(4-(2-(3,5-dichloro-4-(2-chloroethoxy)phenyl)propan-2-yl)phenoxy)ethyl)-4-(methylthio)oxazole (8) (50 mg, 0.1 mmol) was added mCPBA (80% purity, 64 mg, 0.3 mmol) in DCM (3 mL) at 25° C., and the mixture was stirred at the same temperature for 16 hours. LCMS showed the reaction was completed. The reaction was quenched with H₂O (5 mL), extracted with EtOAc (6 mL×3). The combined organic layers were washed with brine (3 mL), dried over Na₂SO₄, filtered and concentrated under reduced pressure. The residue was purified by p-HPLC (TFA) 5-(1-(4-(2-(3,5-dichloro-4-(2-chloroethoxy)phenyl)propan-2-yl)phenoxy)ethyl)-4-(methylsulfonyl)oxazole (21.7 mg, yield: 40.8%) as white solid. HPLC purity (220 nm): 98.5%. ¹H NMR (400 MHz, CHCl₃-d) δ=7.94 (s, 1H), 7.14-7.03 (m, 4H), 6.92 (d, J=8.9 Hz, 2H), 6.10 (q, J=6.5 Hz, 1H), 4.26 (t, J=6.3 Hz, 2H), 3.86 (t, J=6.3 Hz, 2H), 3.06 (s, 3H), 1.74 (d, J=6.7 Hz, 3H), 1.59 (s, 6H). LCMS (M+H⁺) m/z: calcd: 531.0; found 532.0.

### Example 9: Synthesis of N-(4-((4-(2-(3,5-dichloro-4-(2-chloroethoxy)phenyl)propan-2-yl)phenoxy)methyl)oxazol-2-yl)methanesulfonamide (A38)

To a solution of 2-chloro-4-[[4-[1-[3,5-dichloro-4-(2-chloroethoxy)phenyl]-1-methyl-ethyl]phenoxy]methyl]oxazole (5) (10 mg, 0.02 mmol) in 1,4-dioxane (0.2 mL) was added methanesulfonamide (2.4 mg, 0.02 mmol), Brettphos Pd G3 (2 mg, w20%) and t-BuONa (3 mg, 0.03 mmol). The mixture was stirred at 80° C. for 10 hours under N2 atmosphere. LCMS showed 5% desired MS and 90% starting material. The resulting 20 reaction mixtures were cooled down and combined. The mixture was filtered and the filtrate concentrated under reduced pressure. The residue was purified by prep-HPLC (TFA) to give N-[[5-bromo-4-[4-[1-[3,5-dichloro-4-(3-chloropropoxy)phenyl]-1-methyl-ethyl]phenyl]oxazol-2-yl]methyl]methanesulfonamide (2 mg, yield: 1.8%) as pale yellow solid. LCMS (220 nm): 85.79%. ¹H NMR (400 MHz, CHCl₃-d) δ 7.16 (d, J=8.8 Hz, 2H), 7.12 (s, 2H), 7.08 (s, 1H), 6.86 (d, J=8.8 Hz, 2H), 4.86 (s, 2H), 4.27 (t, J=6.4 Hz, 2H), 3.86 (t, J=6.4 Hz, 2H), 3.09 (s, 3H), 1.64 (s, 6H). LCMS (M+H⁺) m/z: calcd: 532.04; found 533.0.

### Example 10: Synthesis of N-[3-[[4-[1-[3,5-dichloro-4-(2-chloroethoxy)phenyl]-1-methyl-ethyl]phenoxy] methyl]-1H-pyrazol-4-yl]methanesulfonamide (A40)

A solution of N-[3-[[4-[1-[3,5-dichloro-4-(2-chloroethoxy)phenyl]-1-methyl-ethyl]phenoxy]methyl]-1-tetrahydropyran-2-yl-pyrazol-4-yl]methanesulfonamide (9) (70 mg, 0.113 mmol) in HCl/EtOAc (4M, 2 mL) was stirred at 20° C. for 2 hours. LCMS showed the reaction was completed. The mixture was concentrated under reduced pressure. The residue was purified by prep-HPLC (FA) to give N-[3-[[4-[1-[3,5-dichloro-4-(2-chloroethoxy)phenyl]-1-methyl-ethyl]phenoxy]methyl]-1H-pyrazol-4-yl]methanesulfonamide(A40) (11.6 mg, yield: 18.2%) as white solid. ¹H NMR (400 MHz, CHCl₃-d) δ p pm 7.71 (s, 1H), 7.10-7.17 (m, 4H), 6.89-6.94 (m, 2H), 6.22 (s, 1H), 5.21 (s, 2H), 4.26 (t, J=6.39 Hz, 2H), 3.86 (t, J=6.28 Hz, 2H), 2.90 (s, 3H), 1.62 (s, 6H). LCMS (M+Na⁺) m/z: calcd: 531.06; found 532.1.

### Example 11: Synthesis of N-(4-((4-(2-(3, 5-dichloro-4-(2-chloroethoxy)phenyl)propan-2-yl)phenoxy) methyl) pyrimidin-5-yl)methanesulfonamide (A41)

A mixture of tert-butyl N-(4-((4-(1-(3,5-dichloror-4-(2-chloroethoxy)phenyl)-1-methyl-ethyl)phenoxy)methyl)pyrimidin-5-yl)-N-methylsulfonyl-carbamate (6) (50 mg, 0.062 mmol) in DCM (5.0 mL) and TFA (0.5 mL) was stirred at 20° C. for 1 hour. LCMS showed the reaction was completed. The resulting mixture was concentrated under reduced pressure. The residue was purified by prep-HPLC (TFA) to give N-(4-((4-(1-(3,5-dichloro-4-(2-chloroethoxy)phenyl)-1-methyl-ethyl)phenoxy)methyl)pyrimidin-5-yl) methanesulfonamide (A41) (8 mg, yield: 23.7%) as yellow oil. ¹H NMR (400 MHz, CHCl₃-d) 6 ppm 9.01 (d, J=4.40 Hz, 2H), 7.81 (br s, 1H), 7.16 (d, J=8.93 Hz, 2H), 7.10 (s, 2H), 6.93 (d, J=8.93 Hz, 2H), 5.36 (s, 2H), 4.26 (t, J=6.36 Hz, 2H), 3.86 (t, J=6.36 Hz, 2H), 3.03 (s, 3H), 1.62 (s, 6H). LCMS (M+H⁺) m/z: 545.05; found 546.0. HPLC purity (220 nm): 84.4%.

### Example 12: Synthesis of N-(4-(2-(3,5-dichloro-4-(2-chloroethoxy)phenyl)propan-2-yl)phenyl)-2-(methylsulfonamido)oxazole-4-carboxamide (A49)

To a solution of 2-(methane-sulfonamido)oxazole-4-carboxylic acid (3) (60 mg, 0.3 mmol) in DMF (3 mL) was added 4-[1-[3,5-dichloro-4-(2-chloroethoxy)phenyl]-1-methyl-ethyl]aniline (4) (104 mg, 0.3 mmol), HATU (133 mg, 0.35 mmol) and TEA (0.12 mL, 0.9 mmol) at 25° C. The mixture was stirred at the same temperature for 3 hours. LCMS showed the reaction was completed, the mixture was quenched with H₂O (1 mL), and directly purified by prep-HPLC (TFA), to give N-[4-[1-[3,5-dichloro-4-(2-chloroethoxy)phenyl]-1-methyl-ethyl]phenyl]-2-(methanesulfonamido)oxazole-4-carboxamide (A49) (23.2 mg, yield: 14.6%) as white solid. ¹H NMR (400 MHz, CHCl₃-d) δ 8.44 (s, 1H), 7.91 (s, 1H), 7.56 (d, J=8.8 Hz, 2H), 7.21 (d, J=8.8 Hz, 2H), 7.13 (s, 2H), 4.27 (t, J=6.4 Hz, 2H), 3.86 (t, J=6.4 Hz, 2H), 3.32 (s, 3H), 1.65 (s, 6H). LCMS (M+H⁺) m/z: clcd 545.03; found 546.0.

### Example 13: Synthesis of 5-((4-(2-(3,5-dichloro-4-(3,3,3-trifluoropropoxy)phenyl)propan-2-yl)phenoxy)methyl)-4-(methylsulfonyl)oxazole (A54)

To a mixture of 4-[1-[3,5-dichloro-4-(3,3,3-trifluoro propoxy)phenyl]-1-methyl-ethyl]phenol (3) (40 mg, 0.10 mmol) and 5-(chloromethyl)-4-methylsulfonyl-oxazole (4) (24 mg, 0.12 mmol) in DMF (0.5 mL) was added Cs₂CO₃ (66 mg, 0.20 mmol) and the mixture was stirred at 25° C. for 16 hours. LCMS showed the reaction was completed. The mixture was poured into water (2 mL) and extracted with EtOAc (5 mL×3). The combined organic layers were washed with brine (5 mL×3), dried over Na₂SO₄, filtered and concentrated under reduced pressure. The residue was purified by p-TLC to give 5-[[4-[1-[3,5-dichloro-4-(3,3,3-trifluoropropoxy)phenyl]-1-methyl-ethyl]phenoxy]methyl]-4-methylsulfonylo-xazole (A54) (18 mg, yield: 29.9%) as yellow oil. LCMS purity: (220 nm): 93.3%. ¹H NMR (400 MHz, CHCl₃-d) δ 8.00 (s, 1H), 7.16-7.12 (m, 4H), 6.94 (d, J=8.8 Hz, 2H), 5.42 (s, 2H), 4.22 (t, J=6.8 Hz, 2H), 3.19 (s, 3H), 2.78-2.64 (m, 2H), 1.62 (s, 6H). LCMS (M+NH₄⁺) m/z: calcd 551.1; found 569.0.

### Example 14: Synthesis of 2-(2-chloroethoxy)-5-(2-(3-cyano-4-((4-(methylsulfonyl)oxazol-5-yl)methoxy) phenyl)propan-2-yl)benzonitrile (A63)

To a solution of 2-(2-chloroethoxy)-5-(2-(3-cyano-4-hydroxyphenyl)propan-2-yl)benzonitrile (7) (130 mg, 0.38 mmol) in DMF (2 mL) was added 5-(chloromethyl)-4-(methylsulfonyl)oxazole (G) (75 mg, 0.38 mol) and Cs₂CO₃ (249 mg, 0.76 mmol) under N2 atmosphere. The reaction was stirred at 0° C. for 3 hours. LCMS showed the reaction was completed. The mixture was diluted with EtOAc (5 mL) and poured into H₂O (5 mL). The aqueous phase was extracted with EtOAc (5 mL×2). The combined organic layers were washed with brine (5 mL×4), then dried over Na₂SO₄, filtered and concentrated under reduced pressure. The crude product was purified by prep-HPLC(TFA) to give 2-(2-chloroethoxy)-5-(2-(3-cyano-4-((4-(methylsulfonyl)oxazol-5-yl)methoxy)phenyl)propan-2-yl)benzonitrile (A63) (53 mg, yield: 27.8%) as white solid. LCMS purity (220 nm): 91.1%. ¹H NMR (400 MHz, CHCl₃-d) δ=8.04 (s, 1H), 7.41 (br s, 2H), 7.39-7.29 (m, 2H), 7.15-7.04 (m, 1H), 6.94-6.85 (d, J=8.9 Hz, 1H), 5.51 (s, 2H), 4.33 (br t, J=6.0 Hz, 2H), 3.87 (br t, J=6.0 Hz, 2H), 3.25 (s, 3H), 1.64 (s, 6H). LCMS (M+H⁺) m/z: calcd 499.1; found 500.1.

### Example 15A: Synthesis of N-((2-((4-(2-(3,5-dichloro-4-(2-chloroethoxy)phenyl)propan-2-yl) phenyl)amino)oxazol-5-yl)methyl)methanesulfonamide (A75)

A solution of tert-butyl N-[[2-[4-[1-[3,5-dichloro-4-(2-chloroethoxy)phenyl]-1-methyl-ethyl]anilino]oxazol-5-yl]methyl]-N-methylsulfonyl-carbamate (5) (25 mg, 0.04 mmol) in DCM (2 mL) and TFA (0.2 mL) was stirred at 25° C. for 3 hours. LCMS showed the reaction was completed. The mixture was concentrated and purified by prep-HPLC (TFA) to give N-[[2-[4-[1-[3,5-dichloro-4-(2-chloroethoxy)phenyl]-1-methyl-ethyl]anilino]oxazol-5-yl]methyl]methanesulfonamide (4.6 mg, yield: 21.9%) as yellow oil. LCMS purity (220 nm): 86%. ¹H NMR (400 MHz, CHCl₃-d) δ 7.36-7.33 (m, 2H), 7.25-7.22 (m, 2H), 7.11 (s, 2H), 7.02 (s, 1H), 5.18 (s, 1H), 4.36 (s, 2H), 4.27 (t, J=6.4 Hz, 2H), 3.87 (t, J=6.4 Hz, 2H), 3.00 (s, 3H), 1.64 (s, 6H). LCMS (M+H⁺) m/z: calcd: 531.0; found 531.6.

### Example 15B: Synthesis of N-(4-((4-(2-(3-chloro-4-(2-chloroethoxy)-5-cyanophenyl)propan-2-yl)phenoxy) methyl)pyrimidin-2-yl)methanesulfonamideN-(4-((4-(2-(3-chloro-4-(2-chloroethoxy)-5-cyanophenyl) propan-2-yl) phenoxy) methyl)pyrimidin-2-yl)methanesulfonamide (A109)

2-chloro-4-(chloromethyl)pyrimidine (2): To a mixture of 2-chloro-4-methyl-pyrimidine (50.0 g, 398 mmol) and NCS (77.9 g, 583 mmol) in MeCN (250 mL) was added benzoyl benzenecarboperoxoate (28.3 g, 117 mmol) in portions at 20° C. and the mixture was stirred at 100° C. for 16 hrs under N2 atmosphere. TLC showed most of the starting material consumed and two new spots appeared. The mixture was cooled down to room temperature, poured into water (500 mL) and extracted with EtOAc (200 mL×3). The organic layers were combined and washed with brine (200 mL×2), dried over Na₂SO₄, filtered and concentrated under reduced pressure. The residue was purified by silica gel column chromatography to give 2-chloro-4-(chloromethyl) pyrimidine (22 g, yield: 31.2%) as yellow oil. ¹H NMR (400 MHz, CDCl3) δ=8.69 (d, J=5.2 Hz, 1H), 7.54 (d, J=5.0 Hz, 1H), 4.61 (s, 2H).

3-chloro-2-(2-chloroethoxy)-5-(2-(4-((2-chloropyrimidin-4-yl)methoxy)phenyl)propan-2-yl)benzonitrile (4): To a mixture of 3-chloro-2-(2-chloroethoxy)-5-(2-(4-hydroxyphenyl)propan-2-yl)benzonitrile (18.0 g, 51.4 mmol) and 2-chloro-4-(chloromethyl) pyrimidine (10.1 g, 61.7 mmol) in DMF (150 mL) was added Cs₂CO₃ (33.5 g, 103.4 mmol) at 20° C. and the mixture was stirred at the same temperature for 16 hrs. LCMS showed the reaction was completed. The reaction mixture was poured into H₂O (300 mL) and extracted with EtOAc (150 mL×3). The combined organic layers were washed with brine (150 mL×3), dried over Na₂SO₄, filtered and concentrated under reduced pressure. The residue was purified by silica gel column chromatography to give 3-chloro-2-(2-chloroethoxy)-5-(2-(4-((2-chloropyrimidin-4-yl)methoxy)phenyl)propan-2-yl)benzonitrile (15.5 g, yield: 63.3%) as white solid. ¹H NMR (400 MHz, CDCl3) δ=8.67 (d, J=5.2 Hz, 1H), 7.56 (d, J=5.2 Hz, 1H), 7.45 (d, J=2.4 Hz, 1H), 7.35-7.29 (m, 1H), 7.13 (d, J=8.8 Hz, 2H), 6.90 (d, J=8.8 Hz, 2H), 5.16 (s, 2H), 4.43 (t, J=6.0 Hz, 2H), 3.88 (t, J=6.0 Hz, 2H), 1.65 (s, 6H).

N-(4-((4-(2-(3-chloro-4-(2-chloroethoxy)-5-cyanophenyl)propan-2-yl)phenoxy)methyl) pyrimidin-2-yl)methanesulfonamide (A109): To a mixture of 3-chloro-2-(2-chloroethoxy)-5-(2-(4-((2-chloropyrimidin-4-yl)methoxy)phenyl)propan-2-yl)benzonitrile (15.5 g, 32.5 mmol), methane sulfonamide (9.3 g, 97.5 mmol), Cs₂CO₃ (21.2 g, 65.0 mmol) and Xantphos (1.88 g, 3.25 mmol) in 1,4-dioxane (450 mL) was added Pd₂(dba)₃ (3.0 g, 3.3 mmol) at 20° C. and the mixture was stirred at 90° C. for 6 hrs under N2 atmosphere. LCMS showed the reaction was completed. The mixture was cooled down to room temperature, poured into water (300 mL) and extracted with EtOAc (300 mL×3). The combined organic layers were washed with brine (300 mL×2), dried over Na₂SO₄, filtered and concentrated under reduced pressure. The residue was purified by silica gel column chromatography to give the crude product and then further purified by p-HPLC (TFA) to give N-(4-((4-(2-(3-chloro-4-(2-chloroethoxy)-5-cyanophenyl)propan-2-yl)phenoxy) methyl)pyrimidin-2-yl)methanesulfonamide (5.30 g, yield: 30.1%) as yellow solid. ¹H NMR (400 MHz, CDCl3) δ=10.02 (br s, 1H), 8.69 (d, J=5.2 Hz, 1H), 7.45 (d, J=2.4 Hz, 1H), 7.34-7.31 (m, 1H), 7.30 (d, J=5.2 Hz, 1H), 7.13 (d, J=8.8 Hz, 2H), 6.91 (d, J=8.8 Hz, 2H), 5.13 (s, 2H), 4.43 (t, J=6.0 Hz, 2H), 3.88 (t, J=6.0 Hz, 2H), 3.47 (s, 3H), 1.65 (s, 6H). LCMS (220 nm): 99.0%. Exact Mass: 534.09; found 535.1, 537.0. See PCT/US2019/057034.

### Example 16: Synthesis of 3-(4-(2-(3,5-dichloro-4-(2-chloroethoxy)phenyl)propan-2-yl)benzyl)-1,5,5-trimethylimidazolidine-2,4-dione (B2)

To a mixture of 1,5,5-trimethylimidazolidine-2,4-dione (5) (20 mg, 0.2 mmol) and K₂CO₃ (70 mg, 0.5 mmol) in DMF (3 mL) was added 1,3-dichloro-2-(2-chloroethoxy)-5-(2-(4-(chloromethyl)phenyl)propan-2-yl)benzene (4) (50 mg, 0.1 mmol) at 25° C. and the mixture was stirred at the same temperature for 2 hours. LCMS showed the reaction was completed. The mixture was poured into H₂O (10 mL), extracted with EtOAc (5 mL×2). The combined organic layers were washed with brine (5 mL), dried over Na₂SO₄, filtered and concentrated under reduced pressure. The residue was purified by prep-HPLC (HCl) to give 3-(4-(2-(3,5-dichloro-4-(2-chloroethoxy)phenyl)propan-2-yl)benzyl)-1,5,5-trimethylimidazolidine-2,4-dione (B2) (20 mg, yield: 31.8%) as colorless oil. LCMS purity (220 nm): 96.1%. ¹H NMR (400 MHz, CHCl₃-d) δ=7.30-7.25 (m, 1H), 7.28-7.25 (m, 1H), 7.30-7.25 (m, 1H), 7.15-7.10 (m, 2H), 7.10-7.08 (m, 2H), 4.66-4.57 (m, 2H), 4.24 (t, J=6.4 Hz, 2H), 3.89-3.77 (m, 2H), 2.87 (s, 3H), 1.65-1.54 (m, 6H), 1.41-1.34 (m, 6H). LCMS (M+H⁺) m/z: calcd: 496.1; found 497.1.

### Example 17: Synthesis of 3-(4-(2-(3,5-dichloro-4-(2-chloroethoxy)phenyl)propan-2-yl)benzyl)-5,5-dimethyl-1-(methylsulfonyl)imidazolidine-2,4-dione (B3)

To a solution of 3-(4-(2-(3,5-dichloro-4-(2-chloroethoxy)phenyl)propan-2-yl)benzyl)-5,5-dimethylimidazolidine-2,4-dione (6) ((40 mg, 0.1 mmol) in THF (2 mL) was added Mesyl chloride (0.1 mL, 0.2 mmol) and NaH (60.0%, 6 mg, 0.2 mmol) at 0° C. and the mixture was stirred at 80° C. for 16 hours. TLC showed the reaction was completed. The reaction was quenched with saturated aqueous NH₄Cl (10 mL) and extracted with EtOAc (3 mL×2). The combined organic layers were washed with brine (3 mL), dried over Na₂SO₄, filtered and concentrated under reduced pressure. The residue was purified by prep-HPLC (HCl) to give 3-(4-(2-(3,5-dichloro-4-(2-chloroethoxy)phenyl)propan-2-yl)benzyl)-5,5-dimethyl-1-(methylsulfonyl)imidazolidine-2,4-dione (5 mg, yield: 10.8%) as yellow oil. LCMS purity (220 nm): 81.8%. ¹H NMR (400 MHz, CHCl₃-d) δ=7.31-7.28 (m, 2H), 7.20-7.14 (m, 2H), 7.14-7.11 (m, 2H), 4.71-4.65 (m, 2H), 4.27 (t, J=6.4 Hz, 2H), 3.86 (t, J=6.4 Hz, 2H), 3.38 (s, 3H), 1.76-1.71 (m, 6H), 1.64 (s, 6H). LCMS (M+H⁺) m/z: calcd: 526, found: 527.

Biological Assays

### Example 18: Activity of Exemplary Compounds in Cellular Assays

LNCaP cells were transiently transfected with the PSA (6.1 kb)-luciferase reporter for 24 h, and then treated with indicated concentration of representative compounds with synthetic androgen, R1881 (1 nM) for 24 h. After 24 h of incubation with R1881, the cells were harvested, and relative luciferase activities were determined. To determine the IC₅₀, treatments were normalized to the maximum activity with androgen-induction (in the absence of test compounds, vehicle only) (Table 1).

Luciferase Assay: Lysates were thawed on ice then collected into V-bottom 96-well tissue culture plates. Lysates were centrifuged at 4° C. for 5 minutes at 4000 rpm. To measure luminescence of LNCaP cell lysates the Firefly Luciferase Assay System (Promega) was employed, according to manufacturer's protocol.

Statistical analyses were performed using GraphPad Prism (Version 6.01 for Windows; La Jolla, Calif., USA). Comparisons between treatment and control groups were compared using Two-Way ANOVA with post-hoc Dunnett's and Tukey's tests. Differences were considered statistically significant at P values less than 0.05. Densitometric quantification of relative AR levels was determined by Image.

Table 1 shows the IC₅₀ of representative Compounds from Tables A-B from androgen-induced PSA luciferase assay. EPI-002 has the following structure:

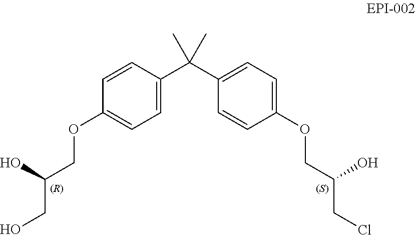

TABLE 1
IC₅₀ of Representative Compounds on Androgen-
Induced PSA Luciferase Activity

| | Androgen-induced | |
|---|---|---|
| Compound | PSA-luciferase | |
| ID | IC₅₀ (nM) | n |
| A13 | 592 | 8 |
| A28 | 400 | 5 |
| A29 | 466 | 5 |
| A35 | 515 | 6 |
| A38 | 631 | 6 |
| A66 | 890 | 6 |
| A74 | 658 | 6 |
| A93 | 205 | 4 |
| A109 | 535 | 2 |
| A122 | 258 | 2 |
| A126 | 629 | 1 |
| A131 | 1100 | 1 |
| A136 | 601 | 2 |
| A170 | 651 | 2 |
| EPI-002 | 9580 | 2 |
| Enzalutamide | 189 | 8 |
| Bicalutamide | 306 | 2 |

### Example 19. In Vivo Pharmakokinetic Properties

The purpose of this study is to determine the pharmacokinetics of the tested compounds in plasma, following oral gavage administration (PO) to male CD-1 mice.

Animal Husbandry: Animals were group housed during acclimation and the study. The animal room environment were controlled (target conditions: temperature 18 to 26° C., relative humidity 30 to 70%, 12 hours artificial light and 12 hours dark). Temperature and relative humidity were monitored daily. Animals were fasted at least 12 hours prior to the administration. All animals had access to Certified Rodent Diet ad libitum 4 hours post dosing.

Dose Formulation: prepared 85-100 uL of a stock solution of representative compounds of the disclosure in DMSO at 50 mM; 1.5% v/v of Tween 80 were added and mixed gently; 95.5% v/v saline was added gently to the organic phase. The solution was mixed slowly by reverse pipetting to get a clear solution.

Dose Administration: the dose formulations were administered via oral gavage per facility SOPs. The dose volume were determined by the animals' body weight collected on the morning of dosing day

Sample Collection: blood collection (about 0.05 mL per time point) was performed from saphenous vein of each animal into polypropylene tubes at each timepoints (0, 0.25, 0.5, 1, 2, 4, 8, 12, and 24 hr). All blood samples were transferred into pre-chilled tubes containing 2 μL of K2-EDTA (0.5M) and placed on wet ice until centrifugation. Each collected blood sample was in the wet-ice before centrifugation. Each collected blood was under centrifugation for 15 minutes at 4° C. and 3000 g for plasma collection. Plasma samples were stored in polypropylene tubes, quickly frozen over dry ice and kept at −70±10° C. until LC/MS/MS analysis.

Bioanalytical analysis: A LC-MS/MS method for the quantitative determination of tested compound in biological matrix were developed under non-GLP compliance. A calibration curve with at least 7 non-zero calibration standards were applied for the method including LLOQ.

Plasma concentration versus time data were analyzed by non-compartmental approaches using the Phoenix WinNonlin 6.3 software program. Cl, Vdss, C0, Cmax, Tmax, T½, AUC(0-t), AUC(0-inf), MRT(0-t), MRT(0-inf), % F (oral availability) and graphs of plasma concentration versus time profile were reported.

Tables 2 shows PK parameters determined from single PO dose of Compound A109 and enzalutamide, dosed as single agent or combination in CD-1 male mice.

TABLE 2
PK Parameters (PO)

| Compound | Dose | Cmax | tmax | | AUC₀₋ₗₐₛₜ |
|---|---|---|---|---|---|
| ID | (mg/kg) | (ng/mL) | (hr) | T 1/2 (hr) | (hr*ng/mL) |
| A109 | 60.00 | 41,667 | 4 | 5.4 | 482,822 |
| Enzalutamide | 15.0 | 21,067 | 9 | ND | 388,202 |
| A109 + | | | | | |
| Enzalutamide | 60.00 | 51,900 | 3 | 6.9 | 603,960 |
| Enzalutamide + | 15.0 | 11,467 | 12 | ND | 231,974 |
| A109 | | | | | |

### Example 20. In Vivo Activity of Representative Compounds in VCaP Xenografts Model

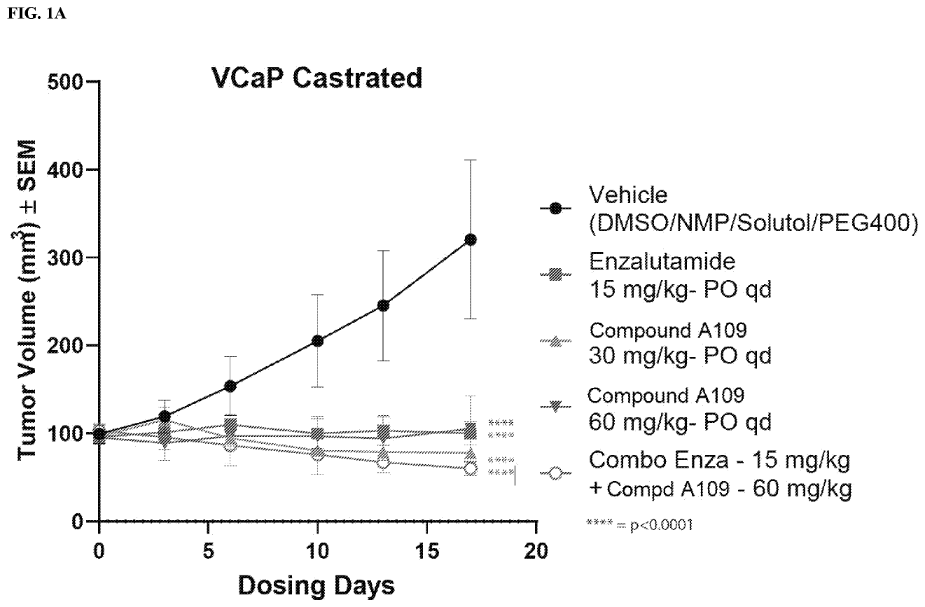

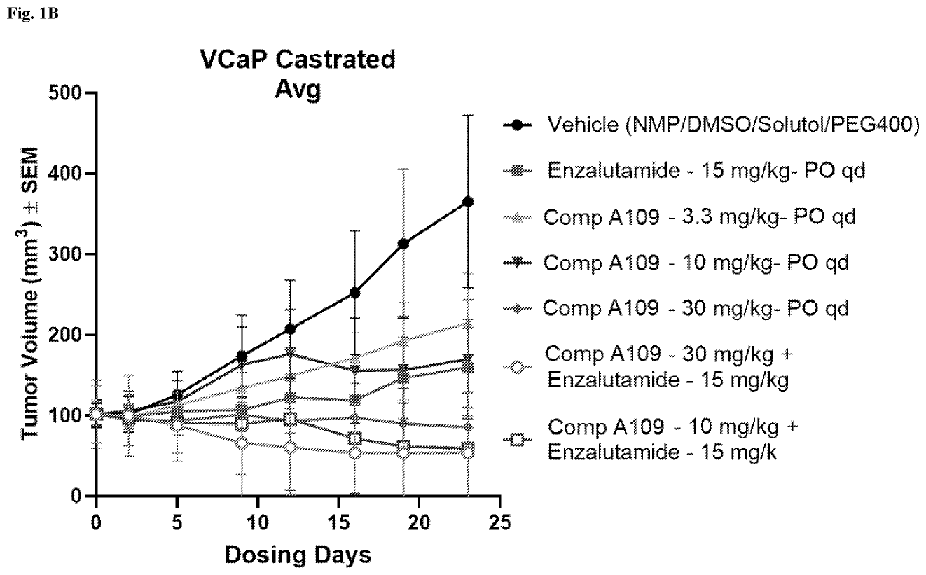

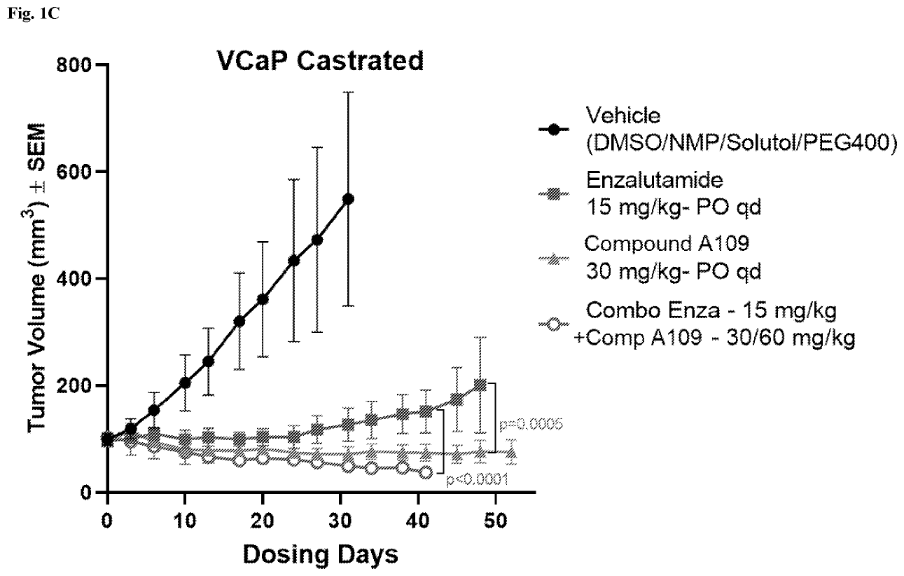

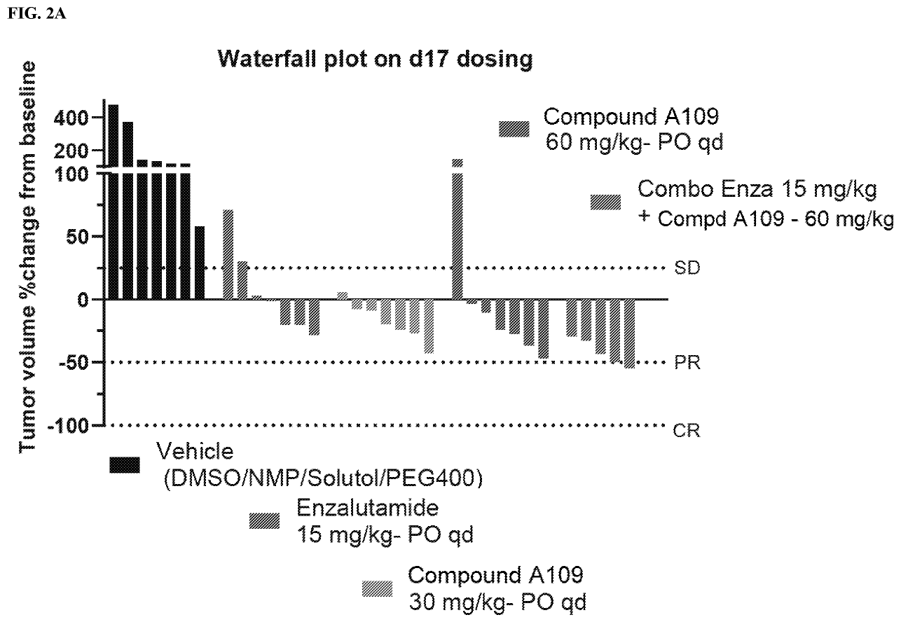

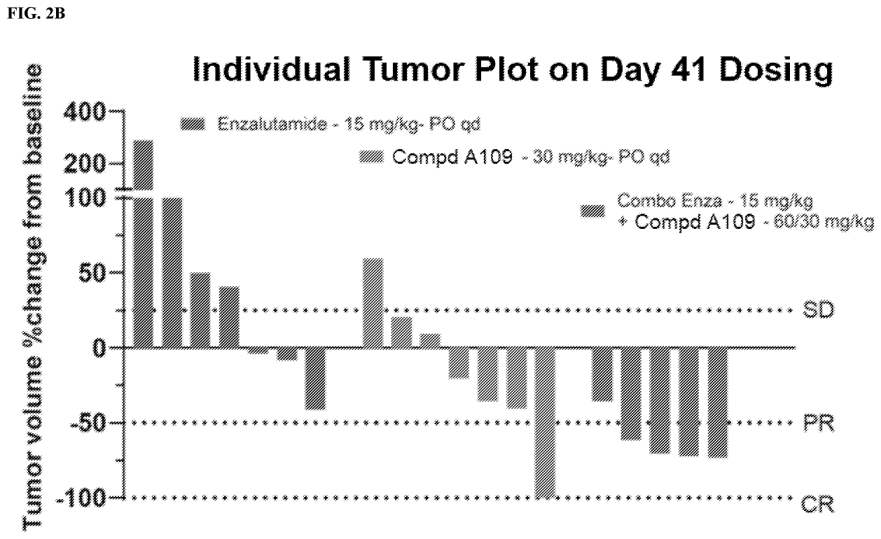

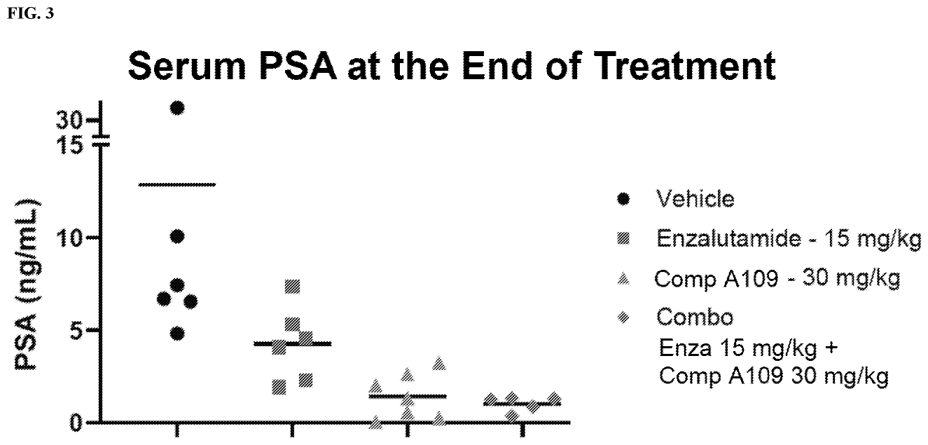

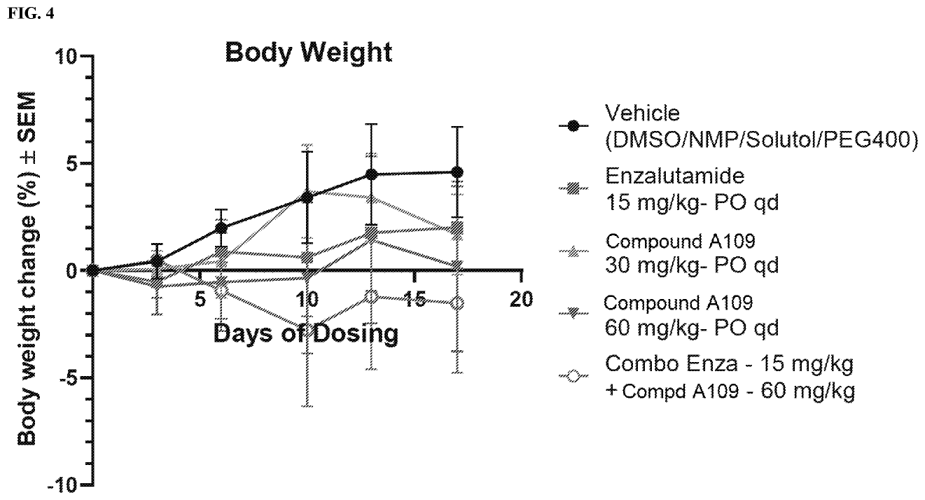

Tumor growth was measured in male SCID Beige mice bearing VCaP tumors. Castration was performed when tumors reached ˜100 mm³ and dosing (as indicated) of enzalutamide, Compound A109, or combination of enzalutamide and Compound A109 started 2 week after castration (FIGS. 1A-1C). Individual tumor volume change from baseline measured on day 17 of the experiment (FIGS. 2A-2B). Serum prostate-specific antigen (PSA) was measured in mice at the end of each treatment (FIG. 3). Body weight of the mice were captured biweekly in the animals and normalized to baseline (FIG. 4). Data demonstrated in FIGS. 1-4 shows that the representative androgen receptor modulators (e.g., Compound A109, an AR N-terminal inhibitor) exhibits excellent anti-tumor response as a monotherapy or in combination with enzalutamide.

### Example 21. Inhibition of Androgen Responsive Genes in LNCaP Cells

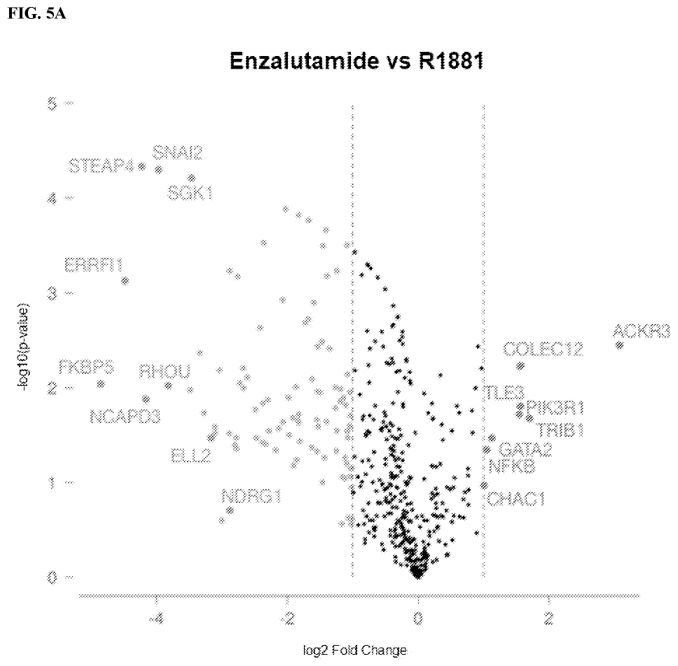

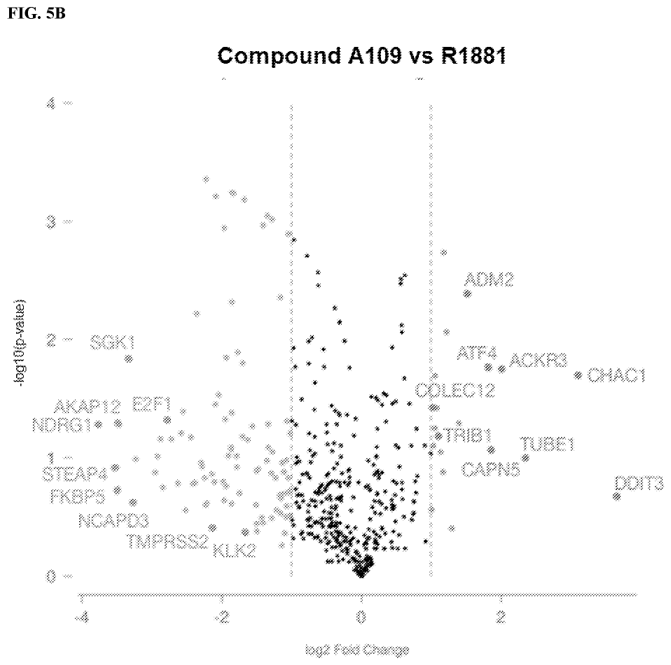

Relative mRNA expression in LNCaP was measured to assess dose-dependent response of Compound A109 alone or with enzalutamide (Enza), apalutamide (Apa), or darolutamide (Daro) in androgen responsive genes. FIGS. 5A and 5B show log-ratio of gene expression values in LNCaP cells treated with enzalutamide compared to synthetic androgen (R1881) and LNCaP cells treated with Compound A109 compared to R1881, respectively.

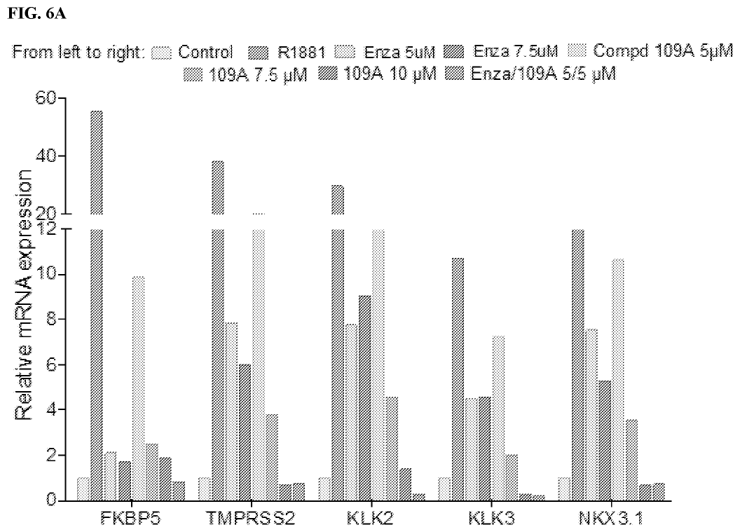

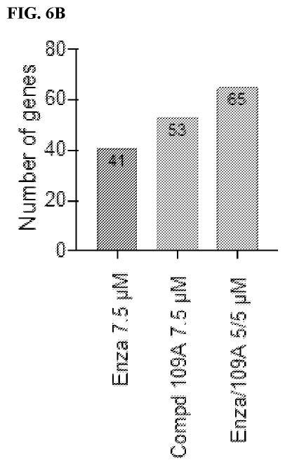

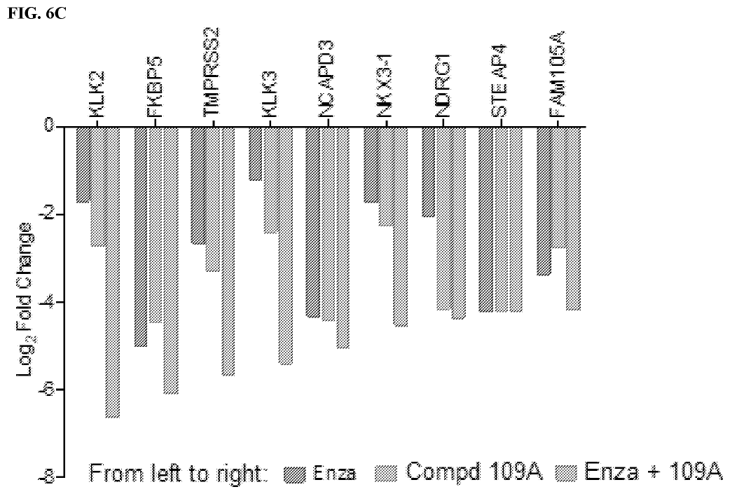

Compound A109 demonstrated dose-dependent response in the androgen response genes FKBP5, TMPRSS2, KLK2, KLK3, and NKX3.1 (FIG. 6A). The combination of enzalutamide and Compound A109 exhibited inhibition of the tested androgen responsive genes.

The number of genes significantly down-regulated with Fold changes ≥4 is shown in FIG. 6B. Top ten down-regulated genes with Enza/Compound A109 5 μM/5 μM combination is shown in FIG. 6C. Top twenty down-regulated genes with Enza/Compound A109 5 μM/5 μM combination is shown in Table 4. FIGS. 5A-5B and Table 4 demonstrate that Compound A109 can modulate androgen receptor0 full-length driven genes similar to enzalutamide in LNCaP.

TABLE 4
Top 20 down-regulated genes with Enza and Compound A109, alone or in
combination

| | | Log2Fold Change | | |
|---|---|---|---|---|
| | | | 7.5 μM | |
| | Gene | 7.5 μM Enza | Compd 109A | 5/5 μM Combo |
| 1. | KLK2 | −1.72 | −2.71 | −6.63 |
| 2. | FKBP5 | −5.01 | −4.47 | −6.09 |
| 3. | TMPRSS2 | −266 | −3.32 | −5.68 |
| 4. | KLK3 | −1.24 | −2.42 | −5.40 |
| 5. | NCAPD3 | −4.32 | −4.40 | −5.03 |
| 6. | NKX3-1 | −1.72 | −2.28 | −4.54 |
| 7. | NDRG1 | −2.04 | −4.17 | −4.37 |
| 8. | STEAP4 | −4.22 | −4.22 | −4.22 |
| 9. | FAM105A | −3.37 | −2.78 | −4.18 |
| 10. | AKAP12 | −1.91 | −3.84 | −4.10 |
| 11. | PMEPA1 | −3.05 | −2.16 | −4.05 |
| 12. | PLPP1 | −2.48 | −3.53 | −3.97 |
| 13. | SNAI2 | −3.97 | −1.79 | −3.97 |
| 14. | ACSL3 | −3.43 | −3.62 | −3.90 |
| 15. | ERRFI1 | −4.51 | −2.76 | −3.90 |
| 16. | CDC6 | −1.20 | −3.60 | −3.86 |
| 17. | ELL2 | −3.39 | −3.05 | −3.81 |
| 18. | CENPN | −3.31 | −2.13 | −3.79 |
| 19. | RHOU | −3.94 | −3.03 | −3.78 |
| 20. | EAF2 | −3.32 | −3.81 | −3.52 |

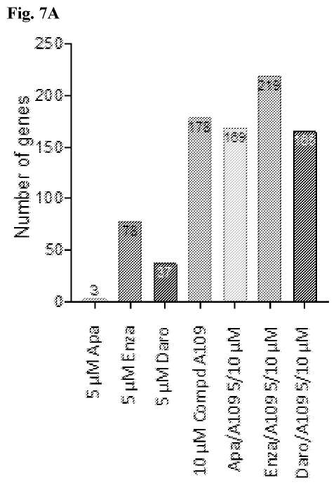

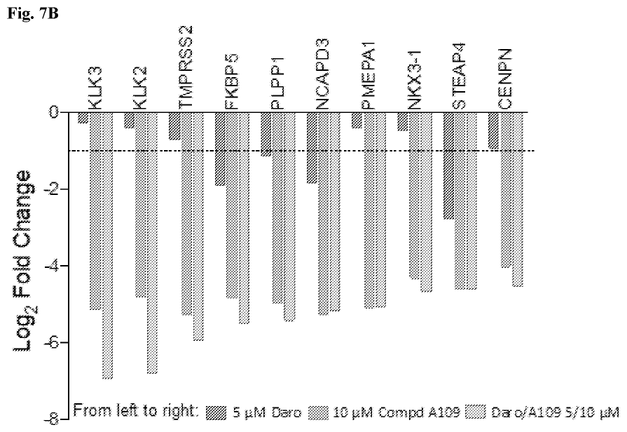

Apalutamide (Apa) or darolutamide (Daro) with or without Compound A109 was also assessed for gene expression in LNCaP cells. The number of genes significantly down regulated with Fold changes ≥4 is shown in FIG. 7A. Top ten down regulated genes with Daro/Compound A109 5 μM/10 μM combination is shown in FIG. 7B. Darolutamide is not very active in LNCaP.

Data shown in FIGS. 6A-6C and 7A-7B and Table 4 were generated using NanoString Androgen Receptor/Prostate Cancer gene panel.

### Example 22. Inhibition of Androgen Responsive Genes in LNCaP95 Cells

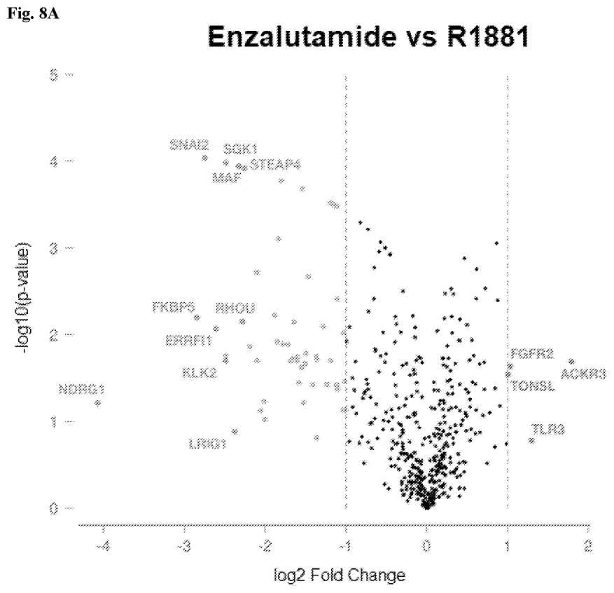

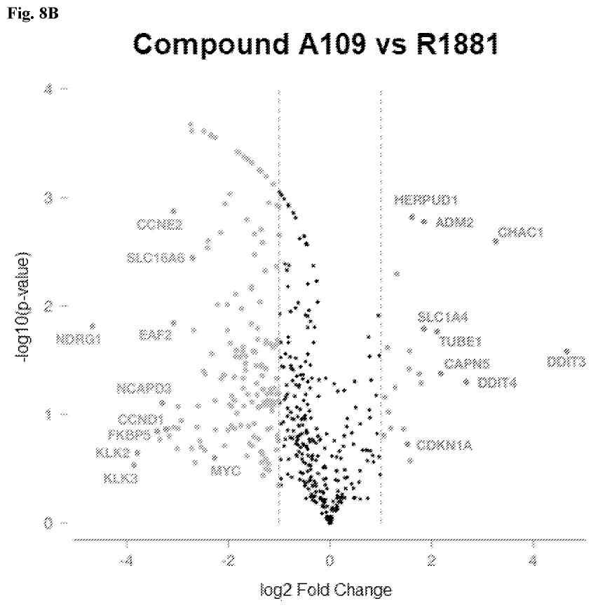

Relative mRNA expression in LNCaP95 (+R1881) was measured to assess response to Compound A109 alone or with enzalutamide (Enza). FIGS. 8A and 8B show log-ratio of gene expression values in LNCaP95 (+R1881) cells treated with enzalutamide compared to synthetic androgen (R1881) and LNCaP95 (+R1881) cells treated with Compound A109 compared to R1881, respectively.

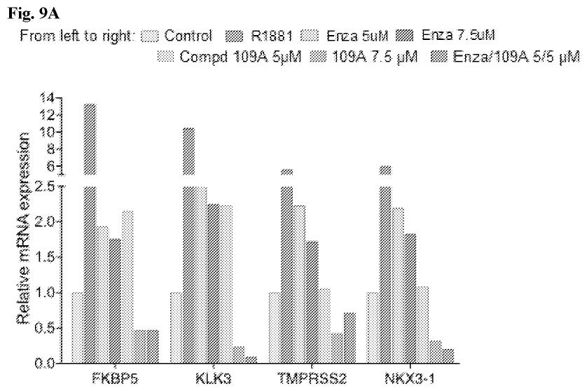

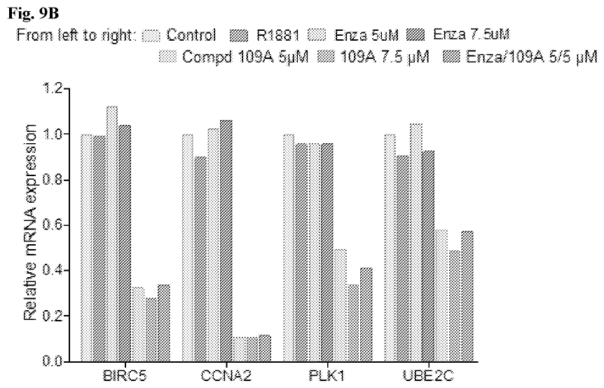

Relative mRNA expression in LNCaP95 (+R1881) was measured for Canonical AR target genes (FIG. 9A) and AR V7-activated genes (FIG. 9B). FIGS. 8A-8B and 9A-9B demonstrates that Compound A109 can modulate AR full-length and AR V7 driven genes. Table 5 shows top ten down-regulated genes with log 2 Fold change.

TABLE 5
Top 10 down-regulated genes (1og2 Fold Change) - LNCaP95 (+R1881)

| | | | Compound A109 |
|---|---|---|---|
| | Enza 7.5 μM | | 7.5 μM |
| NDRG1 | −4.57 | KLK3 | −5.48 |
| FKBP5 | −2.92 | KLK2 | −5.07 |
| SNAI2 | −2.75 | NDRG1 | −4.47 |
| ERRFI1 | −2.69 | NKX3-1 | −4.23 |
| KLK2 | −2.61 | FKBP5 | −4.18 |
| SGK1 | −2.48 | CCND1 | −3.96 |
| SLC16A6 | −2.35 | ADAMTS1 | −3.96 |
| RHOU | −2.35 | PMEPA1 | −3.93 |
| TIPARP | −2.35 | PRR15L | −3.89 |
| IGF1R | −2.34 | NCAPD3 | −3.8 |

Data shown in FIGS. 8A-8B and 9A-9B and Table 5 were generated using NanoString Androgen Receptor/Prostate Cancer gene panel.

### NUMBERED EMBODIMENTS

Embodiment 1: A pharmaceutical composition comprising an androgen receptor modulator and a second therapeutically active agent.

Embodiment 2: The pharmaceutical composition of Embodiment 1, wherein the androgen receptor modulator is a compound of formula (I)

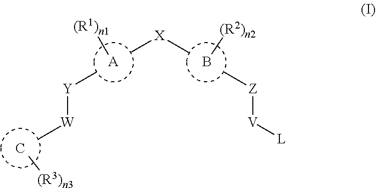

or a pharmaceutically acceptable salt, tautomer, stereoisomer or prodrug thereof, wherein:
A and B are each independently aryl or heteroaryl;
C is a 3- to 10-membered ring;
X is a bond, —(CR⁵R⁶)ₜ—, —O—, —C(═O)—, —S—, —S(═O)—, —SO₂—, —NR⁷—, —N(R⁷)CO—, —CON(R⁷)—, or —NSO₂R⁷—;
Y and Z are each independently a bond, —(CR⁸R⁹)ₘ—, —O—, —C(═O)—, —S—, —S(═O)—, —SO₂—, or —NR⁷—;
W and V are each independently a bond, —(CR^{8a}R^{9a})ₘ—, —C(═O)—, —N(R⁷)CO—, —CONR⁷—, or —NSO₂R⁷—;
L is hydrogen, halogen, —CF₂R¹⁰, —CF₃, —CN, —OR¹⁰; —NR¹¹R¹², or —CONR¹¹R¹²;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, optionally substituted —(C₁-C₆ alkyl)-SO₂R¹⁶, optionally substituted carbocyclyl, optionally substituted heterocyclyl, optionally substituted aryl, or optionally substituted heteroaryl;
R³ is hydrogen, halogen, oxo, ═S, ═NR¹⁶, —CN, —CF₃, —OH, —SR¹⁶, optionally substituted C₁-C₆ alkyl, optionally substituted C₂-C₆ alkenyl, optionally substituted C₂-C₆ alkynyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COOR¹⁶, —NR¹⁴COR¹⁶, —NR¹⁴CONR¹⁴R¹⁵, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, optionally substituted —(C₁-C₆ alkyl)-SO₂R¹⁶, optionally substituted carbocyclyl, optionally substituted heterocyclyl, optionally substituted aryl, or optionally substituted heteroaryl;
R⁵ and R⁶ are each independently hydrogen, halogen, —OH, optionally substituted C₁-C₆ alkyl, optionally substituted C₂-C₆ alkenyl, optionally substituted C₂-C₆ alkynyl, optionally substituted C₁-C₆ alkoxy, —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl; or R⁵ and R⁶ taken together form an optionally substituted carbocyclyl or optionally substituted heterocyclyl;
R⁸ and R⁹ are each independently hydrogen, halogen, or C₁-C₃ alkyl;
R^{8a} and R^{9a} are each independently hydrogen, —OH, halogen, optionally substituted C₁-C₆ alkyl, optionally substituted C₂-C₆ alkenyl, optionally substituted C₂-C₆ alkynyl, optionally substituted C₁-C₆ alkoxy, optionally substituted —OCO(C₁-C₆ alkyl), —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl; or R^{8a} and R^{8b} taken together form an optionally substituted carbocyclyl or optionally substituted heterocyclyl;
R⁷, R¹⁰ and R¹⁶ are each independently hydrogen, optionally substituted C₁-C₆ alkyl, optionally substituted C₂-C₆ alkenyl, optionally substituted C₂-C₆ alkynyl, C₁-C₆ haloalkyl, C₂-C₆ haloalkenyl, C₂-C₆ haloalkynyl, optionally substituted carbocyclyl, optionally substituted —CO(C₁-C₆ alkyl), —CO(optionally substituted heterocyclyl), optionally substituted heterocyclyl, optionally substituted aryl, or optionally substituted heteroaryl; or R⁷ and R^{8a} taken together form an optionally substituted heterocyclyl;
R¹¹, R¹², R¹³, R¹⁴ and R¹⁵ are each independently hydrogen, optionally substituted C₁-C₆ alkyl, optionally substituted C₂-C₆ alkenyl, optionally substituted C₂-C₆ alkynyl, optionally substituted —COO(C₁-C₆ alkyl), optionally substituted carbocyclyl, optionally substituted heterocyclyl, optionally substituted aryl, or optionally substituted heteroaryl; or (R¹¹ and R¹²) or (R¹⁴ and R¹⁵) taken together form an optionally substituted heterocyclyl;
each m is independently 0, 1 or 2;
n1 and n2 are each independently 0, 1, 2, 3, or 4;
n3 is 0, 1, 2, 3, 4 or 5; and
each t is independently 0, 1 or 2.

Embodiment 3: The pharmaceutical composition of Embodiment 2, wherein C is 5- to 7-membered heteroaryl comprising 1, 2, or 3 heteroatoms selected from O, S, or N as a ring member.

Embodiment 4: The pharmaceutical composition of Embodiments 2 or 3, wherein C is pyrazole, imidazole, oxazole, oxadiazole, oxazolone, isoxazole, thiazole, pyridyl, or pyrimidyl.

Embodiment 5: The pharmaceutical composition of any one of Embodiments 2-4, wherein C, which is optionally substituted with R³, is selected from

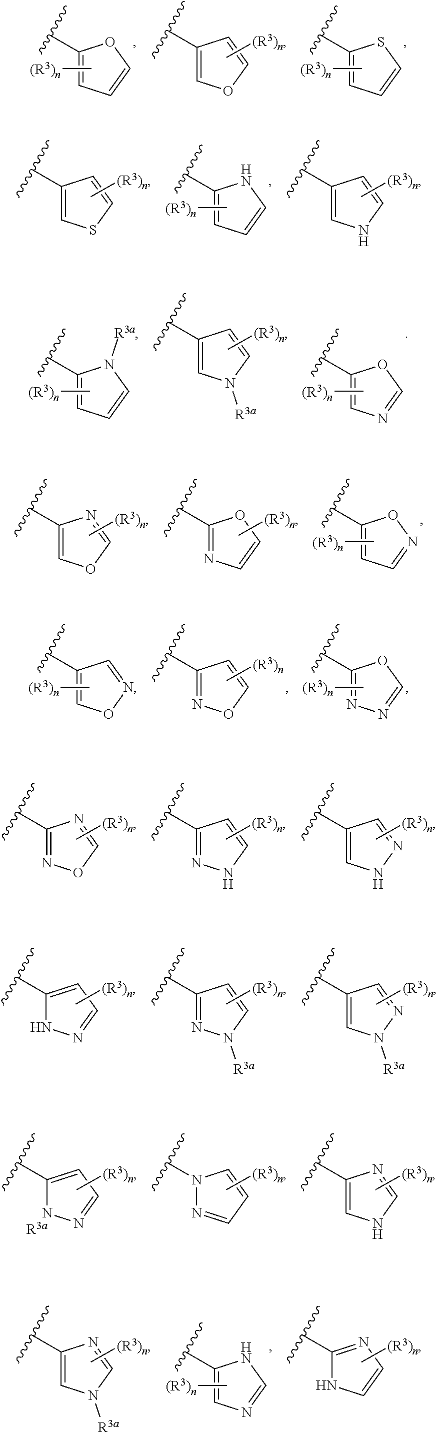

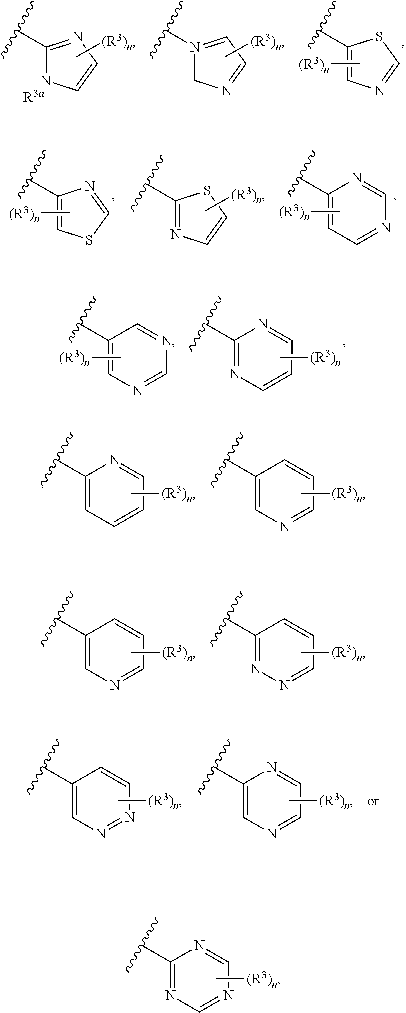

wherein R^{3a} is C₁-C₃ alkyl.

Embodiment 6: The pharmaceutical composition of any one of Embodiments 2-5, wherein:
Z is a bond, —CH₂—, —C(CH₃)H—, —O—, —S—, —NH—, —NCH₃—, or —N(COCH₃)—;
V is —CH₂—, —CH₂CH₂—, —CH₂CH₂CH₂—; and
L is halogen, —NH₂, or —CF₃.

Embodiment 7: The pharmaceutical composition of any one of Embodiments 2-6, wherein X is a bond, —CH₂—, —C(CH₃)H—, —C(CH₃)₂—, or —CH₂CH₂—.

Embodiment 8: The pharmaceutical composition of any one of Embodiments 2-7, wherein R¹ and R² are each independently halogen, —CN, —CF₃, —OH, methyl, methoxy, or —CONH₂.

Embodiment 9: The pharmaceutical composition of any one of Embodiments 2-8, wherein R³ is selected from hydrogen, F, Cl, Br, I, oxo, ═S, ═NR¹⁶, —CN, —CF₃, —OH, C₁-C₃ alkyl, C₂-C₃ alkenyl, C₂-C₃ alkynyl, —S(C₁-C₃ alkyl), —SO(C₁-C₃ alkyl), —SO₂(C₁-C₃ alkyl), —NHSO₂CH₃, —N(CH₃)SO₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), or —N(CH₃)CO(C₁-C₃ alkyl).

Embodiment 10: The pharmaceutical composition of any one of Embodiments 2-9, wherein at least one of R³ is —SO₂CH₃, —NHSO₂CH₃, —CH₂NHSO₂CH₃, —SO₂NH₂, —CONH₂, or —NHCOCH₃.

Embodiment 11: The pharmaceutical composition of Embodiment 1, wherein the androgen receptor modulator is a compound of formula (II):

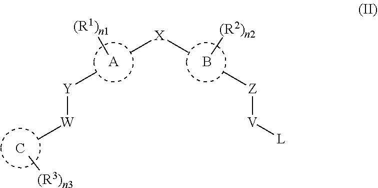

or a pharmaceutically acceptable salt, tautomer, stereoisomer or prodrug thereof, wherein:
A and B are each independently selected from phenyl, pyridyl, pyrimidyl, or thiophene;
C is a 5- to 10-membered heteroaryl or aryl;
X is a bond, —(CR⁵R⁶)ₜ—, or —NR⁷;
Y is a bond, —CH₂—, —C(CH₃)H—, —O—, —S—, —NH—, —NCH₃—, or —N(COCH₃)—;
Z is a bond, —CH₂—, —C(CH₃)H—, —O—, —S—, —NH—, —NCH₃—, or —N(COCH₃)—;
W is a bond, —CH₂—, —C(CH₃)H—, —C(═O)—, —N(R⁷)CO—, or —CONR⁷—;
V is —CH₂—, —CH₂CH₂—, —CH(CH₃)CH₂—, —CH₂CH(CH₃)—, or —CH₂CH₂CH₂—;
L is hydrogen, halogen, —OH, —NH₂, or —CF₃;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;
R³ is selected from hydrogen, halogen, oxo, ═S, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —NR¹⁴C00R¹⁶, —NR¹⁴CONR¹⁴R¹⁵, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);
R⁵ and R⁶ are each independently hydrogen, halogen, —OH, —NH₂, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;
R⁷ is H, C₁-C₆ alkyl, —CO(C₁-C₆ alkyl);
R¹³, R¹⁴ and R¹⁵ are each independently hydrogen, C₁-C₃ alkyl, C₂-C₃ alkenyl, C₂-C₃ alkynyl, or —COO(C₁-C₆ alkyl); or R¹⁴ and R¹⁵ taken together form a 3- to 6-membered heterocyclyl;
R¹⁶ is hydrogen, C₁-C₃ alkyl, C₁-C₃ haloalkyl, C₂-C₃ alkenyl, or C₂-C₃ alkynyl;
n1 and n2 are each independently 0, 1, or 2;
n3 is 0, 1, 2, 3, 4 or 5; and
t is 0, 1 or 2.

Embodiment 12: The pharmaceutical composition of Embodiment 11, wherein C is 5- to 7-membered heteroaryl comprising 1, 2, or 3 heteroatoms selected from O, S, or N as a ring member.

Embodiment 13: The pharmaceutical composition of Embodiment 11 or 12, wherein C is pyrazole, imidazole, oxazole, oxadiazole, oxazolone, isoxazole, thiazole, pyridyl, or pyrimidyl.

Embodiment 14: The pharmaceutical composition of Embodiment 11, wherein C, which is optionally substituted with R³ is selected from

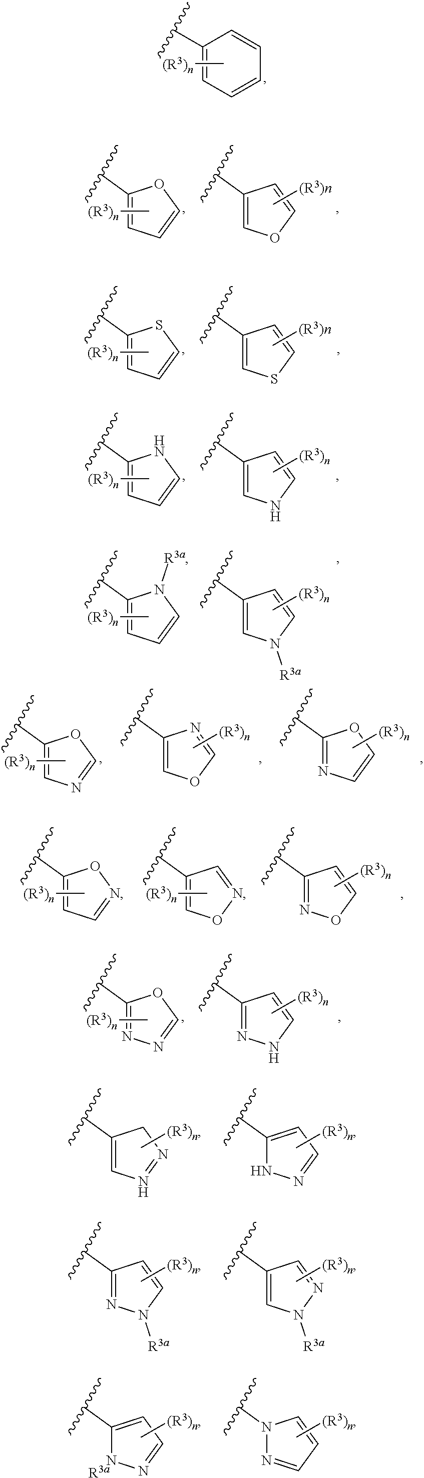

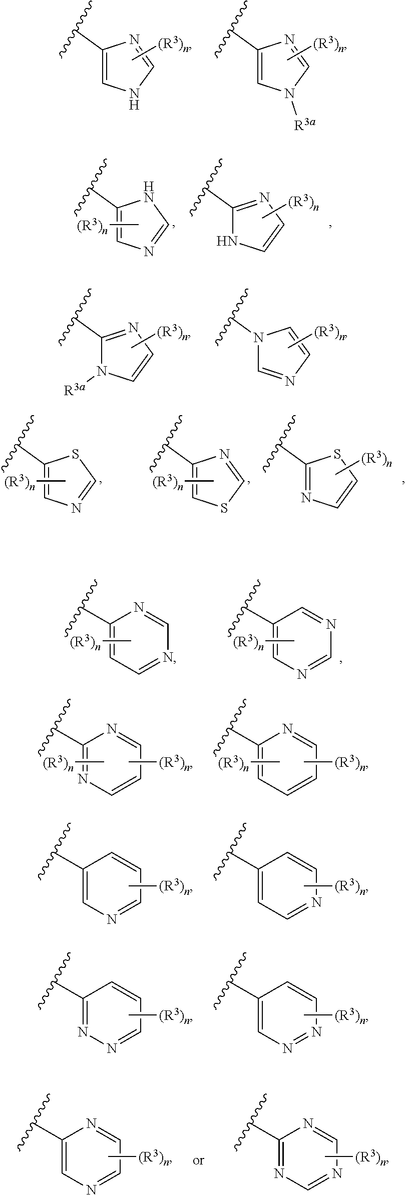

wherein R^{3a} is C₁-C₃ alkyl.

Embodiment 15: The pharmaceutical composition of any one of Embodiments 11-14, wherein A has a meta or para connectivity with X and Y.

Embodiment 16: The pharmaceutical composition of any one of Embodiments 11-15, wherein B has a meta or para connectivity with X and Z.

Embodiment 17: The pharmaceutical composition of any one of Embodiments 11-16, wherein A and B are each phenyl.

Embodiment 18: The pharmaceutical composition of any one of Embodiments 11-17, wherein —Z—V-L is —Z—CH₂CH₂Cl, —Z—CH₂CH₂CH₂Cl, —Z—CH₂CH₂NH₂, or —Z—CH₂CH₂CH₂NH₂, wherein Z is a bond, —O—, —NH—, or —N(COCH₃)—.

Embodiment 19: The pharmaceutical composition of any one of Embodiments 11-18, wherein —Y—W— is a bond, —OCH₂—, —OCH₂CH₂—, —OCH(CH₃)—, —NH—, —NHCH₂—, —NHC(═O)—, or —C(═O)NH—.

Embodiment 20: The pharmaceutical composition of any one of Embodiments 11-19, wherein X is a bond, —CH₂—, —C(CH₃)H—, —C(CH₃)₂—, or —CH₂CH₂—.

Embodiment 21: The pharmaceutical composition of Embodiment 2, wherein the androgen receptor modulator is a compound of formula (III)

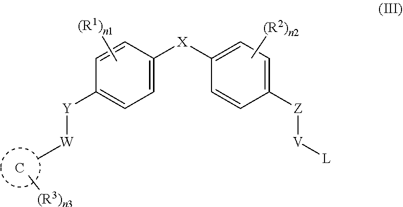

or a pharmaceutically acceptable salt, tautomer, stereoisomer or prodrug thereof, wherein:
C is a phenyl or a 5- to 7-membered monocyclic heteroaryl comprising 1, 2, or 3 heteroatoms selected from O, S, or N as a ring member;
X is a bond, —(CR⁵R⁶)ₜ—, or —NR⁷;
Y is a bond, —CH₂—, —C(CH₃)H—, —O—, —S—, —NH—, —NCH₃—, or —N(COCH₃)—;
Z is a bond, —CH₂—, —O—, or —NH—;
W is a bond, —CH₂—, —C(CH₃)H—, —C(═O)—, —N(R⁷)CO—, or —CONR⁷—;
V is —CH₂—, —CH₂CH₂—, or —CH₂CH₂CH₂—;
L is halogen, —NH₂, or —CF₃;
R¹ and R² are each independently hydrogen, halogen, —CN, —CF₃, methyl, or —CONH₂;
R³ is selected from hydrogen, F, Cl, Br, I, oxo, —CN, —CF₃, —OH, C₁-C₃ alkyl,
C₂-C₃ alkenyl, C₂-C₃ alkynyl, —S(C₁-C₃ alkyl), —SO₂(C₁-C₃ alkyl), —NHSO₂(C₁-C₃ alkyl), —
N(CH₃)SO₂(C₁-C₃ alkyl), —CH₂NHSO₂(C₁-C₃ alkyl), —CH₂N(CH₃)SO₂(C₁-C₃ alkyl), —SO₂NH₂, —CONH₂, —CON(C₁-C₃ alkyl)₂, —CONH(C₁-C₃ alkyl), —NHCO(C₁-C₃ alkyl), or —N(CH₃)CO(C₁-C₃ alkyl);
R⁵ and R⁶ are each independently hydrogen, halogen, —OH, or C₁-C₃ alkyl;
R⁷ is H or C₁-C₆ alkyl;
n1 and n2 are each independently 0, 1, or 2;
n3 is 0, 1, 2, 3, 4 or 5; and
t is 0, 1 or 2.

Embodiment 22: The pharmaceutical composition of Embodiment 21, wherein C is 5- to 7-membered monocyclic heteroaryl comprising 1, 2, or 3 heteroatoms selected from O, S, or N as a ring member.

Embodiment 23: The pharmaceutical composition of Embodiment 21 or 22, wherein —V-L is —CH₂CH₂Cl, —CH₂CH₂CH₂Cl, —CH₂CH₂NH₂, or —CH₂CH₂CH₂NH₂.

Embodiment 24: The pharmaceutical composition of any one of Embodiments 21-23, wherein —Y—W— is a bond, —OCH₂—, —OCH₂CH₂—, —OCH(CH₃)—, —NH—, —NHCH₂—, —NHC(═O)—, or —C(═O)NH—.

Embodiment 25: The pharmaceutical composition of any one of Embodiments 21-24, wherein X is a bond, —CH₂—, —C(CH₃)H—, —C(CH₃)₂—, or —CH₂CH₂—.

Embodiment 26: The pharmaceutical composition of Embodiment 2, wherein the androgen receptor modulator is a compound of formula (IV)

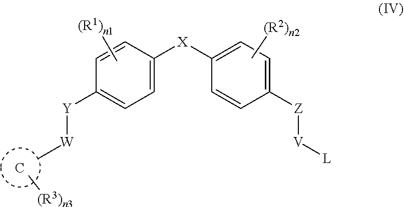

or a pharmaceutically acceptable salt, tautomer, stereoisomer or prodrug thereof, wherein:
C is

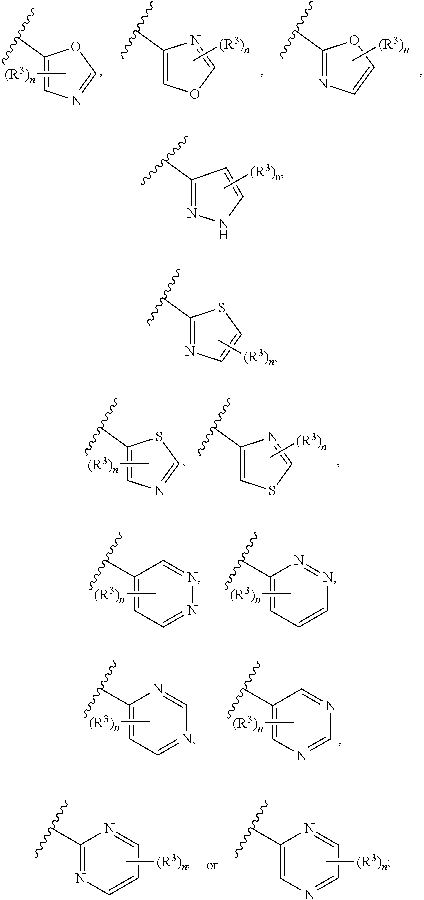

X is —(CR⁵R⁶)ₜ— or —NR⁷—;
Y is a bond, —CH₂—, —O—, or —NH—;
Z is a bond, —CH₂—, —O—, or —NH—;
W is a bond, —CH₂—, or —C(CH₃)H—;
V is —CH₂—, —CH₂CH₂—, —CH₂CH₂CH₂—, or —CH₂CHClCH₂—;
L is hydrogen, —OH, or halogen;
R¹ and R² are each independently hydrogen, halogen, —CN, —CF₃, methyl, or —CONH₂;
R³ is selected from hydrogen, F, Cl, Br, I, oxo, —CN, —CF₃, —OH, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —NHSO₂(C₁-C₃ alkyl), —NHSO₂CF₃, —N(CH₃)SO₂(C₁-C₃ alkyl), —CH₂NHSO₂(C₁-C₃ alkyl), —CH₂N(CH₃)SO₂(C₁-C₃ alkyl), —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);
R⁵ and R⁶ are each independently hydrogen or C₁-C₃ alkyl;
R⁷ is H or C₁-C₆ alkyl;
n1 and n2 are each independently 0, 1, or 2;
n3 is 0, 1, or 2; and
t is 1 or 2.

Embodiment 27: The pharmaceutical composition of Embodiment 26, wherein R³ is selected from hydrogen, F, Cl, Br, I, —CN, —CF₃, —OH, methyl, methoxy, —S(C₁-C₃ alkyl), —SO₂(C₁-C₃ alkyl), —NH₂, —NHSO₂CH₃, —NHSO₂CF₃, —N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), or —NHCO(C₁-C₃ alkyl).

Embodiment 28: The pharmaceutical composition of Embodiment 2, wherein the androgen receptor modulator is a compound of formula (V)

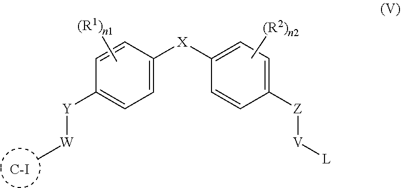

or a pharmaceutically acceptable salt, tautomer, stereoisomer or prodrug thereof, wherein:

C-I is

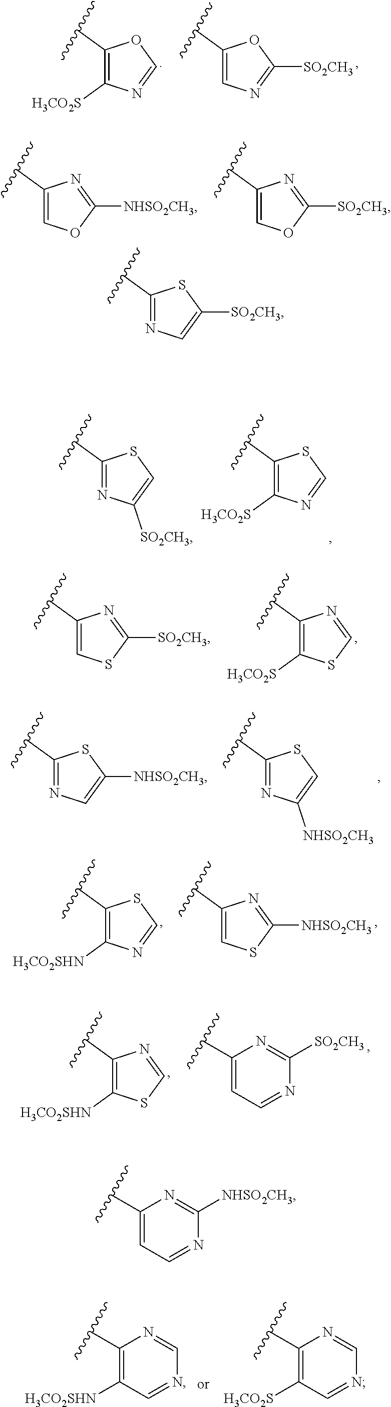

X is —(CR⁵R⁶)ₜ—
Y is —O—;
Z is —O—;
W is —CH₂— or —C(CH₃)H—;
V is —CH₂—, —CH₂CH₂— or —CH₂CH₂CH₂—;
L is halogen;
R¹ and R² are each independently Cl, or —CN;
R⁵ and R⁶ are each independently hydrogen or methyl;
R⁷ is H or C₁-C₆ alkyl;
n1 and n2 are each independently 0, 1, or 2; and
t is 1.

Embodiment 29: The pharmaceutical composition of Embodiment 1, wherein the androgen receptor modulator is selected from Table A.

Embodiment 30: The pharmaceutical composition of Embodiment 1, wherein the androgen receptor modulator is selected from Table B.

Embodiment 31: The pharmaceutical composition of Embodiment 1, wherein the androgen receptor modulators is a compound of formula (VI):

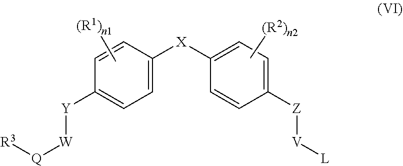

or a pharmaceutically acceptable salt, tautomer, stereoisomer or prodrug thereof, wherein:
Q is a bond, C₁-C₃ alkylene, arylene, heteroarylene, carbocyclylene, or heterocyclylene, each optionally substituted with —OH or R¹;
X is —(CR⁵R⁶)ₜ— or —NR⁷—;
Y is a bond, —CH₂—, —O—, or —NH—;
Z is a bond, —CH₂—, —O—, or —NH—;
W is a bond, or —(CR^{8a}R^{9a})ₘ—;
V is a bond, —(CR^{8a}R^{9a})ₘ—, —C(═O)—, —N(R⁷)CO—, —CONR⁷—, or —NSO₂R⁷—;
L is hydrogen, —OH, or halogen;
R¹ and R² are each independently hydrogen, halogen, —CN, —CF₃, methyl, or —CONH₂;
R³ is selected from —S(C₁-C₃ alkyl), —SO₂(C₁-C₃ alkyl), —NH₂, —NHSO₂(C₁-C₃ alkyl), —NHSO₂CF₃, —N(CH₃)SO₂(C₁-C₃ alkyl), —CH₂NHSO₂(C₁-C₃ alkyl), —CH₂N(CH₃)SO₂(C₁-C₃ alkyl), —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);
R⁵ and R⁶ are each independently hydrogen, halogen, —OH, —NH₂, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;
R⁷ is H or C₁-C₆ alkyl;
R^{8a} and R^{9a} are each independently hydrogen, —OH, halogen, optionally substituted C₁-C₆ alkyl, optionally substituted C₂-C₆ alkenyl, optionally substituted C₂-C₆ alkynyl, optionally substituted C₁-C₆ alkoxy, optionally substituted —OCO(C₁-C₆ alkyl), —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl;
R¹³, R¹⁴ and R¹⁵ are each independently hydrogen, C₁-C₃ alkyl, C₂-C₃ alkenyl, C₂-C₃ alkynyl, or —COO(C₁-C₆ alkyl); or R¹⁴ and R¹⁵ taken together form a 3- to 6-membered heterocyclyl;
R¹⁶ is hydrogen, C₁-C₃ alkyl, C₁-C₃ haloalkyl, C₂-C₃ alkenyl, or C₂-C₃ alkynyl;
n1 and n2 are each independently 0, 1, or 2;
m is 1, 2, 3, or 4; and
t is 1 or 2.

Embodiment 32: The pharmaceutical composition of Embodiment 1, wherein the androgen receptor modulators is a compound of formula (VII):

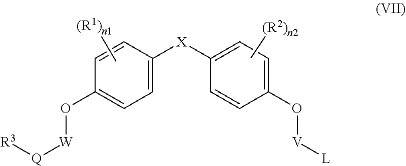

or a pharmaceutically acceptable salt, tautomer, stereoisomer or prodrug thereof, wherein:
Q is a bond, C₁-C₃ alkylene, arylene, or heteroarylene, each optionally substituted with —OH or R¹;
X is —(CR⁵R⁶)ₜ—;
W is a bond, —CH₂—, —C(CH₃)H—, —CH₂CH₂—, —CH₂CH₂CH₂—, or —CH₂CH(OH)CH₂—;
V is a bond or —(CR^{8a}R^{9a})ₘ—;
L is hydrogen, —OH, or halogen;
R¹ and R² are each independently hydrogen, halogen, —CN, —CF₃, methyl, or —CONH₂;
R³ is selected from —S(C₁-C₃ alkyl), —SO₂(C₁-C₃ alkyl), —NH₂, —NHSO₂(C₁-C₃ alkyl), —NHSO₂CF₃, —N(CH₃)SO₂(C₁-C₃ alkyl), —CH₂NHSO₂(C₁-C₃ alkyl), —CH₂N(CH₃)SO₂(C₁-C₃ alkyl), —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);
R⁵ and R⁶ are each independently H, halogen, —OH, —NH₂, or C₁-C₃ alkyl;
R⁷ is H or C₁-C₆ alkyl;
R^{8a} and R^{9a} are each independently hydrogen, —OH, halogen, or C₁-C₃ alkyl;
n1 is 0, 1, or 2;
n2 is 1 or 2; m is 1, 2, 3, or 4; and
t is 1 or 2.

Embodiment 33: The pharmaceutical composition of Embodiment 1, wherein the androgen receptor modulators is a compound of formula (VIII):

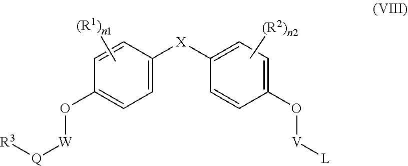

or a pharmaceutically acceptable salt, tautomer, stereoisomer or prodrug thereof, wherein:
Q is a bond, C₁-C₃ alkylene, phenylene, or 5- or 6-membered heteroarylene, each optionally substituted with —OH or R¹;
X is —(CR⁵R⁶)ₜ—;
Y is a bond, —CH₂—, —O—, or —NH—;
Z is a bond, —CH₂—, —O—, or —NH—;
W is a bond, —CH₂—, —C(CH₃)H—, —CH₂CH₂—, —CH₂CH₂CH₂—, or —CH₂CH(OH)CH₂—;
V is a bond, —CH₂—, —CH₂CH₂—, —CH(CH₃)CH₂—, —CH(OH)CH₂—, —CH₂CH(CH₃)—, —CH₂CH₂CH₂—, or —CH₂CH(OH)CH₂—;
L is halogen;
R¹ and R² are each independently hydrogen, halogen, —CN, —CF₃, methyl, or —CONH₂;
R³ is selected from —S(C₁-C₃ alkyl), —SO₂(C₁-C₃ alkyl), —NH₂, —NHSO₂(C₁-C₃ alkyl), —NHSO₂CF₃, —N(CH₃)SO₂(C₁-C₃ alkyl), —CH₂NHSO₂(C₁-C₃ alkyl), —CH₂N(CH₃)SO₂(C₁-C₃ alkyl), —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);
R⁵ and R⁶ are each independently H, halogen, —OH, —NH₂, or C₁-C₃ alkyl;
R⁷ is H or C₁-C₆ alkyl;
R^{8a} and R^{9a} are each independently hydrogen, —OH, halogen, or C₁-C₃ alkyl;
n1 is 0, 1, or 2;
n2 is 1 or 2;
m is 1, 2, 3, or 4; and
t is 1 or 2.

Embodiment 34: The pharmaceutical composition of Embodiment 1, wherein the androgen receptor modulators is a compound of formula (IX):

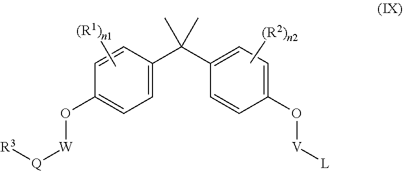

or a pharmaceutically acceptable salt, tautomer, stereoisomer or prodrug thereof, wherein:
Q is a —CH₂—, —CH₂CH₂—, —CH₂CH₂CH₂—,

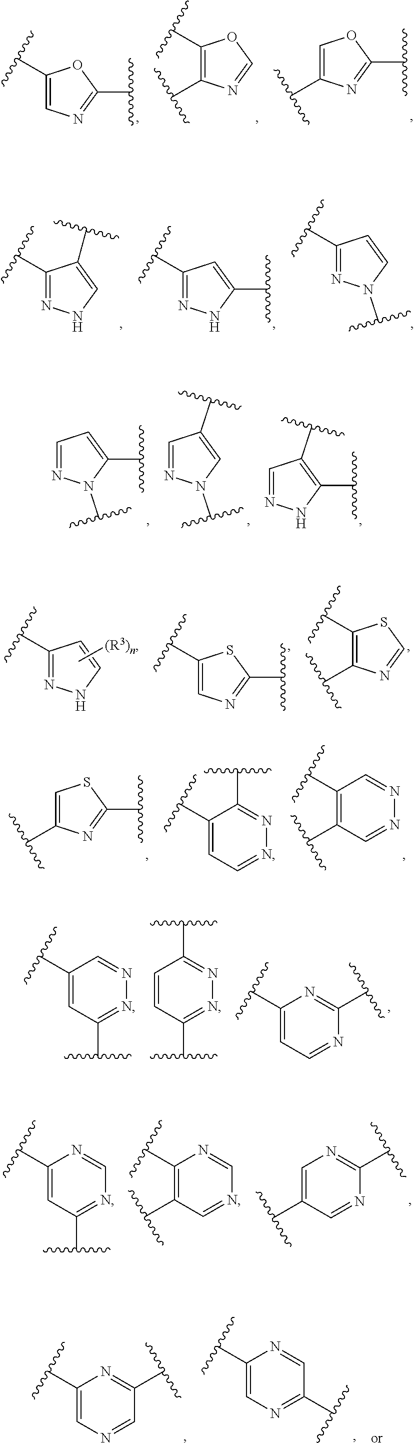

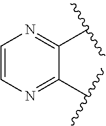 [SMILES: *c1nccnc1*]

each optionally substituted with —OH or R¹;
W is a bond, —CH₂—, —C(CH₃)H—, —CH₂CH₂—, —CH₂CH₂CH₂—, or —CH₂CH(OH)CH₂—;
V is a bond, —CH₂CH₂—, —CH(CH₃)CH₂—, —CH(OH)CH₂—, —CH₂CH(CH₃)—, —CH₂CH₂CH₂—, or —CH₂CH(OH)CH₂—;
L is Cl;
R¹ and R² are each independently hydrogen, halogen, —CN, —CF₃, methyl, or —CONH₂;
R³ is selected from —S(C₁-C₃ alkyl), —SO₂(C₁-C₃ alkyl), —NH₂, —NHSO₂(C₁-C₃ alkyl), —NHSO₂CF₃, —N(CH₃)SO₂(C₁-C₃ alkyl), —CH₂NHSO₂(C₁-C₃ alkyl), —CH₂N(CH₃)SO₂(C₁-C₃ alkyl), —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);
n1 is 0, 1, or 2; and
n2 is 1 or 2.

Embodiment 35: The pharmaceutical composition of Embodiment 1, wherein the androgen receptor modulators is a compound of formula (X):

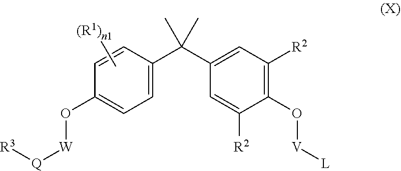

or a pharmaceutically acceptable salt, tautomer, stereoisomer or prodrug thereof, wherein:
Q is a —CH₂—, —CH₂CH₂—, —CH₂CH(OH)CH₂—,

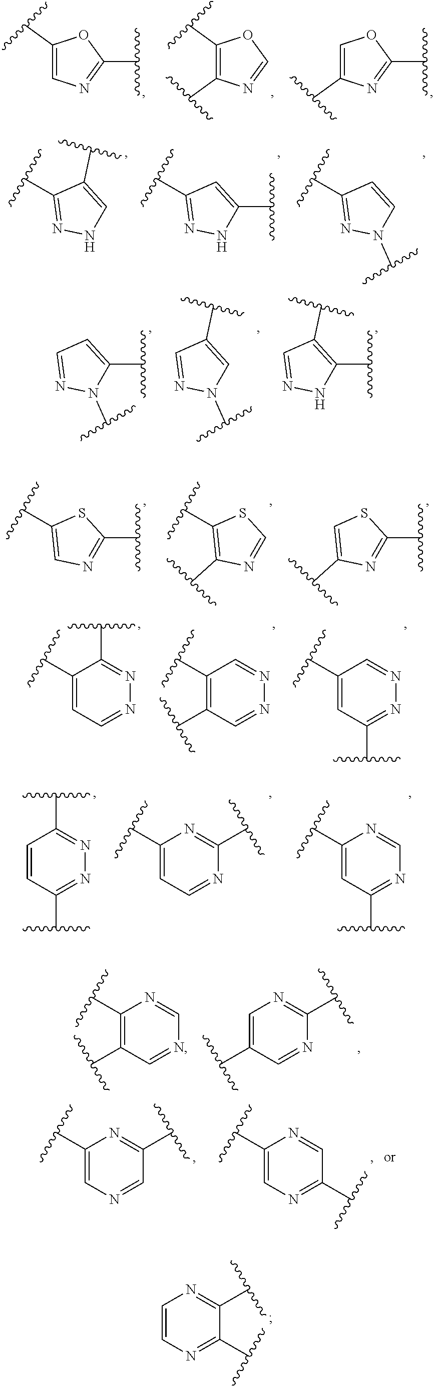

W is a bond, —CH₂—, —C(CH₃)H—, —CH₂CH₂—, —CH₂CH₂CH₂—, or —CH₂CH(OH)CH₂—;
V is a bond, —CH₂CH₂—, —CH(CH₃)CH₂—, —CH(OH)CH₂—, —CH₂CH(CH₃)—, —CH₂CH₂CH₂—, or —CH₂CH(OH)CH₂—;
L is Cl;
R¹ and R² are each independently hydrogen, halogen, —CN, or methyl;
R³ is selected from —SO₂(C₁-C₃ alkyl), —NHSO₂(C₁-C₃ alkyl), —NHSO₂CF₃, —N(CH₃)SO₂(C₁-C₃ alkyl), —CH₂NHSO₂(C₁-C₃ alkyl), —CH₂N(CH₃)SO₂(C₁-C₃ alkyl), or —SO₂NH₂; and
n1 is 0, 1, or 2.

Embodiment 36: The pharmaceutical compositions of any one of Embodiments 31-35, wherein R³ is —SO₂CH₃, —NHSO₂CH₃, —N(CH₃)SO₂CH₃, —CH₂NHSO₂CH₃, —CH₂N(CH₃)SO₂CH₃, or —SO₂NH₂.

Embodiment 37: The pharmaceutical compositions of any one of Embodiments 31-35, wherein R³ is —SO₂CH₃ or —NHSO₂CH₃.

Embodiment 38: The pharmaceutical compositions of any one of Embodiments 31-37, wherein Q is —CH₂—, —CH₂CH₂—, or —CH₂CH₂CH₂—, each optionally substituted with —OH, halogen, or C₁-C₃ alkyl.

Embodiment 39: The pharmaceutical compositions of any one of Embodiments 31-37, wherein Q is —CH₂CH₂CH₂—, optionally substituted with —OH, halogen, or C₁-C₃ alkyl.

Embodiment 40: The pharmaceutical compositions of any one of Embodiments 31-37, wherein Q is —CH₂CHOHCH₂—.

Embodiment 41: The pharmaceutical compositions of any one of Embodiments 31-37, wherein Q is

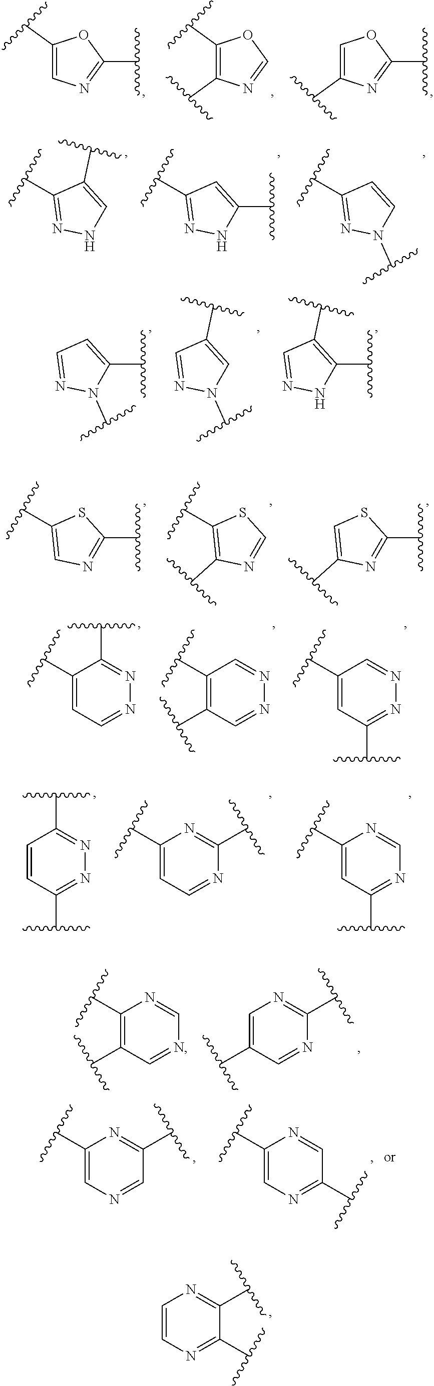

each optionally substituted with —OH, halogen, or C₁-C₃ alkyl.

Embodiment 42: The pharmaceutical compositions of any one of Embodiments 31-36, wherein Q is —CH₂CHOHCH₂—,

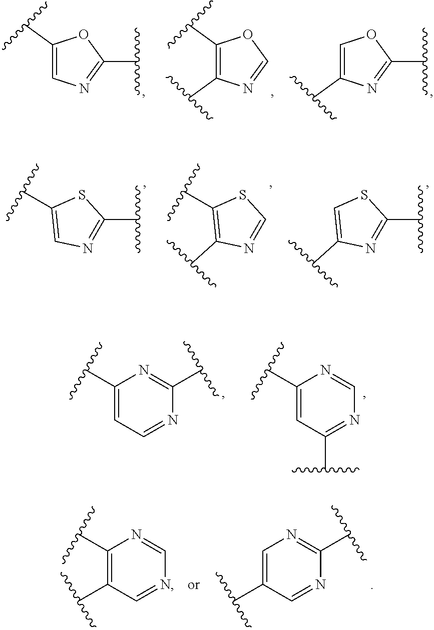

Embodiment 43: The pharmaceutical composition of Embodiment 35, wherein
Q is —CH₂CHOHCH₂—,

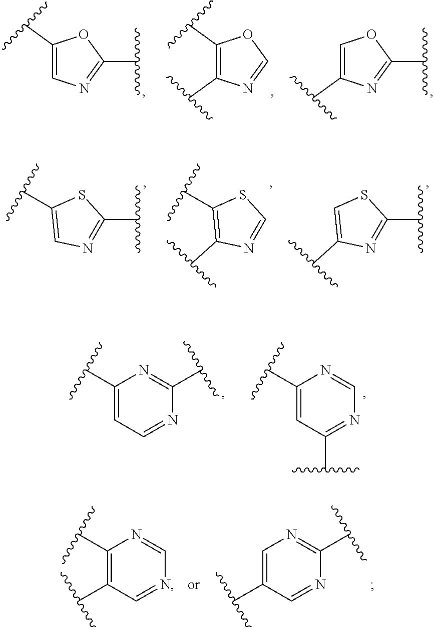

W is a bond or —CH₂—;
R¹ is hydrogen;
R² are each independently —CN or C1;
R³ is selected from —SO₂(C₁-C₃ alkyl), or —NHSO₂(C₁-C₃ alkyl);
L is Cl; and
V is CH₂CH₂—, or CH₂CH(OH)CH₂—.

Embodiment 44: The pharmaceutical composition of any one of Embodiments 1-43, wherein the second therapeutically active agent is selected from a poly (ADP-ribose) polymerase (PARP) inhibitor, an androgen receptor ligand-binding domain inhibitor, an inhibitor of CYP17, a microtubule inhibitor, a modulator of PD-1 or PD-L1, a gonadotropin releasing hormone agonist, a 5-alpha reductase inhibitor, a vascular endothelial growth factor inhibitor, a histone deacetylase inhibitor, an integrin alpha-v-beta-3 inhibitor, a receptor tyrosine kinase, a phosphoinositide 3-kinase inhibitor, an anaplastic lymphoma kinase (ALK) inhibitor, an endothelin receptor A antagonist, an anti-CTLA4 inhibitor, an heat shock protein 27 (HSP27) inhibitor, an androgen receptor degrader, a androgen receptor DNA-binding domain inhibitor, a bromodomain and extra-terminal motif (BET) inhibitor, an androgen receptor N-terminal domain inhibitor, an alpha-particle emitting radioactive therapeutic agent, niclosamide, a selective estrogen receptor modulator (SERM), a selective estrogen receptor degrader (SERD), an aromatase inhibitor, selective progesterone receptor modulator (SPRM), a glucocorticoid receptor inhibitor, a HER2 receptor antagonist, a mammalian target of rapamycin (mTOR) inhibitor, an AKT inhibitor, a B-cell lymphoma-2 (Bcl-2) inhibitor, an aurora kinase inhibitor, a Wnt-targeting antagonist, a CYP11a inhibitor, a selective androgen receptor modulator, or enhancer of zeste homolog 2 (EZH2) inhibitor.

Embodiment 45: The pharmaceutical composition of any one of Embodiments 1-44, wherein the androgen receptor ligand-binding domain inhibitor is enzalutamide, apalutamide, darolutamide, bicalutamide, nilutamide, flutamide, ODM-204, or TAS3681.

Embodiment 46: The pharmaceutical composition of any one of Embodiments 1-44, wherein the androgen receptor ligand-binding domain inhibitor is enzalutamide.

Embodiment 47: The pharmaceutical composition of any one of Embodiments 1-46, further comprising a pharmaceutically acceptable carrier.

Embodiment 48: A method for modulating androgen receptor activity, comprising administering a pharmaceutical composition of any one of Embodiments 1-47, to a subject in need thereof.

Embodiment 49: The method of Embodiment 48, wherein the modulating androgen receptor activity is for treating a condition or disease selected from prostate cancer, breast cancer, ovarian cancer, bladder cancer, pancreatic cancer, hepatocellular cancer, endometrial cancer, salivary gland carcinoma, hair loss, acne, hirsutism, ovarian cysts, polycystic ovary disease, precocious puberty, spinal and bulbar muscular atrophy, or age-related macular degeneration.

Embodiment 51: A method for treating cancer, comprising administering the pharmaceutical composition of any one of Embodiments 1-47, to a subject in need thereof.

Embodiment 52: The method of Embodiment 51, wherein the cancer is breast cancer.

Embodiment 53: The method of Embodiment 52, wherein the breast cancer is triple negative breast cancer.

Embodiment 54: The method of Embodiment 51, wherein the cancer is selected from prostate cancer, breast cancer, ovarian cancer, bladder cancer, pancreatic cancer, hepatocellular cancer, endometrial cancer, or salivary gland carcinoma.

Embodiment 55: The method of Embodiment 51, wherein the cancer is prostate cancer.

Embodiment 56: The method of Embodiment 55, wherein the prostate cancer is primary or localized prostate cancer, locally advanced prostate cancer, recurrent prostate cancer, advanced prostate cancer, metastatic prostate cancer, metastatic castration-resistant prostate cancer, and hormone-sensitive prostate cancer.

Embodiment 57: The method of Embodiment 55, wherein the prostate cancer is metastatic castration-resistant prostate cancer.

Embodiment 58: The method of Embodiment 55, wherein the prostate cancer expresses full-length androgen receptor or truncated androgen receptor splice variant.

Embodiment 59: The method of any one of Embodiments 49-58, wherein the prostate cancer is resistant to enzalutamide monotherapy.

The publications discussed herein are provided solely for their disclosure prior to the filing date of the present application. Nothing herein is to be construed as an admission that the present invention is not entitled to antedate such publication by virtue of prior invention.

While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed is:

1. A pharmaceutical composition comprising an androgen receptor modulator and a second therapeutically active agent.

2. The pharmaceutical composition of claim 1, wherein the androgen receptor modulator is a compound of formula (IIIA): 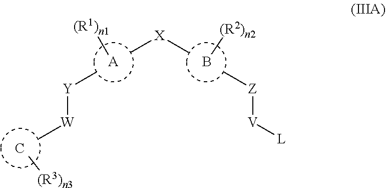
or a pharmaceutically acceptable salt, tautomer, stereoisomer or prodrug thereof, wherein:
A and B are each independently selected from phenyl, pyridyl, pyrimidyl, or thiophene;
C is a 3- to 10-membered ring;
X is a bond, —(CR⁵R⁶)ₜ—, or —NR⁷;
Y is a bond, —(CR⁸R⁹)ₘ—, —O—, —S—, —S(═O)—, —SO₂—, —NR⁷—, or —N(COCH₃)—;
W is a bond, —(CR^{8a}R^{9a})ₘ—, —C(═O)—, —N(R⁷)CO—, —CONR⁷—, or —NSO₂R⁷—;
Z is a bond, —CH₂—, —C(CH₃)H—, —O—, —S—, —NH—, —NCH₃—, or —N(COCH₃)—;
V is —CH₂— and L is halogen, —NH₂, —CHCl₂, —CCl₃, or —CF₃; or
V is —CH₂CH₂— and L is halogen or —NH₂;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;
R³ is selected from halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);
R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;
R⁷ is hydrogen, C₁-C₆ alkyl, C₂-C₆ alkenyl, or C₂-C₆ alkynyl;
R⁸ and R⁹ are each independently hydrogen, halogen, or C₁-C₃ alkyl;
R^{8a} and R^{9a} are each independently hydrogen, —OH, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵; or R^{8a} and R^{8b} taken together form an optionally substituted 3- to 6-membered carbocyclyl or heterocyclyl;
R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form a 3- to 6-membered heterocyclyl;
R¹⁶ is hydrogen, optionally substituted C₁-C₃ alkyl, optionally substituted C₂-C₃ alkenyl, optionally substituted C₂-C₃ alkynyl, C₃-C₆ cycloalky, or phenyl;
each m is independently 0, 1, or 2;
n1 and n2 are each independently 0, 1, or 2;
n3 is 1, 2, 3, 4 or 5; and
t is 0, 1 or 2.

3. The pharmaceutical composition of claim 2, wherein the androgen receptor modulator is a compound of formula (IVA): 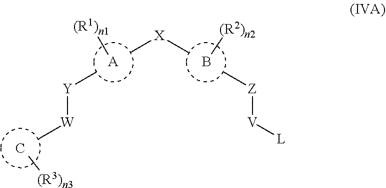
or a pharmaceutically acceptable salt, tautomer, stereoisomer or prodrug thereof, wherein:
A and B are each independently selected from phenyl, pyridyl, pyrimidyl, or thiophene;
C is a 3- to 10-membered ring;
X is a bond, —(CR⁵R⁶)ₜ—, or —NR⁷;
Y and Z are each independently a bond, —CH₂—, —C(CH₃)H—, —O—, —S—, —NH—, —NCH₃—, or —N(COCH₃)—;
W is a bond, —CH₂—, —C(CH₃)H—, —C(═O)—, —N(R⁷)CO—, or —CONR⁷—;
V is —CH₂— and L is halogen, —NH₂, or —CF₃; or
V is —CH₂CH₂— and L is halogen or —NH₂;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, optionally substituted —(C₁-C₆ alkyl)-SO₂R¹⁶;
R³ is selected from halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);
R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;
R⁷ is H or C₁-C₆ alkyl;
R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form a 3- to 6-membered heterocyclyl;
R¹⁶ is hydrogen, C₁-C₃ alkyl, C₂-C₃ alkenyl, or C₂-C₃ alkynyl;
n1 and n2 are each independently 0, 1, or 2;
n3 is 1, 2, 3, 4 or 5; and
t is 0, 1 or 2.

4. The pharmaceutical composition of claim 3, wherein C is 5- to 10-membered heteroaryl or aryl.

5. The pharmaceutical composition of claim 4, wherein C is 5- to 7-membered heteroaryl comprising 1, 2, or 3 heteroatoms selected from O, S, or N as a ring member.

6. The pharmaceutical composition of claim 5, wherein C, which is substituted with (R³)n3, is pyrazole, imidazole, oxazole, oxadiazole, oxazolone, isoxazole, thiazole, pyridyl, pyrazine, furan or pyrimidyl.

7. The pharmaceutical composition of claim 6, wherein C, which is substituted with (R³)n3, is selected from 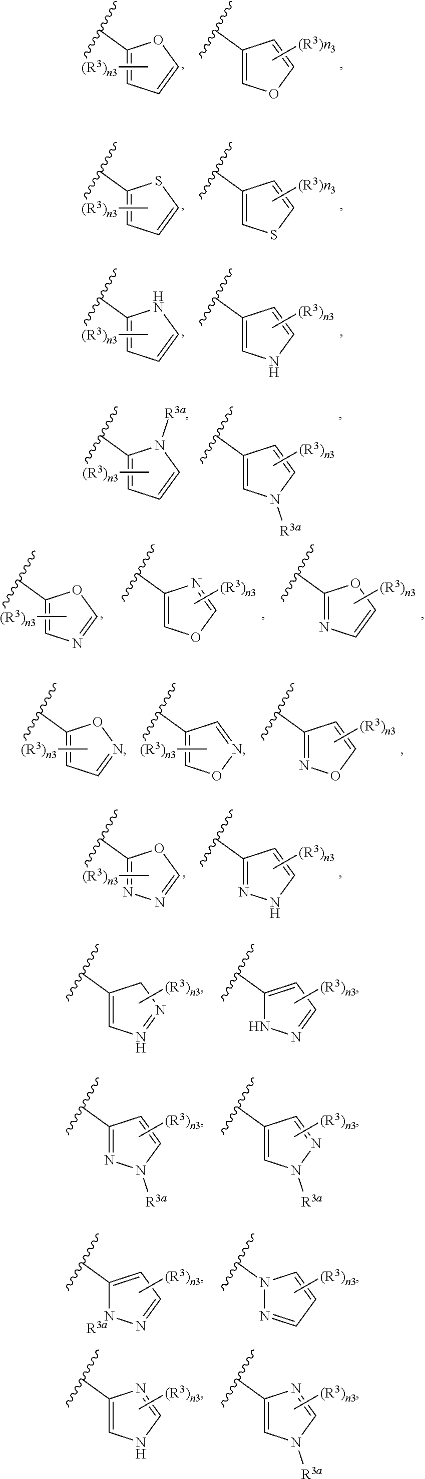 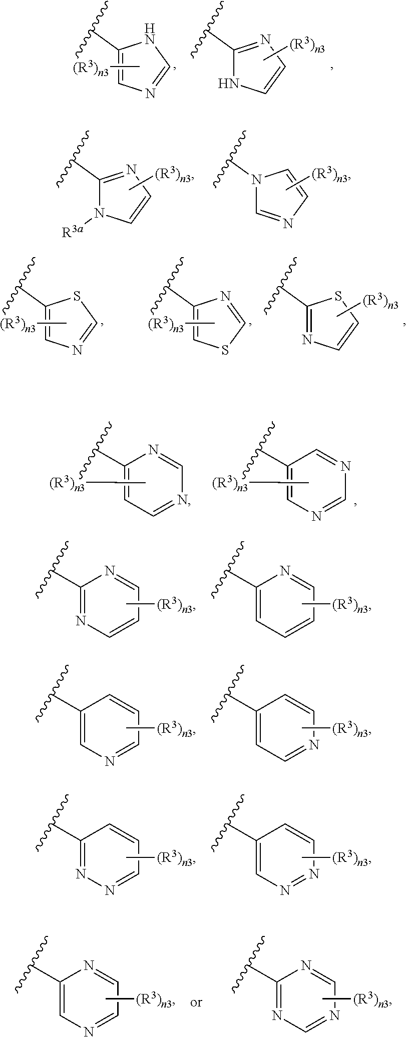
wherein R^{3a} is C₁-C₃ alkyl.

8. The pharmaceutical composition of claim 7, wherein R¹ and R² are each independently Cl, —CN, —CF₃, —OH, methyl, methoxy, or —CONH₂.

9. The pharmaceutical composition of claim 3, wherein:
A and B are phenyl;
X is —(CR⁵R⁶)ₜ—;
Y and Z are each —O—;
V is —CH₂— or —CH₂CH₂—;
L is halogen;
R¹ and R² are each independently hydrogen, halogen, —CN, —CF₃, —OH, or optionally substituted C₁-C₆ alkyl;
R⁵ and R⁶ are each independently hydrogen, halogen, —OH, or C₁-C₃ alkyl; and
R¹⁶ is hydrogen, C₁-C₃ alkyl, C₂-C₃ alkenyl, or C₂-C₃ alkynyl.

10. The pharmaceutical composition of claim 7, wherein:
R⁵ and R⁶ are each independently hydrogen, or C₁-C₃ alkyl;
W is —CH₂— or —C(CH₃)H—;
V is —CH₂CH₂—; and
R¹ and R² are each independently hydrogen, halogen, or —CN.

11. The pharmaceutical composition of claim 2, wherein the androgen receptor modulator is a compound of formula (A-I): 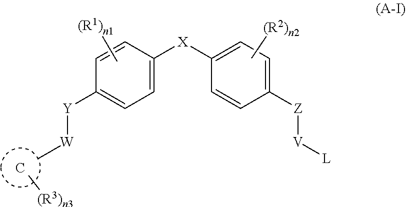
or a pharmaceutically acceptable salt, tautomer, stereoisomer or prodrug thereof, wherein:
C is a 5- to 7-membered monocyclic heteroaryl comprising 1, 2, or 3 heteroatoms selected from O, S, or N as a ring member;
X is a bond, —(CR⁵R⁶)ₜ—, or —NR⁷;
Y is a bond, —CH₂—, —C(CH₃)H—, —O—, —S—, —NH—, —NCH₃—, or —N(COCH₃)—;
Z is a bond, —CH₂—, —O—, or —NH—;
W is a bond, —CH₂—, —C(CH₃)H—, —C(═O)—, —N(R⁷)CO—, or —CONR⁷—;
V is —CH₂— and L is halogen, —NH₂, or —CF₃; or
V is —CH₂CH₂— and L is halogen or —NH₂;
R¹ and R² are each independently hydrogen, halogen, —CN, —CF₃, methyl, or —CONH₂;
R³ is selected from —CN, C₁-C₃ alkoxy, —CF₃, C₁-C₃ alkyl, C₂-C₃ alkenyl, C₂-C₃ alkynyl, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);
R⁵ and R⁶ are each independently hydrogen, halogen, —OH, or C₁-C₃ alkyl;
R⁷ is H or C₁-C₆ alkyl;
n1 and n2 are each independently 0, 1, or 2;
n3 is 1, 2, 3, 4 or 5; and
t is 0, 1 or 2.

12. The pharmaceutical composition of claim 11, wherein:
at least one R³ is selected from —CN, C₁-C₃ alkoxy, —CONH₂, —NHSO₂CH₃, —N(CH₃)SO₂CH₃, —NHSO₂CH₂CH₃, —N(CH₃)SO₂CH₂CH₃, or —SO₂CH₃ and the other R³, if present, is selected from —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl).

13. The pharmaceutical composition of claim 11, wherein:
X is a bond or —(CR⁵R⁶)ₜ;
W is a bond, —CH₂—, or —C(CH₃)H—;
Y is —O—;
Z is —O—;
V is —CH₂— or —CH₂CH₂—; and
L is halogen.

14. The pharmaceutical composition of claim 2, wherein the androgen receptor modulator is a compound of formula (G-II): 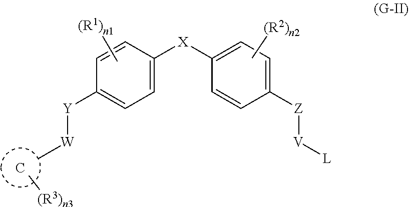
or a pharmaceutically acceptable salt, tautomer, stereoisomer or prodrug thereof, wherein:
C is 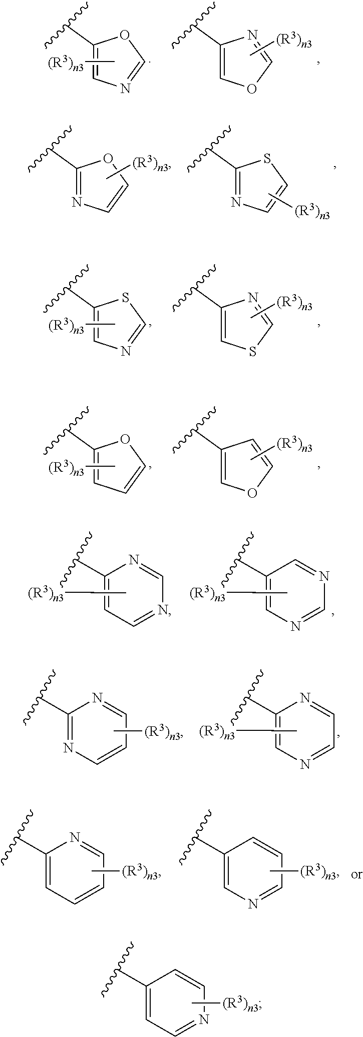
X is —(CR⁵R⁶)ₜ—;
Y is —O—;
Z is —O—;
W is —CH₂— or —C(CH₃)H—;
V is —CH₂CH₂—;
L is halogen;
R¹ and R² are each independently Cl or —CN;
at least one R³ is selected from —CN, C₁-C₃ alkoxy, —CONH₂, —NHSO₂CH₃, —N(CH₃)SO₂CH₃, —NHSO₂CH₂CH₃, —N(CH₃)SO₂CH₂CH₃, or —SO₂CH₃ and the other R³, if present, is selected from —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);
R⁵ and R⁶ are each independently hydrogen or methyl;
n1 and n2 are each independently 0, 1, or 2;
n3 is 1 or 2; and
t is 1.

15. The pharmaceutical composition of claim 14, wherein:
at least one R³ is selected from —NHSO₂CH₃, —NHSO₂CH₂CH₃, or —SO₂CH₃ and the other R³, if present, is selected from —CN, C₁-C₃ alkyl, C₁-C₃ alkoxy, —SO₂(C₁-C₃ alkyl), —NH₂, —(C₁-C₃ alkyl)NH₂, —NHSO₂CH₃, —N(CH₃)SO₂CH₃, —NHSO₂CH₂CH₃, —N(CH₃)SO₂CH₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl).

16. The pharmaceutical composition of claim 1, wherein the androgen receptor modulator is selected from Table A, or a pharmaceutically acceptable salt thereof.

17. The pharmaceutical composition of claim 1, wherein the androgen receptor modulator is selected from Table B, or a pharmaceutically acceptable salt thereof.

18. The pharmaceutical composition of claim 16, wherein the compound is selected from 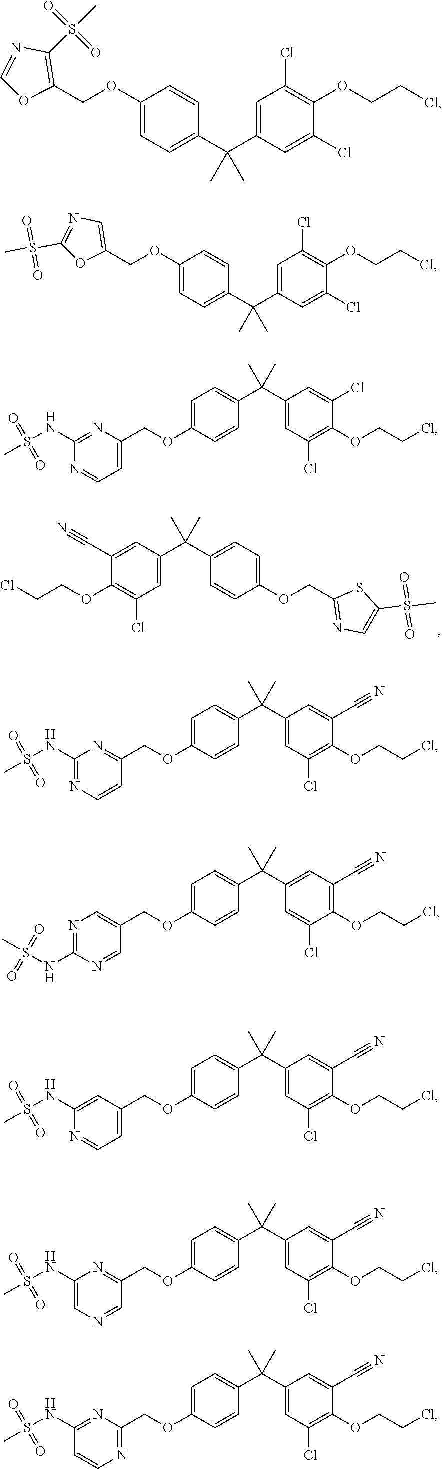 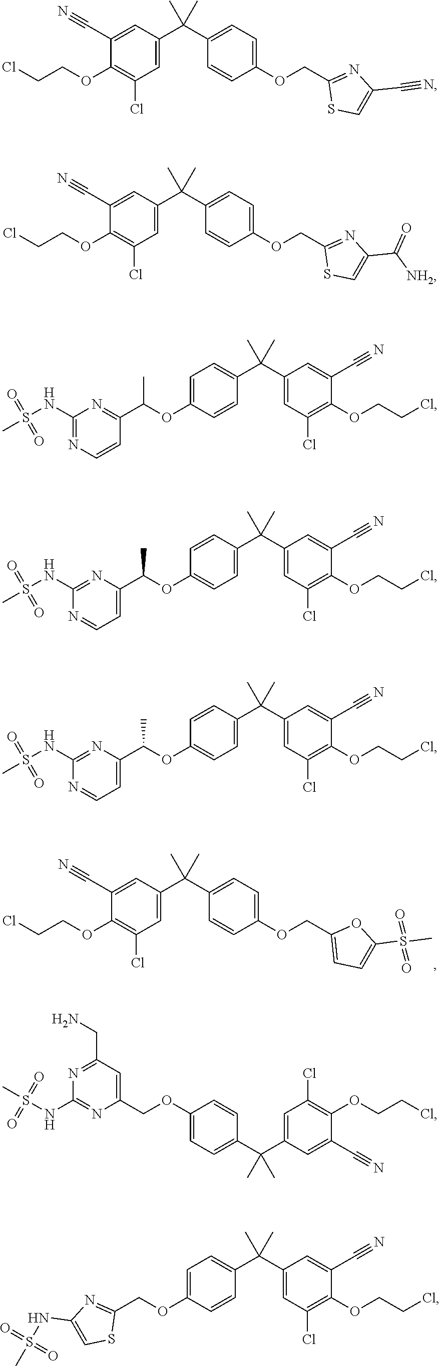 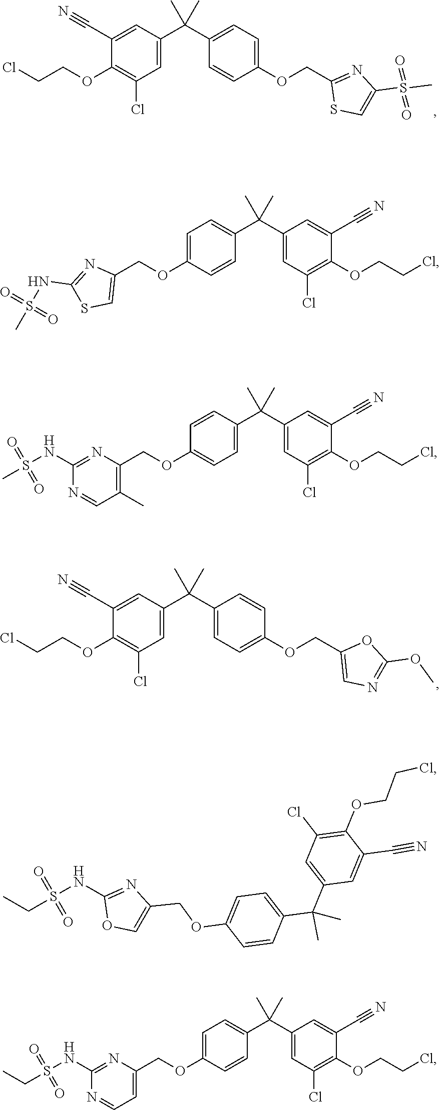
or a pharmaceutically acceptable salt, tautomer, stereoisomer or prodrug thereof.

19. The pharmaceutical composition of any one of claims 1-18, wherein the second therapeutically active agent is selected from a poly (ADP-ribose) polymerase (PARP) inhibitor, an androgen receptor ligand-binding domain inhibitor, an inhibitor of CYP17, a microtubule inhibitor, a modulator of PD-1 or PD-L1, a gonadotropin releasing hormone agonist, a 5-alpha reductase inhibitor, a vascular endothelial growth factor inhibitor, a histone deacetylase inhibitor, an integrin alpha-v-beta-3 inhibitor, a receptor tyrosine kinase, a phosphoinositide 3-kinase inhibitor, an anaplastic lymphoma kinase (ALK) inhibitor, an endothelin receptor A antagonist, an anti-CTLA4 inhibitor, an heat shock protein 27 (HSP27) inhibitor, an androgen receptor degrader, a androgen receptor DNA-binding domain inhibitor, a bromodomain and extra-terminal motif (BET) inhibitor, an androgen receptor N-terminal domain inhibitor, an alpha-particle emitting radioactive therapeutic agent, niclosamide, a selective estrogen receptor modulator (SERM), a selective estrogen receptor degrader (SERD), an aromatase inhibitor, selective progesterone receptor modulator (SPRM), a glucocorticoid receptor inhibitor, a HER2 receptor antagonist, a mammalian target of rapamycin (mTOR) inhibitor, an AKT inhibitor, a B-cell lymphoma-2 (Bcl-2) inhibitor, an aurora kinase inhibitor, a Wnt-targeting antagonist, a CYP11a inhibitor, a selective androgen receptor modulator, or enhancer of zeste homolog 2 (EZH2) inhibitor.

20. The pharmaceutical composition of any one of claims 1-18, wherein the androgen receptor ligand-binding domain inhibitor is enzalutamide, apalutamide, darolutamide, bicalutamide, nilutamide, flutamide, ODM-204, or TAS3681.

21. The pharmaceutical composition of any one of claims 1-18, wherein the androgen receptor ligand-binding domain inhibitor is enzalutamide.

22. The pharmaceutical composition of any one of claims 1-18, wherein the Bcl-2 inhibitor is venetoclax.

23. A pharmaceutical composition comprising an androgen receptor ligand-binding domain inhibitor and a compound is selected from 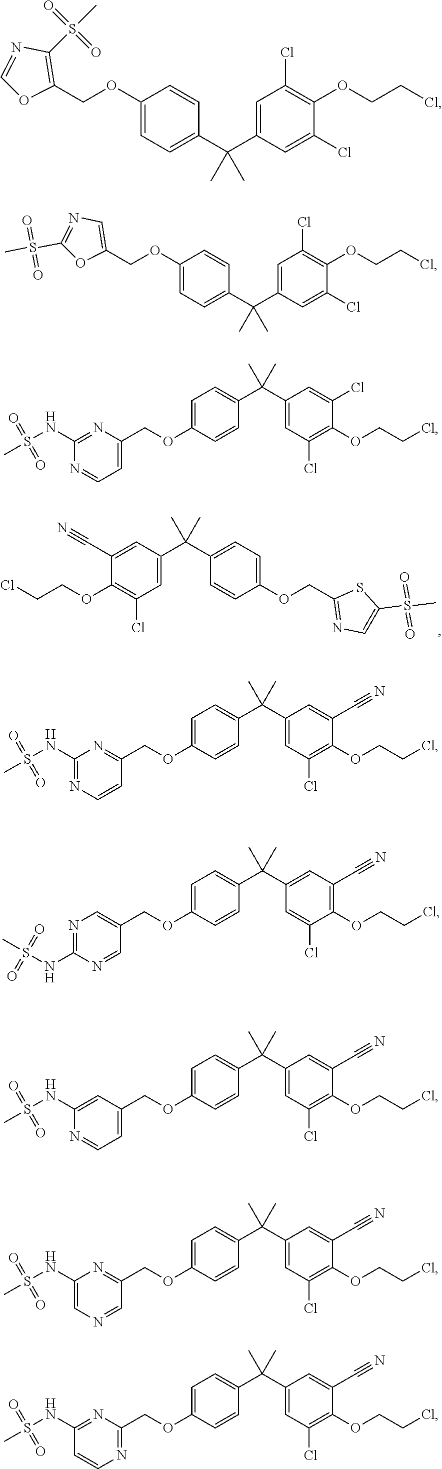 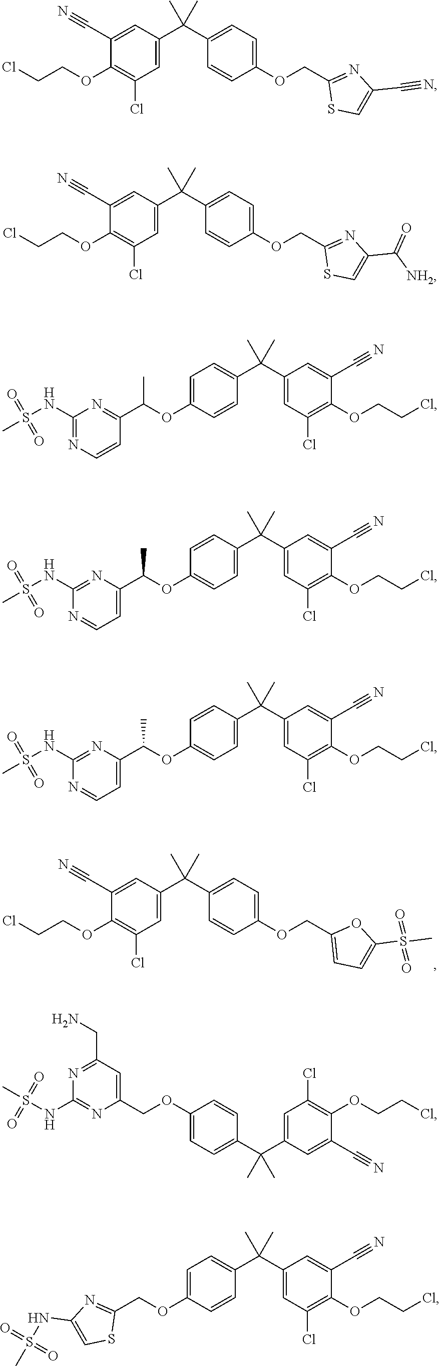 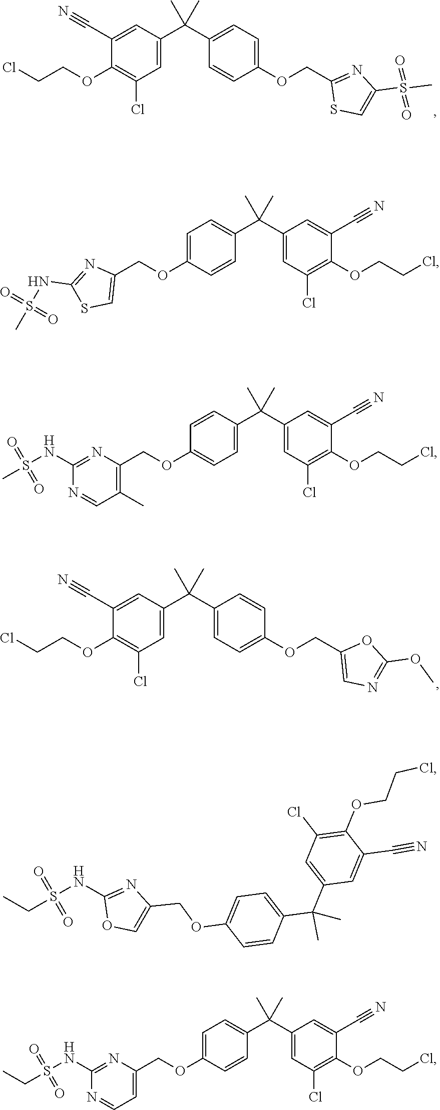
or a pharmaceutically acceptable salt, tautomer, stereoisomer or prodrug thereof.

24. The pharmaceutical composition of of claim 23, wherein the androgen receptor ligand-binding domain inhibitor is enzalutamide.

25. The pharmaceutical composition of any one of claims 1-24, further comprising a pharmaceutically acceptable carrier.

26. A method for modulating androgen receptor activity, comprising administering a pharmaceutical composition of any one of claims 1-25, to a subject in need thereof.

27. The method of claim 26, wherein the modulating androgen receptor activity is for treating a condition or disease selected from prostate cancer, breast cancer, ovarian cancer, bladder cancer, pancreatic cancer, hepatocellular cancer, endometrial cancer, salivary gland carcinoma, hair loss, acne, hirsutism, ovarian cysts, polycystic ovary disease, precocious puberty, spinal and bulbar muscular atrophy, or age-related macular degeneration.

28. A method for treating cancer, comprising administering the pharmaceutical composition of any one of claims 1-25, to a subject in need thereof.

29. The method of claim 28, wherein the cancer is breast cancer.

30. The method of claim 29, wherein the breast cancer is triple negative breast cancer.

31. The method of claim 28, wherein the cancer is selected from prostate cancer, breast cancer, ovarian cancer, bladder cancer, pancreatic cancer, hepatocellular cancer, endometrial cancer, or salivary gland carcinoma.

32. The method of claim 28, wherein the cancer is prostate cancer.

33. The method of claim 32, wherein the prostate cancer is primary or localized prostate cancer, locally advanced prostate cancer, recurrent prostate cancer, advanced prostate cancer, metastatic prostate cancer, metastatic castration-resistant prostate cancer, and hormone-sensitive prostate cancer.

34. The method of claim 32, wherein the prostate cancer is metastatic castration-resistant prostate cancer.

35. The method of claim 32, wherein the prostate cancer expresses full-length androgen receptor or truncated androgen receptor splice variant.

36. The method of claim 32, wherein the prostate cancer is resistant to enzalutamide monotherapy.
